(19) United States
(12) Patent Application Publication
(10) Pub. No.: US 2020/0397798 A1
(43) Pub. Date: Dec. 24, 2020

(54) COMBINATION THERAPIES WITH FARNESOID X RECEPTOR (FXR) MODULATORS

(71) Applicant: Akarna Therapeutics, Ltd., Cambridge (GB)

(72) Inventors: Raju MOHAN, Encinitas, CA (US); Benjamin Anthony PRATT, Encinitas, CA (US)

(73) Assignee: Akarna Therapeutics, Ltd., Cambridge (GB)

(21) Appl. No.: 16/303,741

(22) PCT Filed: May 25, 2017
(86) PCT No.: PCT/US2017/034564

§ 371 Date: Jan. 31, 2020

Related U.S. Application Data
(60) Provisional application No. 62/341,482, filed on May 25, 2016.
(60) Provisional application No. 62/341,487, filed on May 25, 2016.

(51) Int. Cl. A61K 31/55; A61K 45/06; A61P 3/06

(52) U.S. Cl. CPC A61K 31/55; A61P 3/06; A61K 45/06

(57) ABSTRACT

Described herein are methods of treating a metabolic disorder in an individual in need thereof, comprising co-administering to the individual a therapeutically effective amount of an FXR modulator, and at least one second agent that is an CCR2/CCR5 antagonist, ASK1 inhibitor, DPP-IV inhibitor, caspase protease inhibitor, SGLT2 inhibitor, acetyl-CoA carboxylase (ACC) inhibitor, diacylglycerol acyltransferase-1 inhibitor, sodium-bile acid cotransporter-inhibitor, TLR-4 antagonist, PPAR alpha/delta agonist, or GLP-1 agonist, or a combination thereof.

### CROSS-REFERENCE

This application is a national stage of PCT International Application No. PCT/US2017/034564, filed May 25, 2017, which claims the benefit of and priority to U.S. provisional application No. 62/341,482, filed May 25, 2016, and U.S. provisional application No. 62/341,487, filed May 25, 2016. The entire contents of each of which are incorporated herein by reference in their entirety.

### FIELD OF THE INVENTION

There is a need for new therapy regimens for the treatment of metabolic disorders.

### BACKGROUND OF THE INVENTION

Metabolic disease including obesity, diabetes, hypertension, and cardiovascular disease, are diseases driven by both mulitfactorial genetics (thrifty genotypes) as well as lifestyle habits, and are now reaching epidemic proportions in developed nations. It is believed that increasingly high caloric diets combined with sedentary life styles are major contributors to the growing incidence of these diseases. Importantly hyperlipidemia is associated with many types of metabolic disease, and statistics from the American Heart Association indicate that approximately half of the adult population in the United States has plasma cholesterol levels that put individuals at risk for the development of cardiovascular disease (American Heart Association, Heart disease and stroke statistics—2005 update; 2005:1-59). Furthermore, the Third Report of the National Cholesterol Education Program Expert Panel on Detection, Evaluation, and Treatment of High Blood Cholesterol in Adults (Adult Treatment Panel III; ATPIII, National Cholesterol Education Program 2001) has identified elevated triglyceride levels as an independent risk factor for the development of cardiovascular disease. Approximately one third of the adult population in the United States that have elevated cholesterol levels also have increased triglycerides. The elevation in plasma triglycerides has now been recognized as an early and dominant dyslipidemic symptom in patients with obesity, metabolic syndrome and diabetes and has been suggested to play a causative role in the development of insulin resistance and type II diabetes (Hegarty et al., Acta Physiol Scand 2003; 178(4):373-83; Shulman, J Clin Invest 2000; 106(2):171-6).

Current standard of care for hyperlipidemia focuses on lowering low density lipoprotein cholesterol (LDL) using the statin class of hydroxymethy-glutaryl-CoA reductase inhibitors (National Cholesterol Education Program 2001). However, even after statin therapy a significant number of patients still exhibit elevated levels of plasma triglycerides and triglyceride-rich lipoproteins including very low density lipoproteins (VLDL) and intermediate density lipoproteins (IDL) (Friday, Exp Biol Med (Maywood) 2003, 228(7):769-78; Quilliam et al., J Manag Care Pharm 2004, 10(3):244-50). To treat this population of patients with concurrent high plasma triglyceride levels the ATPIII has identified lowering of triglyceride-rich cholesterol fractions (VLDL+IDL) as a secondary target of drug therapy (National Cholesterol Education Program 2001). Unfortunately treatment of such patients with fibrates, an approved class of triglyceride lowering drugs, has potential adverse side effects, including the possibility of increased LDL cholesterol as well as carrying the risk of fatal rhabdomyolysis, so that combination therapy must proceed cautiously (National Cholesterol Education Program 2001). Similarly nicotinic acid, a second approved triglyceride lowering agent, is contraindicated in patients with insulin resistance and type II diabetes (Capuzzi et al., Curr Atheroscler Rep 2000, 2(1):64-71). Taken together these observations highlight the need for an effective therapeutic agent for the lowering of triglycerides and non-HDL cholesterol in patients with cardiovascular disease, diabetes, and metabolic syndrome.

The maintenance of lipid homeostasis requires coordinate control of cholesterol and triglyceride synthesis, transport, up-take, and excretion. Interestingly, studies in human and in animal models have uncovered a link between bile acids, the metabolic end-product of cholesterol metabolism, and lipid homeostasis. Clinical studies in the late 1970s exploring the effect of bile acids on cholesterol gallstones demonstrated that treatment with chenodeoxycholic acid (CDCA) reduces plasma triglyceride levels (Bateson et al., Br J Clin Pharmacol 1978, 5(3):249-54; Iser and Sali, Drugs 1981, 21(2):90-119). In contrast, treatment with bile acid sequestrants, which deplete intestinal bile acids, increase triglycerides (Angelin et al., J Lipid Res 1978; 19(8):1017-24). Importantly the bile acid-dependent decrease in triglycerides is mediated, at least in part, through a reduction in the production of VLDL (Hirokane et al., J Biol Chem 2004, 279(44):45685-92; Post et al., Arterioscler Thromb Vasc Biol 2004, 24(4):768-74; Sirvent et al., FEBS Lett 2004, 566(1-3):173-7; Kang and Davis, Biochim Biophys Acta 2000, 1529(1-3):223-30).

Atherosclerosis and its clinical consequences, including coronary heart disease (CHD), stroke and peripheral vascular disease, represent a truly enormous burden to the health care systems of the industrialized world. In the United States alone, approximately 13 million patients have been diagnosed with CHD, and greater than one half million deaths are attributed to CHD each year. Further, this toll is expected to grow over the next quarter century as an epidemic in obesity and diabetes continues to grow.

It has long been recognized that in mammals, variations in circulating lipoprotein profiles correlate with the risk of atherosclerosis and CHD. The clinical success of HMG-CoA reductase inhibitors, especially the statins, in reducing coronary events is based on the reduction of circulating low density lipoprotein cholesterol (LDL-C), levels of which correlate directly with an increased risk for atherosclerosis. More recently, epidemiologic studies have demonstrated an inverse relationship between high density lipoprotein cholesterol (HDL-C) levels and atherosclerosis, leading to the conclusion that low serum HDL-C levels are associated with an increased risk for CHD.

### SUMMARY OF THE INVENTION

The present application relates to methods of treating a metabolic disorder in an individual in need thereof, comprising co-administering to the individual a therapeutically effective amount of: (a) a first agent that is an FXR modulator; and (b) at least one second agent that is an CCR2/CCR5 antagonist, ASK1 inhibitor, DPP-IV inhibitor, caspase protease inhibitor, SGLT2 inhibitor, acetyl-CoA carboxylase (ACC) inhibitor, diacylglycerol acyltransferase-1 inhibitor, sodium-bile acid cotransporter-inhibitor, TLR-4 antagonist, PPAR alpha/delta agonist, or GLP-1 agonist, or a combination thereof.

The present application also relates to FXR modulators. Disclosed herein are FXR modulatators, and pharmaceutical compositions that include such FXR modulatators, for use in the treatment of diseases, disorders or conditions that would benefit from FXR modulation. In one aspect is the administration of an FXR modulator described herein to a mammal in the treatment of diseases, disorders or conditions that would benefit from FXR modulation.

In an aspect of the invention are methods of treating a metabolic disorder in an individual in need thereof, comprising co-administering to the individual a therapeutically effective amount of: (a) a first agent that is an FXR modulator; and (b) a second agent that is a DPP-IV inhibitor. Disclosed herein, are methods of treating a metabolic disorder in an individual in need thereof, comprising co-administering to the individual a therapeutically effective amount of: (a) a first agent that is an FXR modulator; and (b) a second agent that is an SGLT2 inhibitor. Disclosed herein, are methods of treating a metabolic disorder in an individual in need thereof, comprising co-administering to the individual a therapeutically effective amount of: (a) a first agent that is an FXR modulator; and (b) a second agent that is an ASK1 inhibitor. Disclosed herein, are methods of treating a metabolic disorder in an individual in need thereof, comprising co-administering to the individual a therapeutically effective amount of: (a) a first agent that is an FXR modulator; and (b) a second agent that is a GLP-1 agonist.

In some embodiments, the FXR modulator is a compound of Formula (I):

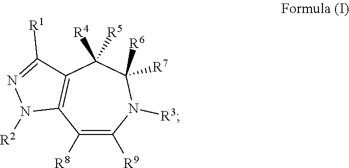

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R² is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of —CN, —C(O)OR²⁵, —C(O)N(R²⁵)R²⁶,

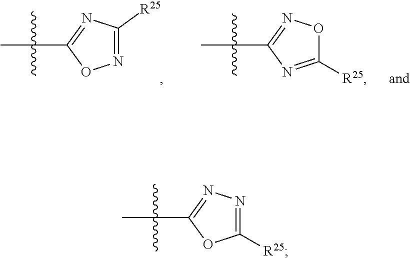

R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R⁸ and R⁹ together with the carbon atoms to which they are attached, form an optionally substituted C₂-C₉heterocycloalkyl ring or an optionally substituted heteroaryl ring;
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or a pharmaceutically acceptable salt, stereoisomer, or solvate thereof
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or
R²⁷ and R²⁸ together with the nitrogen atom to which they are attached, form an optionally substituted C₂-C₉heterocycloalkyl ring; or a pharmaceutically acceptable salt, stereoisomer, or solvate thereof.

In some embodiments, the FXR modulator is a compound of Formula (II):

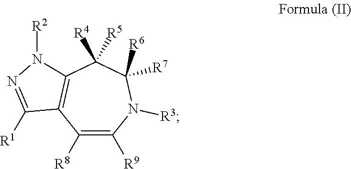

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R² is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of —CN, —C(O)OR²⁵, —C(O)N(R²⁵)R²⁶;

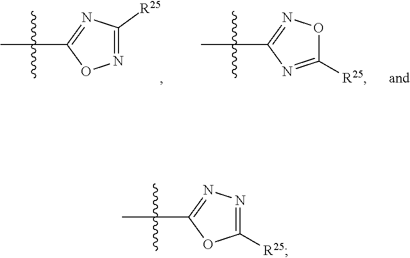

R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R⁸ and R⁹ together with the carbon atoms to which they are attached, form an optionally substituted C₂-C₉heterocycloalkyl ring or an optionally substituted heteroaryl ring;
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or a pharmaceutically acceptable salt, stereoisomer, or solvate thereof
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or
R²⁷ and R²⁸ together with the nitrogen atom to which they are attached, form an optionally substituted C₂-C₉heterocycloalkyl ring; or a pharmaceutically acceptable salt, stereoisomer, or solvate thereof.

In some embodiments of a compound of Formula (I) or (II), R⁶ and R⁷ are hydrogen. In some embodiments of a compound of Formula (I) or (II), R⁴ and R⁵ are each independently optionally substituted C₁-C₆alkyl. In some embodiments of a compound of Formula (I) or (II), R⁴ and R⁵ are methyl. In some embodiments of a compound of Formula (I) or (II), R³ is —C(O)R²⁰. In some embodiments of a compound of Formula (I) or (II), R³ is —S(O)₂R²⁰. In some embodiments of a compound of Formula (I) or (II), R³ is —C(O)R²⁰ or —S(O)₂R²⁰; and R²⁰ is optionally substituted aryl. In some embodiments of a compound of Formula (I) or (II), R³ is —C(O)N(R²¹)R²². In some embodiments of a compound of Formula (I) or (II), R³ is —C(O)N(R²¹)R²²; R²¹ is hydrogen and R²² is optionally substituted aryl. In some embodiments of a compound of Formula (I) or (II), R⁸ is —C(O)OR²⁵. In some embodiments of a compound of Formula (I) or (II), R⁸ is —C(O)OR²⁵ and R²⁵ is optionally substituted C₁-C₆alkyl. In some embodiments of a compound of Formula (I) or (II), R⁸ is —C(O)OR²⁵ and R²⁵ is methyl. In some embodiments of a compound of Formula (I) or (II), R⁸ is —C(O)OR²⁵ and R²⁵ is ethyl. In some embodiments of a compound of Formula (I) or (II), R⁹ is hydrogen or optionally substituted C₁-C₆alkyl. In some embodiments of a compound of Formula (I) or (II), R⁹ is hydrogen. In some embodiments of a compound of Formula (I) or (II), R⁹ is optionally substituted C₁-C₆alkyl. In some embodiments of a compound of Formula (I) or (II), R⁹ is C₁-C₆alkyl. In some embodiments of a compound of Formula (I) or (II), R⁹ is methyl. In some embodiments of a compound of Formula (I) or (II), R² is hydrogen. In some embodiments of a compound of Formula (I) or (II), R¹ is hydrogen. In some embodiments of a compound of Formula (I) or (II), R¹ is C₁-C₆alkyl, optionally substituted C₂-C₆alkenyl, or optionally substituted C₂-C₆alkynyl.

In some embodiments, the FXR modulator is a compound of Formula (III):

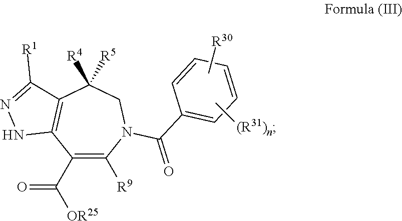

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R⁴ and R⁵ are each independently optionally substituted C₁-C₆alkyl;
R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R²⁵ is C₁-C₆alkyl;
R³⁰ is halogen,

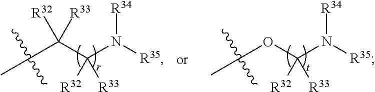

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
n is 0, 1, 2, 3, or 4;
r is 0, 1, 2, 3, or 4;
t is 2, 3, or 4; or a pharmaceutically acceptable salt, stereoisomer, or solvate thereof.

In some embodiments of a compound of Formula (III), R³⁰ is F. In some embodiments of a compound of Formula (III), R³⁰ is

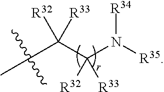

In some embodiments of a compound of Formula (III), R³⁰ is

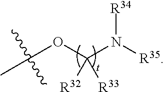

In some embodiments of a compound of Formula (III), t is 2; and each R³² and R³³ are hydrogen. In some embodiments of a compound of Formula (III), R³⁴ and R³⁵ together with the nitrogen atom to which they are attached form a C₂-C₉heterocycloalkyl ring. In some embodiments of a compound of Formula (III), R³⁴ and R³⁵ together with the nitrogen atom to which they are attached form a morpholinyl. In some embodiments of a compound of Formula (III), n is 1. In some embodiments of a compound of Formula (III), R³¹ is halogen. In some embodiments of a compound of Formula (III), R³¹ is F. In some embodiments of a compound of Formula (III), R⁴ and R⁵ are each methyl. In some embodiments of a compound of Formula (III), R⁹ is hydrogen or optionally substituted C₁-C₆alkyl. In some embodiments of a compound of Formula (III), R⁹ is hydrogen. In some embodiments of a compound of Formula (III), R⁹ is optionally substituted C₁-C₆alkyl. In some embodiments of a compound of Formula (III), R⁹ is C₁-C₆alkyl. In some embodiments of a compound of Formula (III), R⁹ is methyl.

In some embodiments of a compound of Formula (III), the FXR modulator is a compound of Formula (IIIa), or a pharmaceutically acceptable salt, stereoisomer, or solvate thereof:

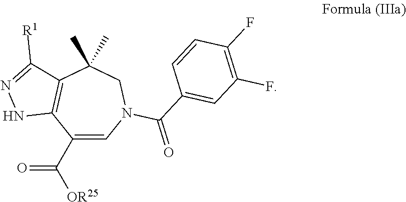

In some embodiments of a compound of Formula (III), the FXR modulator is a compound of Formula (IIIb), or a pharmaceutically acceptable salt, stereoisomer, or solvate thereof:

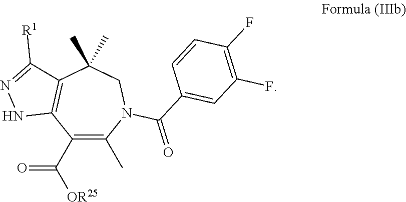

In some embodiments, the FXR modulator is a compound of Formula (IV):

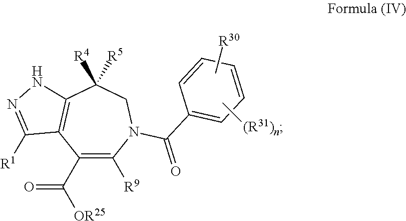

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R⁴ and R⁵ are each independently optionally substituted C₁-C₆alkyl;
R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R²⁵ is C₁-C₆alkyl;
R³⁰ is halogen,

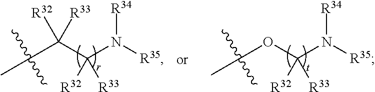

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
n is 0, 1, 2, 3, or 4;
r is 0, 1, 2, 3, or 4;
t is 2, 3, or 4; or a pharmaceutically acceptable salt, stereoisomer, or solvate thereof.

In some embodiments of a compound of Formula (IV), R³⁰ is F. In some embodiments of a compound of Formula (IV), R³⁰ is

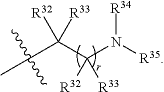

In some embodiments of a compound of Formula (IV), R³⁰ is

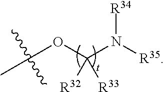

In some embodiments of a compound of Formula (IV), t is 2; and each R³² and R³³ are hydrogen. In some embodiments of a compound of Formula (IV), R³⁴ and R³⁵ together with the nitrogen atom to which they are attached form a C₂-C₉heterocycloalkyl ring. In some embodiments of a compound of Formula (IV), R³⁴ and R³⁵ together with the nitrogen atom to which they are attached form a morpholinyl. In some embodiments of a compound of Formula (IV), n is 1. In some embodiments of a compound of Formula (IV), R³¹ is halogen. In some embodiments of a compound of Formula (IV), R³¹ is F. In some embodiments of a compound of Formula (IV), R⁴ and R⁵ are each methyl. In some embodiments of a compound of Formula (IV), R⁹ is hydrogen or optionally substituted C₁-C₆alkyl. In some embodiments of a compound of Formula (IV), R⁹ is hydrogen. In some embodiments of a compound of Formula (IV), R⁹ is optionally substituted C₁-C₆alkyl. In some embodiments of a compound of Formula (IV), R⁹ is C₁-C₆alkyl. In some embodiments of a compound of Formula (IV), R⁹ is methyl.

In some embodiments of a compound of Formula (IV), the FXR modulator is a compound of Formula (IVa), or a pharmaceutically acceptable salt, stereoisomer, or solvate thereof:

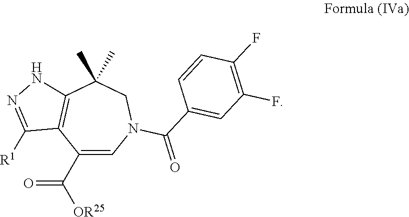

In some embodiments of a compound of Formula (IV), the FXR modulator is a compound of Formula (IVb), or a pharmaceutically acceptable salt, stereoisomer, or solvate thereof:

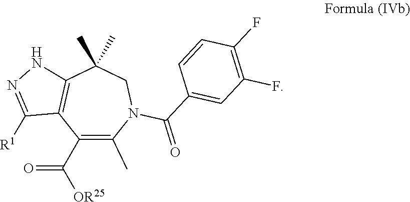

In some embodiments of a compound of Formula (III) or (IV), R¹ is hydrogen. In some embodiments of a compound of Formula (III) or (IV), R¹ is C₁-C₆alkyl, optionally substituted C₂-C₆alkenyl, or optionally substituted C₂-C₆alkynyl. In some embodiments of a compound of Formula (III) or (IV), R²⁵ is methyl. In some embodiments of a compound of Formula (III) or (IV), R²⁵ is ethyl. In some embodiments of a compound of Formula (III) or (IV), R²⁵ is isopropyl.

In some embodiments, the FXR modulator is a compound of Formula (V):

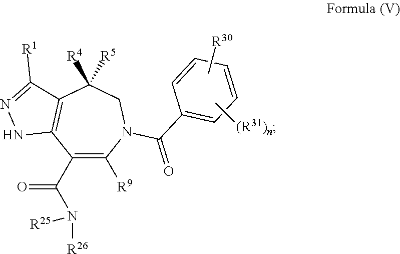

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R⁴ and R⁵ are each independently optionally substituted C₁-C₆alkyl;
R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R²⁵ and R²⁶ are each independently selected from the group consisting of hydrogen, and optionally substituted C₁-C₆alkyl;
R³⁰ is halogen,

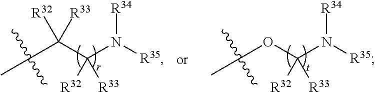

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
n is 0, 1, 2, 3, or 4;
r is 0, 1, 2, 3, or 4;
t is 2, 3, or 4; or a pharmaceutically acceptable salt, stereoisomer, or solvate thereof.

In some embodiments of a compound of Formula (V), R³⁰ is F. In some embodiments of a compound of Formula (V), R³⁰ is

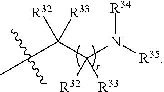

In some embodiments of a compound of Formula (V), R³⁰ is

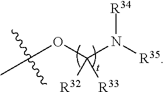

In some embodiments of a compound of Formula (V), t is 2; and each R³² and R³³ are hydrogen. In some embodiments of a compound of Formula (V), R³⁴ and R³⁵ together with the nitrogen atom to which they are attached form a C₂-C₉heterocycloalkyl ring. In some embodiments of a compound of Formula (V), R³⁴ and R³⁵ together with the nitrogen atom to which they are attached form a morpholinyl. In some embodiments of a compound of Formula (V), n is 1. In some embodiments of a compound of Formula (V), R³¹ is halogen. In some embodiments of a compound of Formula (V), R³¹ is F. In some embodiments of a compound of Formula (V), R⁴ and R⁵ are each methyl. In some embodiments of a compound of Formula (V), R⁹ is hydrogen or optionally substituted C₁-C₆alkyl. In some embodiments of a compound of Formula (V), R⁹ is hydrogen. In some embodiments of a compound of Formula (V), R⁹ is optionally substituted C₁-C₆alkyl. In some embodiments of a compound of Formula (V), R⁹ is C₁-C₆alkyl. In some embodiments of a compound of Formula (V), R⁹ is methyl.

In some embodiments of a compound of Formula (V), the FXR modulator is a compound of Formula (Va), or a pharmaceutically acceptable salt, stereoisomer, or solvate thereof:

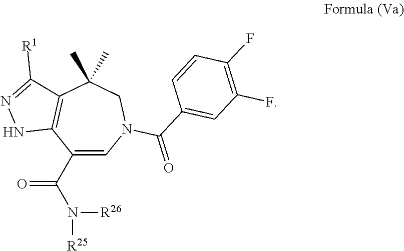

In some embodiments of a compound of Formula (V), the FXR modulator is a compound of Formula (Vb), or a pharmaceutically acceptable salt, stereoisomer, or solvate thereof:

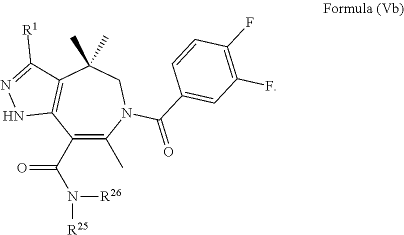

In some embodiments, the FXR modulator is a compound of Formula (VI):

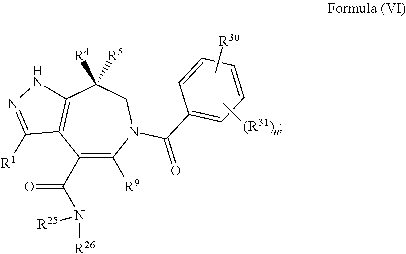

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R⁴ and R⁵ are each independently optionally substituted C₁-C₆alkyl;
R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R²⁵ and R²⁶ are each independently selected from the group consisting of hydrogen, and optionally substituted C₁-C₆alkyl;

R³⁰ is halogen,

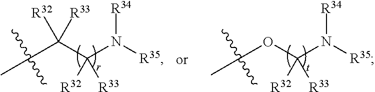

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
n is 0, 1, 2, 3, or 4;
r is 0, 1, 2, 3, or 4;
t is 2, 3, or 4; or a pharmaceutically acceptable salt, stereoisomer, or solvate thereof.

In some embodiments of a compound of Formula (VI), R³⁰ is F. In some embodiments of a compound of Formula (VI), R³⁰ is

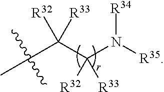

In some embodiments of a compound of Formula (VI), R³⁰ is

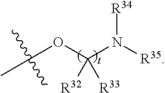

In some embodiments of a compound of Formula (VI), t is 2; and each R³² and R³³ are hydrogen. In some embodiments of a compound of Formula (VI), R³⁴ and R³⁵ together with the nitrogen atom to which they are attached form a C₂-C₉heterocycloalkyl ring. In some embodiments of a compound of Formula (VI), R³⁴ and R³⁵ together with the nitrogen atom to which they are attached form a morpholinyl. In some embodiments of a compound of Formula (VI), n is 1. In some embodiments of a compound of Formula (VI), R³¹ is halogen. In some embodiments of a compound of Formula (VI), R³¹ is F. In some embodiments of a compound of Formula (VI), R⁴ and R⁵ are each methyl. In some embodiments of a compound of Formula (VI), R⁹ is hydrogen or optionally substituted C₁-C₆alkyl. In some embodiments of a compound of Formula (VI), R⁹ is hydrogen. In some embodiments of a compound of Formula (VI), R⁹ is optionally substituted C₁-C₆alkyl. In some embodiments of a compound of Formula (VI), R⁹ is C₁-C₆alkyl. In some embodiments of a compound of Formula (VI), R⁹ is methyl.

In some embodiments of a compound of Formula (VI), the FXR modulator is a compound of Formula (VIa), or a pharmaceutically acceptable salt, stereoisomer, or solvate thereof:

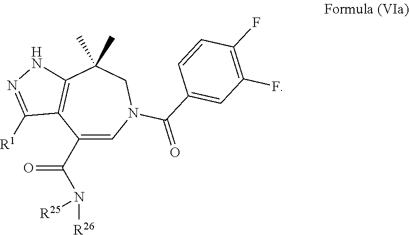

In some embodiments of a compound of Formula (VI), the FXR modulator is a compound of Formula (VIb), or a pharmaceutically acceptable salt, stereoisomer, or solvate thereof:

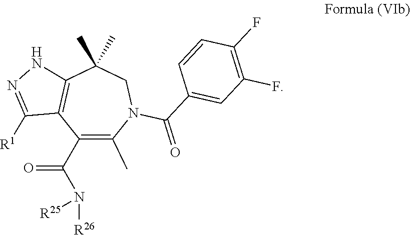

In some embodiments of a compound of Formula (V) or (VI), R¹ is hydrogen. In some embodiments of a compound of Formula (V) or (VI), R¹ is C₁-C₆alkyl, optionally substituted C₂-C₆alkenyl, or optionally substituted C₂-C₆alkynyl. In some embodiments of a compound of Formula (V) or (VI), R²⁵ and R²⁶ are independently hydrogen or C₁-C₆alkyl. In some embodiments of a compound of Formula (V) or (VI), R²⁵ and R²⁶ are hydrogen. In some embodiments of a compound of Formula (V) or (VI), R²⁵ and R²⁶ are independently C₁-C₆alkyl.

In some embodiments, the FXR modulator is a compound of Formula (VII):

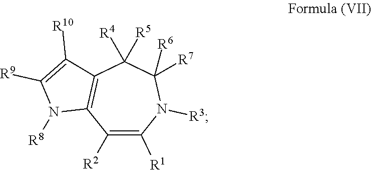

wherein:
R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);

R² is selected from the group consisting of —CN, —C(O)OR²⁵, —C(O)N(R²⁵)R²⁶,

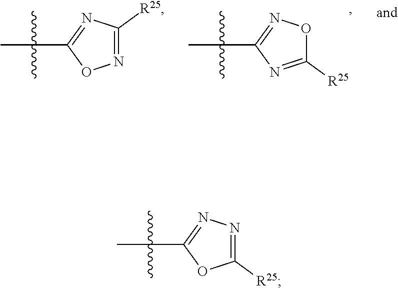

or R¹ and R² together with the carbon atoms to which they are attached, form an optionally substituted C₂-C₉heterocycloalkyl ring or an optionally substituted heteroaryl ring;
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R⁹ and R¹⁰ together with the carbon atoms to which they are attached, form an optionally substituted nitrogen containing 6-membered heteroaryl ring;
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring; or a pharmaceutically acceptable salt, stereoisomer, or solvate thereof.

In some embodiments of a compound of Formula (VII), the FXR modulator is a compound of Formula (VIIa), or a pharmaceutically acceptable salt, stereoisomer, or solvate thereof:

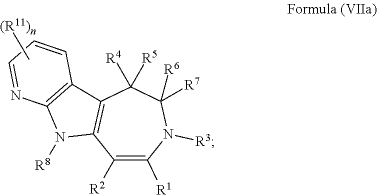

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², and —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In some embodiments of a compound of Formula (VII), the FXR modulator is a compound of Formula (VIIb), or a pharmaceutically acceptable salt, stereoisomer, or solvate thereof:

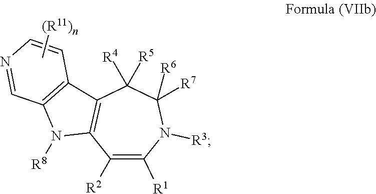

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², and —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In some embodiments of a compound of Formula (VII), the FXR modulator is a compound of Formula (VIIc), or a pharmaceutically acceptable salt, stereoisomer, or solvate thereof:

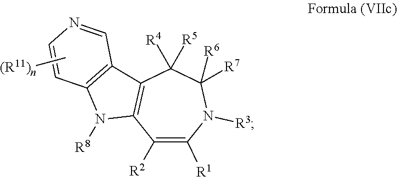

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², and —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In some embodiments of a compound of Formula (VII), the FXR modulator is a compound of Formula (VIId), or a pharmaceutically acceptable salt, stereoisomer, or solvate thereof:

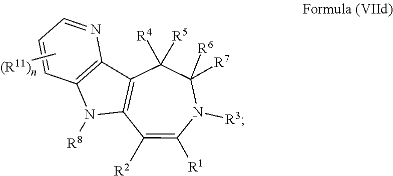

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², and —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In some embodiments of a compound of Formula (VII), the FXR modulator is a compound of Formula (VIIe), or a pharmaceutically acceptable salt, stereoisomer, or solvate thereof:

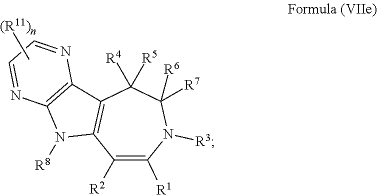

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², and —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VII), the FXR modulator is a compound of Formula (VIIf), or a pharmaceutically acceptable salt, stereoisomer, or solvate thereof:

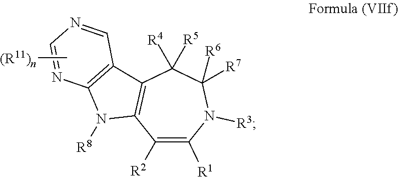

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², and —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VII), the FXR modulator is a compound of Formula (VIIg), or a pharmaceutically acceptable salt, stereoisomer, or solvate thereof:

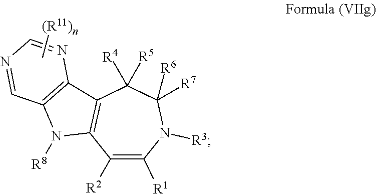

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², and —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VII), the FXR modulator is a compound of Formula (VIIh), or a pharmaceutically acceptable salt, stereoisomer, or solvate thereof:

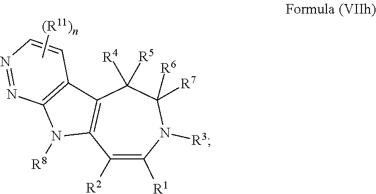

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², and —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VII), the FXR modulator is a compound of Formula (VIIi), or a pharmaceutically acceptable salt, stereoisomer, or solvate thereof:

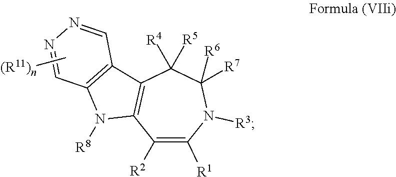

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², and —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VII), the FXR modulator is a compound of Formula (VIIj), or a pharmaceutically acceptable salt, stereoisomer, or solvate thereof:

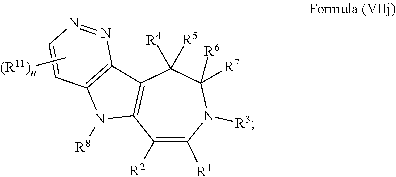

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², and —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VIIa)-(VIIj), n is 0. In some embodiments of a compound of Formula (VII) or (VIIa)-(VIIj), R⁶ and R⁷ are hydrogen. In some embodiments of a compound of Formula (VII) or (VIIa)-(VIIj), R³ is —C(O)N(R²¹)R²². In some embodiments of a compound of Formula (VII) or (VIIa)-(VIIj), R³ is —C(O)N(R²¹)R²²; R²¹ is hydrogen and R²² is optionally substituted aryl. In some embodiments of a compound of Formula (VII) or (VIIa)-(VIIj), wherein R³ is —C(O)R²⁰. In some embodiments of a compound of Formula (VII) or (VIIa)-(VIIj), R³ is —S(O)₂R²⁰. In some embodiments of a compound of Formula (VII) or (VIIa)-(VIIj), R³ is —C(O)R²⁰ or R³ is —S(O)₂R²⁰ and R²⁰ is optionally substituted aryl.

In some embodiments, the FXR modulator is a compound of Formula (VIII):

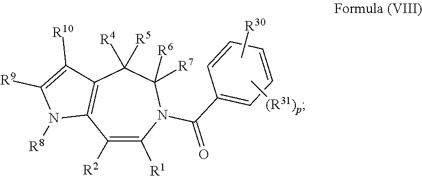

wherein:
R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R² is selected from the group consisting of —CN, —C(O)OR²⁵, —C(O)N(R²⁵)R²⁶,

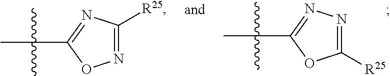

or R¹ and R² together with the carbon atoms to which they are attached, form an optionally substituted C₂-C₉heterocycloalkyl ring or an optionally substituted heteroaryl ring;
R⁴ and R⁵ are each independently selected from the group consisting of hydrogen, halogen, optionally substituted C₁-C₆alkoxy, optionally substituted C₁-C₆alkyl, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R⁹ and R¹⁰ together with the carbon atoms to which they are attached, form an optionally substituted nitrogen containing 6-membered heteroaryl ring;
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring;
R³⁰ is halogen,

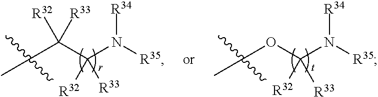

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
p is 0, 1, 2, 3, or 4;
r is 0, 1, 2, 3, or 4; and
t is 2, 3, or 4; or a pharmaceutically acceptable salt, stereoisomer, or solvate thereof.

In some embodiments of a compound of Formula (VIII), the FXR modulator is a compound of Formula (VIIIa), or a pharmaceutically acceptable salt, stereoisomer, or solvate thereof:

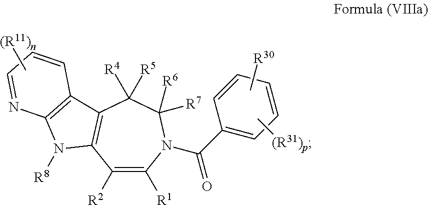

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In some embodiments of a compound of Formula (VIII), the FXR modulator is a compound of Formula (VIIIb), or a pharmaceutically acceptable salt, stereoisomer, or solvate thereof:

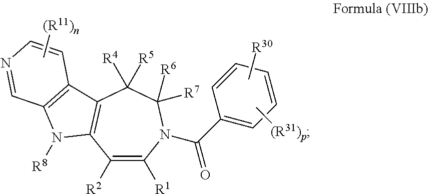

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In some embodiments of a compound of Formula (VIII), the FXR modulator is a compound of Formula (VIIIc), or a pharmaceutically acceptable salt, stereoisomer, or solvate thereof:

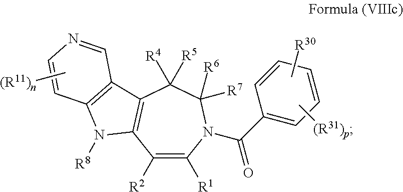

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In some embodiments of a compound of Formula (VIII), the FXR modulator is a compound of Formula (VIIId), or a pharmaceutically acceptable salt, stereoisomer, or solvate thereof:

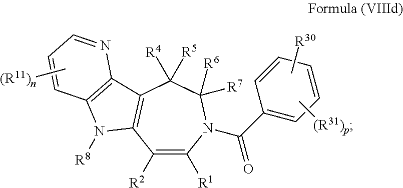

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In some embodiments of a compound of Formula (VIII), the FXR modulator is a compound of Formula (VIIIe), or a pharmaceutically acceptable salt, stereoisomer, or solvate thereof:

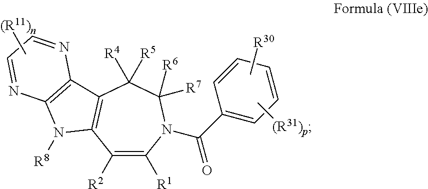

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VIII), the FXR modulator is a compound of Formula (VIIIf), or a pharmaceutically acceptable salt, stereoisomer, or solvate thereof:

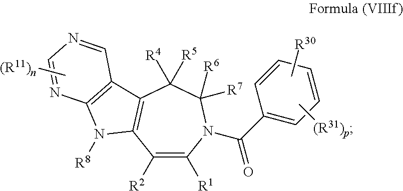

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VIII), the FXR modulator is a compound of Formula (VIIIg), or a pharmaceutically acceptable salt, stereoisomer, or solvate thereof:

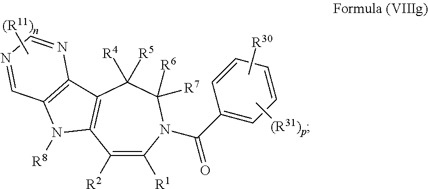

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VIII), the FXR modulator is a compound of Formula (VIIIh), or a pharmaceutically acceptable salt, stereoisomer, or solvate thereof:

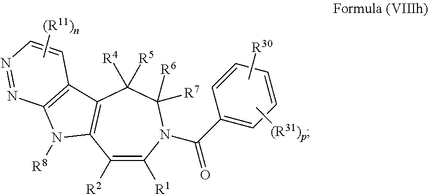

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VIII), the FXR modulator is a compound of Formula (VIIIi), or a pharmaceutically acceptable salt, stereoisomer, or solvate thereof:

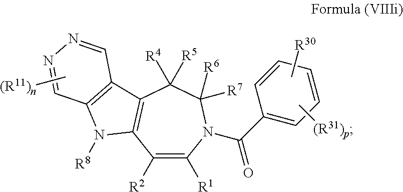

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VIII), the FXR modulator is a compound of Formula (VIIIj), or a pharmaceutically acceptable salt, stereoisomer, or solvate thereof:

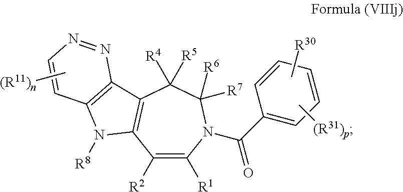

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments of a compound of Formula (VIIIa)-(VIIIj), n is 0. In some embodiments of a compound of Formula (VIII) or (VIIIa)-(VIIIj), R³⁰ is F. In some embodiments of a compound of Formula (VIII) or (VIIIa)-(VIIIj), R³⁰ is

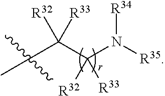

In some embodiments of a compound of Formula (VIII) or (VIIIa)-(VIIIj), R³⁰ is

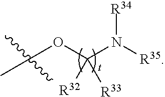

In some embodiments of a compound of Formula (VIII) or (VIIIa)-(VIIIj), t is 2; and each R³² and R³³ are hydrogen. In some embodiments of a compound of Formula (VIII) or (VIIIa)-(VIIIj), R³⁴ and R³⁵ together with the nitrogen atom to which they are attached form a C₂-C₉heterocycloalkyl ring. In some embodiments of a compound of Formula (VIII) or (VIIIa)-(VIIIj), R³⁴ and R³⁵ together with the nitrogen atom to which they are attached form a morpholinyl. In some embodiments of a compound of Formula (VIII) or (VIIIa)-(VIIIj), p is 1. In some embodiments of a compound of Formula (VIII) or (VIIIa)-(VIIIj), R³¹ is halogen. In some embodiments of a compound of Formula (VIII) or (VIIIa)-(VIIIj), R³¹ is F. In some embodiments of a compound of Formula (VIII) or (VIIIa)-(VIIIj), R⁶ and R⁷ are hydrogen. In some embodiments of a compound of Formula (VIII) or (VIIIa)-(VIIIj), R⁴ and R⁵ are each independently optionally substituted C₁-C₆alkyl. In some embodiments of a compound of Formula (VIII) or (VIIIa)-(VIIIj), R⁴ and R⁵ are methyl. In some embodiments of a compound of Formula (VIII) or (VIIIa)-(VIIIj), R² is —C(O)OR²⁵. In some embodiments of a compound of Formula (VIII) or (VIIIa)-(VIIIj), R² is —C(O)OR²⁵ and R²⁵ is optionally substituted C₁-C₆alkyl. In some embodiments of a compound of Formula (VIII) or (VIIIa)-(VIIIj), R² is —C(O)OR²⁵ and R²⁵ is methyl. In some embodiments of a compound of Formula (VIII) or (VIIIa)-(VIIIj), R² is —C(O)OR²⁵ and R²⁵ is ethyl. In some embodiments of a compound of Formula (VIII) or (VIIIa)-(VIIIj), R² is —C(O)OR²⁵ and R²⁵ is isopropyl. In some embodiments of a compound of Formula (VIII) or (VIIIa)-(VIIIj), R² is —C(O)N(R²⁵)R²⁶. In some embodiments of a compound of Formula (VIII) or (VIIIa)-(VIIIj), R¹ is hydrogen or optionally substituted C₁-C₆alkyl. In some embodiments of a compound of Formula (VIII) or (VIIIa)-(VIIIj), R¹ is hydrogen. In some embodiments of a compound of Formula (VIII) or (VIIIa)-(VIIIj), R¹ is optionally substituted C₁-C₆alkyl. In some embodiments of a compound of Formula (VIII) or (VIIIa)-(VIIIj), R¹ is C₁-C₆alkyl. In some embodiments of a compound of Formula (VIII) or (VIIIa)-(VIIIj), R¹ is methyl. In some embodiments of a compound of Formula (VIII) or (VIIIa)-(VIIIj), R⁸ is hydrogen.

In some embodiments, the FXR modulator is a compound of Formula (XI) or a pharmaceutically acceptable salt, stereoisomer, or solvate thereof:

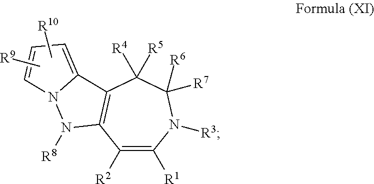

wherein:
R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R² is selected from the group consisting of —CN, —C(O)OR²⁵, —C(O)N(R²⁵)R²⁶,

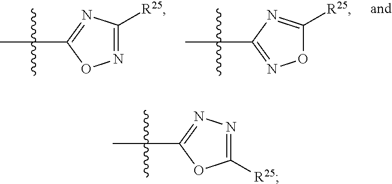

or R¹ and R² together with the carbon atoms to which they are attached, form an optionally substituted C₂-C₉heterocycloalkyl ring or an optionally substituted heteroaryl ring;
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R⁹ and R¹⁰ are each independently selected from the group consisting of hydrogen, halogen, —CN, amino, alkylamino, optionally substituted C₁-C₆alkyl, optionally substituted C₁-C₆alkoxy, optionally substituted C₃-C₈cycloalkyl, optionally substituted C₂-C₉heterocycloalkyl, optionally substituted aryl, and optionally substituted heteroaryl;
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring.

In some embodiments, the FXR modulator is a compound of Formula (XII), or a pharmaceutically acceptable salt, stereoisomer, or solvate thereof:

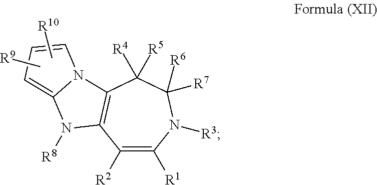

wherein:
R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R² is selected from the group consisting of —CN, —C(O)OR²⁵, —C(O)N(R²⁵)R²⁶,

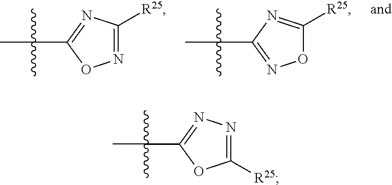

or R¹ and R² together with the carbon atoms to which they are attached, form an optionally substituted C₂-C₉heterocycloalkyl ring or an optionally substituted heteroaryl ring;
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R⁹ and R¹⁰ are each independently selected from the group consisting of hydrogen, halogen, —CN, amino, alkylamino, optionally substituted C₁-C₆alkyl, optionally substituted C₁-C₆alkoxy, optionally substituted C₃-C₈cycloalkyl, optionally substituted C₂-C₉heterocycloalkyl, optionally substituted aryl, and optionally substituted heteroaryl;
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring.

In some embodiments of a compound of Formula (XI) or (XII), or a pharmaceutically acceptable salt, stereoisomer, or solvate thereof, R⁴ and R⁵ are hydrogen. In some embodiments of a compound of Formula (XI) or (XII), or a pharmaceutically acceptable salt, stereoisomer, or solvate thereof, R⁴ and R⁵ are C₁-C₆alkyl. In some embodiments of a compound of Formula (XI) or (XII), or a pharmaceutically acceptable salt, stereoisomer, or solvate thereof, R⁴ and R⁵ are methyl. In some embodiments of a compound of Formula (XI) or (XII), or a pharmaceutically acceptable salt, stereoisomer, or solvate thereof, R⁶ and R⁷ are hydrogen. In some embodiments of a compound of Formula (XI) or (XII) R⁶ is —C(O)N(R²⁷)R²⁸ and R⁷ are hydrogen. In some embodiments of a compound of Formula (XI) or (XII), or a pharmaceutically acceptable salt, stereoisomer, or solvate thereof, R² is —C(O)OR²⁵. In some embodiments of a compound of Formula (XI) or (XII), or a pharmaceutically acceptable salt, stereoisomer, or solvate thereof, R² is —C(O)OR²⁵ and R²⁵ is optionally substituted C₁-C₆alkyl. In some embodiments of a compound of Formula (XI) or (XII), or a pharmaceutically acceptable salt, stereoisomer, or solvate thereof, R² is —C(O)OR²⁵ and R²⁵ is methyl. In some embodiments of a compound of Formula (XI) or (XII), or a pharmaceutically acceptable salt, stereoisomer, or solvate thereof, R² is —C(O)OR²⁵ and R²⁵ is ethyl. In some embodiments of a compound of Formula (XI) or (XII), or a pharmaceutically acceptable salt, stereoisomer, or solvate thereof, R² is —C(O)OR²⁵ and R²⁵ is isopropyl. In some embodiments of a compound of Formula (XI) or (XII), or a pharmaceutically acceptable salt, stereoisomer, or solvate thereof, R² is —C(O)N(R²⁵)R²⁶. In some embodiments of a compound of Formula (XI) or (XII), or a pharmaceutically acceptable salt, stereoisomer, or solvate thereof, R¹ is hydrogen. In some embodiments of a compound of Formula (XI) or (XII), or a pharmaceutically acceptable salt, stereoisomer, or solvate thereof, R¹ is optionally substituted C₁-C₆alkyl. In some embodiments of a compound of Formula (XI) or (XII), or a pharmaceutically acceptable salt, stereoisomer, or solvate thereof, R¹ is —CH₃. In some embodiments of a compound of Formula (XI) or (XII), or a pharmaceutically acceptable salt, stereoisomer, or solvate thereof, R³ is —C(O)N(R²¹)R²². In some embodiments of a compound of Formula (XI) or (XII), or a pharmaceutically acceptable salt, stereoisomer, or solvate thereof, R³ is —C(O)N(R²¹)R²², R²¹ is hydrogen, and R²² is optionally substituted aryl. In some embodiments of a compound of Formula (XI) or (XII), or a pharmaceutically acceptable salt, stereoisomer, or solvate thereof, R³ is —C(O)R²⁰. In some embodiments of a compound of Formula (XI) or (XII), or a pharmaceutically acceptable salt, stereoisomer, or solvate thereof, R³ is —C(O)R²⁰ and R²⁰ is optionally substituted aryl. In some embodiments of a compound of Formula (XI) or (XII), or a pharmaceutically acceptable salt, stereoisomer, or solvate thereof, R³ is —S(O)₂R²⁰. In some embodiments of a compound of Formula (XI) or (XII), or a pharmaceutically acceptable salt, stereoisomer, or solvate thereof, R³ is —S(O)₂R²⁰ and R²⁰ is optionally substituted aryl.

In some embodiments of a compound of Formula (VIII), the FXR modulator is a compound of Formula (VIIIa), or a pharmaceutically acceptable salt, stereoisomer, or solvate thereof, or a compound of Formula (XII), the FXR modulator is a compound of Formula (XIIa), or a pharmaceutically acceptable salt, stereoisomer, or solvate thereof:

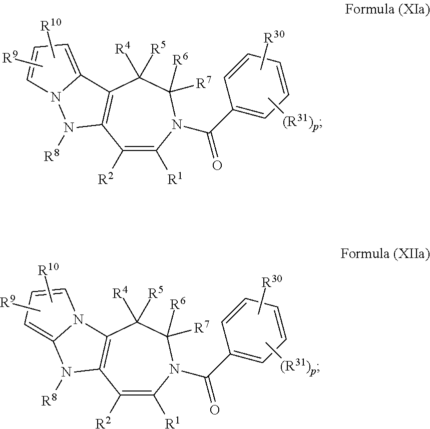

wherein:
R³⁰ is halogen,

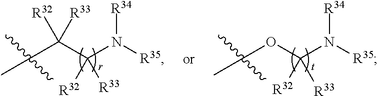

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring;
p is 0, 1, 2, 3, or 4;
r is 0, 1, 2, 3, or 4; and
t is 2, 3, or 4.

In some embodiments of a compound of Formula (XIa) or (XIIa), or a pharmaceutically acceptable salt, stereoisomer, or solvate thereof, R³⁰ is halogen. In some embodiments of a compound of Formula (XIa) or (XIIa), or a pharmaceutically acceptable salt, stereoisomer, or solvate thereof, R³⁰ is F. In some some embodiments of a compound of Formula (XIa) or (XIIa), or a pharmaceutically acceptable salt, stereoisomer, or solvate thereof, R³⁰ is

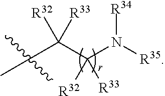

In some embodiments of a compound of Formula (XIa) or (XIIa), or a pharmaceutically acceptable salt, stereoisomer, or solvate thereof, R³⁰ is

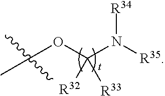

In some embodiments of a compound of Formula (XIa) or (XIIa), or a pharmaceutically acceptable salt, stereoisomer, or solvate thereof, t is 2. In some embodiments of a compound of Formula (XIa) or (XIIa), or a pharmaceutically acceptable salt, stereoisomer, or solvate thereof, p is 0. In some embodiments of a compound of Formula (XIa) or (XIIa), or a pharmaceutically acceptable salt, stereoisomer, or solvate thereof, p is 1. In some embodiments of a compound of some embodiments of a compound of Formula (XIa) or (XIIa), or a pharmaceutically acceptable salt, stereoisomer, or solvate thereof, R³¹ is F.

In an aspect of the invention, are FXR modulatators, and pharmaceutical compositions that include such FXR modulatators, for use in the treatment of diseases, disorders or conditions that would benefit from FXR modulation. In one aspect is the administration of an FXR modulator described herein to a mammal in the treatment of diseases, disorders or conditions that would benefit from FXR modulation. In some embodiments is the administration of an FXR modulator described herein to a mammal in the treatment of diseases, disorders or conditions that would benefit from FXR modulation, wherein the FXR modulator is (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

In an aspect of the invention, are methods of treating a metabolic disorder in an individual in need thereof, comprising co-administering to the individual a therapeutically effective amount of: (a) a first agent that is an FXR modulator; and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, and omarigliptin.

In an aspect of the invention, are methods of treating a metabolic disorder in an individual in need thereof, comprising co-administering to the individual a therapeutically effective amount of: (a) a first agent that is an FXR modulator; and (b) a second agent that is an SGLT2 inhibitor selected from canagliflozin, empagliflozin, dapagliflozin, ipragliflozin, tofogliflozin, sergliflozin etabonate, remogliflozin etabonate, and ertugliflozin.

In an aspect of the invention, is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

In an aspect of the invention, are methods of treating a metabolic disorder in an individual in need thereof, comprising co-administering to the individual a therapeutically effective amount of: (a) a first agent that is an FXR modulator; and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride).

In an aspect of the invention, are methods of treating a metabolic disorder in an individual in need thereof, comprising co-administering to the individual a therapeutically effective amount of: (a) a first agent that is an FXR modulator; and (b) a second agent that is a GLP-1 agonist selected from exenatide, liraglutide, lixisenatide, albiglutide, dulaglutide, taspoglutide, and semaglutide.

In an aspect of the invention, are methods of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof; wherein the metabolic disorder is nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, diabetic nephropathy or nephrotic syndrome. In some embodiments, the metabolic disorder is nonalcoholic steatohepatitis (NASH).

In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, or obesity.

### CROSS-REFERENCE TO RELATED APPLICATIONS

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The present application relates to FXR modulatators, and pharmaceutical compositions that include such FXR modulatators, for use in the treatment of diseases, disorders or conditions that would benefit from FXR modulation. In one aspect is the administration of an FXR modulator described herein to a mammal in the treatment of diseases, disorders or conditions that would benefit from FXR modulation. In some embodiments is the administration of an FXR modulator described herein to a mammal in the treatment of diseases, disorders or conditions that would benefit from FXR modulation, wherein the FXR modulator is (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

Disclosed herein, are methods of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof.

### FXR Biology

The Farnesoid X receptor (FXR; also referred to as NR1H4; nuclear receptor nomenclature committee 1999) is a member of the steroid and thyroid hormone nuclear receptor superfamily of ligand regulated transcription factors. FXR is highly expressed in the liver, kidney, intestines and the adrenals and at lower levels in the vasculature (Forman et al., Cell 1995, 81(5):687-93). Bile acids, the end-products of cholesterol catabolism, bind directly to the ligand binding pocket of FXR and act as agonists to increase the receptor's ability to activate transcription (Makishima et al., Science 1999, 284(5418):1362-5 1999; Mi et al., Mol Cell 2003, 11(4):1093-100; Parks et al., Science 1999, 284(5418):1365-8; Wang et al., Mol Cell 1999, 3(5):543-53). In response to bile acid binding FXR regulates a network of genes that control the synthesis, transport, and catabolism of bile acids, but also triglycerides and cholesterol (Chawla et al., Cell 2000, 103(1):1-4; Repa and Mangelsdorf, Annu Rev Cell Dev Biol 2000, 16:459-81). Thus FXR functions as a regulator of lipid metabolism by modifying gene expression in response to quantitative changes in the metabolism and breakdown of cholesterol. In support of this conclusion, studies in humans and in animals have demonstrated that modifying bile acid levels can have profound effects on plasma triglyceride and cholesterol levels (Angelin et al., J Lipid Res 1978, 19(8):1017-24; Bateson et al., Br J Clin Pharmacol 1978, 5(3):249-54; Iser and Sali, Drugs 1981, 21(2):90-119; Kuroki et al., Lipids 1999, 34(8):817-23).

FXR was originally cloned and classified as an orphan member of the nuclear hormone receptor superfamily based upon DNA sequence homology. Initial studies identified farnesol as a ligand for FXR (Forman et al., Cell 1995, 81(5):687-93), however, subsequent analysis demonstrated that bile acids bind directly to the ligand binding domain of FXR and function as activators of the receptor's transcriptional activity. The binding affinities of bile acids for FXR is near the concentration that these compounds reach in animals (μM) lending support to the idea that bile acids function as endogenous ligands in vivo (Makishima et al., Science 1999, 284(5418):1362-5 1999; Mi et al., Mol Cell 2003, 11(4):1093-100; Parks et al., Science 1999, 284(5418):1365-8; Wang et al., Mol Cell 1999, 3(5):543-53). Activation of FXR upon bile acid binding leads to transcriptional down-regulation of cholesterol 7α-hydroxylase (CYP7A1), the rate limiting enzyme in the conversion of cholesterol to bile acids. Inhibition of CYP7A1 by bile acids occurs via FXR-dependent induction of the small heterodimeric partner (SHP; also referred to as NR0B2, Nuclear Receptor Nomenclature Committee 1999), a transcriptional repressor. Binding sites for FXR have been identified in the SHP promoter indicating that this gene is a direct target of FXR (Lu et al., Mol Cell 2000, 6(3):507-15; Goodwin et al., Mol Cell 2000, 6(3):517-26). Thus bile acid-dependent repression of CYP7A1 is indirect and results from a transcriptional cascade initiated by FXR. A similar SHP-dependent mechanism has been described for the bile acid repression of another gene involved in bile acid synthesis, CYP8B1 (sterol 12a hydroxylase; Yang et al., Biochim Biophys Acta 2002, 1583(1):63-73), and for the sodium/taurocholate cotransporter peptide (NTCP) which is one of two major transporters responsible for bile acid up-take by the liver (Denson et al., Gastroenterology 2001; 121(1):140-7). In contrast the genes encoding the bile salt export pump (BSEP) and the multidrug resistance protein 2 (MDR2) are directly induced by FXR, once again via binding sites in their respective promoter regions (Ananthanarayanan et al., J Biol Chem 2001, 276(31):28857-65; Huang et al., J Biol Chem 2003, 278(51):51085-90; Liu et al., J Clin Invest 2003, 112(11):1678-87). These two transporters are required for the transfer of bile acids (BSEP) and phospholipids (MDR2) out of the hepatocytes into the biliary system. This pattern of FXR-dependent gene expression defines a classic feedback loop where high levels of bile acids inhibit new bile acid synthesis and bile acid uptake while simultaneously promoting their own clearance.

The regulation of bile acid synthesis and transport by FXR has important implications for cholesterol metabolism. Repression of CYP7A1 and CYP8B1 impacts the bile acid synthetic pathway at two important points. First, inhibition of CYP7A1, the rate limiting enzyme, can decrease synthesis and reduce the size of the bile acid pool. Second, inhibition of CYP8B1 alters bile acid composition by favoring the production of more hydrophilic bile acids such as CDCA (muricholic acid/MCA in mice) (Russell, Annu Rev Biochem 2003, 72:137-74). Importantly, studies in mice have demonstrated that the more hydrophilic bile acids are less efficient at promoting intestinal cholesterol absorption (Wang et al., Am J Physiol Gastrointest Liver Physiol 2003, 285(3):G494-502).

Although regulating bile acid synthesis may contribute to the FXR-dependent effects on lipid metabolism, gene expression analysis indicates that FXR also directly influences triglyceride synthesis and VLDL production. FXR agonists induce the genes encoding fibroblast growth factor 19 (Holt et al., Genes Dev 2003, 17(13):1581-91), acylation stimulating protein (a proteolytic product of complement C3; Li et al., J Biol Chem 2005, 280(9):7427-34), apolipoprotein CII (Kast et al., Mol Endocrinol 2001, 15(10):1720-8), and apolipoprotein AV (Prieur et al., J Biol Chem 2003, 278(28):25468-80) all of which are known to promote the clearance and oxidation of fat carried by triglyceride rich lipoproteins. Additionally FXR inhibits expression of the genes encoding apolipoprotein CIII (Claudel et al., Gastroenterology 2003, 125(2):544-55), an inhibitor of lipoprotein lipase, and the sterol response element binding protein 1c (SREBP1c; Watanabe et al., J Clin Invest 2004, 113(10):1408-18). SREBP1c, a member of basic helix-loop-helix family of transcription factors, functions as a master transcriptional regulator of the enzymes required for fatty acid synthesis (Osborne, J Biol Chem 2000, 275(42):32379-82). Taken together the genetic network controlled by FXR defines a signal transduction system poised to respond to changes in fat and carbohydrate dietary intake-driven lipid homeostasis. High levels of cholesterol in the liver will lead to increased production of bile acids and subsequent activation of FXR. In response to this activating signal FXR decreases the absorption of cholesterol in the intestine, favoring excretion, increases the clearance and oxidation of triglycerides and decreases the synthesis of fatty acids leading to a reduction in VLDL production.

The ability of FXR to regulate bile-acid synthesis, clearance and homeostasis as supported by the ability of FXR ligands to promote the transport of bile acid and phospholipids out of the liver suggests a utility for such compounds in diseases of disturbed bile acid and cholesterol flow such as Primary Biliary cirrhosis and NASH. In this regard FXR agonists have been shown to be effective in animal models of cholestasis, gallstones, and liver fibrosis (Liu et al., J Clin Invest 2003, 112(11):1678-87; Fiorocci et al., Gastroenterology 2004, 127(5):1497-512; Fiorocci et al., J Pharmacol Exp Ther 2005, 313(2):604-12; Fiorocci et al., J Pharmacol Exp Ther 2005, 314(2):584-95).

### DPP-IV Biology

Dipeptidyl peptidase-IV (DPP-IV) is a serine protease, which selectively cleaves the N-terminal dipeptide from the penultimate position of Glucose-dependent Insulinotropic Polypeptide (GIP) and Glucagon-Like Peptide (GLP-1) thus making them inactive (Diabetes Obes Metab., 10, 376-387, 2008; Diabetes Care, 30, 1979-1987, 2007). GLP-1 is an incretin hormone secreted by intestinal L-Cells in response to food intake. The active GLP-1 stimulates insulin secretion, inhibits glucagon release and slows gastric emptying, which together contributes for effective glucose homeostasis in patients with type 2 diabetes. Inhibition of DPP-IV activity extends the duration of action of endogenous GLP-1, thereby exhibiting all the favorable attributes of GLP-1 (Lancet, 368, 1696-1705, 2006; Horm Metab Res., 36 (11-12), 867-76, 2004). DPP-IV inhibitors offer a number of potential advantages over existing diabetes therapies, including a lowered risk of hypoglycemia, weight gain and the potential for regeneration and differentiation of pancreatic beta-cells (Handbook Exp Pharmacol., 203, 53-74, 2011; Curr Med Res Opin., 23(4), 919-31, 2007). Because of these multiple benefits of GLP-1 mediated glucose homeostasis, orally bioavailable DPP-IV inhibitors has been developed as promising therapeutic agents for the treatment of type 2 diabetes (Am. J. Ther., 15(5), 484-91, 2008). The therapeutic potential of DPP-IV inhibitors for the treatment of type 2 diabetes have been discussed and reviewed extensively (Exp. Opin. Invest. Drugs, 12, 87-100, 2003; Exp. Opin. Ther. Patents, 13, 499-510, 2003; Exp. Opin. Investig. Drugs, 13, 1091-1102, 2004; Curr. Opin. Drug Discovery Development, 11, 512-532, 2008 and Trends in Molecular Medicine, 14, 161-168, 2008).

### SGLT2 Biology

Sodium-dependent glucose cotransporters (SGLTs) couple the transport of glucose against a concentration gradient with the simultaneous transport of Na+ down a concentration gradient. Two important SGLT isoforms have been cloned and identified as SGLT1 and SGLT2. SGLT1 is located in the gut, kidney, and heart where its expression regulates cardiac glucose transport. SGLT1 is a high-affinity, low-capacity transporter and therefore accounts for only a small fraction of renal glucose reabsorption. In contrast, SGLT2 is a low-affinity, high-capacity transporter located exclusively at the apical domain of the epithelial cells in the early proximal convoluted tubule. In healthy individuals, greater than 99% of the plasma glucose that filtered in the kidney glomerulus is reabsorbed, resulting in less than 1% of the total filtered glucose being excreted in urine. It is estimated that 90% of renal glucose reabsorption is facilitated by SGLT2; the remaining 10% is likely mediated by SGLT1 in the late proximal straight tubule. Genetic mutations in SGLT2 lead to increased renal glucose excretion of as much as 140 g/day depending on the mutation with no apparent adverse effects on carbohydrate metabolism. Since SGLT2 appears to be responsible for the majority of renal glucose reabsorption based on human mutation studies, it has become a target of therapeutic interest (Lee, J. et al. Bioorg. Med. Chem. 2010, 18, 2178-2194; Van den Heuvel, L. P. et al. Hum. Genet. 2020, 111, 544-547).

### ASK1 Biology

Mitogen-activated protein kinase (MAPK) signaling cascades couple diverse extracellular and intracellular queues to appropriate cellular stress responses, including cell growth, differentiation, inflammation, and apoptosis (Kumar, S., et al. (2003) Nat. Rev. Drug Dis. 2:717-726; Pimienta, G., et al. (2007) Cell Cycle, 6: 2826-2632). MAPKs exist in three groups, MAP3Ks, MAP2Ks, and MAPKs, which are sequentially activated. MAPK3s directly respond to environmental signals and phosphorylate MAP2Ks, which in turn phosphorylate specific MAPKs. MAPKs then mediated the appropriate cellular response by phosphorylating cellular substrates, including transcription factors that regulate gene expression. Apoptosis signal-regulating kinase 1 (ASK1) is a member of the mitogen-activated protein kinase kinase kinase (“MAP3K”) family that activates the c-Jun N-terminal protein kinase (“JNK”) and p38 MAP kinase (Ichijo, H., et al. (1997) Science, 275, 90-94). ASK1 is activated by a variety of stimuli including oxidative stress, reactive oxygen species (ROS), LPS, TNF-alpha, FasL, ER stress, and increased intracellular calcium concentrations (Hattori, K., et al. (2009) Cell Comm. Signal. 7:1-10; Takeda, K., et al. (2007) Annu. Rev. Pharmacol. Toxicol. 48: 1-8.27; Nagai, H., et al. (2007) J. Biochem. Mol. Biol. 40:1-6). ASK1 undergoes activation via autophosphorylation at Thr838 in response to these signals and in turn phosphorylates MAP2Ks, such as MKK3/6 and MKK4/7, which then phosphorylate and activate p38 and JNK MAPKs, respectively. ASK2 is a related MAP3K that shares 45% sequence homology with ASK1 (Wang, X. S., et al. (1998) Biochem. Biophys. Res. Commun. 253, 33-37. Although ASK2 tissue distribution is restricted, in some cell types ASK1 and ASK2 have been reported to interact and function together in a protein complex (Takeda, K., et al. (2007) J. Biol. Chem. 282: 7522-7531; Iriyama, T., et al. (2009) Embo J. 28: 843-853) In non stressed conditions, ASK1 is kept in an inactive state through binding to its repressor Thioredoxin (Trx) (Saitoh, M., et al. (1998) Embo J. 17:2596-2606), and through association with AKT (Zhang, L., et al. (1999) Proc. Natl. Acad. Sci. U.S.A 96:8511-8515). Phosphorylation of ASK1 protein can lead to apoptosis or other cellular responses depending on the cell type. ASK1 activation and signaling have been reported to play an important role in a broad range of diseases including neurodegenerative, cardiovascular, inflammatory, autoimmunity, and metabolic disorders. In addition, ASK1 has been implicated in mediating organ damage following ischemia and reperfusion of the heart, brain, and kidney (Watanabe et al. (2005) BBRC 333, 562-567; Zhang et al., (2003) Life Sci 74-37-43; Terada et al. (2007) BBRC 364: 1043-49). Emerging evidence suggests that ASK2, either alone or in a complex with ASK1, may play important roles in human diseases as well. Therefore, therapeutic agents that function as inhibitors of ASK1 and ASK2 signaling complexes have the potential to remedy or improve the lives of patients suffering from such conditions.

### GLP-1 Biology

Glucagon and GLP-1 are members of structurally related peptide hormone family (secretin family). Glucagon and GLP-1 constitute a highly homologous set of peptides because these two hormones originate from a common precursor, preproglucagon, which upon tissue-specific processing leads to production of GLP-1 predominantly in the intestine and glucagon in the pancreas (Jiang, G., et al., Am. J. Physiol. Endocrinol. Metab., 2003, 284, E671-678). The receptors for these two peptides are homologous (58% identity) and belong to the class B family of G-protein coupled receptors (GPCRs). Class-B GPCRS is also called as the secretin receptor family, which consist of 15 peptide-binding receptors in humans. GPCR receptors comprise an extracellular N-terminal domain of 100-160 residues, connected to a juxtamembrane domain (J-domain) of seven membrane-spanning co-helices with intervening loops and a C-terminal tail (Brubaker, P. L., et al., Receptors Channels, 2002, 8, 179). Class B GPCRs are activated by endogenous peptide ligands of intermediate size, typically 30-40 amino acids (Hoare, S. R. J., Drug Discovery Today, 2005, 10, 423; Gether, U., Endocrine Reviews, 2000, 21, 90).

Glucagon is a 29-amino acid peptide hormone processed from proglucagon in pancreatic alpha-cells by PC2. Glucagon acts via a seven transmembrane GPCRs, consisting of 485 amino acids. Glucagon is released into the bloodstream when circulating glucose is low. The main physiological role of glucagon is to stimulate hepatic glucose output, thereby leading to increase in glycemia (Tan, K., et al., Diabetologia, 1985, 28, 435). Glucagon provides the major counter regulatory mechanism for insulin in maintaining glucose homeostasis in vivo. Glucagon and its receptor represent potential targets for the treatment of diabetes. Antagonising glucagon action by blocking the action of the secreted glucagon at glucagon receptor (glucagon antagonist) or by inhibiting (suppressing) the glucagon production itself represents a new avenue for intervention of diabetes and metabolic disorders (Unson, C. G., et al., Peptides, 1989, 10, 1171; Parker, J. C., Diabetes, 2000, 49, 2079; Johnson, D. G., Science, 1982, 215, 1115).

The GLP-1 (7-36) amide is a product of the preproglucagon gene, which is secreted from intestinal L-cells, in response to the ingestion of food. The physiological action of GLP-1 has gained considerable interest. GLP-1 exerts multiple actions by stimulating insulin secretion from pancreatic beta-cells, in a glucose dependent manner (insulinotropic action). GLP-1 lowers circulating plasma glucagon concentration, by inhibiting its secretion (production) from alpha-cells (Drucker D. J., Endocrinology, 2001, 142, 521-527). GLP-1 also exhibits properties like stimulation of beta-cell growth, appetite suppression, delayed gastric emptying and stimulation of insulin sensitivity (Nauck, M. A., Horm. Metab. Res., 2004, 36, 852).

### CCR2/CCR5 Antagonist Biology

The chemokine system comprises more than 20 different chemokine receptors, which belong to the class A or rhodopsin-like family of G protein-coupled receptors (GPCRs). Almost 50 chemokine ligands play a critical role in the immune system, mediating the migration and differentiation of immune cells during homeostasis and inflammation. Dysregulation of this system can lead to a variety of different pathologies, including inflammatory and autoimmune diseases. (Bot et al. Scientific Reports 7, Article number: 52 (2017) doi:10.10381/s41598-017-00104-4

Fibrosis results from a sustained inflammatory response to chronic organ injury and is characterized by the deposition of extracellular matrix proteins, including collagen types 1 and 3. Hepatic fibrosis is associated with chronic liver disease, a significant global burden that contributes to cirrhosis and hepatocellular carcinoma. Likewise, renal fibrosis is a common manifestation of chronic kidney disease. The inflammatory response to hepatocyte injury plays a key role in hepatic fibrogenesis and involves recruitment of bone marrow-derived monocytes and macrophages to the site of injury, which is triggered by the activation of resident macrophages. In turn, infiltrating monocytes/macrophages amplify this immune response by producing inflammatory cytokines and chemokines, which further promote recruitment of inflammatory cells and upregulate the activation of hepatic stellate: cells (HSCs). Fibrogenic cytokines (e.g. transforming growth factor-beta [TGF-beta]), produced by activated macrophages, promote transdifferentiation of HSCs into myofibroblasts, which are the primary source of scar-forming matrix proteins, including fibrillar), collagen types I and 3, and the contractile protein alpha-smooth muscle actin (alpha-SMA). (Lefebvre F, Moyle O, Reshef R, Richman L P, Thompson M, Hong F, et al. (2016) Antifibrotic Effects of the Dual CCR2/CCR5 Antagonist Cenicriviroc in Animal Models of Liver and Kidney Fibrosis. PLoS ONE 11(6): e0158156. https://doi.org/10.1371/journal.pone.0158156)

Recruitment of extra-hepatic inflammatory cells to the site of hepatic injury is largely mediated by interactions between chemokines and their receptors. Monocytes, KCs and HSCs can express C—C chemokine receptor types 2 (CCR2) and 5 (CCR5) on their surface. (Lefebvre et al. 2016 https://doi.org/10.1371/journal.pone.0158156). Examples of drugs which are considered CCR2/CCR5 antagonist therapeutics include, but are not limited to, cenicriviroc (CVC), aplaviroc, vicriviroc (e.g., 5-({4-[(3S)-4-{2-methoxy-1-[4-(trifluoromethyl)phenyl]ethyl}-3-methylpiperazin-1-yl]-4-methylpiperidin-1-yl}carbonyl)-4,6-dimethylpyrimidine), maraviroc (e.g., 4,4′-difluorocyclohexylamide) and cochilioquinone A.

### Caspase Protease Inhibitor Biology

Caspases are the key effector molecules of the physiological death process known as apoptosis, although some are involved in activation of cytokines, rather than cell death. They are one of approximately 20 families of cysteine proteases. Caspases exist in most mammalian cells as inactive precursors (zymogens) that kill the cell once activated and can be controlled in two ways. The processing and activation of a caspase can be regulated by molecules such as FADD, APAF-1, Bcl-2 family members, FLIP and IAPs. Active caspases can be controlled by a variety of inhibitors that directly interact with the protease. (Ekert, P. G.; Silke, J.; Vaux, D. L. Cell Death Differ. 1999; 6(11):1081-6). Examples of drugs considered to be caspase protease inhibitors include, but are not limited to, emricasan, Q-VD-Oph, DEVD-CHO, zVAD-FMK, Pralnacasan (Vertex), and M867 (Merck).

### Acetyl-CoA Carboxylase (ACC) Inhibitor Biology

Acetyl-CoA carboxylase is a biotin-dependent enzyme that catalyzes the irreversible carboxylation of acetyl-CoA to produce malonyl-CoA through its two catalytic activities, biotin carboxylase (BC) and carboxyltransferase (CT). ACC is a large, multi-domain enzyme in the endoplasmic reticulum of most eukaryotes. The most important function of ACC is to provide the malonyl-CoA substrate for the biosynthesis of fatty acids. The human genome contains the genes for two different ACCs: ACACA and ACACB. (Widmer, J. Biochem J. 1996, 15: 316—Part 3, 915-922.) Examples of drugs include, but are not limited to, (R)-2-(1-(2-(2-methoxyphenyl)-2-((tetrahydro-2H-pyran-4-yl)oxy)ethyl)-5-methyl-6-(oxazol-2-yl)-2,4-dioxo-1,4-dihydrothieno[2,3-d]pyrimidin-3(2H)-yl)-2-methylpropanoicacid (“NDI-010976” or “GS-0976”) (Gilead), 5-(tetradecyloxy)-2-furoic acid (“TOFA”), Medica 16, and (3R)-1′-(9-anthracenylcarbonyl)[1,4′-bipiperidin]-3-yl]-4-morpholinyl-methanone (“CP-640186”) (Cayman Chemical).

### Diacylglycerol Acyltransferase-1 Inhibitor Biology

Diacylglycerol acyltransferase-1 is an enzyme involved in the formation of triyglcerides and is highly expressed in human fat metabolism sites such as intesting, liver, and adipose. Dietary triglycerides cannot be absorbed directly in the gastrointestinal tract and are broken down into free fatty acids and monglycerol in the intestine by pancreatic lipase. Inhibition of diacylglycerol acyltransferase-1 has shown to reduce fat storage in mammals leading to reduction of body weight. Examples of drugs include, but are not limited to, pradigastat (“LCQ908”), VK5211, A 922500, amidepsine A, and amidepisne D.

### Apical Sodium-Bile Acid Cotransporter-Inhibitor (ASBT) Biology

Apical sodium-dependent bile acid transporter (ASBT or ABAT, SLC10A2) is the second member of the SLC10A family of solute carrier proteins and has important physiological functions as a bile acid transporter from the lumen of the gastrointestinal tract to the liver via the portal vein. Classes of therapeutics include dihydropyridine calcium channel blockers, and HMG-CoA reductase inhibitors (statins). Examples of drugs include, but are not limited to, volixibat (“LUM-002/SHP626”), LJN 452 (Novartis), GSK2330672 (GSK), AZD-7806, S-8921, AK-105, BARI-1741, SC-435 or SC-635.

### TLR-4 Antagonist Biology

TLR-4 is a protein that in humans is encoded by the TLR4 gene. TLR4 is a transmembrane protein, member of the toll-like receptor family, which belongs to the pattern recognition receptor (PRR) family. Its activation leads to an intracellular signaling pathway NF-κB and inflammatory cytokine production which is responsible for activating the innate immune system. It is most well known for recognizing lipopolysaccharide (LYS), a component present in many Gram-negative bacteria (e.g. Neisseria spp.) and select Gram-positive bacteria. Its ligands also include several viral proteins, polysaccharide, and a variety of endogenous proteins such as low-density lipoprotein, beta-defensins, and heat shock protein. Examples of drugs which are considered TLR-4 antagonists include, but are not limited to, JKB-121, amitriptyline, imipramine, naloxone, LPS-RS, cyclbenzprine, mianserin, naltrexone, propentofylline, ketotitfen, ibudilast, (+)-naltrexone, tapentradol, and eritoran.

### PPAR Alpha/Delta Agonist Biology

Examples of drugs which are considered PPAR alpha/delta agonists include, but are not limited to, GFT505, amphipathic carboxylic acids (e.g., clofibrate, gemfibrozil, ciprofibrate, bezafibrate, and fenofibrate), GW501516, aleglitzar, muraglitazar, tesaglitazar, and saroglitazar.

### Statins

Statins, also known as HMG-CoA reductase inhibitors, are a class of lipid-lowering medications. Statins have been found to reduce cardiovascular disease (CVD) and mortality in those who are at high risk. Statins are effective for treating CVD in the early stages of a disease (secondary prevention) and in those at elevated risk but without CVD (primary prevention). Examples of drugs include, but are not limited to, atorvastatin, fluvastatin, lovastatin, pitavastatin, pravastatin, rosuvastatin, and simvastatin.

### FXR Agonists and Nonalcoholic Fatty Liver Disease (NAFLD) and Nonalcoholic Staetohepatitis (NASH)

NAFLD is a well-recognized component of the metabolic syndrome, characterized by increased serum levels of lipids and glucose, increased incidence of type II diabetes, atherosclerosis, hypertension, and breast and colon cancer. Although many NAFLD cases have benign prognosis, some develop NASH, liver fibrosis, cirrhosis, and tumor. The disruption of the Nr1h4 gene in mice showed that FXR deficiency results in fatty liver formation following feeding with a high-cholesterol diet (Sinal C J et al. Cell. 2000; 102:731-744). In addition, FXR deficiency renders the mice more susceptible to NASH formation in a diet-induced obese mouse model (Kong B et al. J Pharmacol Exp Ther. 2009; 328:116-122). The exact mechanism by which FXR deficiency enhances NAFLD to NASH transition is not clear, but likely involves a FXR-dependent disruption of lipid and bile acid homeostasis, which leads to lipid accumulation and bile acid-induced chronic injury in the liver. FXR deficiency also results in increased collagen expression, and increased collagen expression is an early event in liver fibrosis development. In agreement, activation of FXR has been shown to suppress liver fibrosis development. Advanced liver fibrosis leads to cirrhosis, portal hypertension and liver failure. The treatment of choice is liver transplantation because no effective pharmaceutical agents are available to halt or reverse liver fibrosis.

The effect of FXR activation on the development and protection against NASH has been investigated in animal models. Feeding mice a methionine and choline-deficient (MCD) diet is a well-established nutritional model of NASH resulting in serum elevation of alanine aminotransferase (ALT) and aspartate aminotransferase (AST), and liver histological abnormalities similar to human NASH, including hepatic steatosis, lobular inflammation, and pericellular fibrosis. C57BL/6 mice were fed an MCD diet and treated with or without WAY-362450 (a synthetic FXR agonist) for 4 weeks. The elevations of serum ALT and AST induced by the MCD diet were markedly reduced with WAY-362450 treatment. Moreover, the hepatoprotective effects of WAY-362450 were abolished in FXR^{-/-} mice fed an MCD diet. These results indicate that FXR agonists may be useful for the treatment NASH (Zhang S et al. J. Hepatol 2009; 51:380-8).

In a rabbit model of the metabolic syndrome, a high-fat diet resulted in an increase in visceral fat, fasting glycemia and glucose intolerance. Treatment with OCA (INT-747, an FXR agonist) along with the high-fat diet normalized visceral fat fasting glucose levels, and improved glucose tolerance. The effect of OCA on insulin resistance and development of hepatic steatosis has been studied in Zucker fa/fa obese rats (Cipriani S, Mencarelli A, Palladino G, et al. J Lipid Res 2010; 51:771-84), a model for NAFLD with a loss-of-function mutation of the leptin receptor. These rats exhibit hyperphagia and hyperleptinemia and develop obesity, insulin resistance, diabetes, and hepatic steatosis. In this study, in comparison to lean rats, fa/fa rats on a normal diet developed insulin resistance and hepatic steatosis. Administration of OCA reversed insulin resistance and hepatic steatosis and protected against body weight gain and liver and muscle fat deposition. Moreover, FXR activation resulted in a reduction of liver expression of genes involved in fatty acid synthesis, lipogenesis and gluconeogenesis. In muscle, FXR activation reduced free fatty acid synthesis.

Recently, the results of the Farnesoid X nuclear receptor ligand OCA in NASH treatment (the FLINT) trial were reported (Neuschwander-Tetri B A et al. Farnesoid X nuclear receptor ligand obeticholic acid for non-cirrhotic, non-alcoholic steatohepatitis (FLINT): a multicentre, randomised, placebo-controlled trial. Lancet 2014). In this multicenter, double-blinded, placebo controlled clinical trial, a total of 283 patients with biopsy-proven NASH were randomized to receive either OCA 25 mg orally daily or placebo for 72 weeks. The primary outcome measure was improvement in NAFLD activity score by at least two points without worsening of fibrosis from baseline to the end of treatment. At the time of analysis of the primary outcome, 110 patients in the OCA arm and 109 patients in the placebo arm were included in the analysis. At 72 weeks of treatment, the percentage of patients who demonstrated histological improvement in the OCA and placebo arm was 45% and 21%, respectively. A decrease in the high-density lipoprotein (HDL) and an increase in the total cholesterol and low-density lipoprotein (LDL) was observed in patients in the OCA arm compared to placebo. These results suggest that OCA might be beneficial in preventing progression of NASH.

### FXR and Inflammatory Bowel Disease (IBD)

IBD, which primarily includes ulcerative colitis (UC) and Crohn's disease (CD), represents a group of chronic disorders characterized by gastrointestinal tract inflammation. Although many details of IBD have been explored, the exact pathogenetic mechanisms of IBD have not been fully elucidated. At present, IBD is generally believed to result from imbalance of gut microbiota, epithelial dysfunction, and aberrant mucosal immune response.

Recently, FXR has been implicated to participate in immune modulation and barrier function in the intestine. FXR alleviates inflammation and preserves the integrity of the intestinal epithelial barrier in many ways by regulating the extent of the inflammatory response, maintaining the integrity and function of the intestinal barrier, and preventing bacterial translocation in the intestinal tract.

First, FXR plays an important role in the mucosal immune response, thereby exerting strong influence on immunoregulation. Vavassori et al. (J Immunol. 2009; 183:6251-6261) noticed that Fxr^{-/-} mice displayed significantly elevated pro-inflammatory cytokine mRNA expression in the colon. In two complementary murine models (intra-rectal administration of trinitrobenzensulfonic acid (TNBS) and oral administration of dextrane sodium sulfate (DSS)), concurrent administration of the potent synthetic FXR ligand 6-ECDCA repressed the expression of various proinflammatory cytokines, chemokines and their receptors in wild type, but not Fxr^{-/-} mice. In addition, Raybould et al. (J Physiol. 2012; 590:441-446) showed that FXR activation by INT-747 prevented DSS- and TNBS-induced intestinal inflammation, with improvement of colitis symptoms, inhibition of epithelial permeability, and reduced goblet cell loss. Furthermore, FXR activation inhibited proinflammatory cytokine production in vivo in the mouse colonic mucosa, and ex vivo in different immune cell populations. These results provide strong support for the involvement of FXR in IBD due to counter-regulatory effects on cells of innate immunity. FXR ligands exert anti-inflammatory activities by antagonizing other signaling pathways, in part through the interaction with other transcription factors, including activator protein-1 (AP-1), and signal transducers and activators of transcription 3 (STAT3).

Second, FXR has been implicated in barrier function by regulating intestinal antibacterial growth. Gut microbiota play important roles in pathogen defense, immunity, and nutrient harvest. Recent evidence suggests that there is a regulatory relationship between the development of IBD and altered gut microbiota. It has been demonstrated that BAs and gut microbiota are closely related to each other. Gut microbiota are involved in the biotransformation of BAs through deconjugation, dehydroxylation, and reconjugation of BAs. BAs have antimicrobial activities by damaging the bacterial cell membrane, thus inhibiting bacterial outgrowth.

The administration of bile or conjugated BAs to ascitic cirrhotic rats or obstructive jaundice rats eliminates intestinal bacterial overgrowth, and decreases bacterial translocation and endotoxemia. Inagaki et al. (Proc Natl Acad Sci USA. 2006; 103:3920-3925) provides an explanation for this protective effect of BAs by demonstrating that intestinal FXR has a crucial role in limiting bacterial overgrowth and thus protecting the intestine from bacterial-induced damage. They show that mice lacking FXR experience bacterial overgrowth, increase intestinal permeability and contain large amounts of bacteria in mesenteric lymph nodes, as well as inflammation of the intestinal walls. However, activation of intestinal FXR by GW4064 leads to the identification of several novel intestinal FXR target genes, including those encoding angiogenin, carbonic anhydrase 12 and inducible nitric oxide synthase, which have been reported to have antibacterial properties. The cytokine IL-18 is also induced by FXR stimulation. IL-18 stimulates resistance to an array of pathogens, including intracellular and extracellular bacteria and mycobacteria, and appears to have a protective role during the early, acute phase of mucosal immune response. These results are consistent with the idea that FXR is critical for controlling intestinal bacterial growth, which has significant implications for maintaining a competent barrier, thereby contributing to the prevention of intestinal inflammation.

### FXR and Bile Acid Diarrhea (BAD) and Irritable Bowel Syndrome (IBS)

Bile acids are increasingly implicated in the pathogenesis of functional GI disorders. New mechanisms have recently been described in the irritable bowel syndrome, chronic diarrhea and chronic idiopathic constipation. Identification of bile acid signaling through farnesoid X receptor (FXR) has led to the development of new, directly acting therapeutic agents. Despite these advances primary bile acid diarrhea (BAD) remains under-recognized partly because of the lack of a widely available diagnostic test. Functional gastrointestinal disorders (FGID) are common and constitute a significant proportion of consultations in both primary and secondary care. The most prevalent FGIDs are the irritable bowel syndrome (IBS) and functional dyspepsia, with a prevalence of around 20% each, regardless of the nationality of the population. A recent study using Rome III criteria found that 42% of attendees in the gastroenterology outpatient clinic met the criteria for a functional lower GI diagnosis. Of these patients, 24.5% met the criteria for IBS-diarrhea (IBS-D), 6.1% functional diarrhea (FD), 22.1% IBS-constipation, and 22.1% chronic idiopathic constipation. Over the last decade, understanding of the pathogenesis of these conditions has advanced and a clear relationship between bile acids (BAs) and these FGIDs have become apparent.

FGF-19 stimulation by obeticholic acid (Zhang J H et al. Am. J. Physiol. 2013; 304:G940-G948) provides an opportunity to reverse the deficiency which is considered one of the factors leading to excessive hepatocyte BA synthesis. This treatment was associated with improved stool frequency and consistency in a preliminary study of patients with BAD (Johnston I M et al. Gastroenterology. 2013; 144(Suppl. 144):S60). Given the observation that BAs chronically downregulate colonic secretory function in colonic epithelial cells, an effect that may serve to facilitate normal colonic absorptive function, it is intriguing to note that an FXR agonist, GW4064, induced nuclear translocation of the receptor in T84 cells, attenuated Cl⁻ secretory responses to both Ca²⁺ and cAMP-dependent agonists, and reduced ovalbumin-induced diarrhea and cholera toxin-induced intestinal fluid accumulation secretion in mice in vivo. These observations suggest that FXR agonists may be efficacious in the treatment of BAD through restoration of FGF-19 production and exertion of antisecretory actions on the colonic epithelium (Mroz M S et al. Gut. 2014 May; 63(5):808-17).

### FXR Agonists and Cholestatic Liver Diseases (Primary Biliary Cirrhosis (PBC), Primary Sclerosing Cholangitis (PSC) and Biliary Atresia)

PBC is a chronic, progressive, cholestatic liver disease characterized histologically by destruction of intrahepatic bile ducts and serologically by the presence of the antimitochondrial antibodies (AMAs). AMA is a highly disease-specific autoantibody, rarely found in individuals without PBC. Epidemiological studies have reported a prevalence of PBC ranging from 19 to 365 cases per million, and an incidence of 4 to 58 cases per million persons-years. PBC may lead to hepatic fibrosis, cirrhosis, and eventually liver failure. PBC is an important indication for liver transplantation in the United States and Europe. Currently, the only therapy approved by the United States Food and Drug Administration (US FDA) is ursodeoxycholic acid (UDCA). Several randomized controlled clinical trials have shown that long-term administration of UDCA in PBC patients delays histological progression to cirrhosis and prolongs survival without liver transplantation. However, up to 40% of PBC patients have incomplete response to UDCA (158). Therefore, there is a critical need for other effective therapies for PBC patients who are at high risk for progressive disease.

PSC is a progressive disease of the liver characterized by cholestasis and ongoing destruction of intra- and extra-hepatic bile ducts, leading ultimately to fibrosis, cirrhosis, and liver failure. The diagnosis of PSC is made in the setting of cholestasis and cholangiographic evidence of intra- and/or extra-hepatic biliary ductal structuring. Small-duct PSC is a variant of PSC which is characterized by cholestatic and histological evidence of PSC but normal cholangiography. PSC can progress to liver fibrosis, cirrhosis, and ultimately liver failure. PSC is an important risk factor for cholangiocarcinoma (CCA), which is the most common primary biliary malignancy, and the second most common primary liver cancer after HCC. CCA is a very aggressive disease, often diagnosed in late stages. The percentage of CCA patients who survive 5 years after diagnosis is only 10%.

Biliary atresia is a progressive obliterative cholangiopathy that presents in infancy with jaundice due to biliary obstruction. Despite the use of surgical hepatic portoenterostomy (HPE) to reestablish bile flow, biliary atresia progresses to end-stage liver disease in 80% of patients over a variable length of time. Approximately one-half of affected infants will require liver transplantation in the first two years of life due to complications of cirrhosis and cholestasis, including severe malnutrition, ascites, portal hypertension and coagulopathy. The remainder of children with biliary atresia may live many years with their native livers, despite the chronic, progressive cirrhosis that develops.

In a Wistar rat model of cholestasis, OCA promoted bile flow and protected the hepatocytes against acute necrosis caused by administration of LCA (Pellicciari R et al. J Med Chem 2002; 45:3569-72). In another rodent model of bile duct ligated (BDL) rats, the administration of OCA reduced liver fibrosis and α-collagen 1, transforming growth factor-β1 (TGF-β1), and tissue metalloproteinase inhibitor-1 (Fiorucci S et al. Gastroenterology 2004; 127:1497-512). Collectively, these results indicate that FXR activation could be beneficial in patients with cholestatic liver diseases.

Initial results from a 1-year phase III clinical trial of OCA in PBC patients were reported in the International Liver Congress in April 2014. A total of 217 patients with PBC whom previously had an inadequate response to UDCA were randomly assigned to receive placebo, OCA 10 mg daily, or OCA 5 mg daily titrated to 10 mg daily. The primary endpoint was a composite endpoint of achieving a serum ALP activity of less than 1.67 times the upper limit of normal, a total bilirubin within normal limits, and at least a 15% decrease in serum ALP. The proportion of patients meeting the primary endpoint was: 47% in the 10 mg OCA group and 46% in the 5-10 mg OCA group vs. only 10% in the placebo group. In addition, both OCA dose groups met secondary endpoints of improvements in other liver function parameters, GG, ALT and total bilirubin. Together, these results indicate that OCA could be an effective therapy for patients with PBC. Currently, a phase II clinical trial of OCA in PSC is ongoing.

### FXR and Atherosclerosis

FXR regulates lipid homeostasis and deficiency of FXR in mice increases systemic and liver lipid levels. However, FXR deficiency has been shown to increase atherosclerotic plaque formation in male ApoE knockout mice but protect female ApoE mice from atherosclerotic plaque formation (Guo G L et al. Biochim Biophys Acta. 2006; 1761:1401-1409; Zhang Y et al. Arterioscler Thromb Vasc Biol. 2006; 26:2316-2321; and Hanniman E A et al. J Lipid Res. 2005; 46:2595-2604). The reduction of atherosclerotic plaque in the aorta area of female mice may be due to a decreased CD36 expression and foam cell formation. CD36 is a long-chain fatty acid transporter and is mainly responsible for taking up oxidized LDL into macrophages. Lipid-laden macrophages become foam cells, the hall mark for atherosclerosis plaque development. This gender difference in the role of FXR in atherosclerosis development indicates again that FXR may interact with estrogen-related pathway(s) to modulate biological responses.

### FXR and Hypertriglyceridemia

The relationship between BAs (bile acids) and TG (triglyceride) metabolism was identified in the 1970s. The first evidence came from the observation that the administration of BAs, such as CDCA for the treatment of gallstones, resulted in decreased circulating TG levels; conversely, patients treated with BA-sequestering resins were found to have increased serum TG and VLDL levels. Moreover, patients with monogenic familial hypertriglyceridemia displayed a defect in ileal BA absorption, whereas individuals with decreased BA synthesis due to a CYP7A1 deficiency exhibited elevated serum TG concentrations. These clinical observations suggest a direct relationship between BAs and TG metabolism. The importance of FXR in TG metabolism was further confirmed in FXR-deficient mice, which exhibited marked hepatosteatosis and hypertriglyceridemia. In addition, FXR heterozygous mice demonstrated hepatosteatosis and hyperlipidemia following short-time high-fat diet (HFD) feeding. The TG lowering effects of endogenous and synthetic FXR agonists have been evaluated in other rodent models as well. For instance, CA prevented hepatic TG accumulation and VLDL secretion in KK-A(y) mice, a mouse model of hypertriglyceridemia (Watanabe M et al. J Clin Invest 2004; 113: 1408-18). Moreover, the synthetic FXR agonist GW4064 was able to prevent liver steatosis in obese mice, such as the ob/ob and db/db models (Zhang Y et al. Proc Natl Acad Sci USA 2006; 103: 1006-11).

### FXR and Diabetes, Diabetic Nephropathy and Glomerulosclerosis

Diabetes is the leading cause of end-stage renal disease in developed countries. In spite of excellent glucose and blood pressure control, including administration of angiotensin converting enzyme inhibitors and/or angiotensin II receptor blockers, diabetic nephropathy still develops and progresses. Diabetic nephropathy is the most common renal complication of diabetes and the leading cause of end-stage renal disease. The pathogenesis of diabetic nephropathy is complex and involves activation of multiple pathways leading to kidney damage, including the polyol pathway, advanced glycation end products, oxidative stress, proinflammatory cytokines, and profibrotic growth factors. In addition, an important role for altered lipid metabolism has been recently recognized in diabetic kidney disease. In this condition, there is increased renal expression of sterol regulatory element binding proteins 1 and 2 (SREBP-1 and SREBP-2), transcription factors that mediate increased fatty acid and cholesterol synthesis, resulting in triglyceride and cholesterol accumulation in the kidney and are associated with inflammation, oxidative stress, fibrosis, and proteinuria. A critical role for SREBP-1 was established by determining that SREBP-1 transgenic mice develop glomerulosclerosis and proteinuria in the absence of alterations in serum glucose or lipids, and that SREBP-1c knockout mice are protected from the renal effects of a high-fat diet (Sun L et al. J Biol Chem 2002; 277:18919-18927 and Jiang T et al. J Biol Chem 2005; 280:32317-32325). Modulation of SREBPs may therefore represent a rational approach to prevent diabetic renal complications. Previous studies have shown that FXR agonists decrease SREBP-1c expression in the kidney (Jiang T et al. Diabetes 2007; 56:2485-2493 and Wang X X et al. Am J Physiol Renal Physiol 2009; 297:F1587-F1596).

Treatment of db/db mice with type 2 diabetes (Jiang T et al. Diabetes. 2007; 56:2485-2493), DBA/2J mice with diet-induced obesity and insulin resistance (Wang X X et al. Am J Physiol Renal Physiol. 2009; 297:F1587-1596), and DBA/2J mice with streptozotocin-induced type 1 diabetes (Wang X X, et al. Diabetes 2010; 59:2916-2927) with FXR agonists have shown renal protective effects. These experimental models of diabetic nephropathy showed improvements in proteinuria, glomerulosclerosis, tubulointerstitial fibrosis, and macrophage infiltration following treatment with FXR activating agonists. These renal protective effects are mediated by effects on lipid metabolism, oxidative stress, and on the production of proinflammatory cytokines and profibrotic growth factors. FXR agonists inhibit expression of SREBP-1 and carbohydrate response element binding protein (ChREBP) in the kidney resulting in decreased fatty acid synthesis and triglyceride accumulation. FXR agonists also inhibit SREBP-2 resulting in decreased cholesterol synthesis and accumulation in the kidney. These studies suggest that FXR agonists can prevent the progression of kidney disease in mouse models of type 1 diabetes mellitus, diet induced obesity and insulin resistance, and type 2 diabetes mellitus.

### FXR and Cholesterol Gallstone Disease (CGD)

Gallstone disease is one of the most frequent and costly digestive diseases in western countries, as its prevalence in adults ranges from 10% to 15%. About 75% of the gallstones in the United States and westernized countries, including Italy are cholesterol gallstones. Cholesterol gallstones are associated with well-known risk factors, such as obesity, type 2 diabetes, dyslipidaemia, and hyperinsulinaemia, which are often components of the metabolic syndrome epidemic, which prevalence is greater than 35% in the adult pupulation and continues to rise in westernized countries. A complex genetic basis plays a key role in determining individual predisposition to develop cholesterol gallstones in response to environmental factors. Some “gallstone genes” might also play a potential role, including some genes governing the nuclear bile acid receptors such as farnesoid X receptor (FXR).

Moschetta et al. (Nat Med 2004; 10:1352-1358) hypothesized that FXR may play a critical role in the prevention of CGD by helping to maintain the proper solubilization of cholesterol in bile. To this end, stimulation of FXR using synthetic ligands could be useful in the prevention and treatment of CGD. In the first part of the study, Moschetta et al. demonstrates the role of FXR in the development of CGD. Age-matched wild-type and FXR^{-/-} mice were fed a lithogenic diet for 1 week, after which the gallbladder bile and expression of known FXR and LXR target genes were analyzed. Inspection of the gallbladder and bile showed increases in inflammation, bile salt hydrophobicity, bile turbidity, and presence of cholesterol monohydrate crystals in the FXR null mice, all phenotypical of CGD. Furthermore, bile salt and phospholipid levels were found to be significantly lower in the FXR null mice due to a lack of FXR-mediated expression of Abcb11 and Abcb4. Conversely, cholesterol levels were not significantly altered, because regulation of the cholesterol transporters Abcg5 and Abcg8 through LXR occurred independently of FXR. Consequently, the cholesterol saturation index was increased in the FXR null mice, driving the early development of cholesterol monohydrate crystals. In the second part of the study, Moschetta et al. expanded their findings by demonstrating that stimulation of FXR with a synthetic agonist can prevent the onset of CGD. Here, CGD-susceptible C57L and FXR^{-/-} mice were fed a lithogenic diet supplemented with the synthetic FXR ligand GW4064 or vehicle control for 1 week. As expected, examination of the gallbladder and bile indicated onset of CGD in the vehicle-treated C57L and FXR null mice as well as the GW4064-treated FXR null mice. Interestingly, two of the five vehicle-treated C57L mice evidenced more advanced disease sequelae compared with the FXR null mice, suggesting that mechanisms in addition to those mediated by FXR may contribute to the increased susceptibility of these mice to CGD. However, GW4064 treatment prevented CGD onset in the C57L mice through FXR-mediated upregulation of Abcb11 and Abcb4, increasing transport of bile salts and phospholipids to the bile, reducing of the cholesterol saturation index, and providing protection from cholesterol monohydrate crystal formation.

However, maintenance of cholesterol and bile acid homeostasis in mice is somewhat different from that of humans. The bile acid pool of mice is more hydrophilic than that of man and thus is less effective in activating FXR. Control of CYP7A1-mediated bile acid synthesis from cholesterol in mice is dominated by feed-forward activation through LXR, whereas in humans LXR is not functional in this capacity. Instead, control of bile acid synthesis in humans is dominated by feedback repression of CYP7A1 through FXR and other means. Thus in humans bile acid synthesis from cholesterol is primarily a means to maintain bile acid homeostasis, whereas in the mouse it is a means for removal of cholesterol.

### Definitions

As used in the specification and appended claims, unless specified to the contrary, the following terms have the meaning indicated below.

“Amino” refers to the —NH₂ radical.

“Cyano” or “nitrile” refers to the —CN radical.

“Hydroxy” or “hydroxyl” refers to the —OH radical.

“Nitro” refers to the —NO₂ radical.

“Oxo” refers to the ═O substituent.

“Oxime” refers to the ═N—OH substituent.

“Thioxo” refers to the ═S substituent.

“Alkyl” refers to a linear or branched hydrocarbon chain radical, which is fully saturated, has from one to thirty carbon atoms, and is attached to the rest of the molecule by a single bond. Alkyls are linear or branched. Alkyls comprising any number of carbon atoms from 1 to 30 are included. An alkyl comprising up to 30 carbon atoms is referred to as a C₁-C₃₀ alkyl, likewise, for example, an alkyl comprising up to 12 carbon atoms is a C₁-C₁₂ alkyl. An alkyl comprising up to 6 carbons is a C₁-C₆ alkyl. Alkyls (and other moieties defined herein) comprising other numbers of carbon atoms are represented similarly. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆ alkyl, C₁-C₄alkyl, C₁-C₃ alkyl, C₁-C₂ alkyl, C₂-C₈ alkyl, C₃-C₈ alkyl, C₄-C₈ alkyl, and C₅-C₁₂ alkyl. Representative alkyl groups include, but are not limited to, methyl, ethyl, n-propyl, 1-methylethyl (isopropyl), n-butyl, i-butyl, s-butyl, n-pentyl, 1,1-dimethylethyl (t-butyl), 2-ethylpropyl, and the like. Representative linear alkyl groups include, but are not limited to, methyl, ethyl, n-propyl, n-butyl, n-pentyl and the like. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In certain embodiments, an alkenyl comprises two to six carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, trimethylsilanyl, —OR^{a}, —SR^{a}, —OC(O)—R^{a}, —N(R^{a})₂, —C(O)R^{a}, —C(O)OR^{a}, oximo, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In certain embodiments, an alkynyl comprises two to six carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Aminoalkyl” refers to a radical of the formula —R^{c}—N(R^{a})₂ or —R^{c}—N(R^{a})—R^{c}, where each R^{c} is independently an alkylene chain as defined above, for example, methylene, ethylene, and the like; and each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl.

“Alkoxy” refers to a radical of the formula —OR^{a} where R^{a} is an alkyl radical as defined. Unless stated otherwise specifically in the specification, an alkoxy group may be optionally substituted as described above for alkyl.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted by one or more of the following substituents: alkyl, alkenyl, alkynyl, halo, fluoroalkyl, cyano, nitro, aryl, aralkyl, aralkenyl, aralkynyl, cycloalkyl, cycloalkylalkyl, heterocycloalkyl, heterocycloalkylalkyl, heteroarylalkyl, —R^{b}—OR^{a}, —R^{b}—OC(O)—R^{a}, heteroaryl, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2), and —R^{b}—S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocycloalkyl (optionally substituted with one or more alkyl groups), heterocycloalkylalkyl, heteroaryl or heteroarylalkyl, or two R^{a} attached to the same nitrogen atom are combined to form a heterocycloalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aryloxy” refers to a radical bonded through an oxygen atom of the formula —O-aryl, where aryl is as defined above.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —Re-aryl, where Re is an alkynylene chain as defined above. The aryl part of the aralkynyl radical is optionally substituted as described above for an aryl group. The alkynylene chain part of the aralkynyl radical is optionally substituted as defined above for an alkynylene chain.

“Cycloalkyl” or “carbocycle” refers to a stable, non-aromatic, monocyclic or polycyclic carbocyclic ring, which may include fused (when fused with an aryl or a heteroaryl ring, the cycloalkyl is bonded through a non-aromatic ring atom) or bridged ring systems, which is saturated or unsaturated. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Unless otherwise stated specifically in the specification, the cycloalkyl is optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Cycloalkylalkyl” refers to a radical of the formula —R^{c}-cycloalkyl where R^{c} is an alkylene chain as defined above. The alkylene chain and the cycloalkyl radical are optionally substituted as defined above.

“Fused” refers to any ring structure described herein which is fused to an existing ring structure. When the fused ring is a heretocycloalkyl ring or a heteroaryl ring, any carbon atom on the existing ring structure which becomes part of the fused heretocycloalkyl ring or the fused heteroaryl ring may be replaced with a nitrogen atom.

“Heteroalkyl” refers to a straight or branched hydrocarbon chain alkyl radical containing no unsaturation, having from one to fifteen carbon atoms (e.g., C₁-C₁₅ alkyl) consisting of carbon and hydrogen atoms and one or two heteroatoms selected from O, N, and S, wherein the nitrogen or sulfur atoms may be optionally oxidized and the nitrogen atom may be quaternized. The heteroatom(s) may be placed at any position of the heteroalkyl group including between the rest of the heteroalkyl group and the fragment to which it is attached. The heteroalkyl is attached to the rest of the molecule by a single bond. Unless stated otherwise specifically in the specification, a heteroalkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{f}, —OC(O)—NR^{a}R^{f}, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, and each R^{f} is independently alkyl, fluoroalkyl, cycloalkyl, cycloalkylalkyl, aryl, aralkyl, heterocycloalkyl, heterocycloalkylalkyl, heteroaryl or heteroarylalkyl.

“Halo” or “halogen” refers to bromo, chloro, fluoro or iodo. In some embodiments, halogen refers to chloro or fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” similarly refers to a radical of the formula —OR^{a} where R^{a} is a haloalkyl radical as defined. Unless stated otherwise specifically in the specification, a haloalkoxy group may be optionally substituted as described below.

“Heterocycloalkyl” or “heterocycle” refers to a stable 3- to 24-membered non-aromatic ring radical comprising 2 to 23 carbon atoms and from one to 8 heteroatoms selected from the group consisting of nitrogen, oxygen, phosphorous and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized; and the heterocycloalkyl radical may be partially or fully saturated. Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, 2-oxo-1,3-dioxol-4-yl, 1,1-dioxidotetrahydro-2H-thiopyranyl, tetrahydro-2H-thiopyranyl, and tetrahydro-2H-pyranyl. The term heterocycloalkyl also includes all ring forms of the carbohydrates, including but not limited to the monosaccharides, the disaccharides and the oligosaccharides. Unless otherwise noted, heterocycloalkyls have from 2 to 8 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group is optionally substituted by one or more of the following substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heterocycloalkyllalkyl” refers to a radical of the formula —R^{c}-heterocycloalkyl where R^{c} is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylslkyl radical is optionally substituted as defined above for an alkylene chain. The heterocycloalkyl part of the heterocycloalkylalkyl radical is optionally substituted as defined above for a heterocycloalkyl group.

“Heterocycloalkylalkoxy” refers to a radical bonded through an oxygen atom of the formula —O—R^{c}-heterocycloalkyl where R^{c} is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylalkoxy radical is optionally substituted as defined above for an alkylene chain. The heterocycloalkyl part of the heterocycloalkylalkoxy radical is optionally substituted as defined above for a heterocycloalkyl group.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. In some embodiments, the heteroaryl is a 5-membered heteroaryl. In some embodiments, the heteroaryl is a 6-membered heteroaryl. For purposes of this invention, the heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group is optionally substituted by one or more of the following substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜOR^{a} (where t is 1 or 2), —R^{b}—S(O)ₜR^{a} (where t is 1 or 2) and —R^{b}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroaryloxy” refers to radical bonded through an oxygen atom of the formula —O— heteroaryl, where heteroaryl is as defined above.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

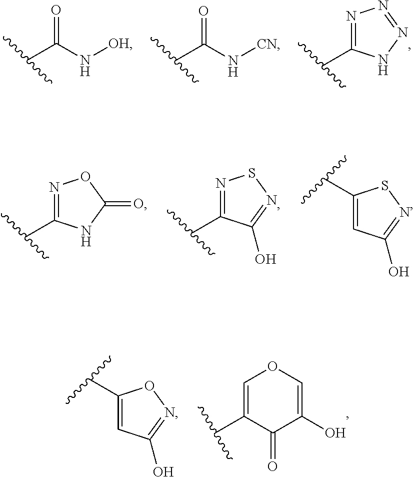

and the like.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

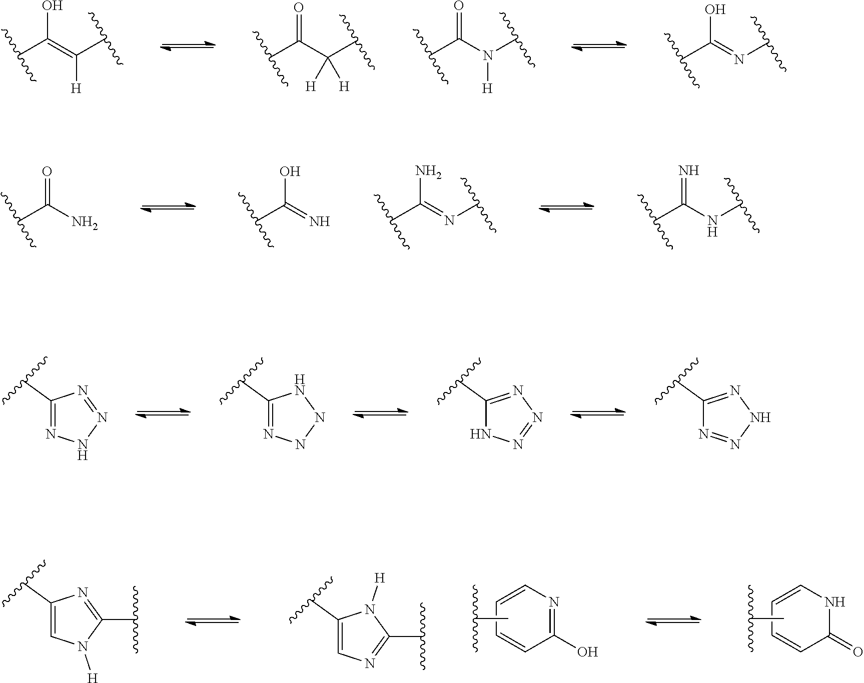

“Optional” or “optionally” means that a subsequently described event or circumstance may or may not occur and that the description includes instances when the event or circumstance occurs and instances in which it does not. For example, “optionally substituted aryl” means that the aryl radical may or may not be substituted and that the description includes both substituted aryl radicals and aryl radicals having no substitution. “Optionally substituted” and “substituted or unsubstituted” and “unsubstituted or substituted” are used interchangeably herein.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and, aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof.

When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range varies between 1% and 15% of the stated number or numerical range.

The term “comprising” (and related terms such as “comprise” or “comprises” or “having” or “including”) is not intended to exclude that which in other certain embodiments, for example, an embodiment of any composition of matter, composition, method, or process, or the like, described herein, “consist of” or “consist essentially of” the described features.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has been made.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and, aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “pharmaceutical combination” as used herein, means a product that results from the mixing or combining of more than one active ingredient and includes both fixed and non-fixed combinations of the active ingredients. The term “fixed combination” means that the active ingredients, are both administered to a patient simultaneously in the form of a single entity or dosage. The term “non-fixed combination” means that the active ingredients, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “co-administration” or the like, as used herein, are meant to encompass administration of the selected therapeutic agents to a single patient, and are intended to include treatment regimens in which the agents are administered by the same or different route of administration or at the same or different time.

The term “activator” is used in this specification to denote any molecular species that results in activation of the indicated receptor, regardless of whether the species itself binds to the receptor or a metabolite of the species binds to the receptor when the species is administered topically. Thus, the activator can be a ligand of the receptor or it can be an activator that is metabolized to the ligand of the receptor, i.e., a metabolite that is formed in tissue and is the actual ligand.

The term “antagonist” as used herein, refers to a small-molecule agent that binds to a nuclear hormone receptor and subsequently decreases the agonist induced transcriptional activity of the nuclear hormone receptor.

The term “agonist” as used herein, refers to a small-molecule agent that binds to a nuclear hormone receptor and subsequently increases nuclear hormone receptor transcriptional activity in the absence of a known agonist.

The term “inverse agonist” as used herein, refers to a small-molecule agent that binds to a nuclear hormone receptor and subsequently decreases the basal level of nuclear hormone receptor transcriptional activity that is present in the absence of a known agonist.

The term “modulate,” as used herein, means to interact with a target protein either directly or indirectly so as to alter the activity of the target protein, including, by way of example only, to inhibit the activity of the target, or to limit or reduce the activity of the target.

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target. Compounds

### FXR Modulators

Described herein are FXR modulatators, and pharmaceutical compositions that include such FXR modulatators, for use in the treatment of diseases, disorders or conditions that would benefit from FXR modulation. In some embodiments is the administration of an FXR modulator described herein to a mammal in the treatment of diseases, disorders or conditions that would benefit from FXR modulation. In some embodiments is the administration of an FXR modulator described herein to a mammal in the treatment of diseases, disorders or conditions that would benefit from FXR modulation, wherein the FXR modulator is (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

In some embodiments is the administration of an FXR modulator described herein to a mammal in the treatment of diseases, disorders or conditions that would benefit from FXR modulation, wherein the FXR modulator is (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof. Compound 1 has the structure:

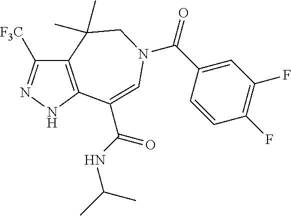 [SMILES: CC(C)NC(=O)C1=CN(C(=O)c2ccc(F)c(F)c2)CC(C)(C)c2c(C(F)(F)F)n[nH]c21]

In some embodiments, a pharmaceutically acceptable salt of Compound 1 is a hydrochloride salt. In further embodiments, the pharmaceutically acceptable salt of Compound 1 is a mono-hydrochloride salt.

In some embodiments is the administration of an FXR modulator described herein to a mammal in the treatment of diseases, disorders or conditions that would benefit from FXR modulation, wherein the FXR modulator is (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof. Compound 2 has the structure:

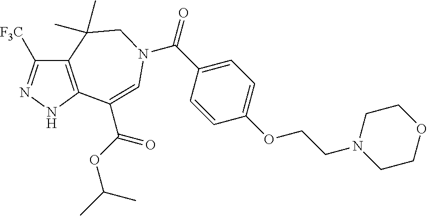 [SMILES: CC(C)OC(=O)C1=CN(C(=O)c2ccc(OCCN3CCOCC3)cc2)CC(C)(C)c2c(C(F)(F)F)n[nH]c21]

In some embodiments, a pharmaceutically acceptable salt of Compound 2 is a hydrochloride salt. In further embodiments, the pharmaceutically acceptable salt of Compound 2 is a mono-hydrochloride salt.

In some embodiments is the administration of an FXR modulator described herein to a mammal in the treatment of diseases, disorders or conditions that would benefit from FXR modulation, wherein the FXR modulator is (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof. Compound 3 has the structure:

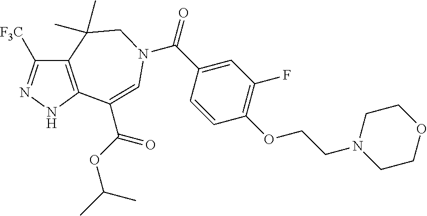 [SMILES: CC(C)OC(=O)C1=CN(C(=O)c2ccc(OCCN3CCOCC3)c(F)c2)CC(C)(C)c2c(C(F)(F)F)n[nH]c21]

In some embodiments, a pharmaceutically acceptable salt of Compound 3 is a hydrochloride salt. In further embodiments, the pharmaceutically acceptable salt of Compound 3 is a mono-hydrochloride salt.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, ²H, ³H, ¹³C, ¹⁴C, ¹⁵N, ¹⁸O, ¹⁷O, ³¹P, ³²P, ³⁵S, ¹⁸F, and ³⁶Cl, sulfur, fluorine and chloride, such as respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. Increased levels of deuterium incorporation produce a detectable kinetic isotope effect (KIE) that may affect the pharmacokinetic, pharmacologic and/or toxicologic parameters of Compound 1, Compound 2, or Compound 3 in comparison to Compound 1, Compound 2, or Compound 3 having naturally occurring levels of deuterium. In some embodiments, the isotopically labeled compound, or a pharmaceutically acceptable salt thereof, is prepared by any suitable method.

In some embodiments, at least one hydrogen in Compound 1 is replaced with deuterium. In some embodiments of the methods described herein, at least one hydrogen in Compound 1 is replaced with deuterium. In some embodiments of the pharmaceutical compositions described herein, at least one hydrogen in Compound 1 is replaced with deuterium.

In some embodiments, at least one hydrogen in Compound 2 is replaced with deuterium. In some embodiments of the methods described herein, at least one hydrogen in Compound 2 is replaced with deuterium. In some embodiments of the pharmaceutical compositions described herein, at least one hydrogen in Compound 2 is replaced with deuterium.

In some embodiments, at least one hydrogen in Compound 3 is replaced with deuterium. In some embodiments of the methods described herein, at least one hydrogen in Compound 3 is replaced with deuterium. In some embodiments of the pharmaceutical compositions described herein, at least one hydrogen in Compound 3 is replaced with deuterium.

In some embodiments, the compounds described herein are labeled by other means, including, but not limited to, the use of chromophores or fluorescent moieties, bioluminescent labels, or chemiluminescent labels.

### Methods of Treatment

In some embodiments is a method of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof; wherein the FXR modulator is a compound of Formula (I), or a pharmaceutically acceptable salt, stereoisomer, or solvate thereof:

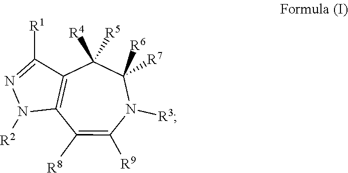

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R² is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of —CN, —C(O)OR²⁵, —C(O)N(R²⁵)R²⁶,

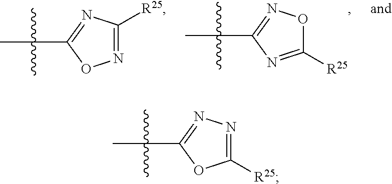

R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R⁸ and R⁹ together with the carbon atoms to which they are attached, form an optionally substituted C₂-C₉heterocycloalkyl ring or an optionally substituted heteroaryl ring;
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl)
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring; or a pharmaceutically acceptable salt, stereoisomer, or solvate thereof.

In some embodiments is a method of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof; wherein the FXR modulator is a compound of Formula (II), or a pharmaceutically acceptable salt, stereoisomer, or solvate thereof:

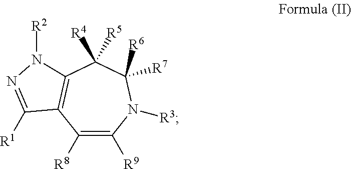

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R² is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of —CN, —C(O)OR²⁵, —C(O)N(R²⁵)R²⁶,

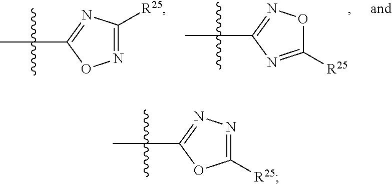

R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R⁸ and R⁹ together with the carbon atoms to which they are attached, form an optionally substituted C₂-C₉heterocycloalkyl ring or an optionally substituted heteroaryl ring;
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring; or a pharmaceutically acceptable salt, stereoisomer, or solvate thereof.

In one embodiment, the FXR modulator is a compound of Formula (I) or (II) wherein R⁴ and R⁵ are each independently selected from the group consisting of hydrogen, halogen, and optionally substituted C₁-C₆alkyl. In another embodiment, the FXR modulator is a compound of Formula (I) or (II) wherein R⁴ and R⁵ are each independently selected from the group consisting of hydrogen and optionally substituted C₁-C₆alkyl. In another embodiment, the FXR modulator is a compound of Formula (I) or (II) wherein R⁴ and R⁵ are each hydrogen. In another embodiment, the FXR modulator is a compound of Formula (I) or (II) wherein R⁴ and R⁵ are each independently optionally substituted C₁-C₆alkyl. In another embodiment, the FXR modulator is a compound of Formula (I) or (II) wherein R⁴ and R⁵ are each methyl.

In another embodiment, the FXR modulator is a compound of Formula (I) or (II) wherein R⁶ and R⁷ are each independently selected from the group consisting of hydrogen, halogen, and optionally substituted C₁-C₆alkyl. In another embodiment, the FXR modulator is a compound of Formula (I) or (II) wherein R⁶ and R⁷ are each independently selected from the group consisting of hydrogen and optionally substituted C₁-C₆alkyl. In another embodiment, the FXR modulator is a compound of Formula (I) or (II) wherein R⁶ and R⁷ are each independently optionally substituted C₁-C₆alkyl. In another embodiment, the FXR modulator is a compound of Formula (I) or (II) wherein R⁶ and R⁷ are each methyl. In another embodiment, the FXR modulator is a compound of Formula (I) or (II) wherein R⁶ and R⁷ are each hydrogen.

In another embodiment, the FXR modulator is a compound of Formula (I) or (II) wherein R⁶ and R⁷ are hydrogen, R⁴ and R⁵ are independently optionally substituted C₁-C₆alkyl, R³ is —C(O)R²⁰, and R²⁰ is optionally substituted aryl. In another embodiment, the FXR modulator is a compound of Formula (I) or (II) wherein R⁶ and R⁷ are hydrogen, R⁴ and R⁵ are independently optionally substituted C₁-C₆alkyl, R³ is —C(O)R²⁰, and R²⁰ is optionally substituted heteroaryl. In another embodiment, the FXR modulator is a compound of Formula (I) or (II) wherein R⁶ and R⁷ are hydrogen, R⁴ and R⁵ are methyl, R³ is —C(O)R²⁰, and R²⁰ is optionally substituted aryl. In another embodiment, the FXR modulator is a compound of Formula (I) or (II) wherein R⁶ and R⁷ are hydrogen, R⁴ and R⁵ are methyl, R³ is —C(O)R²⁰, and R²⁰ is optionally substituted heteroaryl.

In another embodiment, the FXR modulator is a compound of Formula (I) or (II) wherein R⁶ and R⁷ are hydrogen, R⁴ and R⁵ are independently optionally substituted C₁-C₆alkyl, R³ is —S(O)₂R²⁰, and R²⁰ is optionally substituted aryl. In another embodiment, the FXR modulator is a compound of Formula (I) or (II) wherein R⁶ and R⁷ are hydrogen, R⁴ and R⁵ are independently optionally substituted C₁-C₆alkyl, R³ is —S(O)₂R²⁰, and R²⁰ is optionally substituted heteroaryl. In another embodiment, the FXR modulator is a compound of Formula (I) or (II) wherein R⁶ and R⁷ are hydrogen, R⁴ and R⁵ are methyl, R³ is —S(O)₂R²⁰, and R²⁰ is optionally substituted aryl. In another embodiment, the FXR modulator is a compound of Formula (I) or (II) wherein R⁶ and R⁷ are hydrogen, R⁴ and R⁵ are methyl, R³ is —S(O)₂R²⁰, and R²⁰ is optionally substituted heteroaryl.

In another embodiment, the FXR modulator is a compound of Formula (I) or (II) wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted aryl. In another embodiment, the FXR modulator is a compound of Formula (I) or (II) wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted heteroaryl. In another embodiment, the FXR modulator is a compound of Formula (I) or (II) wherein R⁶ and R⁷ are hydrogen, R⁴ and R⁵ are methyl, R³ is —C(O)N(R²¹)R²², R²¹ is hydrogen and R²² is optionally substituted aryl. In another embodiment, the FXR modulator is a compound of Formula (I) or (II) wherein R⁶ and R⁷ are hydrogen, R⁴ and R⁵ are methyl, R³ is —C(O)N(R²¹)R²², R²¹ is hydrogen and R²² is optionally substituted heteroaryl.

In a further embodiment of the aforementioned embodiments is a compound of Formula (I) or (II) wherein R⁸ is selected from the group consisting of —CN, —C(O)OR²⁵, —C(O)N(R²⁵)R²⁶,

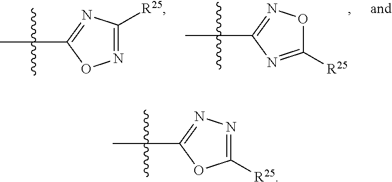

In a further embodiment of the aforementioned embodiments, the FXR modulator is a compound of Formula (I) or (II) wherein R⁸ is —CN.

In a further embodiment of the aforementioned embodiments, the FXR modulator is a compound of Formula (I) or (II) wherein R⁸ is —C(O)OR²⁵. In a further embodiment of the aforementioned embodiments, the FXR modulator is a compound of Formula (I) or (II) wherein R⁸ is —C(O)OR²⁵, and R²⁵ is independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (I) or (II) wherein R⁸ is —C(O)OR²⁵, and R²⁵ is independently selected from the group consisting of hydrogen, and optionally substituted C₁-C₆alkyl. In a further embodiment of the aforementioned embodiments, the FXR modulator is a compound of Formula (I) or (II) wherein R⁸ is —C(O)OR²⁵, and R²⁵ is hydrogen. In a further embodiment of the aforementioned embodiments, the FXR modulator is a compound of Formula (I) or (II) wherein R⁸ is —C(O)OR²⁵, and R²⁵ is optionally substituted C₁-C₆alkyl. In a further embodiment of the aforementioned embodiments, the FXR modulator is a compound of Formula (I) or (II) wherein R⁸ is —C(O)OR²⁵, and R²⁵ is unsubstituted C₁-C₆alkyl. In a further embodiment of the aforementioned embodiments, the FXR modulator is a compound of Formula (I) or (II) wherein R⁸ is —C(O)OR²⁵, and R²⁵ is methyl. In a further embodiment of the aforementioned embodiments, the FXR modulator is a compound of Formula (I) or (II) wherein R⁸ is —C(O)OR²⁵, and R²⁵ is ethyl. In a further embodiment of the aforementioned embodiments, the FXR modulator is a compound of Formula (I) or (II) wherein R⁸ is —C(O)OR²⁵, and R²⁵ is isopropyl.

In a further embodiment of the aforementioned embodiments, the FXR modulator is a compound of Formula (I) or (II) wherein R⁸ is —C(O)N(R²⁵)R²⁶. In a further embodiment of the aforementioned embodiments, the FXR modulator is a compound of Formula (I) or (II) wherein R⁸ is —C(O)N(R²⁵)R²⁶, and R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (I) or (II) wherein R⁸ is —C(O)N(R²⁵)R²⁶, and R²⁵ and R²⁶ are each independently selected from the group consisting of hydrogen, and optionally substituted C₁-C₆alkyl. In a further embodiment of the aforementioned embodiments, the FXR modulator is a compound of Formula (I) or (II) wherein R⁸ is —C(O)N(R²⁵)R²⁶, and R²⁵ and R²⁶ are hydrogen. In a further embodiment of the aforementioned embodiments, the FXR modulator is a compound of Formula (I) or (II) wherein R⁸ is —C(O)N(R²⁵)R²⁶, and R²⁵ and R²⁶ are each independently optionally substituted C₁-C₆alkyl. In a further embodiment of the aforementioned embodiments, the FXR modulator is a compound of Formula (I) or (II) wherein R⁸ is —C(O)N(R²⁵)R²⁶, R²⁵ is hydrogen, and R²⁶ is optionally substituted C₁-C₆alkyl. In a further embodiment of the aforementioned embodiments, the FXR modulator is a compound of Formula (I) or (II) wherein R⁸ is —C(O)N(R²⁵)R²⁶, and R²⁵ and R²⁶ are each independently unsubstituted C₁-C₆alkyl. In a further embodiment of the aforementioned embodiments, the FXR modulator is a compound of Formula (I) or (II) wherein R⁸ is —C(O)N(R²⁵)R²⁶, R²⁵ is hydrogen, and R²⁶ are methyl. In a further embodiment of the aforementioned embodiments, the FXR modulator is a compound of Formula (I) or (II) wherein R⁸ is —C(O)N(R²⁵)R²⁶, and R²⁵ and R²⁶ are methyl. In a further embodiment of the aforementioned embodiments, the FXR modulator is a compound of Formula (I) or (II) wherein R⁸ is —C(O)N(R²⁵)R²⁶, and R²⁵ and R²⁶ are ethyl.

In a further embodiment of the aforementioned embodiments, the FXR modulator is a compound of Formula (I) or (II) wherein R⁸ is

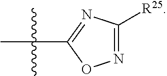

In a further embodiment of the aforementioned embodiments, the FXR modulator is a compound of Formula (I) or (II) wherein R⁸ is

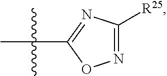

and R²⁵ is optionally substituted C₁-C₆alkyl. In a further embodiment of the aforementioned embodiments, the FXR modulator is a compound of Formula (I) or (II) wherein R⁸ is

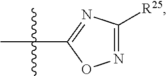

and R²⁵ is methyl. In a further embodiment of the aforementioned embodiments, the FXR modulator is a compound of Formula (I) or (II) wherein R⁸ is

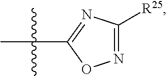

and R²⁵ is ethyl.

In a further embodiment of the aforementioned embodiments, the FXR modulator is a compound of Formula (I) or (II) wherein R⁸ is

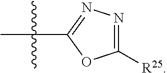

In a further embodiment of the aforementioned embodiments, the FXR modulator is a compound of Formula (I) or (II) wherein R⁸ is

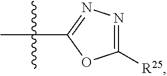

and R²⁵ is optionally substituted C₁-C₆alkyl. In a further embodiment of the aforementioned embodiments, the FXR modulator is a compound of Formula (I) or (II) wherein R⁸ is

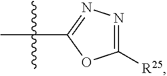

and R²⁵ is methyl. In a further embodiment of the aforementioned embodiments, the FXR modulator is a compound of Formula (I) or (II) wherein R⁸ is

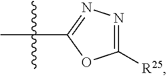

and R²⁵ is ethyl.

In a further embodiment of the aforementioned embodiments, the FXR modulator is a compound of Formula (I) or (II) wherein R⁸ is

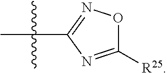

In a further embodiment of the aforementioned embodiments, the FXR modulator is a compound of Formula (I) or (II) wherein R⁸ is

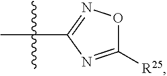

and R²⁵ is optionally substituted C₁-C₆alkyl. In a further embodiment of the aforementioned embodiments, the FXR modulator is a compound of Formula (I) or (II) wherein R⁸ is

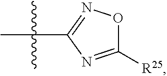

and R²⁵ is methyl. In a further embodiment of the aforementioned embodiments, the FXR modulator is a compound of Formula (I) or (II) wherein R⁸ is

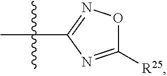

and R²⁵ is ethyl.

In a further embodiment of the aforementioned embodiments, the FXR modulator is a compound of Formula (I) or (II) wherein R⁹ is selected from the group consisting of hydrogen and optionally substituted C₁-C₆alkyl. In a further embodiment of the aforementioned embodiments, the FXR modulator is a compound of Formula (I) or (II) wherein R⁹ is hydrogen. In a further embodiment of the aforementioned embodiments, the FXR modulator is a compound of Formula (I) or (II) wherein R⁹ is optionally substituted C₁-C₆alkyl. In a further embodiment of the aforementioned embodiments, the FXR modulator is a compound of Formula (I) or (II) wherein R⁹ is unsubstituted C₁-C₆alkyl. In a further embodiment of the aforementioned embodiments, the FXR modulator is a compound of Formula (I) or (II) wherein R⁹ is methyl.

In a further embodiment of the aforementioned embodiments, the FXR modulator is a compound of Formula (I) or (II) wherein R⁸ and R⁹ together with the carbon atoms to which they are attached, form an optionally substituted C₂-C₉heterocycloalkyl ring or an optionally substituted heteroaryl ring. In a further embodiment of the aforementioned embodiments, the FXR modulator is a compound of Formula (I) or (II) wherein R⁸ and R⁹ together with the carbon atoms to which they are attached, form an optionally substituted C₂-C₉heterocycloalkyl ring. In a further embodiment of the aforementioned embodiments, the FXR modulator is a compound of Formula (I) or (II) wherein R⁸ and R⁹ together with the carbon atoms to which they are attached, form an optionally substituted heteroaryl ring.

In a further embodiment of the aforementioned embodiments, the FXR modulator is a compound of Formula (I) or (II) wherein R² is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl). In a further embodiment of the aforementioned embodiments, the FXR modulator is a compound of Formula (I) or (II) wherein R² is selected from the group consisting of hydrogen, and optionally substituted C₁-C₆alkyl. In a further embodiment of the aforementioned embodiments, the FXR modulator is a compound of Formula (I) or (II) wherein R² is optionally substituted C₁-C₆alkyl. In a further embodiment of the aforementioned embodiments, the FXR modulator is a compound of Formula (I) or (II) wherein R² is methyl. In a further embodiment of the aforementioned embodiments, the FXR modulator is a compound of Formula (I) or (II) wherein R² is ethyl. In a further embodiment of the aforementioned embodiments, the FXR modulator is a compound of Formula (I) or (II) wherein R² is hydrogen.

In a further embodiment of the aforementioned embodiments, the FXR modulator is a compound of Formula (I) or (II) wherein R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), and —OR¹⁰. In a further embodiment of the aforementioned embodiments, the FXR modulator is a compound of Formula (I) or (II) wherein R¹ is hydrogen. In a further embodiment of the aforementioned embodiments, the FXR modulator is a compound of Formula (I) or (II) wherein R¹ is halogen. In a further embodiment of the aforementioned embodiments, the FXR modulator is a compound of Formula (I) or (II) wherein R¹ is optionally substituted C₁-C₆alkyl. In a further embodiment of the aforementioned embodiments, the FXR modulator is a compound of Formula (I) or (II) wherein R¹ is optionally substituted C₂-C₆alkenyl. In a further embodiment of the aforementioned embodiments, the FXR modulator is a compound of Formula (I) or (II) wherein R¹ is optionally substituted C₂-C₆alkynyl. In a further embodiment of the aforementioned embodiments, the FXR modulator is a compound of Formula (I) or (II) wherein R¹ is —OR¹⁰ and R¹⁰ is hydrogen. In a further embodiment of the aforementioned embodiments, the FXR modulator is a compound of Formula (I) or (II) wherein R¹ is —OR¹⁰ and R¹⁰ is optionally substituted C₁-C₆alkyl. In a further embodiment of the aforementioned embodiments, the FXR modulator is a compound of Formula (I) or (II) wherein R¹ is —OR¹⁰ and R¹⁰ is methyl.

In some embodiments is a method of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof; wherein the FXR modulator is a compound of Formula (III), or a pharmaceutically acceptable salt, stereoisomer, or solvate thereof:

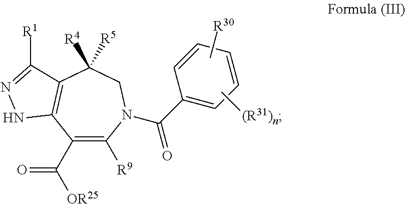

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R⁴ and R⁵ are each independently optionally substituted C₁-C₆alkyl;
R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R²⁵ is C₁-C₆alkyl;
R³⁰ is halogen,

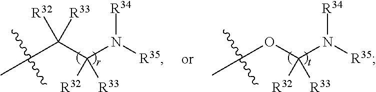

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
n is 0, 1, 2, 3, or 4;
r is 0, 1, 2, 3, or 4;
t is 2, 3, or 4.

In one embodiment, the FXR modulator is a compound of Formula (III) wherein n is 0. In another embodiment, the FXR modulator is a compound of Formula (III) wherein n is 1. In another embodiment, the FXR modulator is a compound of Formula (III) wherein n is 2. In another embodiment, the FXR modulator is a compound of Formula (III) wherein n is 3. In another embodiment, the FXR modulator is a compound of Formula (III) wherein n is 4.

In another embodiment, the FXR modulator is a compound of Formula (III) wherein n is 2 and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (III) wherein n is 2 and each R³¹ is independently halogen, or optionally substituted C₁-C₆alkyl. In another embodiment, the FXR modulator is a compound of Formula (III) wherein n is 2 and each R³¹ is halogen. In another embodiment, the FXR modulator is a compound of Formula (III) wherein n is 2 and each R³¹ is F.

In another embodiment, the FXR modulator is a compound of Formula (III) wherein R³⁰ is F, n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (III) wherein R³⁰ is F, n is 2 and each R³¹ is independently halogen, or optionally substituted C₁-C₆alkyl. In another embodiment, the FXR modulator is a compound of Formula (III) wherein R³⁰ is F, n is 2 and each R³¹ is halogen. In another embodiment, the FXR modulator is a compound of Formula (III) wherein R³⁰ is F, n is 2 and each R³¹ is F.

In another embodiment, the FXR modulator is a compound of Formula (III) wherein n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (III) wherein n is 1 and R³¹ is halogen, or optionally substituted C₁-C₆alkyl. In another embodiment, the FXR modulator is a compound of Formula (III) wherein n is 1 and R³¹ is halogen. In another embodiment, the FXR modulator is a compound of Formula (III) wherein n is 1 and R³¹ is F.

In another embodiment, the FXR modulator is a compound of Formula (III) wherein R³⁰ is F, n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (III) wherein R³⁰ is F, n is 1 and R³¹ is halogen, or optionally substituted C₁-C₆alkyl. In another embodiment, the FXR modulator is a compound of Formula (III) wherein R³⁰ is F, n is 1 and R³¹ is halogen. In another embodiment, the FXR modulator is a compound of Formula (III) wherein R³⁰ is F, n is 1 and R³¹ is F.

In another embodiment, the FXR modulator is a compound of Formula (III) wherein R³⁰ is

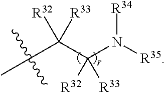

In one embodiment, the FXR modulator is a compound of Formula (III) wherein r is 0. In another embodiment, the FXR modulator is a compound of Formula (III) wherein r is 1. In another embodiment, the FXR modulator is a compound of Formula (III) wherein r is 2. In another embodiment, the FXR modulator is a compound of Formula (III) wherein r is 3. In another embodiment, the FXR modulator is a compound of Formula (III) wherein r is 4. In one embodiment, the FXR modulator is a compound of Formula (III) wherein each R³² and R³³ are hydrogen. In one embodiment, the FXR modulator is a compound of Formula (III) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (III) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (III) wherein R³⁴ and R³⁵ together with the nitrogen atom to which they are attached form an optionally substituted C₂-C₉heterocycloalkyl ring. In another embodiment, the FXR modulator is a compound of Formula (III) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (III) wherein R³⁴ and R³⁵ together with the nitrogen atom to which they are attached form a morpholinyl. In another embodiment, the FXR modulator is a compound of Formula (III) wherein R³⁴ and R³⁵ together with the nitrogen atom to which they are attached form an optionally substituted heteroaryl ring. In another embodiment, the FXR modulator is a compound of Formula (III) wherein R³⁴ and R³⁵ together with the nitrogen atom to which they are attached form an imidazolyl, pyrazolyl, or pyrrolyl.

In another embodiment, the FXR modulator is a compound of Formula (III) wherein R³⁰ is

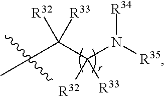

n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (III) wherein R³⁰ is

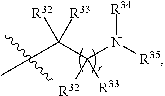

n is 2 and each R³¹ is independently halogen, or optionally substituted C₁-C₆alkyl. In another embodiment, the FXR modulator is a compound of Formula (III) wherein R³⁰ is

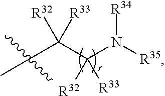

n is 2 and each R³¹ is halogen. In another embodiment, the FXR modulator is a compound of Formula (III) wherein R³⁰ is

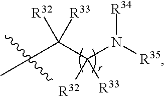

n is 2 and each R³¹ is F.

In another embodiment, the FXR modulator is a compound of Formula (III) wherein R³⁰ is

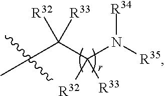

n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (III) wherein R³⁰ is

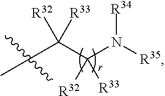

n is 1 and R³¹ is halogen, or optionally substituted C₁-C₆alkyl. In another embodiment, the FXR modulator is a compound of Formula (III) wherein R³⁰ is

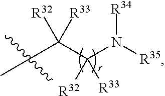

n is 1 and R³¹ is halogen. In another embodiment, the FXR modulator is a compound of Formula (III) wherein R³⁰ is

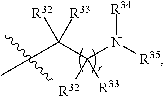

### n is 1 and R³¹ is F.

In another embodiment, the FXR modulator is a compound of Formula (III) wherein R³⁰ is

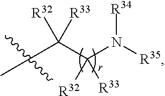

and n is 0.

In another embodiment, the FXR modulator is a compound of Formula (III) wherein R³⁰ is

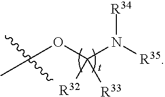

In one embodiment, the FXR modulator is a compound of Formula (III) wherein t is 2. In another embodiment, the FXR modulator is a compound of Formula (III) wherein t is 3. In another embodiment, the FXR modulator is a compound of Formula (III) wherein t is 4. In one embodiment, the FXR modulator is a compound of Formula (III) wherein each R³² and R³³ are hydrogen. In one embodiment, the FXR modulator is a compound of Formula (III) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (III) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (III) wherein R³⁴ and R³⁵ together with the nitrogen atom to which they are attached form an optionally substituted C₂-C₉heterocycloalkyl ring. In another embodiment, the FXR modulator is a compound of Formula (III) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (III) wherein R³⁴ and R³⁵ together with the nitrogen atom to which they are attached form a morpholinyl. In another embodiment, the FXR modulator is a compound of Formula (III) wherein R³⁴ and R³⁵ together with the nitrogen atom to which they are attached form an optionally substituted heteroaryl ring. In another embodiment, the FXR modulator is a compound of Formula (III) wherein R³⁴ and R³⁵ together with the nitrogen atom to which they are attached form an imidazolyl, pyrazolyl, or pyrrolyl.

In another embodiment, the FXR modulator is a compound of Formula (III) wherein R³⁰ is

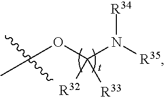

n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (III) wherein R³⁰ is

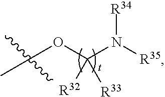

n is 2 and each R³¹ is independently halogen, or optionally substituted C₁-C₆alkyl. In another embodiment, the FXR modulator is a compound of Formula (III) wherein R³⁰ is

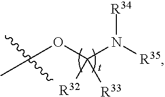

n is 2 and each R³¹ is halogen. In another embodiment, the FXR modulator is a compound of Formula (III) wherein R³⁰ is

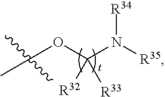

n is 2 and each R³¹ is F.

In another embodiment, the FXR modulator is a compound of Formula (III) wherein R³⁰ is

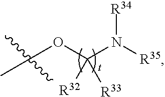

n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (III) wherein R³⁰ is

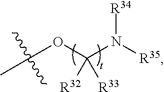

n is 1 and R³¹ is halogen, or optionally substituted C₁-C₆alkyl. In another embodiment, the FXR modulator is a compound of Formula (III) wherein R³⁰ is

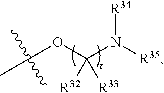

n is 1 and R³¹ is halogen. In another embodiment, the FXR modulator is a compound of Formula (III) wherein R³⁰ is

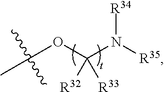

### n is 1 and R³¹ is F.

In another embodiment, the FXR modulator is a compound of Formula (III) wherein R³⁰ is

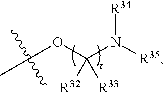

and n is 0.

In a further embodiment of the aforementioned embodiments, the FXR modulator is a compound of Formula (III) wherein R⁴ and R⁵ are each methyl. In a further embodiment of the aforementioned embodiments, the FXR modulator is a compound of Formula (III) wherein R⁴ and R⁵ are each ethyl. In a further embodiment of the aforementioned embodiments, the FXR modulator is a compound of Formula (III) wherein R¹ is hydrogen. In a further embodiment of the aforementioned embodiments, the FXR modulator is a compound of Formula (III) wherein R¹ is halogen. In a further embodiment of the aforementioned embodiments, the FXR modulator is a compound of Formula (III) wherein R¹ is optionally substituted C₁-C₆alkyl. In a further embodiment of the aforementioned embodiments, the FXR modulator is a compound of Formula (III) wherein R¹ is methyl. In a further embodiment of the aforementioned embodiments, the FXR modulator is a compound of Formula (III) wherein R¹ is optionally substituted C₂-C₆alkenyl. In a further embodiment of the aforementioned embodiments, the FXR modulator is a compound of Formula (III) wherein R¹ is optionally substituted C₂-C₆alkynyl. In a further embodiment of the aforementioned embodiments, the FXR modulator is a compound of Formula (III) wherein R¹ is —OR¹⁰ and R¹⁰ is hydrogen. In a further embodiment of the aforementioned embodiments, the FXR modulator is a compound of Formula (III) wherein R¹ is —OR¹⁰ and R¹⁰ is optionally substituted C₁-C₆alkyl. In a further embodiment of the aforementioned embodiments, the FXR modulator is a compound of Formula (III) wherein R¹ is —OR¹⁰ and R¹⁰ is methyl. In a further embodiment of the aforementioned embodiments, the FXR modulator is a compound of Formula (III) wherein R²⁵ is methyl. In a further embodiment of the aforementioned embodiments, the FXR modulator is a compound of Formula (III) wherein R²⁵ is ethyl. In a further embodiment of the aforementioned embodiments, the FXR modulator is a compound of Formula (III) wherein R²⁵ is isopropyl.

In a further embodiment of the aforementioned embodiments, the FXR modulator is a compound of Formula (III) wherein R⁹ is selected from the group consisting of hydrogen and optionally substituted C₁-C₆alkyl. In a further embodiment of the aforementioned embodiments, the FXR modulator is a compound of Formula (III) wherein R⁹ is hydrogen. In a further embodiment of the aforementioned embodiments, the FXR modulator is a compound of Formula (III) wherein R⁹ is optionally substituted C₁-C₆alkyl. In a further embodiment of the aforementioned embodiments, the FXR modulator is a compound of Formula (III) wherein R⁹ is unsubstituted C₁-C₆alkyl. In a further embodiment of the aforementioned embodiments, the FXR modulator is a compound of Formula (III) wherein R⁹ is methyl.

In yet another embodiment, provided herein the FXR modulator is a compound having the structure of Formula (IIIa), or a pharmaceutically acceptable salt, stereoisomer, or solvate thereof:

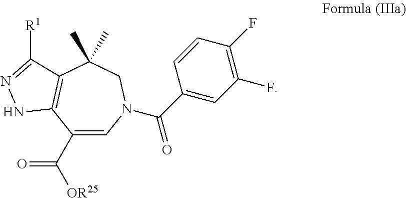

In yet another embodiment, provided herein the FXR modulator is a compound having the structure of Formula (IIIb), or a pharmaceutically acceptable salt, stereoisomer, or solvate thereof:

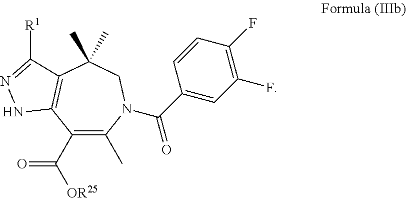

In some embodiments, the FXR modulator is a compound of Formula (IIIa) or (IIIb) wherein R¹ is hydrogen. In some embodiments, the FXR modulator is a compound of Formula (IIIa) or (IIIb) wherein R¹ is halogen. In some embodiments, the FXR modulator is a compound of Formula (IIIa) or (IIIb) wherein R¹ is optionally substituted C₁-C₆alkyl. In some embodiments, the FXR modulator is a compound of Formula (IIIa) or (IIIb) wherein R¹ is optionally substituted C₂-C₆alkenyl. In some embodiments, the FXR modulator is a compound of Formula (IIIa) or (IIIb) wherein R¹ is optionally substituted C₂-C₆alkynyl. In some embodiments, the FXR modulator is a compound of Formula (IIIa) or (IIIb) wherein R¹ is —OR¹⁰ and R¹⁰ is hydrogen. In some embodiments, the FXR modulator is a compound of Formula (IIIa) or (IIIb) wherein R¹ is —OR¹⁰ and R¹⁰ is optionally substituted C₁-C₆alkyl. In some embodiments, the FXR modulator is a compound of Formula (IIIa) or (IIIb) wherein R¹ is —OR¹⁰ and R¹⁰ is methyl. In a further embodiment of the aforementioned embodiments, the FXR modulator is a compound of Formula (IIIa) or (IIIb) wherein R²⁵ is methyl. In a further embodiment of the aforementioned embodiments, the FXR modulator is a compound of Formula (IIIa) or (IIIb) wherein R²⁵ is ethyl. In a further embodiment of the aforementioned embodiments, the FXR modulator is a compound of Formula (IIIa) or (IIIb) wherein R²⁵ is isopropyl.

In some embodiments is a method of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof; wherein the FXR modulator is a compound of Formula (IV), or a pharmaceutically acceptable salt, stereoisomer, or solvate thereof:

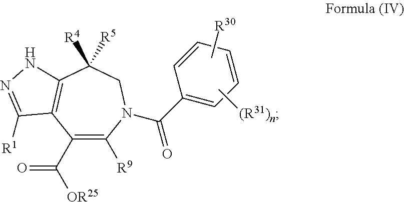

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R⁴ and R⁵ are each independently optionally substituted C₁-C₆alkyl;
R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R²⁵ is C₁-C₆alkyl;
R³⁰ is halogen,

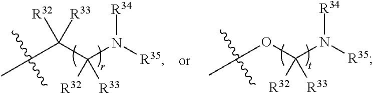

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
n is 0, 1, 2, 3, or 4;
r is 0, 1, 2, 3, or 4;
t is 2, 3, or 4.

In one embodiment, the FXR modulator is a compound of Formula (IV) wherein n is 0. In another embodiment, the FXR modulator is a compound of Formula (IV) wherein n is 1. In another embodiment, the FXR modulator is a compound of Formula (IV) wherein n is 2. In another embodiment, the FXR modulator is a compound of Formula (IV) wherein n is 3. In another embodiment, the FXR modulator is a compound of Formula (IV) wherein n is 4.

In another embodiment, the FXR modulator is a compound of Formula (IV) wherein n is 2 and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (IV) wherein n is 2 and each R³¹ is independently halogen, or optionally substituted C₁-C₆alkyl. In another embodiment, the FXR modulator is a compound of Formula (IV) wherein n is 2 and each R³¹ is halogen. In another embodiment, the FXR modulator is a compound of Formula (IV) wherein n is 2 and each R³¹ is F.

In another embodiment, the FXR modulator is a compound of Formula (IV) wherein R³⁰ is F, n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (IV) wherein R³⁰ is F, n is 2 and each R³¹ is independently halogen, or optionally substituted C₁-C₆alkyl. In another embodiment, the FXR modulator is a compound of Formula (IV) wherein R³⁰ is F, n is 2 and each R³¹ is halogen. In another embodiment, the FXR modulator is a compound of Formula (IV) wherein R³⁰ is F, n is 2 and each R³¹ is F.

In another embodiment, the FXR modulator is a compound of Formula (IV) wherein n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (IV) wherein n is 1 and R³¹ is halogen, or optionally substituted C₁-C₆alkyl. In another embodiment, the FXR modulator is a compound of Formula (IV) wherein n is 1 and R³¹ is halogen. In another embodiment, the FXR modulator is a compound of Formula (IV) wherein n is 1 and R³¹ is F.

In another embodiment, the FXR modulator is a compound of Formula (IV) wherein R³⁰ is F, n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (IV) wherein R³⁰ is F, n is 1 and R³¹ is halogen, or optionally substituted C₁-C₆alkyl. In another embodiment, the FXR modulator is a compound of Formula (IV) wherein R³⁰ is F, n is 1 and R³¹ is halogen. In another embodiment, the FXR modulator is a compound of Formula (IV) wherein R³⁰ is F, n is 1 and R³¹ is F.

In another embodiment, the FXR modulator is a compound of Formula (IV) wherein R³⁰ is

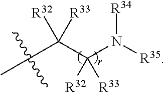

In one embodiment, the FXR modulator is a compound of Formula (IV) wherein r is 0. In another embodiment, the FXR modulator is a compound of Formula (IV) wherein r is 1. In another embodiment, the FXR modulator is a compound of Formula (IV) wherein r is 2. In another embodiment, the FXR modulator is a compound of Formula (IV) wherein r is 3. In another embodiment, the FXR modulator is a compound of Formula (IV) wherein r is 4. In one embodiment, the FXR modulator is a compound of Formula (IV) wherein each R³² and R³³ are hydrogen. In one embodiment, the FXR modulator is a compound of Formula (IV) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (IV) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (IV) wherein R³⁴ and R³⁵ together with the nitrogen atom to which they are attached form an optionally substituted C₂-C₉heterocycloalkyl ring. In another embodiment, the FXR modulator is a compound of Formula (IV) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (IV) wherein R³⁴ and R³⁵ together with the nitrogen atom to which they are attached form a morpholinyl. In another embodiment, the FXR modulator is a compound of Formula (IV) wherein R³⁴ and R³⁵ together with the nitrogen atom to which they are attached form an optionally substituted heteroaryl ring. In another embodiment, the FXR modulator is a compound of Formula (IV) wherein R³⁴ and R³⁵ together with the nitrogen atom to which they are attached form an imidazolyl, pyrazolyl, or pyrrolyl.

In another embodiment, the FXR modulator is a compound of Formula (IV) wherein R³⁰ is

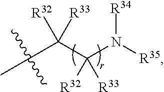

n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (IV) wherein R³⁰ is

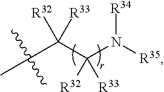

n is 2 and each R³¹ is independently halogen, or optionally substituted C₁-C₆alkyl. In another embodiment, the FXR modulator is a compound of Formula (IV) wherein R³⁰ is

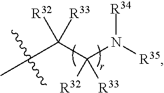

n is 2 and each R³¹ is halogen. In another embodiment FXR modulator is a compound of Formula (IV) wherein R³⁰ is

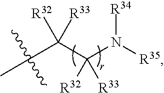

n is 2 and each R³¹ is F.

In another embodiment, the FXR modulator is a compound of Formula (IV) wherein R³⁰ is

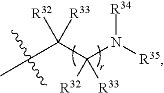

n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (IV) wherein R³⁰ is

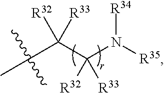

n is 1 and R³¹ is halogen, or optionally substituted C₁-C₆alkyl. In another embodiment, the FXR modulator is a compound of Formula (IV) wherein R³⁰ is

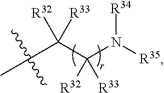

n is 1 and R³¹ is halogen. In another embodiment, the FXR modulator is a compound of Formula (IV) wherein R³⁰ is

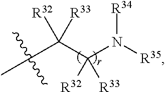

### n is 1 and R³¹ is F.

In another embodiment, the FXR modulator is a compound of Formula (IV) wherein R³⁰ is

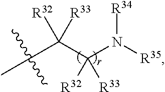

and n is 0.

In another embodiment, the FXR modulator is a compound of Formula (IV) wherein R³⁰ is

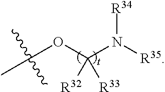

In one embodiment, the FXR modulator is a compound of Formula (IV) wherein t is 2. In another embodiment, the FXR modulator is a compound of Formula (IV) wherein t is 3. In another embodiment, the FXR modulator is a compound of Formula (IV) wherein t is 4. In one embodiment, the FXR modulator is a compound of Formula (IV) wherein each R³² and R³³ are hydrogen. In one embodiment, the FXR modulator is a compound of Formula (IV) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (IV) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (IV) wherein R³⁴ and R³⁵ together with the nitrogen atom to which they are attached form an optionally substituted C₂-C₉heterocycloalkyl ring. In another embodiment, the FXR modulator is a compound of Formula (IV) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (IV) wherein R³⁴ and R³⁵ together with the nitrogen atom to which they are attached form a morpholinyl. In another embodiment, the FXR modulator is a compound of Formula (IV) wherein R³⁴ and R³⁵ together with the nitrogen atom to which they are attached form an optionally substituted heteroaryl ring. In another embodiment, the FXR modulator is a compound of Formula (IV) wherein R³⁴ and R³⁵ together with the nitrogen atom to which they are attached form an imidazolyl, pyrazolyl, or pyrrolyl.

In another embodiment, the FXR modulator is a compound of Formula (IV) wherein R³⁰ is

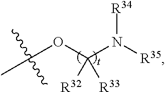

n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (IV) wherein R³⁰ is

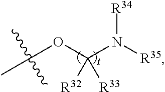

n is 2 and each R³¹ is independently halogen, or optionally substituted C₁-C₆alkyl. In another embodiment, the FXR modulator is a compound of Formula (IV) wherein R³⁰ is

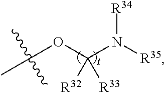

n is 2 and each R³¹ is halogen. In another embodiment, the FXR modulator is a compound of Formula (IV) wherein R³⁰ is

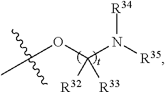

n is 2 and each R³¹ is F.

In another embodiment, the FXR modulator is a compound of Formula (IV) wherein R³⁰ is

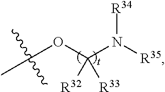

n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (IV) wherein R³⁰ is

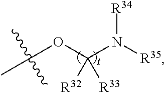

n is 1 and R³¹ is halogen, or optionally substituted C₁-C₆alkyl. In another embodiment, the FXR modulator is a compound of Formula (IV) wherein R³⁰ is

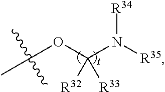

n is 1 and R³¹ is halogen. In another embodiment, the FXR modulator is a compound of Formula (IV) wherein R³⁰ is

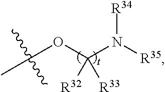

### n is 1 and R³¹ is F.

In another embodiment, the FXR modulator is a compound of Formula (IV) wherein R³⁰ is

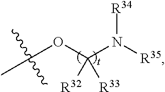

and n is 0.

In a further embodiment of the aforementioned embodiments, the FXR modulator is a compound of Formula (IV) wherein R⁴ and R⁵ are each methyl. In a further embodiment of the aforementioned embodiments, the FXR modulator is a compound of Formula (IV) wherein R⁴ and R⁵ are each ethyl. In a further embodiment of the aforementioned embodiments, the FXR modulator is a compound of Formula (IV) wherein R¹ is hydrogen. In a further embodiment of the aforementioned embodiments, the FXR modulator is a compound of Formula (IV) wherein R¹ is halogen. In a further embodiment of the aforementioned embodiments, the FXR modulator is a compound of Formula (IV) wherein R¹ is optionally substituted C₁-C₆alkyl. In a further embodiment of the aforementioned embodiments, the FXR modulator is a compound of Formula (IV) wherein R¹ is methyl. In a further embodiment of the aforementioned embodiments, the FXR modulator is a compound of Formula (IV) wherein R¹ is optionally substituted C₂-C₆alkenyl. In a further embodiment of the aforementioned embodiments, the FXR modulator is a compound of Formula (IV) wherein R¹ is optionally substituted C₂-C₆alkynyl. In a further embodiment of the aforementioned embodiments, the FXR modulator is a compound of Formula (IV) wherein R¹ is —OR¹⁰ and R¹⁰ is hydrogen. In a further embodiment of the aforementioned embodiments, the FXR modulator is a compound of Formula (IV) wherein R¹ is —OR¹⁰ and R¹⁰ is optionally substituted C₁-C₆alkyl. In a further embodiment of the aforementioned embodiments, the FXR modulator is a compound of Formula (IV) wherein R¹ is —OR¹⁰ and R¹⁰ is methyl. In a further embodiment of the aforementioned embodiments, the FXR modulator is a compound of Formula (IV) wherein R²⁵ is methyl. In a further embodiment of the aforementioned embodiments, the FXR modulator is a compound of Formula (IV) wherein R²⁵ is ethyl. In a further embodiment of the aforementioned embodiments, the FXR modulator is a compound of Formula (IV) wherein R²⁵ is isopropyl.

In a further embodiment of the aforementioned embodiments, the FXR modulator is a compound of Formula (IV) wherein R⁹ is selected from the group consisting of hydrogen and optionally substituted C₁-C₆alkyl. In a further embodiment of the aforementioned embodiments, the FXR modulator is a compound of Formula (IV) wherein R⁹ is hydrogen. In a further embodiment of the aforementioned embodiments, the FXR modulator is a compound of Formula (IV) wherein R⁹ is optionally substituted C₁-C₆alkyl. In a further embodiment of the aforementioned embodiments, the FXR modulator is a compound of Formula (IV) wherein R⁹ is unsubstituted C₁-C₆alkyl. In a further embodiment of the aforementioned embodiments, the FXR modulator is a compound of Formula (IV) wherein R⁹ is methyl.

In yet another embodiment, provided herein the FXR modulator is a compound having the structure of Formula (IVa), or a pharmaceutically acceptable salt, stereoisomer, or solvate thereof:

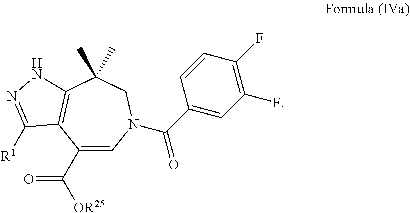

In yet another embodiment, provided herein the FXR modulator is a compound having the structure of Formula (IVb), or a pharmaceutically acceptable salt, stereoisomer, or solvate thereof:

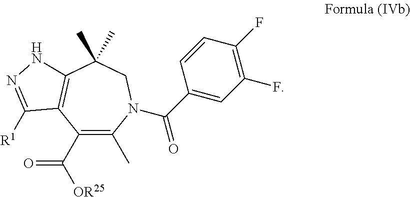

In some embodiments, the FXR modulator is a compound of Formula (IVa) or (IVb) wherein R¹ is hydrogen. In some embodiments, the FXR modulator is a compound of Formula (IVa) or (IVb) wherein R¹ is halogen. In some embodiments, the FXR modulator is a compound of Formula (IVa) or (IVb) wherein R¹ is optionally substituted C₁-C₆alkyl. In some embodiments, the FXR modulator is a compound of Formula (IVa) or (IVb) wherein R¹ is optionally substituted C₂-C₆alkenyl. In some embodiments, the FXR modulator is a compound of Formula (IVa) or (IVb) wherein R¹ is optionally substituted C₂-C₆alkynyl. In some embodiments, the FXR modulator is a compound of Formula (IVa) or (IVb) wherein R¹ is —OR¹⁰ and R¹⁰ is hydrogen. In some embodiments, the FXR modulator is a compound of Formula (IVa) or (IVb) wherein R¹ is —OR¹⁰ and R¹⁰ is optionally substituted C₁-C₆alkyl. In some embodiments, the FXR modulator is a compound of Formula (IVa) or (IVb) wherein R¹ is —OR¹⁰ and R¹⁰ is methyl. In a further embodiment of the aforementioned embodiments, the FXR modulator is a compound of Formula (IVa) or (IVb) wherein R²⁵ is methyl. In a further embodiment of the aforementioned embodiments, the FXR modulator is a compound of Formula (IVa) or (IVb) wherein R²⁵ is ethyl. In a further embodiment of the aforementioned embodiments, the FXR modulator is a compound of Formula (IVa) or (IVb) wherein R²⁵ is isopropyl.

In some embodiments is a method of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof; wherein the FXR modulator is a compound of Formula (V), or a pharmaceutically acceptable salt, stereoisomer, or solvate thereof:

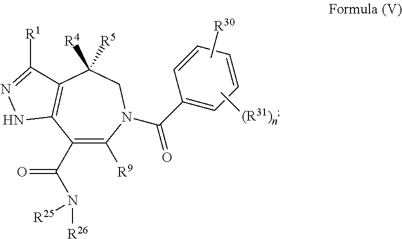

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R⁴ and R⁵ are each independently optionally substituted C₁-C₆alkyl;
R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R²⁵ and R²⁶ are each independently selected from the group consisting of hydrogen, and optionally substituted C₁-C₆alkyl;
R³⁰ is halogen,

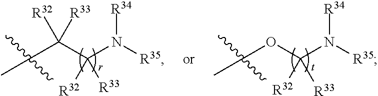

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
n is 0, 1, 2, 3, or 4;
r is 0, 1, 2, 3, or 4;
t is 2, 3, or 4.

In one embodiment, the FXR modulator is a compound of Formula (V) wherein n is 0. In another embodiment, the FXR modulator is a compound of Formula (V) wherein n is 1. In another embodiment, the FXR modulator is a compound of Formula (V) wherein n is 2. In another embodiment, the FXR modulator is a compound of Formula (V) wherein n is 3. In another embodiment, the FXR modulator is a compound of Formula (V) wherein n is 4.

In another embodiment, the FXR modulator is a compound of Formula (V) wherein n is 2 and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (V) wherein n is 2 and each R³¹ is independently halogen, or optionally substituted C₁-C₆alkyl. In another embodiment, the FXR modulator is a compound of Formula (V) wherein n is 2 and each R³¹ is halogen. In another embodiment, the FXR modulator is a compound of Formula (V) wherein n is 2 and each R³¹ is F.

In another embodiment, the FXR modulator is a compound of Formula (V) wherein R³⁰ is F, n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (V) wherein R³⁰ is F, n is 2 and each R³¹ is independently halogen, or optionally substituted C₁-C₆alkyl. In another embodiment, the FXR modulator is a compound of Formula (V) wherein R³⁰ is F, n is 2 and each R³¹ is halogen. In another embodiment, the FXR modulator is a compound of Formula (V) wherein R³⁰ is F, n is 2 and each R³¹ is F.

In another embodiment, the FXR modulator is a compound of Formula (V) wherein n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (V) wherein n is 1 and R³¹ is halogen, or optionally substituted C₁-C₆alkyl. In another embodiment, the FXR modulator is a compound of Formula (V) wherein n is 1 and R³¹ is halogen. In another embodiment, the FXR modulator is a compound of Formula (V) wherein n is 1 and R³¹ is F.

In another embodiment, the FXR modulator is a compound of Formula (V) wherein R³⁰ is F, n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (V) wherein R³⁰ is F, n is 1 and R³¹ is halogen, or optionally substituted C₁-C₆alkyl. In another embodiment, the FXR modulator is a compound of Formula (V) wherein R³⁰ is F, n is 1 and R³¹ is halogen. In another embodiment, the FXR modulator is a compound of Formula (V) wherein R³⁰ is F, n is 1 and R³¹ is F.

In another embodiment, the FXR modulator is a compound of Formula (V) wherein R³⁰ is

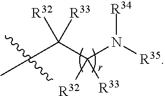

In one embodiment, the FXR modulator is a compound of Formula (V) wherein r is 0. In another embodiment, the FXR modulator is a compound of Formula (V) wherein r is 1. In another embodiment, the FXR modulator is a compound of Formula (V) wherein r is 2. In another embodiment, the FXR modulator is a compound of Formula (V) wherein r is 3. In another embodiment, the FXR modulator is a compound of Formula (V) wherein r is 4. In one embodiment, the FXR modulator is a compound of Formula (V) wherein each R³² and R³³ are hydrogen. In one embodiment, the FXR modulator is a compound of Formula (V) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (V) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (V) wherein R³⁴ and R³⁵ together with the nitrogen atom to which they are attached form an optionally substituted C₂-C₉heterocycloalkyl ring. In another embodiment, the FXR modulator is a compound of Formula (V) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (V) wherein R³⁴ and R³⁵ together with the nitrogen atom to which they are attached form a morpholinyl. In another embodiment, the FXR modulator is a compound of Formula (V) wherein R³⁴ and R³⁵ together with the nitrogen atom to which they are attached form an optionally substituted heteroaryl ring. In another embodiment, the FXR modulator is a compound of Formula (V) wherein R³⁴ and R³⁵ together with the nitrogen atom to which they are attached form an imidazolyl, pyrazolyl, or pyrrolyl.

In another embodiment, the FXR modulator is a compound of Formula (V) wherein R³⁰ is

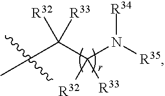

n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (V) wherein R³⁰ is

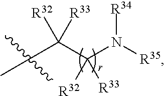

n is 2 and each R³¹ is independently halogen, or optionally substituted C₁-C₆alkyl. In another embodiment, the FXR modulator is a compound of Formula (V) wherein R³⁰ is

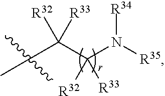

n is 2 and each R³¹ is halogen. In another embodiment, the FXR modulator is a compound of Formula (V) wherein R³⁰ is

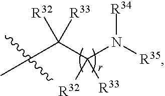

n is 2 and each R³¹ is F.

In another embodiment, the FXR modulator is a compound of Formula (V) wherein R³⁰ is

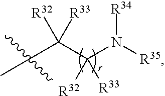

n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (V) wherein R³⁰ is

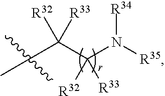

n is 1 and R³¹ is halogen, or optionally substituted C₁-C₆alkyl. In another embodiment, the FXR modulator is a compound of Formula (V) wherein R³⁰ is

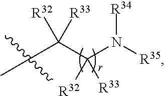

n is 1 and R³¹ is halogen. In another embodiment, the FXR modulator is a compound of Formula (V) wherein R³⁰ is

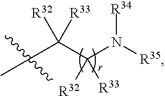

### n is 1 and R³¹ is F.

In another embodiment, the FXR modulator is a compound of Formula (V) wherein R³⁰ is

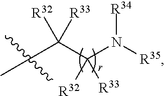

and n is 0.

In another embodiment, the FXR modulator is a compound of Formula (V) wherein R³⁰ is

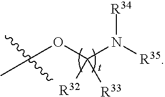

In one embodiment, the FXR modulator is a compound of Formula (V) wherein t is 2. In another embodiment, the FXR modulator is a compound of Formula (V) wherein t is 3. In another embodiment, the FXR modulator is a compound of Formula (V) wherein t is 4. In one embodiment, the FXR modulator is a compound of Formula (V) wherein each R³² and R³³ are hydrogen. In one embodiment, the FXR modulator is a compound of Formula (V) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (V) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (V) wherein R³⁴ and R³⁵ together with the nitrogen atom to which they are attached form an optionally substituted C₂-C₉heterocycloalkyl ring. In another embodiment, the FXR modulator is a compound of Formula (V) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (V) wherein R³⁴ and R³⁵ together with the nitrogen atom to which they are attached form a morpholinyl. In another embodiment, the FXR modulator is a compound of Formula (V) wherein R³⁴ and R³⁵ together with the nitrogen atom to which they are attached form an optionally substituted heteroaryl ring. In another embodiment, the FXR modulator is a compound of Formula (V) wherein R³⁴ and R³⁵ together with the nitrogen atom to which they are attached form an imidazolyl, pyrazolyl, or pyrrolyl.

In another embodiment, the FXR modulator is a compound of Formula (V) wherein R³⁰ is

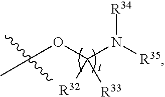

n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (V) wherein R³⁰ is

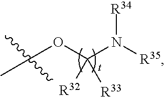

n is 2 and each R³¹ is independently halogen, or optionally substituted C₁-C₆alkyl. In another embodiment, the FXR modulator is a compound of Formula (V) wherein R³⁰ is

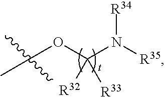

n is 2 and each R³¹ is halogen. In another embodiment, the FXR modulator is a compound of Formula (V) wherein R³⁰ is

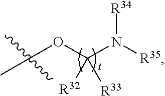

n is 2 and each R³¹ is F.

In another embodiment, the FXR modulator is a compound of Formula (V) wherein R³⁰ is

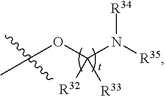

n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (V) wherein R³⁰ is

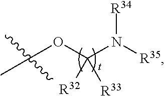

n is 1 and R³¹ is halogen, or optionally substituted C₁-C₆alkyl. In another embodiment, the FXR modulator is a compound of Formula (V) wherein R³⁰ is

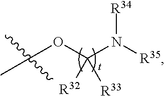

n is 1 and R³¹ is halogen. In another embodiment, the FXR modulator is a compound of Formula (V) wherein R³⁰ is

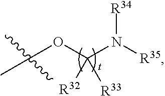

### n is 1 and R³¹ is F.

In another embodiment, the FXR modulator is a compound of Formula (V) wherein R³⁰ is

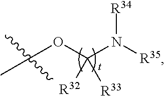

and n is 0.

In a further embodiment of the aforementioned embodiments, the FXR modulator is a compound of Formula (V) wherein R⁴ and R⁵ are each methyl. In a further embodiment of the aforementioned embodiments, the FXR modulator is a compound of Formula (V) wherein R⁴ and R⁵ are each ethyl. In a further embodiment of the aforementioned embodiments, the FXR modulator is a compound of Formula (V) wherein R¹ is hydrogen. In a further embodiment of the aforementioned embodiments, the FXR modulator is a compound of Formula (V) wherein R¹ is halogen. In a further embodiment of the aforementioned embodiments, the FXR modulator is a compound of Formula (V) wherein R¹ is optionally substituted C₁-C₆alkyl. In a further embodiment of the aforementioned embodiments, the FXR modulator is a compound of Formula (V) wherein R¹ is methyl. In a further embodiment of the aforementioned embodiments, the FXR modulator is a compound of Formula (V) wherein R¹ is optionally substituted C₂-C₆alkenyl. In a further embodiment of the aforementioned embodiments, the FXR modulator is a compound of Formula (V) wherein R¹ is optionally substituted C₂-C₆alkynyl. In a further embodiment of the aforementioned embodiments, the FXR modulator is a compound of Formula (V) wherein R¹ is —OR¹⁰ and R¹⁰ is hydrogen. In a further embodiment of the aforementioned embodiments, the FXR modulator is a compound of Formula (V) wherein R¹ is —OR¹⁰ and R¹⁰ is optionally substituted C₁-C₆alkyl. In a further embodiment of the aforementioned embodiments, the FXR modulator is a compound of Formula (V) wherein R¹ is —OR¹⁰ and R¹⁰ is methyl. In a further embodiment of the aforementioned embodiments, the FXR modulator is a compound of Formula (V) wherein R²⁵ and R²⁶ are hydrogen. In a further embodiment of the aforementioned embodiments, the FXR modulator is a compound of Formula (V) wherein R²⁵ is hydrogen and R²⁶ is optionally substituted C₁-C₆alkyl. In a further embodiment of the aforementioned embodiments, the FXR modulator is a compound of Formula (V) wherein R²⁵ is hydrogen and R²⁶ is methyl. In a further embodiment of the aforementioned embodiments, the FXR modulator is a compound of Formula (V) wherein R²⁵ is hydrogen and R²⁶ is ethyl. In a further embodiment of the aforementioned embodiments, the FXR modulator is a compound of Formula (V) wherein R²⁵ is hydrogen and R²⁶ is isopropyl. In a further embodiment of the aforementioned embodiments, the FXR modulator is a compound of Formula (V) wherein R²⁵ and R²⁶ are each optionally substituted C₁-C₆alkyl. In a further embodiment of the aforementioned embodiments, the FXR modulator is a compound of Formula (V) wherein R²⁵ and R²⁶ are methyl. In a further embodiment of the aforementioned embodiments, the FXR modulator is a compound of Formula (V) wherein R²⁵ and R²⁶ are ethyl.

In a further embodiment of the aforementioned embodiments, the FXR modulator is a compound of Formula (V) wherein R⁹ is selected from the group consisting of hydrogen and optionally substituted C₁-C₆alkyl. In a further embodiment of the aforementioned embodiments, the FXR modulator is a compound of Formula (V) wherein R⁹ is hydrogen. In a further embodiment of the aforementioned embodiments, the FXR modulator is a compound of Formula (V) wherein R⁹ is optionally substituted C₁-C₆alkyl. In a further embodiment of the aforementioned embodiments, the FXR modulator is a compound of Formula (V) wherein R⁹ is unsubstituted C₁-C₆alkyl. In a further embodiment of the aforementioned embodiments, the FXR modulator is a compound of Formula (V) wherein R⁹ is methyl.

In yet another embodiment, provided herein the FXR modulator is a compound having the structure of Formula (Va), or a pharmaceutically acceptable salt, stereoisomer, or solvate thereof:

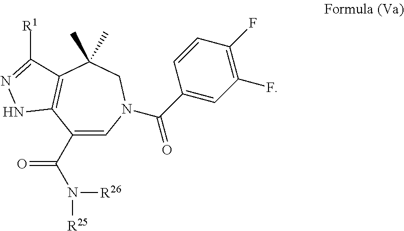

In yet another embodiment, provided herein the FXR modulator is a compound having the structure of Formula (Vb), or a pharmaceutically acceptable salt, stereoisomer, or solvate thereof:

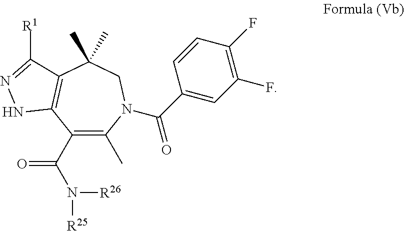

In some embodiments, the FXR modulator is a compound of Formula (Va) or (Vb) wherein R¹ is hydrogen. In some embodiments, the FXR modulator is a compound of Formula (Va) or (Vb) wherein R¹ is halogen. In some embodiments, the FXR modulator is a compound of Formula (Va) or (Vb) wherein R¹ is optionally substituted C₁-C₆alkyl. In some embodiments, the FXR modulator is a compound of Formula (Va) or (Vb) wherein R¹ is optionally substituted C₂-C₆alkenyl. In some embodiments, the FXR modulator is a compound of Formula (Va) or (Vb) wherein R¹ is optionally substituted C₂-C₆alkynyl. In some embodiments, the FXR modulator is a compound of Formula (Va) or (Vb) wherein R¹ is —OR¹⁰ and R¹⁰ is hydrogen. In some embodiments, the FXR modulator is a compound of Formula (Va) or (Vb) wherein R¹ is —OR¹⁰ and R¹⁰ is optionally substituted C₁-C₆alkyl. In some embodiments, the FXR modulator is a compound of Formula (Va) or (Vb) wherein R¹ is —OR¹⁰ and R¹⁰ is methyl. In a further embodiment of the aforementioned embodiments, the FXR modulator is a compound of Formula (Va) or (Vb) wherein R²⁵ and R²⁶ are hydrogen. In a further embodiment of the aforementioned embodiments, the FXR modulator is a compound of Formula (Va) or (Vb) wherein R²⁵ is hydrogen and R²⁶ is optionally substituted C₁-C₆alkyl. In a further embodiment of the aforementioned embodiments, the FXR modulator is a compound of Formula (Va) or (Vb) wherein R²⁵ is hydrogen and R²⁶ is methyl. In a further embodiment of the aforementioned embodiments, the FXR modulator is a compound of Formula (Va) or (Vb) wherein R²⁵ is hydrogen and R²⁶ is ethyl. In a further embodiment of the aforementioned embodiments, the FXR modulator is a compound of Formula (Va) or (Vb) wherein R²⁵ is hydrogen and R²⁶ is isopropyl. In a further embodiment of the aforementioned embodiments, the FXR modulator is a compound of Formula (Va) or (Vb) wherein R²⁵ and R²⁶ are each optionally substituted C₁-C₆alkyl. In a further embodiment of the aforementioned embodiments, the FXR modulator is a compound of Formula (Va) or (Vb) wherein R²⁵ and R²⁶ are methyl. In a further embodiment of the aforementioned embodiments, the FXR modulator is a compound of Formula (Va) or (Vb) wherein R²⁵ and R²⁶ are ethyl.

In some embodiments is a method of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof; wherein the FXR modulator is a compound of Formula (VI), or a pharmaceutically acceptable salt, stereoisomer, or solvate thereof:

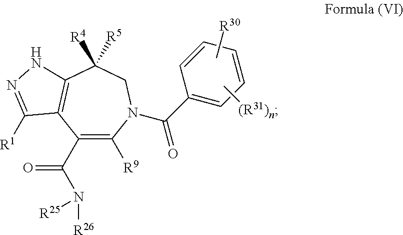

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R⁴ and R⁵ are each independently optionally substituted C₁-C₆alkyl;
R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R²⁵ and R²⁶ are each independently selected from the group consisting of hydrogen, and optionally substituted C₁-C₆alkyl;
R³⁰ is halogen,

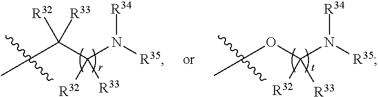

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
n is 0, 1, 2, 3, or 4;
r is 0, 1, 2, 3, or 4;
t is 2, 3, or 4.

In one embodiment, the FXR modulator is a compound of Formula (VI) wherein n is 0. In another embodiment, the FXR modulator is a compound of Formula (VI) wherein n is 1. In another embodiment, the FXR modulator is a compound of Formula (VI) wherein n is 2. In another embodiment, the FXR modulator is a compound of Formula (VI) wherein n is 3. In another embodiment, the FXR modulator is a compound of Formula (VI) wherein n is 4.

In another embodiment, the FXR modulator is a compound of Formula (VI) wherein n is 2 and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VI) wherein n is 2 and each R³¹ is independently halogen, or optionally substituted C₁-C₆alkyl. In another embodiment, the FXR modulator is a compound of Formula (VI) wherein n is 2 and each R³¹ is halogen. In another embodiment, the FXR modulator is a compound of Formula (VI) wherein n is 2 and each R³¹ is F.

In another embodiment, the FXR modulator is a compound of Formula (VI) wherein R³⁰ is F, n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VI) wherein R³⁰ is F, n is 2 and each R³¹ is independently halogen, or optionally substituted C₁-C₆alkyl. In another embodiment, the FXR modulator is a compound of Formula (VI) wherein R³⁰ is F, n is 2 and each R³¹ is halogen. In another embodiment, the FXR modulator is a compound of Formula (VI) wherein R³⁰ is F, n is 2 and each R³¹ is F.

In another embodiment, the FXR modulator is a compound of Formula (VI) wherein n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VI) wherein n is 1 and R³¹ is halogen, or optionally substituted C₁-C₆alkyl. In another embodiment, the FXR modulator is a compound of Formula (VI) wherein n is 1 and R³¹ is halogen. In another embodiment, the FXR modulator is a compound of Formula (VI) wherein n is 1 and R³¹ is F.

In another embodiment, the FXR modulator is a compound of Formula (VI) wherein R³⁰ is F, n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VI) wherein R³⁰ is F, n is 1 and R³¹ is halogen, or optionally substituted C₁-C₆alkyl. In another embodiment, the FXR modulator is a compound of Formula (VI) wherein R³⁰ is F, n is 1 and R³¹ is halogen. In another embodiment, the FXR modulator is a compound of Formula (VI) wherein R³⁰ is F, n is 1 and R³¹ is F.

In another embodiment, the FXR modulator is a compound of Formula (VI) wherein R³⁰ is

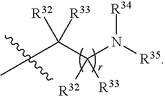

In one embodiment, the FXR modulator is a compound of Formula (VI) wherein r is 0. In another embodiment, the FXR modulator is a compound of Formula (VI) wherein r is 1. In another embodiment, the FXR modulator is a compound of Formula (VI) wherein r is 2. In another embodiment, the FXR modulator is a compound of Formula (VI) wherein r is 3. In another embodiment, the FXR modulator is a compound of Formula (VI) wherein r is 4. In one embodiment, the FXR modulator is a compound of Formula (VI) wherein each R³² and R³³ are hydrogen. In one embodiment, the FXR modulator is a compound of Formula (VI) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (VI) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (VI) wherein R³⁴ and R³⁵ together with the nitrogen atom to which they are attached form an optionally substituted C₂-C₉heterocycloalkyl ring. In another embodiment, the FXR modulator is a compound of Formula (VI) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (VI) wherein R³⁴ and R³⁵ together with the nitrogen atom to which they are attached form a morpholinyl. In another embodiment, the FXR modulator is a compound of Formula (VI) wherein R³⁴ and R³⁵ together with the nitrogen atom to which they are attached form an optionally substituted heteroaryl ring. In another embodiment, the FXR modulator is a compound of Formula (VI) wherein R³⁴ and R³⁵ together with the nitrogen atom to which they are attached form an imidazolyl, pyrazolyl, or pyrrolyl.

In another embodiment, the FXR modulator is a compound of Formula (VI) wherein R³⁰ is

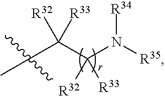

n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VI) wherein R³⁰ is

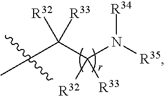

n is 2 and each R³¹ is independently halogen, or optionally substituted C₁-C₆alkyl. In another embodiment, the FXR modulator is a compound of Formula (VI) wherein R³⁰ is

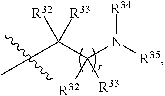

n is 2 and each R³¹ is halogen. In another embodiment, the FXR modulator is a compound of Formula (VI) wherein R³⁰ is

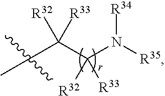

n is 2 and each R³¹ is F.

In another embodiment, the FXR modulator is a compound of Formula (VI) wherein R³⁰ is

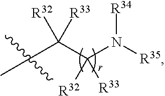

n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VI) wherein R³⁰ is

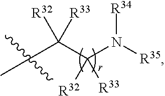

n is 1 and R³¹ is halogen, or optionally substituted C₁-C₆alkyl. In another embodiment, the FXR modulator is a compound of Formula (VI) wherein R³⁰ is

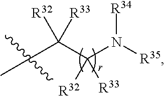

n is 1 and R³¹ is halogen. In another embodiment, the FXR modulator is a compound of Formula (VI) wherein R³⁰ is

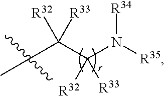

### n is 1 and R³¹ is F.

In another embodiment, the FXR modulator is a compound of Formula (VI) wherein R³⁰ is

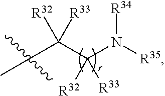

and n is 0.

In another embodiment, the FXR modulator is a compound of Formula (VI) wherein R³⁰ is

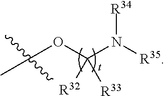

In one embodiment, the FXR modulator is a compound of Formula (VI) wherein t is 2. In another embodiment, the FXR modulator is a compound of Formula (VI) wherein t is 3. In another embodiment, the FXR modulator is a compound of Formula (VI) wherein t is 4. In one embodiment, the FXR modulator is a compound of Formula (VI) wherein each R³² and R³³ are hydrogen. In one embodiment, the FXR modulator is a compound of Formula (VI) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (VI) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (VI) wherein R³⁴ and R³⁵ together with the nitrogen atom to which they are attached form an optionally substituted C₂-C₉heterocycloalkyl ring. In another embodiment, the FXR modulator is a compound of Formula (VI) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (VI) wherein R³⁴ and R³⁵ together with the nitrogen atom to which they are attached form a morpholinyl. In another embodiment, the FXR modulator is a compound of Formula (VI) wherein R³⁴ and R³⁵ together with the nitrogen atom to which they are attached form an optionally substituted heteroaryl ring. In another embodiment, the FXR modulator is a compound of Formula (VI) wherein R³⁴ and R³⁵ together with the nitrogen atom to which they are attached form an imidazolyl, pyrazolyl, or pyrrolyl.

In another embodiment, the FXR modulator is a compound of Formula (VI) wherein R³⁰ is

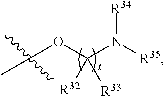

n is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VI) wherein R³⁰ is

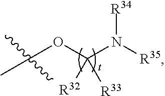

n is 2 and each R³¹ is independently halogen, or optionally substituted C₁-C₆alkyl. In another embodiment, the FXR modulator is a compound of Formula (VI) wherein R³⁰ is

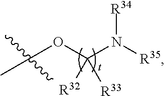

n is 2 and each R³¹ is halogen. In another embodiment, the FXR modulator is a compound of Formula (VI) wherein R³⁰ is

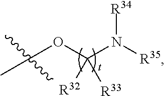

n is 2 and each R³¹ is F.

In another embodiment, the FXR modulator is a compound of Formula (VI) wherein R³⁰ is

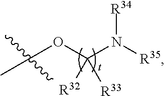

n is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VI) wherein R³⁰ is

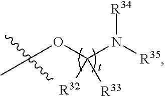

n is 1 and R³¹ is halogen, or optionally substituted C₁-C₆alkyl. In another embodiment, the FXR modulator is a compound of Formula (VI) wherein R³⁰ is

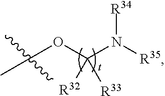

n is 1 and R³¹ is halogen. In another embodiment, the FXR modulator is a compound of Formula (VI) wherein R³⁰ is

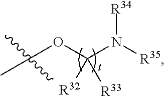

### n is 1 and R³¹ is F.

In another embodiment, the FXR modulator is a compound of Formula (VI) wherein R³⁰ is

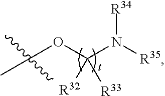

and n is 0.

In a further embodiment of the aforementioned embodiments, the FXR modulator is a compound of Formula (VI) wherein R⁴ and R⁵ are each methyl. In a further embodiment of the aforementioned embodiments, the FXR modulator is a compound of Formula (VI) wherein R⁴ and R⁵ are each ethyl. In a further embodiment of the aforementioned embodiments, the FXR modulator is a compound of Formula (VI) wherein R¹ is hydrogen. In a further embodiment of the aforementioned embodiments, the FXR modulator is a compound of Formula (VI) wherein R¹ is halogen. In a further embodiment of the aforementioned embodiments, the FXR modulator is a compound of Formula (VI) wherein R¹ is optionally substituted C₁-C₆alkyl. In a further embodiment of the aforementioned embodiments, the FXR modulator is a compound of Formula (VI) wherein R¹ is methyl. In a further embodiment of the aforementioned embodiments, the FXR modulator is a compound of Formula (VI) wherein R¹ is optionally substituted C₂-C₆alkenyl. In a further embodiment of the aforementioned embodiments, the FXR modulator is a compound of Formula (VI) wherein R¹ is optionally substituted C₂-C₆alkynyl. In a further embodiment of the aforementioned embodiments, the FXR modulator is a compound of Formula (VI) wherein R¹ is —OR¹⁰ and R¹⁰ is hydrogen. In a further embodiment of the aforementioned embodiments, the FXR modulator is a compound of Formula (VI) wherein R¹ is —OR¹⁰ and R¹⁰ is optionally substituted C₁-C₆alkyl. In a further embodiment of the aforementioned embodiments, the FXR modulator is a compound of Formula (VI) wherein R¹ is —OR¹⁰ and R¹⁰ is methyl. In a further embodiment of the aforementioned embodiments, the FXR modulator is a compound of Formula (VI) wherein R²⁵ and R²⁶ are hydrogen. In a further embodiment of the aforementioned embodiments, the FXR modulator is a compound of Formula (VI) wherein R²⁵ is hydrogen and R²⁶ is optionally substituted C₁-C₆alkyl. In a further embodiment of the aforementioned embodiments, the FXR modulator is a compound of Formula (VI) wherein R²⁵ is hydrogen and R²⁶ is methyl. In a further embodiment of the aforementioned embodiments, the FXR modulator is a compound of Formula (VI) wherein R²⁵ is hydrogen and R²⁶ is ethyl. In a further embodiment of the aforementioned embodiments, the FXR modulator is a compound of Formula (VI) wherein R²⁵ is hydrogen and R²⁶ is isopropyl. In a further embodiment of the aforementioned embodiments, the FXR modulator is a compound of Formula (VI) wherein R²⁵ and R²⁶ are each optionally substituted C₁-C₆alkyl. In a further embodiment of the aforementioned embodiments, the FXR modulator is a compound of Formula (VI) wherein R²⁵ and R²⁶ are methyl. In a further embodiment of the aforementioned embodiments, the FXR modulator is a compound of Formula (VI) wherein R²⁵ and R²⁶ are ethyl.

In a further embodiment of the aforementioned embodiments, the FXR modulator is a compound of Formula (VI) wherein R⁹ is selected from the group consisting of hydrogen and optionally substituted C₁-C₆alkyl. In a further embodiment of the aforementioned embodiments, the FXR modulator is a compound of Formula (VI) wherein R⁹ is hydrogen. In a further embodiment of the aforementioned embodiments, the FXR modulator is a compound of Formula (VI) wherein R⁹ is optionally substituted C₁-C₆alkyl. In a further embodiment of the aforementioned embodiments, the FXR modulator is a compound of Formula (V VI wherein R⁹ is unsubstituted C₁-C₆alkyl. In a further embodiment of the aforementioned embodiments, the FXR modulator is a compound of Formula (VI) wherein R⁹ is methyl.

In yet another embodiment, provided herein the FXR modulator is a compound having the structure of Formula (VIa), or a pharmaceutically acceptable salt, stereoisomer, or solvate thereof:

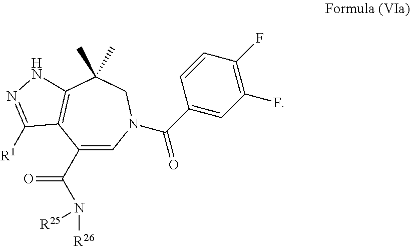

In yet another embodiment, provided herein the FXR modulator is a compound having the structure of Formula (VIb), or a pharmaceutically acceptable salt, stereoisomer, or solvate thereof:

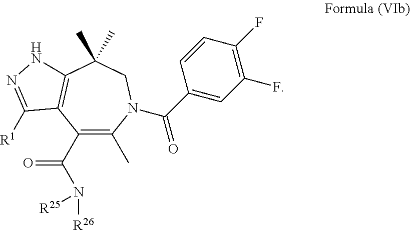

In some embodiments, the FXR modulator is a compound of Formula (VIa) or (VIb) wherein R¹ is hydrogen. In some embodiments, the FXR modulator is a compound of Formula (VIa) or (VIb) wherein R¹ is halogen. In some embodiments, the FXR modulator is a compound of Formula (VIa) or (VIb) wherein R¹ is optionally substituted C₁-C₆alkyl. In some embodiments, the FXR modulator is a compound of Formula (VIa) or (VIb) wherein R¹ is optionally substituted C₂-C₆alkenyl. In some embodiments, the FXR modulator is a compound of Formula (VIa) wherein R¹ is optionally substituted C₂-C₆alkynyl. In some embodiments, the FXR modulator is a compound of Formula (VIa) or (VIb) wherein R¹ is —OR¹⁰ and R¹⁰ is hydrogen. In some embodiments, the FXR modulator is a compound of Formula (VIa) or (VIb) wherein R¹ is —OR¹⁰ and R¹⁰ is optionally substituted C₁-C₆alkyl. In some embodiments, the FXR modulator is a compound of Formula (VIa) or (VIb) wherein R¹ is —OR¹⁰ and R¹⁰ is methyl. In a further embodiment of the aforementioned embodiments, the FXR modulator is a compound of Formula (VIa) or (VIb) wherein R²⁵ and R²⁶ are hydrogen. In a further embodiment of the aforementioned embodiments, the FXR modulator is a compound of Formula (VIa) or (VIb) wherein R²⁵ is hydrogen and R²⁶ is optionally substituted C₁-C₆alkyl. In a further embodiment of the aforementioned embodiments, the FXR modulator is a compound of Formula (VIa) or (VIb) wherein R²⁵ is hydrogen and R²⁶ is methyl. In a further embodiment of the aforementioned embodiments, the FXR modulator is a compound of Formula (VIa) or (VIb) wherein R²⁵ is hydrogen and R²⁶ is ethyl. In a further embodiment of the aforementioned embodiments, the FXR modulator is a compound of Formula (VIa) or (VIb) wherein R²⁵ is hydrogen and R²⁶ is isopropyl. In a further embodiment of the aforementioned embodiments, the FXR modulator is a compound of Formula (VIa) or (VIb) wherein R²⁵ and R²⁶ are each optionally substituted C₁-C₆alkyl. In a further embodiment of the aforementioned embodiments, the FXR modulator is a compound of Formula (VIa) or (VIb) wherein R²⁵ and R²⁶ are methyl. In a further embodiment of the aforementioned embodiments, the FXR modulator is a compound of Formula (VIa) or (VIb) wherein R²⁵ and R²⁶ are ethyl.

In some embodiments is a method of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof; wherein the FXR modulator is a compound of Formula (VII), or a pharmaceutically acceptable salt, stereoisomer, or solvate thereof:

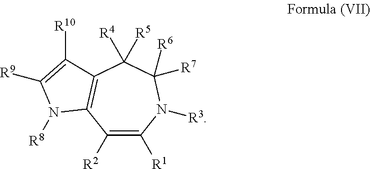

wherein:
R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R² is selected from the group consisting of —CN, —C(O)OR²⁵, —C(O)N(R²⁵)R²⁶,

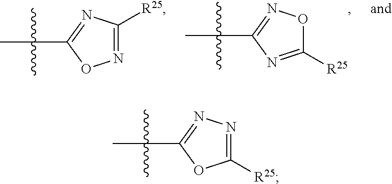

or R¹ and R² together with the carbon atoms to which they are attached, form an optionally substituted C₂-C₉heterocycloalkyl ring or an optionally substituted heteroaryl ring;
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇cycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R⁹ and R¹⁰ together with the carbon atoms to which they are attached, form an optionally substituted nitrogen containing 6-membered heteroaryl ring;
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring.

In one embodiment, the FXR modulator is a compound of Formula (VII) wherein R⁴ and R⁵ are each independently selected from the group consisting of hydrogen, halogen, and optionally substituted C₁-C₆alkyl. In another embodiment, the FXR modulator is a compound of Formula (VII) wherein R⁴ and R⁵ are each independently selected from the group consisting of hydrogen and optionally substituted C₁-C₆alkyl. In another embodiment, the FXR modulator is a compound of Formula (VII) wherein R⁴ and R⁵ are each hydrogen. In another embodiment, the FXR modulator is a compound of Formula (VII) wherein R⁴ and R⁵ are each independently optionally substituted C₁-C₆alkyl. In another embodiment, the FXR modulator is a compound of Formula (VII) wherein R⁴ and R⁵ are each methyl. In another embodiment, the FXR modulator is a compound of Formula (VII) wherein R⁴ and R⁵ form an optionally substituted C₃-C₆cycloalkyl ring or an optionally substituted C₂-C₇heterocycloalkyl ring. In some embodiments, the FXR modulator is a compound of Formula (VII) wherein R⁴ and R⁵ form an optionally substituted C₃-C₆cycloalkyl ring. In some embodiments, the FXR modulator is a compound of Formula (VII) wherein R⁴ and R⁵ form an optionally substituted C₂-C₇heterocycloalkyl ring.

In another embodiment, the FXR modulator is a compound of Formula (VII) wherein R⁶ and R⁷ are each independently selected from the group consisting of hydrogen, halogen, and optionally substituted C₁-C₆alkyl. In another embodiment, the FXR modulator is a compound of Formula (VII) wherein R⁶ and R⁷ are each independently selected from the group consisting of hydrogen and optionally substituted C₁-C₆alkyl. In another embodiment, the FXR modulator is a compound of Formula (VII) wherein R⁶ and R⁷ are each independently optionally substituted C₁-C₆alkyl. In another embodiment, the FXR modulator is a compound of Formula (VII) wherein R⁶ and R⁷ are each methyl. In another embodiment, the FXR modulator is a compound of Formula (VII) wherein R⁶ and R⁷ are each hydrogen.

In another embodiment, the FXR modulator is a compound of Formula (VII) wherein R⁶ and R⁷ are hydrogen, R⁴ and R⁵ are independently optionally substituted C₁-C₆alkyl, R³ is —C(O)R²⁰, and R²⁰ is optionally substituted aryl. In another embodiment, the FXR modulator is a compound of Formula (VII) wherein R⁶ and R⁷ are hydrogen, R⁴ and R⁵ are independently optionally substituted C₁-C₆alkyl, R³ is —C(O)R²⁰, and R²⁰ is optionally substituted heteroaryl. In another embodiment, the FXR modulator is a compound of Formula (VII) wherein R⁶ and R⁷ are hydrogen, R⁴ and R⁵ are methyl, R³ is —C(O)R²⁰, and R²⁰ is optionally substituted aryl. In another embodiment, the FXR modulator is a compound of Formula (VII) wherein R⁶ and R⁷ are hydrogen, R⁴ and R⁵ are methyl, R³ is —C(O)R²⁰, and R²⁰ is optionally substituted heteroaryl.

In another embodiment, the FXR modulator is a compound of Formula (VII) wherein R⁶ and R⁷ are hydrogen, R⁴ and R⁵ are independently optionally substituted C₁-C₆alkyl, R³ is —S(O)₂R²⁰, and R²⁰ is optionally substituted aryl. In another embodiment, the FXR modulator is a compound of Formula (VII) wherein R⁶ and R⁷ are hydrogen, R⁴ and R⁵ are independently optionally substituted C₁-C₆alkyl, R³ is —S(O)₂R²⁰, and R²⁰ is optionally substituted heteroaryl. In another embodiment, the FXR modulator is a compound of Formula (VII) wherein R⁶ and R⁷ are hydrogen, R⁴ and R⁵ are methyl, R³ is —S(O)₂R²⁰, and R²⁰ is optionally substituted aryl. In another embodiment, the FXR modulator is a compound of Formula (VII) wherein R⁶ and R⁷ are hydrogen, R⁴ and R⁵ are methyl, R³ is —S(O)₂R²⁰, and R²⁰ is optionally substituted heteroaryl.

In another embodiment, the FXR modulator is a compound of Formula (VII) wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted aryl. In another embodiment, the FXR modulator is a compound of Formula (VII) wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted heteroaryl. In another embodiment, the FXR modulator is a compound of Formula (VII) wherein R⁶ and R⁷ are hydrogen, R⁴ and R⁵ are methyl, R³ is —C(O)N(R²¹)R²², R²¹ is hydrogen and R²² is optionally substituted aryl. In another embodiment, the FXR modulator is a compound of Formula (VII) wherein R⁶ and R⁷ are hydrogen, R⁴ and R⁵ are methyl, R³ is —C(O)N(R²¹)R²², R²¹ is hydrogen and R²² is optionally substituted heteroaryl.

In a further embodiment of the aforementioned embodiments is a compound of Formula (I) wherein R² is selected from the group consisting of —CN, —C(O)OR²⁵, —C(O)N(R²⁵)R²⁶,

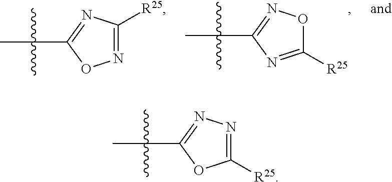

In a further embodiment of the aforementioned embodiments, the FXR modulator is a compound of Formula (VII) wherein R² is —CN.

In a further embodiment of the aforementioned embodiments, the FXR modulator is a compound of Formula (VII) wherein R² is —C(O)OR²⁵. In a further embodiment of the aforementioned embodiments, the FXR modulator is a compound of Formula (VII) wherein R² is —C(O)OR²⁵, and R²⁵ is independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (VII) wherein R² is —C(O)OR²⁵, and R²⁵ is independently selected from the group consisting of hydrogen, and optionally substituted C₁-C₆alkyl. In a further embodiment of the aforementioned embodiments, the FXR modulator is a compound of Formula (VII) wherein R² is —C(O)OR²⁵, and R²⁵ is hydrogen. In a further embodiment of the aforementioned embodiments, the FXR modulator is a compound of Formula (VII) wherein R² is —C(O)OR²⁵, and R²⁵ is optionally substituted C₁-C₆alkyl. In a further embodiment of the aforementioned embodiments, the FXR modulator is a compound of Formula (VII) wherein R² is —C(O)OR²⁵, and R²⁵ is unsubstituted C₁-C₆alkyl. In a further embodiment of the aforementioned embodiments, the FXR modulator is a compound of Formula (VII) wherein R² is —C(O)OR²⁵, and R²⁵ is methyl. In a further embodiment of the aforementioned embodiments, the FXR modulator is a compound of Formula (VII) wherein R² is —C(O)OR²⁵, and R²⁵ is ethyl.

In a further embodiment of the aforementioned embodiments, the FXR modulator is a compound of Formula (VII) wherein R² is —C(O)N(R²⁵)R²⁶. In a further embodiment of the aforementioned embodiments, the FXR modulator is a compound of Formula (VII) wherein R² is —C(O)N(R²⁵)R²⁶, and R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (VII) wherein R² is —C(O)N(R²⁵)R²⁶, and R²⁵ and R²⁶ are each independently selected from the group consisting of hydrogen, and optionally substituted C₁-C₆alkyl. In a further embodiment of the aforementioned embodiments, the FXR modulator is a compound of Formula (VII) wherein R² is —C(O)N(R²⁵)R²⁶, and R²⁵ and R²⁶ are hydrogen. In a further embodiment of the aforementioned embodiments, the FXR modulator is a compound of Formula (VII) wherein R² is —C(O)N(R²⁵)R²⁶, and R²⁵ and R²⁶ are each independently optionally substituted C₁-C₆alkyl. In a further embodiment of the aforementioned embodiments, the FXR modulator is a compound of Formula (VII) wherein R² is —C(O)N(R²⁵)R²⁶, R²⁵ is hydrogen, and R²⁶ is optionally substituted C₁-C₆alkyl. In a further embodiment of the aforementioned embodiments, the FXR modulator is a compound of Formula (VII) wherein R² is —C(O)N(R²⁵)R²⁶, and R²⁵ and R²⁶ are each independently unsubstituted C₁-C₆alkyl. In a further embodiment of the aforementioned embodiments, the FXR modulator is a compound of Formula (VII) wherein R² is —C(O)N(R²⁵)R²⁶, R²⁵ is hydrogen, and R²⁶ are methyl. In a further embodiment of the aforementioned embodiments, the FXR modulator is a compound of Formula (VII) wherein R² is —C(O)N(R²⁵)R²⁶, and R²⁵ and R²⁶ are methyl. In a further embodiment of the aforementioned embodiments, the FXR modulator is a compound of Formula (VII) wherein R² is —C(O)N(R²⁵)R²⁶, and R²⁵ and R²⁶ are ethyl.

In a further embodiment of the aforementioned embodiments, the FXR modulator is a compound of Formula (VII) wherein R² is

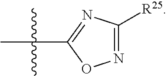

In a further embodiment of the aforementioned embodiments, the FXR modulator is a compound of Formula (VII) wherein R² is

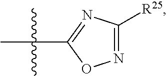

and R²⁵ is optionally substituted C₁-C₆alkyl. In a further embodiment of the aforementioned embodiments, the FXR modulator is a compound of Formula (VII) wherein R² is

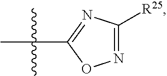

and R²⁵ is methyl. In a further embodiment of the aforementioned embodiments, the FXR modulator is a compound of Formula (VII) wherein R² is

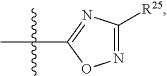

and R²⁵ is ethyl.

In a further embodiment of the aforementioned embodiments, the FXR modulator is a compound of Formula (VII) wherein R² is

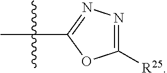

In a further embodiment of the aforementioned embodiments, the FXR modulator is a compound of Formula (VII) wherein R² is

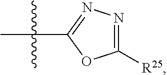

and R²⁵ is optionally substituted C₁-C₆alkyl. In a further embodiment of the aforementioned embodiments, the FXR modulator is a compound of Formula (VII) wherein R² is

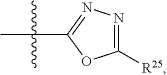

and R²⁵ is methyl. In a further embodiment of the aforementioned embodiments, the FXR modulator is a compound of Formula (VII) wherein R² is

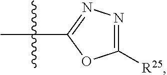

and R²⁵ is ethyl.

In a further embodiment of the aforementioned embodiments, the FXR modulator is a compound of Formula (VII) wherein R² is

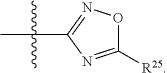

In a further embodiment of the aforementioned embodiments, the FXR modulator is a compound of Formula (VII) wherein R² is

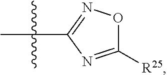

and R²⁵ is optionally substituted C₁-C₆alkyl. In a further embodiment of the aforementioned embodiments, the FXR modulator is a compound of Formula (VII) wherein R² is

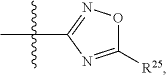

and R²⁵ is methyl. In a further embodiment of the aforementioned embodiments, the FXR modulator is a compound of Formula (VII) wherein R² is

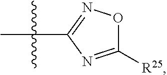

and R²⁵ is ethyl.

In a further embodiment of the aforementioned embodiments, the FXR modulator is a compound of Formula (VII) wherein R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (VII) wherein R¹ is hydrogen. In a further embodiment of the aforementioned embodiments, the FXR modulator is a compound of Formula (VII) wherein R¹ is optionally substituted C₁-C₆alkyl. In a further embodiment of the aforementioned embodiments, the FXR modulator is a compound of Formula (VII) wherein R¹ is methyl. In a further embodiment of the aforementioned embodiments, the FXR modulator is a compound of Formula (VII) wherein R¹ is optionally substituted C₂-C₆alkenyl. In a further embodiment of the aforementioned embodiments, the FXR modulator is a compound of Formula (VII) wherein R¹ is optionally substituted C₂-C₆alkynyl.

In a further embodiment of the aforementioned embodiments, the FXR modulator is a compound of Formula (VII) wherein R¹ and R² together with the carbon atoms to which they are attached, form an optionally substituted C₂-C₉heterocycloalkyl ring or an optionally substituted heteroaryl ring. In a further embodiment of the aforementioned embodiments, the FXR modulator is a compound of Formula (VII) wherein R¹ and R² together with the carbon atoms to which they are attached, form an optionally substituted C₂-C₉heterocycloalkyl ring. In a further embodiment of the aforementioned embodiments, the FXR modulator is a compound of Formula (VII) wherein R¹ and R² together with the carbon atoms to which they are attached, form an optionally substituted heteroaryl ring.

In a further embodiment of the aforementioned embodiments, the FXR modulator is a compound of Formula (VII) wherein R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl). In a further embodiment of the aforementioned embodiments, the FXR modulator is a compound of Formula (VII) wherein R⁸ is selected from the group consisting of hydrogen, and optionally substituted C₁-C₆alkyl. In a further embodiment of the aforementioned embodiments, the FXR modulator is a compound of Formula (VII) wherein R⁸ is optionally substituted C₁-C₆alkyl. In a further embodiment of the aforementioned embodiments, the FXR modulator is a compound of Formula (VII) wherein R⁸ is methyl. In a further embodiment of the aforementioned embodiments, the FXR modulator is a compound of Formula (VII) wherein R⁸ is optionally substituted C₁-C₆alkyl. In a further embodiment of the aforementioned embodiments, the FXR modulator is a compound of Formula (VII) wherein R⁸ is ethyl. In a further embodiment of the aforementioned embodiments, the FXR modulator is a compound of Formula (VII) wherein R⁸ is optionally substituted C₁-C₆alkyl. In a further embodiment of the aforementioned embodiments, the FXR modulator is a compound of Formula (VII) wherein R⁸ is hydrogen.

In a further embodiment of the aforementioned embodiments provided herein, the FXR modulator compound of Formula (VII) has the structure of Formula (VIIa), or a pharmaceutically acceptable salt, stereoisomer, or solvate thereof:

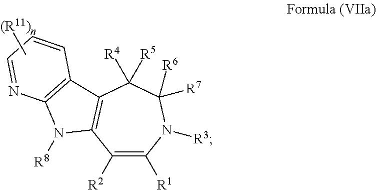

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In a further embodiment of the aforementioned embodiments provided herein, the FXR modulator compound of Formula (VII) has the structure of Formula (VIIb), or a pharmaceutically acceptable salt, stereoisomer, or solvate thereof:

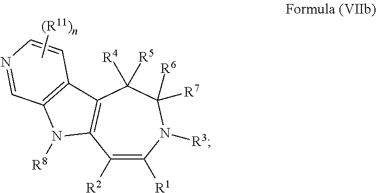

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In a further embodiment of the aforementioned embodiments provided herein, the FXR modulator compound of Formula (VII) has the structure of Formula (VIIc), or a pharmaceutically acceptable salt, stereoisomer, or solvate thereof:

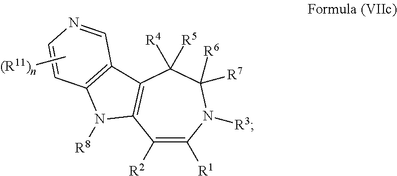

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In a further embodiment of the aforementioned embodiments provided herein, the FXR modulator compound of Formula (VII) has the structure of Formula (VIId), or a pharmaceutically acceptable salt, stereoisomer, or solvate thereof:

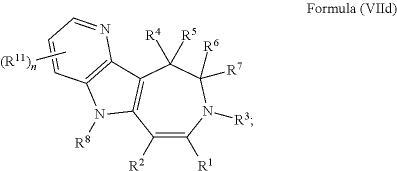

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In a further embodiment of the aforementioned embodiments provided herein, the FXR modulator compound of Formula (VII) has the structure of Formula (VIIe), or a pharmaceutically acceptable salt, stereoisomer, or solvate thereof:

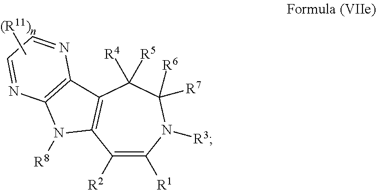

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In a further embodiment of the aforementioned embodiments provided herein, the FXR modulator compound of Formula (VII) has the structure of Formula (VIIf), or a pharmaceutically acceptable salt, stereoisomer, or solvate thereof:

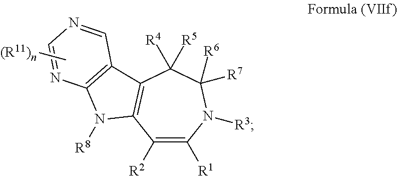

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In a further embodiment of the aforementioned embodiments provided herein, the FXR modulator compound of Formula (VII) has the structure of Formula (VIIg), or a pharmaceutically acceptable salt, stereoisomer, or solvate thereof:

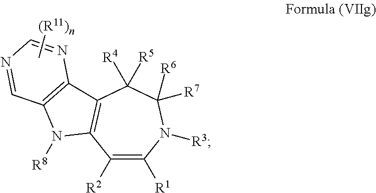

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In a further embodiment of the aforementioned embodiments provided herein, the FXR modulator compound of Formula (VII) has the structure of Formula (VIIh), or a pharmaceutically acceptable salt, stereoisomer, or solvate thereof:

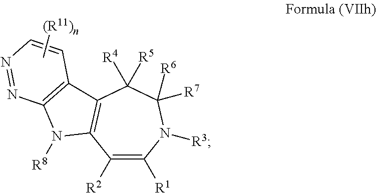

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In a further embodiment of the aforementioned embodiments provided herein, the FXR modulator compound of Formula (VII) has the structure of Formula (VIIi), or a pharmaceutically acceptable salt, stereoisomer, or solvate thereof:

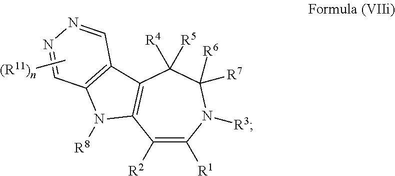

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In a further embodiment of the aforementioned embodiments provided herein, the FXR modulator compound of Formula (VII) has the structure of Formula (VIIj), or a pharmaceutically acceptable salt, stereoisomer, or solvate thereof:

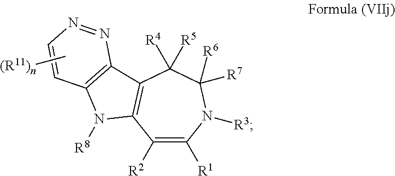

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In some embodiments, the FXR modulator is a compound of Formula (VIIa)-(VIIj) wherein n is 0. In a further embodiment of the aforementioned embodiments, the FXR modulator is a compound of Formula (VIIa)-(VIIj) wherein n is 1. In a further embodiment of the aforementioned embodiments, the FXR modulator is a compound of Formula (VIIa)-(VIIj) wherein n is 2. In a further embodiment of the aforementioned embodiments, the FXR modulator is a compound of Formula (VIIa)-(VIIj) wherein n is 1 and R¹¹ is selected from the group consisting of halogen, —CN, amino, alkylamino, C₁-C₆alkyl, C₁-C₆haloalkyl, C₁-C₆alkoxy, and C₁-C₆haloalkoxy. In a further embodiment of the aforementioned embodiments, the FXR modulator is a compound of Formula (VIIa)-(VIIj) wherein n is 1 and R¹¹ is selected from the group consisting of halogen, —CN, C₁-C₆alkyl, and C₁-C₆alkoxy. In a further embodiment of the aforementioned embodiments, the FXR modulator is a compound of Formula (VIIa)-(VIIj) wherein n is 1 and R¹¹ is halogen.

In another embodiment of the aforementioned embodiments, the FXR modulator is a compound of Formula (VIIa)-(VIIj) wherein R⁶ and R⁷ are hydrogen, R⁴ and R⁵ are independently optionally substituted C₁-C₆alkyl, R³ is —C(O)R²⁰, and R²⁰ is optionally substituted aryl. In another embodiment of the aforementioned embodiments, the FXR modulator is a compound of Formula (VIIa)-(VIIj) wherein R⁶ and R⁷ are hydrogen, R⁴ and R⁵ are independently optionally substituted C₁-C₆alkyl, R³ is —C(O)R²⁰, and R²⁰ is optionally substituted heteroaryl. In another embodiment of the aforementioned embodiments, the FXR modulator is a compound of Formula (VIIa)-(VIIj) wherein R⁶ and R⁷ are hydrogen, R⁴ and R⁵ are methyl, R³ is —C(O)R²⁰, and R²⁰ is optionally substituted aryl. In another embodiment of the aforementioned embodiments, the FXR modulator is a compound of Formula (VIIa)-(VIIj) wherein R⁶ and R⁷ are hydrogen, R⁴ and R⁵ are methyl, R³ is —C(O)R²⁰, and R²⁰ is optionally substituted heteroaryl.

In another embodiment of the aforementioned embodiments, the FXR modulator is a compound of Formula (VIIa)-(VIIj) wherein R⁶ and R⁷ are hydrogen, R⁴ and R⁵ are independently optionally substituted C₁-C₆alkyl, R³ is —S(O)₂R²⁰, and R²⁰ is optionally substituted aryl. In another embodiment of the aforementioned embodiments, the FXR modulator is a compound of Formula (VIIa)-(VIIj) wherein R⁶ and R⁷ are hydrogen, R⁴ and R⁵ are independently optionally substituted C₁-C₆alkyl, R³ is —S(O)₂R²⁰, and R²⁰ is optionally substituted heteroaryl. In another embodiment of the aforementioned embodiments, the FXR modulator is a compound of Formula (VIIa)-(VIIj) wherein R⁶ and R⁷ are hydrogen, R⁴ and R⁵ are methyl, R³ is —S(O)₂R²⁰, and R²⁰ is optionally substituted aryl. In another embodiment of the aforementioned embodiments, the FXR modulator is a compound of Formula (VIIa)-(VIIj) wherein R⁶ and R⁷ are hydrogen, R⁴ and R⁵ are methyl, R³ is —S(O)₂R²⁰, and R²⁰ is optionally substituted heteroaryl.

In another embodiment of the aforementioned embodiments, the FXR modulator is a compound of Formula (VIIa)-(VIIj) wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted aryl. In another embodiment of the aforementioned embodiments, the FXR modulator is a compound of Formula (VIIa)-(VIIj) wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted heteroaryl. In another embodiment of the aforementioned embodiments, the FXR modulator is a compound of Formula (VIIa)-(VIIj) wherein R⁶ and R⁷ are hydrogen, R⁴ and R⁵ are methyl, R³ is —C(O)N(R²¹)R²², R²¹ is hydrogen and R²² is optionally substituted aryl. In another embodiment of the aforementioned embodiments, the FXR modulator is a compound of Formula (VIIa)-(VIIj) wherein R⁶ and R⁷ are hydrogen, R⁴ and R⁵ are methyl, R³ is —C(O)N(R²¹)R²², R²¹ is hydrogen and R²² is optionally substituted heteroaryl.

In a further embodiment of the aforementioned embodiments, the FXR modulator is a compound of Formula (VIIa)-(VIIj) wherein R² is selected from the group consisting of —CN, —C(O)OR²⁵, —C(O)N(R²⁵)R²⁶,

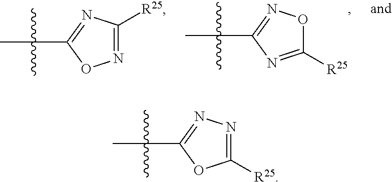

In a further embodiment of the aforementioned embodiments, the FXR modulator is a compound of Formula (VIIa)-(VIIj) wherein R² is —CN.

In a further embodiment of the aforementioned embodiments, the FXR modulator is a compound of Formula (VIIa)-(VIIj) wherein R² is —C(O)OR²⁵. In a further embodiment of the aforementioned embodiments, the FXR modulator is a compound of Formula (VIIa)-(VIIj) wherein R² is —C(O)OR²⁵, and R²⁵ is independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (VIIa)-(VIIj) wherein R² is —C(O)OR²⁵, and R²⁵ is independently selected from the group consisting of hydrogen, and optionally substituted C₁-C₆alkyl. In a further embodiment of the aforementioned embodiments, the FXR modulator is a compound of Formula (VIIa)-(VIIj) wherein R² is —C(O)OR²⁵, and R²⁵ is hydrogen. In a further embodiment of the aforementioned embodiments, the FXR modulator is a compound of Formula (VIIa)-(VIIj) wherein R² is —C(O)OR²⁵, and R²⁵ is optionally substituted C₁-C₆alkyl. In a further embodiment of the aforementioned embodiments, the FXR modulator is a compound of Formula (VIIa)-(VIIj) wherein R² is —C(O)OR²⁵, and R²⁵ is unsubstituted C₁-C₆alkyl. In a further embodiment of the aforementioned embodiments, the FXR modulator is a compound of Formula (VIIa)-(VIIj) wherein R² is —C(O)OR²⁵, and R²⁵ is methyl. In a further embodiment of the aforementioned embodiments, the FXR modulator is a compound of Formula (VIIa)-(VIIj) wherein R² is —C(O)OR²⁵, and R²⁵ is ethyl. In a further embodiment of the aforementioned embodiments, the FXR modulator is a compound of Formula (VIIa)-(VIIj) wherein R² is —C(O)OR²⁵, and R²⁵ is isopropyl.

In a further embodiment of the aforementioned embodiments, the FXR modulator is a compound of Formula (VIIa)-(VIIj) wherein R² is —C(O)N(R²⁵)R²⁶. In a further embodiment of the aforementioned embodiments, the FXR modulator is a compound of Formula (VIIa)-(VIIj) wherein R² is —C(O)N(R²⁵)R²⁶, and R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (VIIa)-(VIIj) wherein R² is —C(O)N(R²⁵)R²⁶, and R²⁵ and R²⁶ are each independently selected from the group consisting of hydrogen, and optionally substituted C₁-C₆alkyl. In a further embodiment of the aforementioned embodiments, the FXR modulator is a compound of Formula (VIIa)-(VIIj) wherein R² is —C(O)N(R²⁵)R²⁶, and R²⁵ and R²⁶ are hydrogen. In a further embodiment of the aforementioned embodiments, the FXR modulator is a compound of Formula (VIIa)-(VIIj) wherein R² is —C(O)N(R²⁵)R²⁶, and R²⁵ and R²⁶ are each independently optionally substituted C₁-C₆alkyl. In a further embodiment of the aforementioned embodiments, the FXR modulator is a compound of Formula (VIIa)-(VIIj) wherein R² is —C(O)N(R²⁵)R²⁶, R²⁵ is hydrogen, and R²⁶ is optionally substituted C₁-C₆alkyl. In a further embodiment of the aforementioned embodiments, the FXR modulator is a compound of Formula (VIIa)-(VIIj) wherein R² is —C(O)N(R²⁵)R²⁶, and R²⁵ and R²⁶ are each independently unsubstituted C₁-C₆alkyl. In a further embodiment of the aforementioned embodiments, the FXR modulator is a compound of Formula (VIIa)-(VIIj) wherein R² is —C(O)N(R²⁵)R²⁶, R²⁵ is hydrogen, and R²⁶ are methyl. In a further embodiment of the aforementioned embodiments, the FXR modulator is a compound of Formula (VIIa)-(VIIj) wherein R² is —C(O)N(R²⁵)R²⁶, and R²⁵ and R²⁶ are methyl. In a further embodiment of the aforementioned embodiments, the FXR modulator is a compound of Formula (VIIa)-(VIIj) wherein R² is —C(O)N(R²⁵)R²⁶, and R²⁵ and R²⁶ are ethyl.

In a further embodiment of the aforementioned embodiments, the FXR modulator is a compound of Formula (VIIa)-(VIIj) wherein R² is

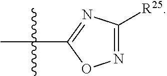

In a further embodiment of the aforementioned embodiments, the FXR modulator is a compound of Formula (VIIa)-(VIIj) wherein R² is

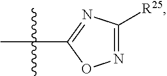

and R²⁵ is optionally substituted C₁-C₆alkyl. In a further embodiment of the aforementioned embodiments, the FXR modulator is a compound of Formula (VIIa)-(VIIj) wherein R² is

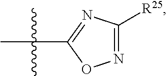

and R²⁵ is methyl. In a further embodiment of the aforementioned embodiments, the FXR modulator is a compound of Formula (VIIa)-(VIIj) wherein R² is

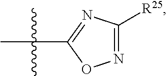

and R²⁵ is ethyl.

In a further embodiment of the aforementioned embodiments, the FXR modulator is a compound of Formula (VIIa)-(VIIj) wherein R² is

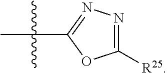

In a further embodiment of the aforementioned embodiments, the FXR modulator is a compound of Formula (VIIa)-(VIIj) wherein R² is

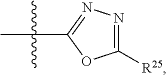

and R²⁵ is optionally substituted C₁-C₆alkyl. In a further embodiment of the aforementioned embodiments, the FXR modulator is a compound of Formula (VIIa)-(VIIj) wherein R² is

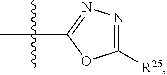

and R²⁵ is methyl. In a further embodiment of the aforementioned embodiments, the FXR modulator is a compound of Formula (VIIa)-(VIIj) wherein R² is

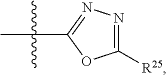

and R²⁵ is ethyl.

In a further embodiment of the aforementioned embodiments, the FXR modulator is a compound of Formula (VIIa)-(VIIj) wherein R² is

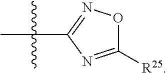

In a further embodiment of the aforementioned embodiments, the FXR modulator is a compound of Formula (VIIa)-(VIIj) wherein R² is

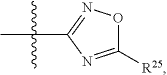

and R²⁵ is optionally substituted C₁-C₆alkyl. In a further embodiment of the aforementioned embodiments, the FXR modulator is a compound of Formula (VIIa)-(VIIj) wherein R² is

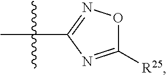

and R²⁵ is methyl. In a further embodiment of the aforementioned embodiments, the FXR modulator is a compound of Formula (VIIa)-(VIIj) wherein R² is

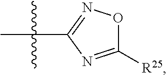

and R²⁵ is ethyl.

In a further embodiment of the aforementioned embodiments, the FXR modulator is a compound of Formula (VIIa)-(VIIj) wherein R¹ is selected from the group consisting of hydrogen and optionally substituted C₁-C₆alkyl. In a further embodiment of the aforementioned embodiments, the FXR modulator is a compound of Formula (VIIa)-(VIIj) wherein R¹ is hydrogen. In a further embodiment of the aforementioned embodiments, the FXR modulator is a compound of Formula (VIIa)-(VIIj) wherein R¹ is optionally substituted C₁-C₆alkyl. In a further embodiment of the aforementioned embodiments, the FXR modulator is a compound of Formula (VIIa)-(VIIj) wherein R¹ is unsubstituted C₁-C₆alkyl. In a further embodiment of the aforementioned embodiments, the FXR modulator is a compound of Formula (VIIa)-(VIIj) wherein R¹ is methyl.

In a further embodiment of the aforementioned embodiments, the FXR modulator is a compound of Formula (VIIa)-(VIIj) wherein R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl). In a further embodiment of the aforementioned embodiments, the FXR modulator is a compound of Formula (VIIa)-(VIIj) wherein R⁸ is selected from the group consisting of hydrogen, and optionally substituted C₁-C₆alkyl. In a further embodiment of the aforementioned embodiments, the FXR modulator is a compound of Formula (VIIa)-(VIIj) wherein R⁸ is optionally substituted C₁-C₆alkyl. In a further embodiment of the aforementioned embodiments, the FXR modulator is a compound of Formula (VIIa)-(VIIj) wherein R⁸ is methyl. In a further embodiment of the aforementioned embodiments, the FXR modulator is a compound of Formula (VIIa)-(VIIj) wherein R⁸ is optionally substituted C₁-C₆alkyl. In a further embodiment of the aforementioned embodiments, the FXR modulator is a compound of Formula (VIIa)-(VIIj) wherein R⁸ is methyl. In a further embodiment of the aforementioned embodiments, the FXR modulator is a compound of Formula (VIIa)-(VIIj) wherein R⁸ is ethyl. In a further embodiment of the aforementioned embodiments, the FXR modulator is a compound of Formula (VIIa)-(VIIj) wherein R⁸ is hydrogen.

In some embodiments is a method of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof; wherein the FXR modulator is a compound of Formula (VIII), or a pharmaceutically acceptable salt, stereoisomer, or solvate thereof:

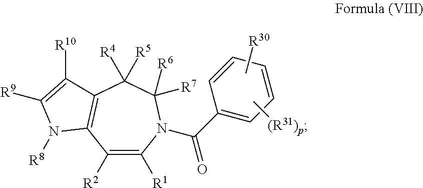

wherein:
R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R² is selected from the group consisting of —CN, —C(O)OR²⁵, —C(O)N(R²⁵)R²⁶,

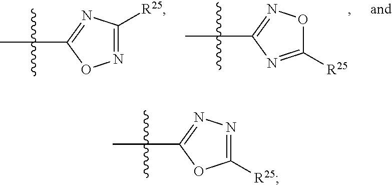

or R¹ and R² together with the carbon atoms to which they are attached, form an optionally substituted C₂-C₉heterocycloalkyl ring or an optionally substituted heteroaryl ring;
R⁴ and R⁵ are each independently selected from the group consisting of hydrogen, halogen, optionally substituted C₁-C₆alkoxy, optionally substituted C₁-C₆alkyl, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);

R⁹ and R¹⁰ together with the carbon atoms to which they are attached, form an optionally substituted nitrogen containing 6-membered heteroaryl ring;
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring;

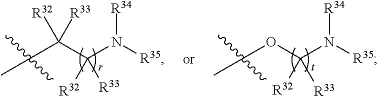

R³⁰ is halogen, each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
p is 0, 1, 2, 3, or 4;
r is 0, 1, 2, 3, or 4; and
t is 2, 3, or 4.

In one embodiment, the FXR modulator is a compound of Formula (VIII) wherein p is 0. In another embodiment, the FXR modulator is a compound of Formula (VIII) wherein p is 1. In another embodiment, the FXR modulator is a compound of Formula (VIII) wherein p is 2. In another embodiment, the FXR modulator is a compound of Formula (VIII) wherein p is 3. In another embodiment, the FXR modulator is a compound of Formula (VIII) wherein p is 4.

In another embodiment, the FXR modulator is a compound of Formula (VIII) wherein p is 2 and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIII) wherein p is 2 and each R³¹ is independently halogen, or optionally substituted C₁-C₆alkyl. In another embodiment, the FXR modulator is a compound of Formula (VIII) wherein p is 2 and each R³¹ is halogen. In another embodiment, the FXR modulator is a compound of Formula (VIII) wherein p is 2 and each R³¹ is F.

In another embodiment, the FXR modulator is a compound of Formula (VIII) wherein R³⁰ is F, p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIII) wherein R³⁰ is F, p is 2 and each R³¹ is independently halogen, or optionally substituted C₁-C₆alkyl. In another embodiment, the FXR modulator is a compound of Formula (VIII) wherein R³⁰ is F, p is 2 and each R³¹ is halogen. In another embodiment, the FXR modulator is a compound of Formula (VIII) wherein R³⁰ is F, p is 2 and each R³¹ is F.

In another embodiment, the FXR modulator is a compound of Formula (VIII) wherein p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIII) wherein p is 1 and R³¹ is halogen, or optionally substituted C₁-C₆alkyl. In another embodiment, the FXR modulator is a compound of Formula (VIII) wherein p is 1 and R³¹ is halogen. In another embodiment, the FXR modulator is a compound of Formula (VIII) wherein p is 1 and R³¹ is F.

In another embodiment, the FXR modulator is a compound of Formula (VIII) wherein R³⁰ is F, p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIII) wherein R³⁰ is F, p is 1 and R³¹ is halogen, or optionally substituted C₁-C₆alkyl. In another embodiment, the FXR modulator is a compound of Formula (VIII) wherein R³⁰ is F, p is 1 and R³¹ is halogen. In another embodiment, the FXR modulator is a compound of Formula (VIII) wherein R³⁰ is F, p is 1 and R³¹ is F.

In another embodiment, the FXR modulator is a compound of Formula (VIII) wherein R³⁰ is

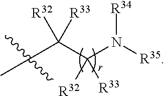

In one embodiment, the FXR modulator is a compound of Formula (VIII) wherein r is 0. In another embodiment, the FXR modulator is a compound of Formula (VIII) wherein r is 1. In another embodiment, the FXR modulator is a compound of Formula (VIII) wherein r is 2. In another embodiment, the FXR modulator is a compound of Formula (VIII) wherein r is 3. In another embodiment, the FXR modulator is a compound of Formula (VIII) wherein r is 4. In one embodiment, the FXR modulator is a compound of Formula (VIII) wherein each R³² and R³³ are hydrogen. In one embodiment, the FXR modulator is a compound of Formula (VIII) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (VIII) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (VIII) wherein R³⁴ and R³⁵ together with the nitrogen atom to which they are attached form an optionally substituted C₂-C₉heterocycloalkyl ring. In another embodiment, the FXR modulator is a compound of Formula (VIII) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (VIII) wherein R³⁴ and R³⁵ together with the nitrogen atom to which they are attached form a morpholinyl. In another embodiment, the FXR modulator is a compound of Formula (VIII) wherein R³⁴ and R³⁵ together with the nitrogen atom to which they are attached form an optionally substituted heteroaryl ring. In another embodiment, the FXR modulator is a compound of Formula (VIII) wherein R³⁴ and R³⁵ together with the nitrogen atom to which they are attached form an imidazolyl, pyrazolyl, or pyrrolyl.

In another embodiment, the FXR modulator is a compound of Formula (VIII) wherein R³⁰ is

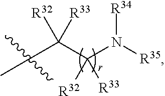

p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIII) wherein R³⁰ is

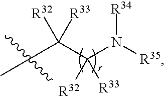

p is 2 and each R³¹ is independently halogen, or optionally substituted C₁-C₆alkyl. In another embodiment, the FXR modulator is a compound of Formula (VIII) wherein R³⁰ is

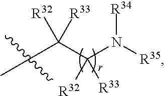

p is 2 and each R³¹ is halogen. In another embodiment, the FXR modulator is a compound of Formula (VIII) wherein R³⁰ is

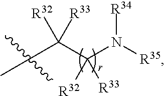

p is 2 and each R³¹ is F.

In another embodiment, the FXR modulator is a compound of Formula (VIII) wherein R³⁰ is

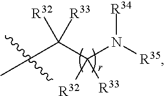

p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIII) wherein R³⁰ is

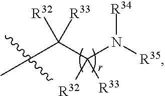

p is 1 and R³¹ is halogen, or optionally substituted C₁-C₆alkyl. In another embodiment, the FXR modulator is a compound of Formula (VIII) wherein R³⁰ is

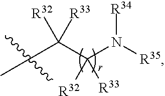

p is 1 and R³¹ is halogen. In another embodiment, the FXR modulator is a compound of Formula (VIII) wherein R³⁰ is

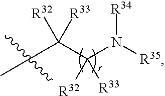

### p is 1 and R³¹ is F.

In another embodiment, the FXR modulator is a compound of Formula (VIII) wherein R³⁰ is

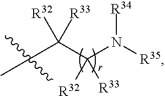

and p is 0.

In another embodiment, the FXR modulator is a compound of Formula (VIII) wherein R³⁰ is

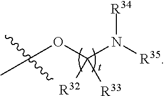

In one embodiment, the FXR modulator is a compound of Formula (VIII) wherein t is 2. In another embodiment, the FXR modulator is a compound of Formula (VIII) wherein t is 3. In another embodiment, the FXR modulator is a compound of Formula (VIII) wherein t is 4. In one embodiment, the FXR modulator is a compound of Formula (VIII) wherein each R³² and R³³ are hydrogen. In one embodiment, the FXR modulator is a compound of Formula (VIII) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (VIII) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (VIII) wherein R³⁴ and R³⁵ together with the nitrogen atom to which they are attached form an optionally substituted C₂-C₉heterocycloalkyl ring. In another embodiment, the FXR modulator is a compound of Formula (VIII) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (VIII) wherein R³⁴ and R³⁵ together with the nitrogen atom to which they are attached form a morpholinyl. In another embodiment, the FXR modulator is a compound of Formula (VIII) wherein R³⁴ and R³⁵ together with the nitrogen atom to which they are attached form an optionally substituted heteroaryl ring. In another embodiment, the FXR modulator is a compound of Formula (VIII) wherein R³⁴ and R³⁵ together with the nitrogen atom to which they are attached form an imidazolyl, pyrazolyl, or pyrrolyl.

In another embodiment, the FXR modulator is a compound of Formula (VIII) wherein R³⁰ is

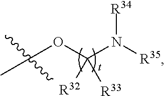

p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIII) wherein R³⁰ is

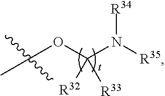

p is 2 and each R³¹ is independently halogen, or optionally substituted C₁-C₆alkyl. In another embodiment, the FXR modulator is a compound of Formula (VIII) wherein R³⁰ is

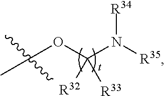

p is 2 and each R³¹ is halogen. In another embodiment, the FXR modulator is a compound of Formula (VIII) wherein R³⁰ is

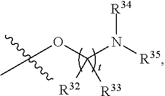

p is 2 and each R³¹ is F.

In another embodiment, the FXR modulator is a compound of Formula (VIII) wherein R³⁰ is

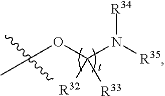

p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIII) wherein R³⁰ is

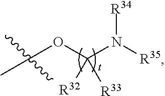

p is 1 and R³¹ is halogen, or optionally substituted C₁-C₆alkyl. In another embodiment, the FXR modulator is a compound of Formula (VIII) wherein R³⁰ is

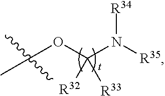

p is 1 and R³¹ is halogen. In another embodiment, the FXR modulator is a compound of Formula (VIII) wherein R³⁰ is

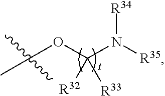

### p is 1 and R³¹ is F.

In another embodiment, the FXR modulator is a compound of Formula (VIII) wherein R³⁰ is

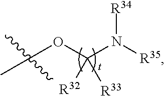

and p is 0.

In another embodiment, the FXR modulator is a compound of Formula (VIII) wherein R⁴ and R⁵ are each independently selected from the group consisting of hydrogen, halogen, and optionally substituted C₁-C₆alkyl. In another embodiment, the FXR modulator is a compound of Formula (VIII) wherein R⁴ and R⁵ are each independently selected from the group consisting of hydrogen and optionally substituted C₁-C₆alkyl. In another embodiment, the FXR modulator is a compound of Formula (VIII) wherein R⁴ and R⁵ are each hydrogen. In another embodiment, the FXR modulator is a compound of Formula (VIII) wherein R⁴ and R⁵ are each independently optionally substituted C₁-C₆alkyl. In another embodiment, the FXR modulator is a compound of Formula (VIII) wherein R⁴ and R⁵ are each methyl. In another embodiment, the FXR modulator is a compound of Formula (VIII) wherein R⁴ and R⁵ form an optionally substituted C₃-C₆cycloalkyl ring or an optionally substituted C₂-C₇heterocycloalkyl ring. In some embodiments, the FXR modulator is a compound of Formula (VIII) wherein R⁴ and R⁵ form an optionally substituted C₃-C₆cycloalkyl ring. In some embodiments, the FXR modulator is a compound of Formula (VIII) wherein R⁴ and R⁵ form an optionally substituted C₂-C₇heterocycloalkyl ring.

In another embodiment, the FXR modulator is a compound of Formula (VIII) wherein R⁶ and R⁷ are each independently selected from the group consisting of hydrogen, halogen, and optionally substituted C₁-C₆alkyl. In another embodiment, the FXR modulator is a compound of Formula (VIII) wherein R⁶ and R⁷ are each independently selected from the group consisting of hydrogen and optionally substituted C₁-C₆alkyl. In another embodiment, the FXR modulator is a compound of Formula (VIII) wherein R⁶ and R⁷ are each independently optionally substituted C₁-C₆alkyl. In another embodiment, the FXR modulator is a compound of Formula (VIII) wherein R⁶ and R⁷ are each methyl. In another embodiment, the FXR modulator is a compound of Formula (VIII) wherein R⁶ and R⁷ are each hydrogen.

In a further embodiment of the aforementioned embodiments is a compound of Formula (VIII) wherein R² is selected from the group consisting of —CN, —C(O)OR²⁵, —C(O)N(R²⁵)R²⁶,

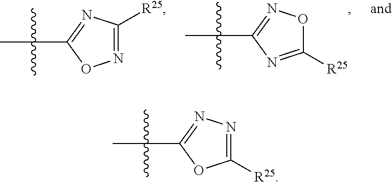

In a further embodiment of the aforementioned embodiments, the FXR modulator is a compound of Formula (VIII) wherein R² is —CN.

In a further embodiment of the aforementioned embodiments, the FXR modulator is a compound of Formula (VIII) wherein R² is —C(O)OR²⁵. In a further embodiment of the aforementioned embodiments, the FXR modulator is a compound of Formula (VIII) wherein R² is —C(O)OR²⁵, and R²⁵ is independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (VIII) wherein R² is —C(O)OR²⁵, and R²⁵ is independently selected from the group consisting of hydrogen, and optionally substituted C₁-C₆alkyl. In a further embodiment of the aforementioned embodiments, the FXR modulator is a compound of Formula (VIII) wherein R² is —C(O)OR²⁵, and R²⁵ is hydrogen. In a further embodiment of the aforementioned embodiments, the FXR modulator is a compound of Formula (VIII) wherein R² is —C(O)OR²⁵, and R²⁵ is optionally substituted C₁-C₆alkyl. In a further embodiment of the aforementioned embodiments, the FXR modulator is a compound of Formula (VIII) wherein R² is —C(O)OR²⁵, and R²⁵ is unsubstituted C₁-C₆alkyl. In a further embodiment of the aforementioned embodiments, the FXR modulator is a compound of Formula (VIII) wherein R² is —C(O)OR²⁵, and R²⁵ is methyl. In a further embodiment of the aforementioned embodiments, the FXR modulator is a compound of Formula (VIII) wherein R² is —C(O)OR²⁵, and R²⁵ is ethyl.

In a further embodiment of the aforementioned embodiments, the FXR modulator is a compound of Formula (VIII) wherein R² is —C(O)N(R²⁵)R²⁶. In a further embodiment of the aforementioned embodiments, the FXR modulator is a compound of Formula (VIII) wherein R² is —C(O)N(R²⁵)R²⁶, and R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (VIII) wherein R² is —C(O)N(R²⁵)R²⁶, and R²⁵ and R²⁶ are each independently selected from the group consisting of hydrogen, and optionally substituted C₁-C₆alkyl. In a further embodiment of the aforementioned embodiments, the FXR modulator is a compound of Formula (VIII) wherein R² is —C(O)N(R²⁵)R²⁶, and R²⁵ and R²⁶ are hydrogen. In a further embodiment of the aforementioned embodiments, the FXR modulator is a compound of Formula (VIII) wherein R² is —C(O)N(R²⁵)R²⁶, and R²⁵ and R²⁶ are each independently optionally substituted C₁-C₆alkyl. In a further embodiment of the aforementioned embodiments, the FXR modulator is a compound of Formula (VIII) wherein R² is —C(O)N(R²⁵)R²⁶, R²⁵ is hydrogen, and R²⁶ is optionally substituted C₁-C₆alkyl. In a further embodiment of the aforementioned embodiments, the FXR modulator is a compound of Formula (VIII) wherein R² is —C(O)N(R²⁵)R²⁶, and R²⁵ and R²⁶ are each independently unsubstituted C₁-C₆alkyl. In a further embodiment of the aforementioned embodiments, the FXR modulator is a compound of Formula (VIII) wherein R² is —C(O)N(R²⁵)R²⁶, R²⁵ is hydrogen, and R²⁶ are methyl. In a further embodiment of the aforementioned embodiments, the FXR modulator is a compound of Formula (VIII) wherein R² is —C(O)N(R²⁵)R²⁶, and R²⁵ and R²⁶ are methyl. In a further embodiment of the aforementioned embodiments, the FXR modulator is a compound of Formula (VIII) wherein R² is —C(O)N(R²⁵)R²⁶, and R²⁵ and R²⁶ are ethyl.

In a further embodiment of the aforementioned embodiments, the FXR modulator is a compound of Formula (VIII) wherein R² is

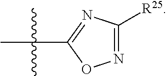

In a further embodiment of the aforementioned embodiments, the FXR modulator is a compound of Formula (VIII) wherein R² is

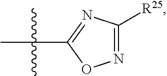

and R²⁵ is optionally substituted C₁-C₆alkyl. In a further embodiment of the aforementioned embodiments, the FXR modulator is a compound of Formula (VIII) wherein R² is

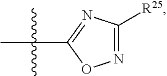

and R²⁵ is methyl. In a further embodiment of the aforementioned embodiments, the FXR modulator is a compound of Formula (VIII) wherein R² is

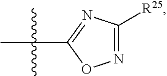

and R²⁵ is ethyl.

In a further embodiment of the aforementioned embodiments, the FXR modulator is a compound of Formula (VIII) wherein R² is

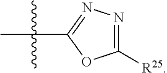

In a further embodiment of the aforementioned embodiments, the FXR modulator is a compound of Formula (VIII) wherein R² is

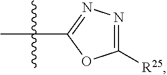

and R²⁵ is optionally substituted C₁-C₆alkyl. In a further embodiment of the aforementioned embodiments, the FXR modulator is a compound of Formula (VIII) wherein R² is

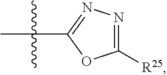

and R²⁵ is methyl. In a further embodiment of the aforementioned embodiments, the FXR modulator is a compound of Formula (VIII) wherein R² is

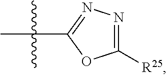

and R²⁵ is ethyl.

In a further embodiment of the aforementioned embodiments, the FXR modulator is a compound of Formula (VIII) wherein R² is

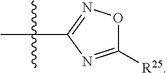

In a further embodiment of the aforementioned embodiments, the FXR modulator is a compound of Formula (VIII) wherein R² is

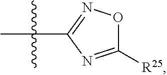

and R²⁵ is optionally substituted C₁-C₆alkyl. In a further embodiment of the aforementioned embodiments, the FXR modulator is a compound of Formula (VIII) wherein R² is

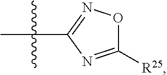

and R²⁵ is methyl. In a further embodiment of the aforementioned embodiments, the FXR modulator is a compound of Formula (VIII) wherein R² is

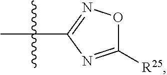

and R²⁵ is ethyl.

In a further embodiment of the aforementioned embodiments, the FXR modulator is a compound of Formula (VIII) wherein R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (VIII) wherein R¹ is hydrogen. In a further embodiment of the aforementioned embodiments, the FXR modulator is a compound of Formula (VIII) wherein R¹ is optionally substituted C₁-C₆alkyl. In a further embodiment of the aforementioned embodiments, the FXR modulator is a compound of Formula (VIII) wherein R¹ is methyl. In a further embodiment of the aforementioned embodiments, the FXR modulator is a compound of Formula (VIII) wherein R¹ is optionally substituted C₂-C₆alkenyl. In a further embodiment of the aforementioned embodiments, the FXR modulator is a compound of Formula (VIII) wherein R¹ is optionally substituted C₂-C₆alkynyl.

In a further embodiment of the aforementioned embodiments, the FXR modulator is a compound of Formula (VIII) wherein R¹ and R² together with the carbon atoms to which they are attached, form an optionally substituted C₂-C₉heterocycloalkyl ring or an optionally substituted heteroaryl ring. In a further embodiment of the aforementioned embodiments, the FXR modulator is a compound of Formula (VIII) wherein R¹ and R² together with the carbon atoms to which they are attached, form an optionally substituted C₂-C₉heterocycloalkyl ring. In a further embodiment of the aforementioned embodiments, the FXR modulator is a compound of Formula (VIII) wherein R¹ and R² together with the carbon atoms to which they are attached, form an optionally substituted heteroaryl ring.

In a further embodiment of the aforementioned embodiments, the FXR modulator is a compound of Formula (VIII) wherein R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl). In a further embodiment of the aforementioned embodiments, the FXR modulator is a compound of Formula (VIII) wherein R⁸ is selected from the group consisting of hydrogen, and optionally substituted C₁-C₆alkyl. In a further embodiment of the aforementioned embodiments, the FXR modulator is a compound of Formula (VIII) wherein R⁸ is optionally substituted C₁-C₆alkyl. In a further embodiment of the aforementioned embodiments, the FXR modulator is a compound of Formula (VIII) wherein R⁸ is methyl. In a further embodiment of the aforementioned embodiments, the FXR modulator is a compound of Formula (VIII) wherein R⁸ is optionally substituted C₁-C₆alkyl. In a further embodiment of the aforementioned embodiments, the FXR modulator is a compound of Formula (VIII) wherein R⁸ is ethyl. In a further embodiment of the aforementioned embodiments, the FXR modulator is a compound of Formula (VIII) wherein R⁸ is optionally substituted C₁-C₆alkyl. In a further embodiment of the aforementioned embodiments, the FXR modulator is a compound of Formula (VIII) wherein R⁸ is hydrogen.

In a further embodiment of the aforementioned embodiments provided herein, the FXR modulator compound of Formula (VIII) has the structure of Formula (VIIIa), or a pharmaceutically acceptable salt, stereoisomer, or solvate thereof:

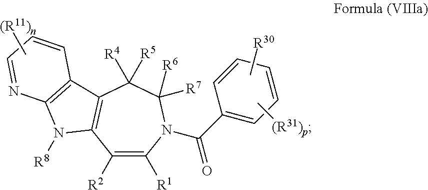

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In a further embodiment of the aforementioned embodiments provided herein, the FXR modulator compound of Formula (VIII) has the structure of Formula (VIIIb), or a pharmaceutically acceptable salt, stereoisomer, or solvate thereof:

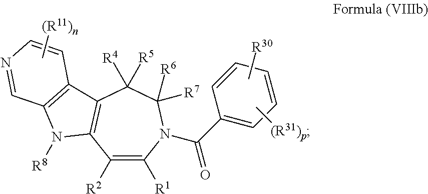

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In a further embodiment of the aforementioned embodiments provided herein, the FXR modulator compound of Formula (VIII) has the structure of Formula (VIIIc), or a pharmaceutically acceptable salt, stereoisomer, or solvate thereof:

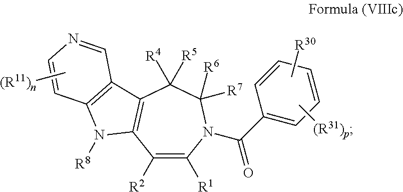

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In a further embodiment of the aforementioned embodiments provided herein, the FXR modulator compound of Formula (VIII) has the structure of Formula (VIIId), or a pharmaceutically acceptable salt, stereoisomer, or solvate thereof:

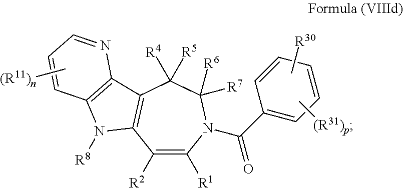

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In a further embodiment of the aforementioned embodiments provided herein, the FXR modulator compound of Formula (VIII) has the structure of Formula (VIIIe), or a pharmaceutically acceptable salt, stereoisomer, or solvate thereof:

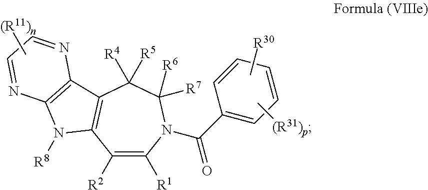

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In a further embodiment of the aforementioned embodiments provided herein, the FXR modulator compound of Formula (VIII) has the structure of Formula (VIIIf), or a pharmaceutically acceptable salt, stereoisomer, or solvate thereof:

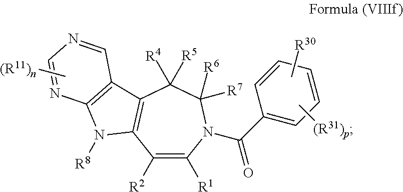

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In a further embodiment of the aforementioned embodiments provided herein, the FXR modulator compound of Formula (VIII) has the structure of Formula (VIIIg), or a pharmaceutically acceptable salt, stereoisomer, or solvate thereof:

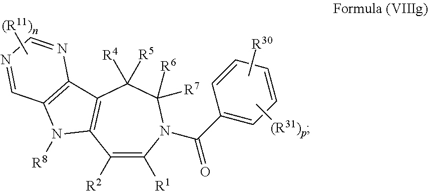

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, or 2.

In a further embodiment of the aforementioned embodiments provided herein, the FXR modulator compound of Formula (VIII) has the structure of Formula (VIIIh), or a pharmaceutically acceptable salt, stereoisomer, or solvate thereof:

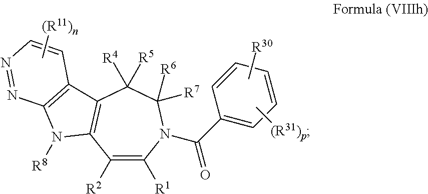

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In a further embodiment of the aforementioned embodiments provided herein, the FXR modulator compound of Formula (VIII) has the structure of Formula (VIIIi), or a pharmaceutically acceptable salt, stereoisomer, or solvate thereof:

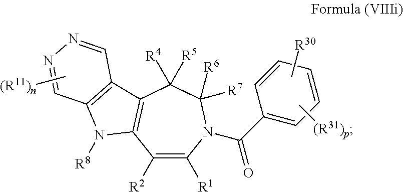

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In a further embodiment of the aforementioned embodiments provided herein, the FXR modulator compound of Formula (VIII) has the structure of Formula (VIIIj), or a pharmaceutically acceptable salt, stereoisomer, or solvate thereof:

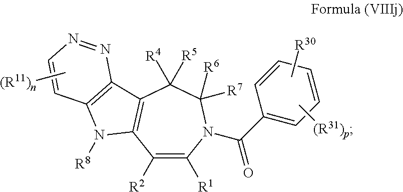

wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

In some embodiments, the FXR modulator is a compound of Formula (VIIIa)-(VIIIj) wherein n is 0. In a further embodiment of the aforementioned embodiments, the FXR modulator is a compound of Formula (VIIIa)-(VIIIj) wherein n is 1. In a further embodiment of the aforementioned embodiments, the FXR modulator is a compound of Formula (VIIIa)-(VIIIj) wherein n is 2. In a further embodiment of the aforementioned embodiments, the FXR modulator is a compound of Formula (VIIIa)-(VIIIj) wherein n is 1 and R¹¹ is selected from the group consisting of halogen, —CN, amino, alkylamino, C₁-C₆alkyl, C₁-C₆haloalkyl, C₁-C₆alkoxy, and C₁-C₆haloalkoxy. In a further embodiment of the aforementioned embodiments, the FXR modulator is a compound of Formula (VIIIa)-(VIIIj) wherein n is 1 and R¹¹ is selected from the group consisting of halogen, —CN, C₁-C₆alkyl, and C₁-C₆alkoxy. In a further embodiment of the aforementioned embodiments, the FXR modulator is a compound of Formula (VIIIa)-(VIIIj) wherein n is 1 and R¹¹ is halogen.

In one embodiment, the FXR modulator is a compound of Formula (VIIIa)-(VIIIj) wherein p is 0. In another embodiment, the FXR modulator is a compound of Formula (VIIIa)-(VIIIj) wherein p is 1. In another embodiment, the FXR modulator is a compound of Formula (VIIIa)-(VIIIj) wherein p is 2. In another embodiment, the FXR modulator is a compound of Formula (VIIIa)-(VIIIj) wherein p is 3. In another embodiment, the FXR modulator is a compound of Formula (VIIIa)-(VIIIj) wherein p is 4.

In another embodiment, the FXR modulator is a compound of Formula (VIIIa)-(VIIIj) wherein p is 2 and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIIIa)-(VIIIj) wherein p is 2 and each R³¹ is independently halogen, or optionally substituted C₁-C₆alkyl. In another embodiment, the FXR modulator is a compound of Formula (VIIIa)-(VIIIj) wherein p is 2 and each R³¹ is halogen. In another embodiment, the FXR modulator is a compound of Formula (VIIIa)-(VIIIj) wherein p is 2 and each R³¹ is F.

In another embodiment, the FXR modulator is a compound of Formula (VIIIa)-(VIIIj) wherein R³⁰ is F, p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIIIa)-(VIIIj) wherein R³⁰ is F, p is 2 and each R³¹ is independently halogen, or optionally substituted C₁-C₆alkyl. In another embodiment, the FXR modulator is a compound of Formula (VIIIa)-(VIIIj) wherein R³⁰ is F, p is 2 and each R³¹ is halogen. In another embodiment, the FXR modulator is a compound of Formula (VIIIa)-(VIIIj) wherein R³⁰ is F, p is 2 and each R³¹ is F.

In another embodiment, the FXR modulator is a compound of Formula (VIIIa)-(VIIIj) wherein p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIIIa)-(VIIIj) wherein p is 1 and R³¹ is halogen, or optionally substituted C₁-C₆alkyl. In another embodiment, the FXR modulator is a compound of Formula (VIIIa)-(VIIIj) wherein p is 1 and R³¹ is halogen. In another embodiment, the FXR modulator is a compound of Formula (VIIIa)-(VIIIj) wherein p is 1 and R³¹ is F.

In another embodiment, the FXR modulator is a compound of Formula (VIIIa)-(VIIIj) wherein R³⁰ is F, p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIIIa)-(VIIIj) wherein R³⁰ is F, p is 1 and R³¹ is halogen, or optionally substituted C₁-C₆alkyl. In another embodiment, the FXR modulator is a compound of Formula (VIIIa)-(VIIIj) wherein R³⁰ is F, p is 1 and R³¹ is halogen. In another embodiment, the FXR modulator is a compound of Formula (VIIIa)-(VIIIj) wherein R³⁰ is F, p is 1 and R³¹ is F.

In another embodiment, the FXR modulator is a compound of Formula (VIIIa)-(VIIIj) wherein R³⁰ is

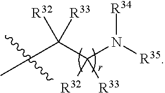

In one embodiment, the FXR modulator is a compound of Formula (VIIIa)-(VIIIj) wherein r is 0. In another embodiment, the FXR modulator is a compound of Formula (VIIIa)-(VIIIj) wherein r is 1. In another embodiment, the FXR modulator is a compound of Formula (VIIIa)-(VIIIj) wherein r is 2. In another embodiment, the FXR modulator is a compound of Formula (VIIIa)-(VIIIj) wherein r is 3. In another embodiment, the FXR modulator is a compound of Formula (VIIIa)-(VIIIj) wherein r is 4. In one embodiment, the FXR modulator is a compound of Formula (VIIIa)-(VIIIj) wherein each R³² and R³³ are hydrogen. In one embodiment, the FXR modulator is a compound of Formula (VIIIa)-(VIIIj) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (VIIIa)-(VIIIj) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (VIIIa)-(VIIIj) wherein R³⁴ and R³⁵ together with the nitrogen atom to which they are attached form an optionally substituted C₂-C₉heterocycloalkyl ring. In another embodiment, the FXR modulator is a compound of Formula (VIIIa)-(VIIIj) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (VIIIa)-(VIIIj) wherein R³⁴ and R³⁵ together with the nitrogen atom to which they are attached form a morpholinyl. In another embodiment, the FXR modulator is a compound of Formula (VIIIa)-(VIIIj) wherein R³⁴ and R³⁵ together with the nitrogen atom to which they are attached form an optionally substituted heteroaryl ring. In another embodiment, the FXR modulator is a compound of Formula (VIIIa)-(VIIIj) wherein R³⁴ and R³⁵ together with the nitrogen atom to which they are attached form an imidazolyl, pyrazolyl, or pyrrolyl.

In another embodiment, the FXR modulator is a compound of Formula (VIIIa)-(VIIIj) wherein R³⁰ is

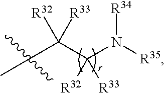

p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIIIa)-(VIIIj) wherein R³⁰ is

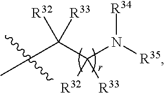

p is 2 and each R³¹ is independently halogen, or optionally substituted C₁-C₆alkyl. In another embodiment, the FXR modulator is a compound of Formula (VIIIa)-(VIIIj) wherein R³⁰ is

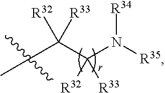

p is 2 and each R³¹ is halogen. In another embodiment, the FXR modulator is a compound of Formula (VIIIa)-(VIIIj) wherein R³⁰ is

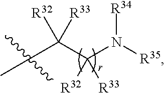

p is 2 and each R³¹ is F.

In another embodiment, the FXR modulator is a compound of Formula (VIIIa)-(VIIIj) wherein R³⁰ is

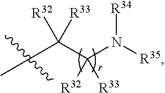

p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIIIa)-(VIIIj) wherein R³⁰ is

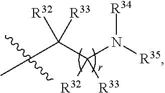

p is 1 and R³¹ is halogen, or optionally substituted C₁-C₆alkyl. In another embodiment, the FXR modulator is a compound of Formula (VIIIa)-(VIIIj) wherein R³⁰ is

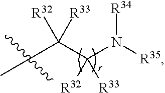

p is 1 and R³¹ is halogen. In another embodiment, the FXR modulator is a compound of Formula (VIIIa)-(VIIIj) wherein R³⁰ is

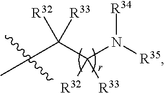

### p is 1 and R³¹ is F.

In another embodiment, the FXR modulator is a compound of Formula (VIIIa)-(VIIIj) wherein R³⁰ is

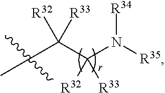

and p is 0.

In another embodiment, the FXR modulator is a compound of Formula (VIIIa)-(VIIIj) wherein R³⁰ is

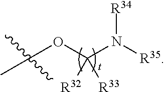

In one embodiment, the FXR modulator is a compound of Formula (VIIIa)-(VIIIj) wherein t is 2. In another embodiment, the FXR modulator is a compound of Formula (VIIIa)-(VIIIj) wherein t is 3. In another embodiment, the FXR modulator is a compound of Formula (VIIIa)-(VIIIj) wherein t is 4. In one embodiment, the FXR modulator is a compound of Formula (VIIIa)-(VIIIj) wherein each R³² and R³³ are hydrogen. In one embodiment, the FXR modulator is a compound of Formula (VIIIa)-(VIIIj) wherein R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl. In another embodiment, the FXR modulator is a compound of Formula (VIIIa)-(VIIIj) wherein R³⁴ and R³⁵ are each independently selected from the group consisting of hydrogen, C₁-C₆alkyl, C₃-C₈cycloalkyl, and C₂-C₉heterocycloalkyl. In another embodiment, the FXR modulator is a compound of Formula (VIIIa)-(VIIIj) wherein R³⁴ and R³⁵ together with the nitrogen atom to which they are attached form an optionally substituted C₂-C₉heterocycloalkyl ring. In another embodiment, the FXR modulator is a compound of Formula (VIIIa)-(VIIIj) wherein R³⁴ and R³⁵ together with the nitrogen atom to which they are attached form a pyrrolidinyl, piperidinyl, morpholinyl, piperazinyl, or methylpiperazinyl. In another embodiment, the FXR modulator is a compound of Formula (VIIIa)-(VIIIj) wherein R³⁴ and R³⁵ together with the nitrogen atom to which they are attached form a morpholinyl. In another embodiment, the FXR modulator is a compound of Formula (VIIIa)-(VIIIj) wherein R³⁴ and R³⁵ together with the nitrogen atom to which they are attached form an optionally substituted heteroaryl ring. In another embodiment, the FXR modulator is a compound of Formula (VIIIa)-(VIIIj) wherein R³⁴ and R³⁵ together with the nitrogen atom to which they are attached form an imidazolyl, pyrazolyl, pyrrolyl.

In another embodiment, the FXR modulator is a compound of Formula (VIIIa)-(VIIIj) wherein R³⁰ is

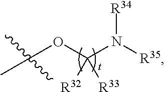

p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIIIa)-(VIIIj) wherein R³⁰ is

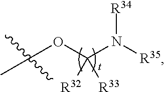

p is 2 and each R³¹ is independently halogen, or optionally substituted C₁-C₆alkyl. In another embodiment, the FXR modulator is a compound of Formula (VIIIa)-(VIIIj) wherein R³⁰ is

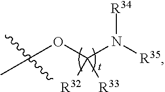

p is 2 and each R³¹ is halogen. In another embodiment, the FXR modulator is a compound of Formula (VIIIa)-(VIIIj) wherein R³⁰ is

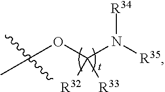

p is 2 and each R³¹ is F.

In another embodiment, the FXR modulator is a compound of Formula (VIIIa)-(VIIIj) wherein R³⁰ is

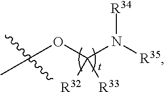

p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In another embodiment, the FXR modulator is a compound of Formula (VIIIa)-(VIIIj) wherein R³⁰ is

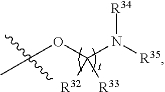

p is 1 and R³¹ is halogen, or optionally substituted C₁-C₆alkyl. In another embodiment, the FXR modulator is a compound of Formula (VIIIa)-(VIIIj) wherein R³⁰ is

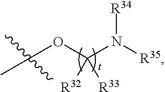

p is 1 and R³¹ is halogen. In another embodiment, the FXR modulator is a compound of Formula (VIIIa)-(VIIIj) wherein R³⁰ is

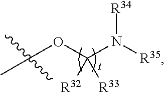

### p is 1 and R³¹ is F.

In another embodiment, the FXR modulator is a compound of Formula (VIIIa)-(VIIIj) wherein R³⁰ is

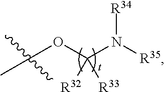

and p is 0.

In another embodiment, the FXR modulator is a compound of Formula (VIIIa)-(VIIIj) wherein R⁴ and R⁵ are each independently selected from the group consisting of hydrogen, halogen, and optionally substituted C₁-C₆alkyl. In another embodiment, the FXR modulator is a compound of Formula (VIIIa)-(VIIIj) wherein R⁴ and R⁵ are each independently selected from the group consisting of hydrogen and optionally substituted C₁-C₆alkyl. In another embodiment, the FXR modulator is a compound of Formula (VIIIa)-(VIIIj) wherein R⁴ and R⁵ are each hydrogen. In another embodiment, the FXR modulator is a compound of Formula (VIIIa)-(VIIIj) wherein R⁴ and R⁵ are each independently optionally substituted C₁-C₆alkyl. In another embodiment, the FXR modulator is a compound of Formula (VIIIa)-(VIIIj) wherein R⁴ and R⁵ are each methyl. In another embodiment, the FXR modulator is a compound of Formula (VIIIa)-(VIIIj) wherein R⁴ and R⁵ form an optionally substituted C₃-C₆cycloalkyl ring or an optionally substituted C₂-C₇heterocycloalkyl ring. In some embodiments, the FXR modulator is a compound of Formula (VIIIa)-(VIIIj) wherein R⁴ and R⁵ form an optionally substituted C₃-C₆cycloalkyl ring. In some embodiments, the FXR modulator is a compound of Formula (VIIIa)-(VIIIj) wherein R⁴ and R⁵ form an optionally substituted C₂-C₇heterocycloalkyl ring.

In another embodiment, the FXR modulator is a compound of Formula (VIIIa)-(VIIIj) wherein R⁶ and R⁷ are each independently selected from the group consisting of hydrogen, halogen, and optionally substituted C₁-C₆alkyl. In another embodiment, the FXR modulator is a compound of Formula (VIIIa)-(VIIIj) wherein R⁶ and R⁷ are each independently selected from the group consisting of hydrogen and optionally substituted C₁-C₆alkyl. In another embodiment, the FXR modulator is a compound of Formula (VIIIa)-(VIIIj) wherein R⁶ and R⁷ are each independently optionally substituted C₁-C₆alkyl. In another embodiment, the FXR modulator is a compound of Formula (VIIIa)-(VIIIj) wherein R⁶ and R⁷ are each methyl. In another embodiment, the FXR modulator is a compound of Formula (VIIIa)-(VIIIj) wherein R⁶ and R⁷ are each hydrogen.

In a further embodiment of the aforementioned embodiments, the FXR modulator is a compound of Formula (VIIIa)-(VIIIj) wherein R² is selected from the group consisting of —CN, —C(O)OR²⁵, —C(O)N(R²⁵)R²⁶,

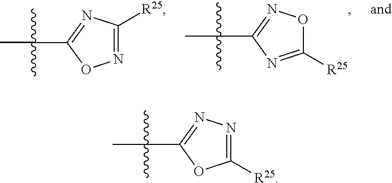

In a further embodiment of the aforementioned embodiments, the FXR modulator is a compound of Formula (VIIIa)-(VIIIj) wherein R² is —CN.

In a further embodiment of the aforementioned embodiments, the FXR modulator is a compound of Formula (VIIIa)-(VIIIj) wherein R² is —C(O)OR²⁵. In a further embodiment of the aforementioned embodiments, the FXR modulator is a compound of Formula (VIIIa)-(VIIIj) wherein R² is —C(O)OR²⁵, and R²⁵ is independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (VIIIa)-(VIIIj) wherein R² is —C(O)OR²⁵, and R²⁵ is independently selected from the group consisting of hydrogen, and optionally substituted C₁-C₆alkyl. In a further embodiment of the aforementioned embodiments, the FXR modulator is a compound of Formula (VIIIa)-(VIIIj) wherein R² is —C(O)OR²⁵, and R²⁵ is hydrogen. In a further embodiment of the aforementioned embodiments, the FXR modulator is a compound of Formula (VIIIa)-(VIIIj) wherein R² is —C(O)OR²⁵, and R²⁵ is optionally substituted C₁-C₆alkyl. In a further embodiment of the aforementioned embodiments, the FXR modulator is a compound of Formula (VIIIa)-(VIIIj) wherein R² is —C(O)OR²⁵, and R²⁵ is unsubstituted C₁-C₆alkyl. In a further embodiment of the aforementioned embodiments, the FXR modulator is a compound of Formula (VIIIa)-(VIIIj) wherein R² is —C(O)OR²⁵, and R²⁵ is methyl. In a further embodiment of the aforementioned embodiments, the FXR modulator is a compound of Formula (VIIIa)-(VIIIj) wherein R² is —C(O)OR²⁵, and R²⁵ is ethyl. In a further embodiment of the aforementioned embodiments, the FXR modulator is a compound of Formula (VIIIa)-(VIIIj) wherein R² is —C(O)OR²⁵, and R²⁵ is isopropyl.

In a further embodiment of the aforementioned embodiments, the FXR modulator is a compound of Formula (VIIIa)-(VIIIj) wherein R² is —C(O)N(R²⁵)R²⁶. In a further embodiment of the aforementioned embodiments, the FXR modulator is a compound of Formula (VIIIa)-(VIIIj) wherein R² is —C(O)N(R²⁵)R²⁶, and R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (VIIIa)-(VIIIj) wherein R² is —C(O)N(R²⁵)R²⁶, and R²⁵ and R²⁶ are each independently selected from the group consisting of hydrogen, and optionally substituted C₁-C₆alkyl. In a further embodiment of the aforementioned embodiments, the FXR modulator is a compound of Formula (VIIIa)-(VIIIj) wherein R² is —C(O)N(R²⁵)R²⁶, and R²⁵ and R²⁶ are hydrogen. In a further embodiment of the aforementioned embodiments, the FXR modulator is a compound of Formula (VIIIa)-(VIIIj) wherein R² is —C(O)N(R²⁵)R²⁶, and R²⁵ and R²⁶ are each independently optionally substituted C₁-C₆alkyl. In a further embodiment of the aforementioned embodiments, the FXR modulator is a compound of Formula (VIIIa)-(VIIIj) wherein R² is —C(O)N(R²⁵)R²⁶, R²⁵ is hydrogen, and R²⁶ is optionally substituted C₁-C₆alkyl. In a further embodiment of the aforementioned embodiments, the FXR modulator is a compound of Formula (VIIIa)-(VIIIj) wherein R² is —C(O)N(R²⁵)R²⁶, and R²⁵ and R²⁶ are each independently unsubstituted C₁-C₆alkyl. In a further embodiment of the aforementioned embodiments, the FXR modulator is a compound of Formula (VIIIa)-(VIIIj) wherein R² is —C(O)N(R²⁵)R²⁶, R²⁵ is hydrogen, and R²⁶ are methyl. In a further embodiment of the aforementioned embodiments, the FXR modulator is a compound of Formula (VIIIa)-(VIIIj) wherein R² is —C(O)N(R²⁵)R²⁶, and R²⁵ and R²⁶ are methyl. In a further embodiment of the aforementioned embodiments, the FXR modulator is a compound of Formula (VIIIa)-(VIIIj) wherein R² is —C(O)N(R²⁵)R²⁶, and R²⁵ and R²⁶ are ethyl.

In a further embodiment of the aforementioned embodiments, the FXR modulator is a compound of Formula (VIIIa)-(VIIIj) wherein R² is

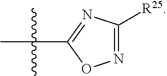

In a further embodiment of the aforementioned embodiments, the FXR modulator is a compound of Formula (VIIIa)-(VIIIj) wherein R² is

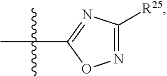

and R²⁵ is optionally substituted C₁-C₆alkyl. In a further embodiment of the aforementioned embodiments, the FXR modulator is a compound of Formula (VIIIa)-(VIIIj) wherein R² is

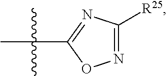

and R²⁵ is methyl. In a further embodiment of the aforementioned embodiments, the FXR modulator is a compound of Formula (VIIIa)-(VIIIj) wherein R² is

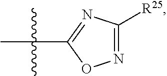

and R²⁵ is ethyl.

In a further embodiment of the aforementioned embodiments, the FXR modulator is a compound of Formula (VIIIa)-(VIIIj) wherein R² is

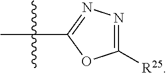

In a further embodiment of the aforementioned embodiments, the FXR modulator is a compound of Formula (VIIIa)-(VIIIj) wherein R² is

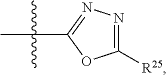

and R²⁵ is optionally substituted C₁-C₆alkyl. In a further embodiment of the aforementioned embodiments, the FXR modulator is a compound of Formula (VIIIa)-(VIIIj) wherein R² is

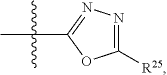

and R²⁵ is methyl. In a further embodiment of the aforementioned embodiments, the FXR modulator is a compound of Formula (VIIIa)-(VIIIj) wherein R² is

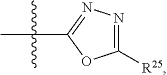

and R²⁵ is ethyl.

In a further embodiment of the aforementioned embodiments, the FXR modulator is a compound of Formula (VIIIa)-(VIIIj) wherein R² is

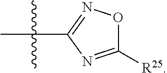

In a further embodiment of the aforementioned embodiments, the FXR modulator is a compound of Formula (VIIIa)-(VIIIj) wherein R² is

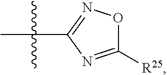

and R²⁵ is optionally substituted C₁-C₆alkyl. In a further embodiment of the aforementioned embodiments, the FXR modulator is a compound of Formula (VIIIa)-(VIIIj) wherein R² is

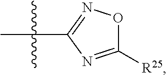

and R²⁵ is methyl. In a further embodiment of the aforementioned embodiments, the FXR modulator is a compound of Formula (VIIIa)-(VIIIj) wherein R² is

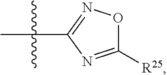

and R²⁵ is ethyl.

In a further embodiment of the aforementioned embodiments, the FXR modulator is a compound of Formula (VIIIa)-(VIIIj) wherein R¹ is selected from the group consisting of hydrogen and optionally substituted C₁-C₆alkyl. In a further embodiment of the aforementioned embodiments, the FXR modulator is a compound of Formula (VIIIa)-(VIIIj) wherein R¹ is hydrogen. In a further embodiment of the aforementioned embodiments, the FXR modulator is a compound of Formula (VIIIa)-(VIIIj) wherein R¹ is optionally substituted C₁-C₆alkyl. In a further embodiment of the aforementioned embodiments, the FXR modulator is a compound of Formula (VIIIa)-(VIIIj) wherein R¹ is unsubstituted C₁-C₆alkyl. In a further embodiment of the aforementioned embodiments, the FXR modulator is a compound of Formula (VIIIa)-(VIIIj) wherein R¹ is methyl.

In a further embodiment of the aforementioned embodiments, the FXR modulator is a compound of Formula (VIIIa)-(VIIIj) wherein R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl). In a further embodiment of the aforementioned embodiments, the FXR modulator is a compound of Formula (VIIIa)-(VIIIj) wherein R⁸ is selected from the group consisting of hydrogen, and optionally substituted C₁-C₆alkyl. In a further embodiment of the aforementioned embodiments, the FXR modulator is a compound of Formula (VIIIa)-(VIIIj) wherein R⁸ is optionally substituted C₁-C₆alkyl. In a further embodiment of the aforementioned embodiments, the FXR modulator is a compound of Formula (VIIIa)-(VIIIj) wherein R⁸ is methyl. In a further embodiment of the aforementioned embodiments, the FXR modulator is a compound of Formula (VIIIa)-(VIIIj) wherein R⁸ is optionally substituted C₁-C₆alkyl. In a further embodiment of the aforementioned embodiments, the FXR modulator is a compound of Formula (VIIIa)-(VIIIj) wherein R⁸ is methyl. In a further embodiment of the aforementioned embodiments, the FXR modulator is a compound of Formula (VIIIa)-(VIIIj) wherein R⁸ is ethyl. In a further embodiment of the aforementioned embodiments, the FXR modulator is a compound of Formula (VIIIa)-(VIIIj) wherein R⁸ is hydrogen.

In some embodiments is a method of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof; wherein the FXR modulator is a compound of Formula (IX), or a pharmaceutically acceptable salt, stereoisomer, or solvate thereof:

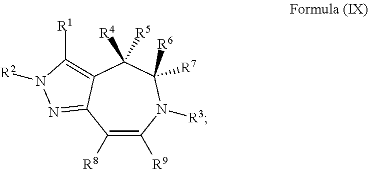

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R² is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of —CN, —C(O)OR²⁵, —C(O)N(R²⁵)R²⁶,

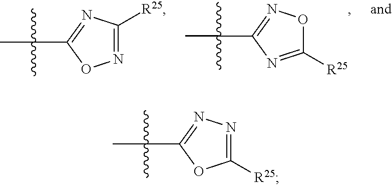

R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R⁸ and R⁹ together with the carbon atoms to which they are attached, form an optionally substituted C₂-C₉heterocycloalkyl ring or an optionally substituted heteroaryl ring;
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring; or a pharmaceutically acceptable salt, stereoisomer, or solvate thereof.

In some embodiments is a method of treating a metabolic disorder in an individual in need thereof, comprising co-administering to the individual a therapeutically effective amount of: (a) a first agent that is an FXR modulator; and (b) at least one second agent that is a DPP-IV inhibitor, an SGLT2 inhibitor, an ASK1 inhibitor, a GLP-1 agonist, or a combination thereof; wherein the FXR modulator is a compound of Formula (X), or a pharmaceutically acceptable salt, stereoisomer, or solvate thereof:

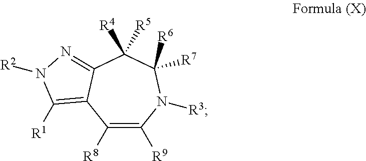

wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R² is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of —CN, —C(O)OR²⁵, —C(O)N(R²⁵)R²⁶;

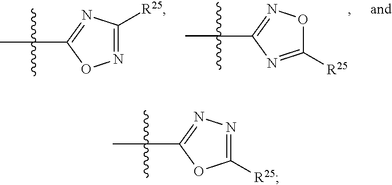

R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R⁸ and R⁹ together with the carbon atoms to which they are attached, form an optionally substituted C₂-C₉heterocycloalkyl ring or an optionally substituted heteroaryl ring;
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring; or a pharmaceutically acceptable salt, stereoisomer, or solvate thereof.

In one embodiment, the FXR modulator is a compound of Formula (IX) or (X) wherein R⁴ and R⁵ are each independently selected from the group consisting of hydrogen, halogen, and optionally substituted C₁-C₆alkyl. In another embodiment, the FXR modulator is a compound of Formula (IX) or (X) wherein R⁴ and R⁵ are each independently selected from the group consisting of hydrogen and optionally substituted C₁-C₆alkyl. In another embodiment, the FXR modulator is a compound of Formula (IX) or (X) wherein R⁴ and R⁵ are each hydrogen. In another embodiment, the FXR modulator is a compound of Formula (IX) or (X) wherein R⁴ and R⁵ are each independently optionally substituted C₁-C₆alkyl. In another embodiment, the FXR modulator is a compound of Formula (IX) or (X) wherein R⁴ and R⁵ are each methyl.

In another embodiment, the FXR modulator is a compound of Formula (IX) or (X) wherein R⁶ and R⁷ are each independently selected from the group consisting of hydrogen, halogen, and optionally substituted C₁-C₆alkyl. In another embodiment, the FXR modulator is a compound of Formula (IX) or (X) wherein R⁶ and R⁷ are each independently selected from the group consisting of hydrogen and optionally substituted C₁-C₆alkyl. In another embodiment, the FXR modulator is a compound of Formula (IX) or (X) wherein R⁶ and R⁷ are each independently optionally substituted C₁-C₆alkyl. In another embodiment, the FXR modulator is a compound of Formula (IX) or (X) wherein R⁶ and R⁷ are each methyl. In another embodiment, the FXR modulator is a compound of Formula (IX) or (X) wherein R⁶ and R⁷ are each hydrogen.

In another embodiment, the FXR modulator is a compound of Formula (IX) or (X) wherein R⁶ and R⁷ are hydrogen, R⁴ and R⁵ are independently optionally substituted C₁-C₆alkyl, R³ is —C(O)R²⁰, and R²⁰ is optionally substituted aryl. In another embodiment, the FXR modulator is a compound of Formula (IX) or (X) wherein R⁶ and R⁷ are hydrogen, R⁴ and R⁵ are independently optionally substituted C₁-C₆alkyl, R³ is —C(O)R²⁰, and R²⁰ is optionally substituted heteroaryl. In another embodiment, the FXR modulator is a compound of Formula (IX) or (X) wherein R⁶ and R⁷ are hydrogen, R⁴ and R⁵ are methyl, R³ is —C(O)R²⁰, and R²⁰ is optionally substituted aryl. In another embodiment, the FXR modulator is a compound of Formula (IX) or (X) wherein R⁶ and R⁷ are hydrogen, R⁴ and R⁵ are methyl, R³ is —C(O)R²⁰, and R²⁰ is optionally substituted heteroaryl.

In another embodiment, the FXR modulator is a compound of Formula (IX) or (X) wherein R⁶ and R⁷ are hydrogen, R⁴ and R⁵ are independently optionally substituted C₁-C₆alkyl, R³ is —S(O)₂R²⁰, and R²⁰ is optionally substituted aryl. In another embodiment, the FXR modulator is a compound of Formula (IX) or (X) wherein R⁶ and R⁷ are hydrogen, R⁴ and R⁵ are independently optionally substituted C₁-C₆alkyl, R³ is —S(O)₂R²⁰, and R²⁰ is optionally substituted heteroaryl. In another embodiment, the FXR modulator is a compound of Formula (IX) or (X) wherein R⁶ and R⁷ are hydrogen, R⁴ and R⁵ are methyl, R³ is —S(O)₂R²⁰, and R²⁰ is optionally substituted aryl. In another embodiment, the FXR modulator is a compound of Formula (IX) or (X) wherein R⁶ and R⁷ are hydrogen, R⁴ and R⁵ are methyl, R³ is —S(O)₂R²⁰, and R²⁰ is optionally substituted heteroaryl.

In another embodiment, the FXR modulator is a compound of Formula (IX) or (X) wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted aryl. In another embodiment, the FXR modulator is a compound of Formula (IX) or (X) wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted heteroaryl. In another embodiment, the FXR modulator is a compound of Formula (IX) or (X) wherein R⁶ and R⁷ are hydrogen, R⁴ and R⁵ are methyl, R³ is —C(O)N(R²¹)R²², R²¹ is hydrogen and R²² is optionally substituted aryl. In another embodiment, the FXR modulator is a compound of Formula (IX) or (X) wherein R⁶ and R⁷ are hydrogen, R⁴ and R⁵ are methyl, R³ is —C(O)N(R²¹)R²², R²¹ is hydrogen and R²² is optionally substituted heteroaryl.

In a further embodiment of the aforementioned embodiments is a compound of Formula (VIII) wherein R⁸ is selected from the group consisting of —CN, —C(O)OR²⁵, —C(O)N(R²⁵)R²⁶,

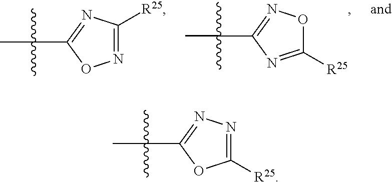

In a further embodiment of the aforementioned embodiments, the FXR modulator is a compound of Formula (IX) or (X) wherein R⁸ is —CN.

In a further embodiment of the aforementioned embodiments, the FXR modulator is a compound of Formula (IX) or (X) wherein R⁸ is —C(O)OR²⁵. In a further embodiment of the aforementioned embodiments, the FXR modulator is a compound of Formula (IX) or (X) wherein R⁸ is —C(O)OR²⁵, and R²⁵ is independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (IX) or (X) wherein R⁸ is —C(O)OR²⁵, and R²⁵ is independently selected from the group consisting of hydrogen, and optionally substituted C₁-C₆alkyl. In a further embodiment of the aforementioned embodiments, the FXR modulator is a compound of Formula (IX) or (X) wherein R⁸ is —C(O)OR²⁵, and R²⁵ is hydrogen. In a further embodiment of the aforementioned embodiments, the FXR modulator is a compound of Formula (IX) or (X) wherein R⁸ is —C(O)OR²⁵, and R²⁵ is optionally substituted C₁-C₆alkyl. In a further embodiment of the aforementioned embodiments, the FXR modulator is a compound of Formula (IX) or (X) wherein R⁸ is —C(O)OR²⁵, and R²⁵ is unsubstituted C₁-C₆alkyl. In a further embodiment of the aforementioned embodiments, the FXR modulator is a compound of Formula (IX) or (X) wherein R⁸ is —C(O)OR²⁵, and R²⁵ is methyl. In a further embodiment of the aforementioned embodiments, the FXR modulator is a compound of Formula (IX) or (X) wherein R⁸ is —C(O)OR²⁵, and R²⁵ is ethyl.

In a further embodiment of the aforementioned embodiments, the FXR modulator is a compound of Formula (IX) or (X) wherein R⁸ is —C(O)N(R²⁵)R²⁶. In a further embodiment of the aforementioned embodiments, the FXR modulator is a compound of Formula (IX) or (X) wherein R⁸ is —C(O)N(R²⁵)R²⁶, and R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (IX) or (X) wherein R⁸ is —C(O)N(R²⁵)R²⁶, and R²⁵ and R²⁶ are each independently selected from the group consisting of hydrogen, and optionally substituted C₁-C₆alkyl. In a further embodiment of the aforementioned embodiments, the FXR modulator is a compound of Formula (IX) or (X) wherein R⁸ is —C(O)N(R²⁵)R²⁶, and R²⁵ and R²⁶ are hydrogen. In a further embodiment of the aforementioned embodiments, the FXR modulator is a compound of Formula (IX) or (X) wherein R⁸ is —C(O)N(R²⁵)R²⁶, and R²⁵ and R²⁶ are each independently optionally substituted C₁-C₆alkyl. In a further embodiment of the aforementioned embodiments, the FXR modulator is a compound of Formula (IX) or (X) wherein R⁸ is —C(O)N(R²⁵)R²⁶, R²⁵ is hydrogen, and R²⁶ is optionally substituted C₁-C₆alkyl. In a further embodiment of the aforementioned embodiments, the FXR modulator is a compound of Formula (IX) or (X) wherein R⁸ is —C(O)N(R²⁵)R²⁶, and R²⁵ and R²⁶ are each independently unsubstituted C₁-C₆alkyl. In a further embodiment of the aforementioned embodiments, the FXR modulator is a compound of Formula (IX) or (X) wherein R⁸ is —C(O)N(R²⁵)R²⁶, R²⁵ is hydrogen, and R²⁶ are methyl. In a further embodiment of the aforementioned embodiments, the FXR modulator is a compound of Formula (IX) or (X) wherein R⁸ is —C(O)N(R²⁵)R²⁶, and R²⁵ and R²⁶ are methyl. In a further embodiment of the aforementioned embodiments, the FXR modulator is a compound of Formula (IX) or (X) wherein R⁸ is —C(O)N(R²⁵)R²⁶, and R²⁵ and R²⁶ are ethyl.

In a further embodiment of the aforementioned embodiments, the FXR modulator is a compound of Formula (IX) or (X) wherein R⁸ is

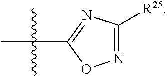

In a further embodiment of the aforementioned embodiments, the FXR modulator is a compound of Formula (IX) or (X) wherein R⁸ is

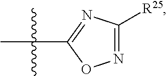

and R²⁵ is optionally substituted C₁-C₆alkyl. In a further embodiment of the aforementioned embodiments, the FXR modulator is a compound of Formula (IX) or (X) wherein R⁸ is

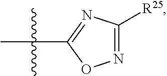

and R²⁵ is methyl. In a further embodiment of the aforementioned embodiments, the FXR modulator is a compound of Formula (IX) or (X) wherein R⁸ is

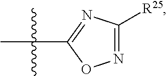

and R²⁵ is ethyl.

In a further embodiment of the aforementioned embodiments, the FXR modulator is a compound of Formula (IX) or (X) wherein R⁸ is

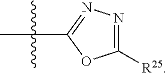

In a further embodiment of the aforementioned embodiments, the FXR modulator is a compound of Formula (IX) or (X) wherein R⁸ is

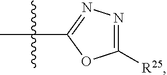

and R²⁵ is optionally substituted C₁-C₆alkyl. In a further embodiment of the aforementioned embodiments, the FXR modulator is a compound of Formula (IX) or (X) wherein R⁸ is

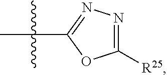

and R²⁵ is methyl. In a further embodiment of the aforementioned embodiments, the FXR modulator is a compound of Formula (IX) or (X) wherein R⁸ is

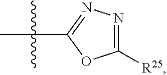

and R²⁵ is ethyl.

In a further embodiment of the aforementioned embodiments, the FXR modulator is a compound of Formula (IX) or (X) wherein R⁸ is

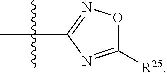

In a further embodiment of the aforementioned embodiments, the FXR modulator is a compound of Formula (IX) or (X) wherein R⁸ is

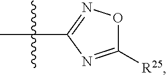

and R²⁵ is optionally substituted C₁-C₆alkyl. In a further embodiment of the aforementioned embodiments, the FXR modulator is a compound of Formula (IX) or (X) wherein R⁸ is

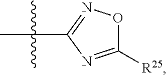

and R²⁵ is methyl. In a further embodiment of the aforementioned embodiments, the FXR modulator is a compound of Formula (IX) or (X) wherein R⁸ is

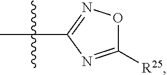

and R²⁵ is ethyl.

In a further embodiment of the aforementioned embodiments, the FXR modulator is a compound of Formula (IX) or (X) wherein R² is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl). In a further embodiment of the aforementioned embodiments, the FXR modulator is a compound of Formula (IX) or (X) wherein R² is selected from the group consisting of hydrogen, and optionally substituted C₁-C₆alkyl. In a further embodiment of the aforementioned embodiments, the FXR modulator is a compound of Formula (IX) or (X) wherein R² is optionally substituted C₁-C₆alkyl. In a further embodiment of the aforementioned embodiments, the FXR modulator is a compound of Formula (IX) or (X) wherein R² is methyl. In a further embodiment of the aforementioned embodiments, the FXR modulator is a compound of Formula (IX) or (X) wherein R² is ethyl. In a further embodiment of the aforementioned embodiments, the FXR modulator is a compound of Formula (IX) or (X) wherein R² is hydrogen.

In a further embodiment of the aforementioned embodiments, the FXR modulator is a compound of Formula (IX) or (X) wherein R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), and —OR¹⁰. In a further embodiment of the aforementioned embodiments, the FXR modulator is a compound of Formula (IX) or (X) wherein R¹ is hydrogen. In a further embodiment of the aforementioned embodiments, the FXR modulator is a compound of Formula (IX) or (X) wherein R¹ is halogen. In a further embodiment of the aforementioned embodiments, the FXR modulator is a compound of Formula (IX) or (X) wherein R¹ is optionally substituted C₁-C₆alkyl. In a further embodiment of the aforementioned embodiments, the FXR modulator is a compound of Formula (IX) or (X) wherein R¹ is optionally substituted C₂-C₆alkenyl. In a further embodiment of the aforementioned embodiments, the FXR modulator is a compound of Formula (IX) or (X) wherein R¹ is optionally substituted C₂-C₆alkynyl. In a further embodiment of the aforementioned embodiments, the FXR modulator is a compound of Formula (IX) or (X) wherein R¹ is —OR¹⁰ and R¹⁰ is hydrogen. In a further embodiment of the aforementioned embodiments, the FXR modulator is a compound of Formula (IX) or (X) wherein R¹ is —OR¹⁰ and R¹⁰ is optionally substituted C₁-C₆alkyl. In a further embodiment of the aforementioned embodiments, the FXR modulator is a compound of Formula (IX) or (X) wherein R¹ is —OR¹⁰ and R¹⁰ is methyl.

In some embodiments, the FXR modulator is a compound of Formula (XI):

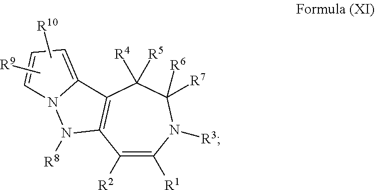

wherein:
R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R² is selected from the group consisting of —CN, —C(O)OR²⁵, —C(O)N(R²⁵)R²⁶,

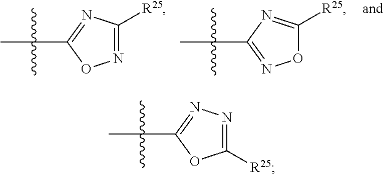

or R¹ and R² together with the carbon atoms to which they are attached, form an optionally substituted C₂-C₉heterocycloalkyl ring or an optionally substituted heteroaryl ring;
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R⁹ and R¹⁰ are each independently selected from the group consisting of hydrogen, halogen, —CN, amino, alkylamino, optionally substituted C₁-C₆alkyl, optionally substituted C₁-C₆alkoxy, optionally substituted C₃-C₈cycloalkyl, optionally substituted C₂-C₉heterocycloalkyl, optionally substituted aryl, and optionally substituted heteroaryl;
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring.

In some embodiments the FXR modulator is a compound of Formula (XI), wherein R⁴ and R⁵ are each independently selected from the group consisting of hydrogen, halogen, and optionally substituted C₁-C₆alkyl. In some embodiments the FXR modulator is a compound of Formula (XI), wherein R⁴ and R⁵ are each independently selected from the group consisting of hydrogen and optionally substituted C₁-C₆alkyl. In some embodiments the FXR modulator is a compound of Formula (XI), wherein R⁴ and R⁵ are each hydrogen. In some embodiments the FXR modulator is a compound of Formula (XI), wherein R⁴ and R⁵ are each independently optionally substituted C₁-C₆alkyl. In some embodiments the FXR modulator is a compound of Formula (XI), wherein R⁴ and R⁵ are each methyl. In some embodiments the FXR modulator is a compound of Formula (XI), wherein R⁴ and R⁵ form an optionally substituted C₃-C₆cycloalkyl ring or an optionally substituted C₂-C₇heterocycloalkyl ring. In some embodiments is a compound of Formula (XI) wherein R⁴ and R⁵ form an optionally substituted C₃-C₆cycloalkyl ring. In some embodiments is a compound of Formula (XI) wherein R⁴ and R⁵ form an optionally substituted C₂-C₇heterocycloalkyl ring.

In some embodiments the FXR modulator is a compound of Formula (XI), wherein R⁶ and R⁷ are each independently selected from the group consisting of hydrogen, halogen, and optionally substituted C₁-C₆alkyl. In some embodiments the FXR modulator is a compound of Formula (XI), wherein R⁶ and R⁷ are each independently selected from the group consisting of hydrogen and optionally substituted C₁-C₆alkyl. In some embodiments the FXR modulator is a compound of Formula (XI), wherein R⁶ and R⁷ are each independently optionally substituted C₁-C₆alkyl. In some embodiments the FXR modulator is a compound of Formula (XI), wherein R⁶ and R⁷ are each methyl. In some embodiments the FXR modulator is a compound of Formula (XI), wherein R⁶ and R⁷ are each hydrogen.

In some embodiments the FXR modulator is a compound of Formula (XI), wherein R⁶ and R⁷ are hydrogen, R⁴ and R⁵ are independently optionally substituted C₁-C₆alkyl, R³ is —C(O)R²⁰, and R²⁰ is optionally substituted aryl. In some embodiments the FXR modulator is a compound of Formula (XI), wherein R⁶ and R⁷ are hydrogen, R⁴ and R⁵ are independently optionally substituted C₁-C₆alkyl, R³ is —C(O)R²⁰, and R²⁰ is optionally substituted heteroaryl. In some embodiments the FXR modulator is a compound of Formula (XI), wherein R⁶ and R⁷ are hydrogen, R⁴ and R⁵ are methyl, R³ is —C(O)R²⁰, and R²⁰ is optionally substituted aryl. In some embodiments the FXR modulator is a compound of Formula (XI), wherein R⁶ and R⁷ are hydrogen, R⁴ and R⁵ are methyl, R³ is —C(O)R²⁰, and R²⁰ is optionally substituted heteroaryl.

In some embodiments the FXR modulator is a compound of Formula (XI), wherein R⁶ and R⁷ are hydrogen, R⁴ and R⁵ are independently optionally substituted C₁-C₆alkyl, R³ is —S(O)₂R²⁰, and R²⁰ is optionally substituted aryl. In some embodiments the FXR modulator is a compound of Formula (XI), wherein R⁶ and R⁷ are hydrogen, R⁴ and R⁵ are independently optionally substituted C₁-C₆alkyl, R³ is —S(O)₂R²⁰, and R²⁰ is optionally substituted heteroaryl. In some embodiments the FXR modulator is a compound of Formula (XI), wherein R⁶ and R⁷ are hydrogen, R⁴ and R⁵ are methyl, R³ is —S(O)₂R²⁰, and R²⁰ is optionally substituted aryl. In some embodiments the FXR modulator is a compound of Formula (XI), wherein R⁶ and R⁷ are hydrogen, R⁴ and R⁵ are methyl, R³ is —S(O)₂R²⁰, and R²⁰ is optionally substituted heteroaryl.

In some embodiments the FXR modulator is a compound of Formula (XI), wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted aryl. In some embodiments the FXR modulator is a compound of Formula (XI), wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted heteroaryl. In some embodiments the FXR modulator is a compound of Formula (XI), wherein R⁶ and R⁷ are hydrogen, R⁴ and R⁵ are methyl, R³ is —C(O)N(R²¹)R²², R²¹ is hydrogen and R²² is optionally substituted aryl. In some embodiments the FXR modulator is a compound of Formula (XI), wherein R⁶ and R⁷ are hydrogen, R⁴ and R⁵ are methyl, R³ is —C(O)N(R²¹)R²², R²¹ is hydrogen and R²² is optionally substituted heteroaryl.

In a further embodiment of the aforementioned embodiments, the FXR modulator is a compound of Formula (XI), wherein R² is selected from the group consisting of —CN, —C(O)OR²⁵, —C(O)N(R²⁵)R²⁶,

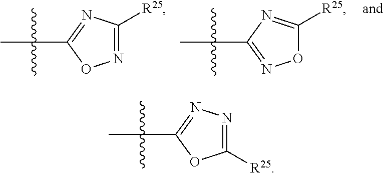

In a further embodiment of the aforementioned embodiments, the FXR modulator is a compound of Formula (XI), wherein R² is —CN.

In a further embodiment of the aforementioned embodiments, the FXR modulator is a compound of Formula (XI), wherein R² is —C(O)OR²⁵. In a further embodiment of the aforementioned embodiments, the FXR modulator is a compound of Formula (XI), wherein R² is —C(O)OR²⁵, and R²⁵ is independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XI), wherein R² is —C(O)OR²⁵, and R²⁵ is independently selected from the group consisting of hydrogen, and optionally substituted C₁-C₆alkyl. In a further embodiment of the aforementioned embodiments, the FXR modulator is a compound of Formula (XI), wherein R² is —C(O)OR²⁵, and R²⁵ is hydrogen. In a further embodiment of the aforementioned embodiments, the FXR modulator is a compound of Formula (XI), wherein R² is —C(O)OR²⁵, and R²⁵ is optionally substituted C₁-C₆alkyl. In a further embodiment of the aforementioned embodiments, the FXR modulator is a compound of Formula (XI), wherein R² is —C(O)OR²⁵, and R²⁵ is unsubstituted C₁-C₆alkyl. In a further embodiment of the aforementioned embodiments, the FXR modulator is a compound of Formula (XI), wherein R² is —C(O)OR²⁵, and R²⁵ is methyl. In a further embodiment of the aforementioned embodiments, the FXR modulator is a compound of Formula (XI), wherein R² is —C(O)OR²⁵, and R²⁵ is ethyl.

In a further embodiment of the aforementioned embodiments, the FXR modulator is a compound of Formula (XI), wherein R² is —C(O)N(R²⁵)R²⁶. In a further embodiment of the aforementioned embodiments, the FXR modulator is a compound of Formula (XI), wherein R² is —C(O)N(R²⁵)R²⁶, and R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XI), wherein R² is —C(O)N(R²⁵)R²⁶, and R²⁵ and R²⁶ are each independently selected from the group consisting of hydrogen, and optionally substituted C₁-C₆alkyl. In a further embodiment of the aforementioned embodiments, the FXR modulator is a compound of Formula (XI), wherein R² is —C(O)N(R²⁵)R²⁶, and R²⁵ and R²⁶ are hydrogen. In a further embodiment of the aforementioned embodiments, the FXR modulator is a compound of Formula (XI), wherein R² is —C(O)N(R²⁵)R²⁶, and R²⁵ and R²⁶ are each independently optionally substituted C₁-C₆alkyl. In a further embodiment of the aforementioned embodiments, the FXR modulator is a compound of Formula (XI), wherein R² is —C(O)N(R²⁵)R²⁶, R²⁵ is hydrogen, and R²⁶ is optionally substituted C₁-C₆alkyl. In a further embodiment of the aforementioned embodiments, the FXR modulator is a compound of Formula (XI), wherein R² is —C(O)N(R²⁵)R²⁶, and R²⁵ and R²⁶ are each independently unsubstituted C₁-C₆alkyl. In a further embodiment of the aforementioned embodiments, the FXR modulator is a compound of Formula (XI), wherein R² is —C(O)N(R²⁵)R²⁶, R²⁵ is hydrogen, and R²⁶ are methyl. In a further embodiment of the aforementioned embodiments, the FXR modulator is a compound of Formula (XI), wherein R² is —C(O)N(R²⁵)R²⁶, and R²⁵ and R²⁶ are methyl. In a further embodiment of the aforementioned embodiments, the FXR modulator is a compound of Formula (XI), wherein R² is —C(O)N(R²⁵)R²⁶, and R²⁵ and R²⁶ are ethyl.

In a further embodiment of the aforementioned embodiments, the FXR modulator is a compound of Formula (XI), wherein R² is

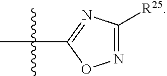

In a further embodiment of the aforementioned embodiments, the FXR modulator is a compound of Formula (XI), wherein R² is

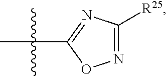

and R²⁵ is optionally substituted C₁-C₆alkyl. In a further embodiment of the aforementioned embodiments, the FXR modulator is a compound of Formula (XI), wherein R² is

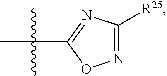

and R²⁵ is methyl. In a further embodiment of the aforementioned embodiments, the FXR modulator is a compound of Formula (XI), wherein R² is

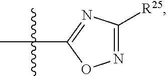

and R²⁵ is ethyl.

In a further embodiment of the aforementioned embodiments, the FXR modulator is a compound of Formula (XI), wherein R² is

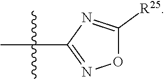

In a further embodiment of the aforementioned embodiments, the FXR modulator is a compound of Formula (XI), wherein R² is

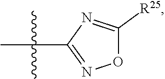

and R²⁵ is optionally substituted C₁-C₆alkyl. In a further embodiment of the aforementioned embodiments, the FXR modulator is a compound of Formula (XI), wherein R² is

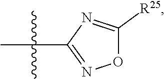

and R²⁵ is methyl. In a further embodiment of the aforementioned embodiments, the FXR modulator is a compound of Formula (XI), wherein R² is

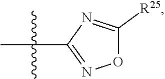

and R²⁵ is ethyl.

In a further embodiment of the aforementioned embodiments, the FXR modulator is a compound of Formula (XI), wherein R² is

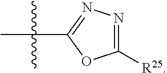

In a further embodiment of the aforementioned embodiments, the FXR modulator is a compound of Formula (XI), wherein R² is

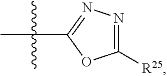

and R²⁵ is optionally substituted C₁-C₆alkyl. In a further embodiment of the aforementioned embodiments, the FXR modulator is a compound of Formula (XI), wherein R² is

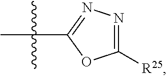

and R²⁵ is methyl. In a further embodiment of the aforementioned embodiments, the FXR modulator is a compound of Formula (XI), wherein R² is

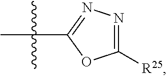

and R²⁵ is ethyl.

In a further embodiment of the aforementioned embodiments, the FXR modulator is a compound of Formula (XI), wherein R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XI), wherein R¹ is hydrogen. In a further embodiment of the aforementioned embodiments, the FXR modulator is a compound of Formula (XI), wherein R¹ is optionally substituted C₁-C₆alkyl. In a further embodiment of the aforementioned embodiments, the FXR modulator is a compound of Formula (XI), wherein R¹ is methyl. In a further embodiment of the aforementioned embodiments, the FXR modulator is a compound of Formula (XI), wherein R¹ is optionally substituted C₂-C₆alkenyl. In a further embodiment of the aforementioned embodiments, the FXR modulator is a compound of Formula (XI), wherein R¹ is optionally substituted C₂-C₆alkynyl.

In a further embodiment of the aforementioned embodiments, the FXR modulator is a compound of Formula (XI), wherein R¹ and R² together with the carbon atoms to which they are attached, form an optionally substituted C₂-C₉heterocycloalkyl ring or an optionally substituted heteroaryl ring. In a further embodiment of the aforementioned embodiments, the FXR modulator is a compound of Formula (XI), wherein R¹ and R² together with the carbon atoms to which they are attached, form an optionally substituted C₂-C₉heterocycloalkyl ring. In a further embodiment of the aforementioned embodiments, the FXR modulator is a compound of Formula (XI), wherein R¹ and R² together with the carbon atoms to which they are attached, form an optionally substituted heteroaryl ring.

In a further embodiment of the aforementioned embodiments, the FXR modulator is a compound of Formula (XI), wherein R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl). In a further embodiment of the aforementioned embodiments, the FXR modulator is a compound of Formula (XI), wherein R⁸ is selected from the group consisting of hydrogen, and optionally substituted C₁-C₆alkyl. In a further embodiment of the aforementioned embodiments, the FXR modulator is a compound of Formula (XI), wherein R⁸ is optionally substituted C₁-C₆alkyl. In a further embodiment of the aforementioned embodiments, the FXR modulator is a compound of Formula (XI), wherein R⁸ is methyl. In a further embodiment of the aforementioned embodiments, the FXR modulator is a compound of Formula (XI), wherein R⁸ is optionally substituted C₁-C₆alkyl. In a further embodiment of the aforementioned embodiments, the FXR modulator is a compound of Formula (XI), wherein R⁸ is ethyl. In a further embodiment of the aforementioned embodiments, the FXR modulator is a compound of Formula (XI), wherein R⁸ is optionally substituted C₁-C₆alkyl. In a further embodiment of the aforementioned embodiments, the FXR modulator is a compound of Formula (XI), wherein R⁸ is hydrogen.

In some embodiments of a compound of Formula (XI), the FXR modulator is a compound of Formula (XIa), or a pharmaceutically acceptable salt, stereoisomer, or solvate thereof:

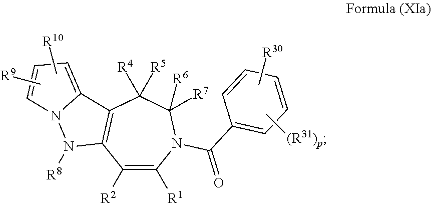

wherein:
R³⁰ is halogen,

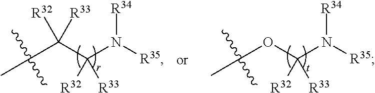

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring;
p is 0, 1, 2, 3, or 4;
r is 0, 1, 2, 3, or 4; and
t is 2, 3, or 4.

In some embodiments the FXR modulator is a compound of Formula (XIa), wherein R⁴ and R⁵ are each independently selected from the group consisting of hydrogen, halogen, and optionally substituted C₁-C₆alkyl. In some embodiments the FXR modulator is a compound of Formula (XIa), wherein R⁴ and R⁵ are each independently selected from the group consisting of hydrogen and optionally substituted C₁-C₆alkyl. In some embodiments the FXR modulator is a compound of Formula (XIa), wherein R⁴ and R⁵ are each hydrogen. In some embodiments the FXR modulator is a compound of Formula (XIa), wherein R⁴ and R⁵ are each independently optionally substituted C₁-C₆alkyl. In some embodiments the FXR modulator is a compound of Formula (XIa), wherein R⁴ and R⁵ are each methyl. In some embodiments the FXR modulator is a compound of Formula (XIa), wherein R⁴ and R⁵ form an optionally substituted C₃-C₆cycloalkyl ring or an optionally substituted C₂-C₇heterocycloalkyl ring. In some embodiments is a compound of Formula (XIa) wherein R⁴ and R⁵ form an optionally substituted C₃-C₆cycloalkyl ring. In some embodiments is a compound of Formula (XIa) wherein R⁴ and R⁵ form an optionally substituted C₂-C₇heterocycloalkyl ring.

In some embodiments the FXR modulator is a compound of Formula (XIa), wherein R⁶ and R⁷ are each independently selected from the group consisting of hydrogen, halogen, and optionally substituted C₁-C₆alkyl. In some embodiments the FXR modulator is a compound of Formula (XIa), wherein R⁶ and R⁷ are each independently selected from the group consisting of hydrogen and optionally substituted C₁-C₆alkyl. In some embodiments the FXR modulator is a compound of Formula (XIa), wherein R⁶ and R⁷ are each independently optionally substituted C₁-C₆alkyl. In some embodiments the FXR modulator is a compound of Formula (XIa), wherein R⁶ and R⁷ are each methyl. In some embodiments the FXR modulator is a compound of Formula (XIa), wherein R⁶ and R⁷ are each hydrogen.

In some embodiments the FXR modulator is a compound of Formula (XIa), wherein R⁶ and R⁷ are hydrogen, R⁴ and R⁵ are independently optionally substituted C₁-C₆alkyl, R³ is —C(O)R²⁰, and R²⁰ is optionally substituted aryl. In some embodiments the FXR modulator is a compound of Formula (XIa), wherein R⁶ and R⁷ are hydrogen, R⁴ and R⁵ are independently optionally substituted C₁-C₆alkyl, R³ is —C(O)R²⁰, and R²⁰ is optionally substituted heteroaryl. In some embodiments the FXR modulator is a compound of Formula (XIa), wherein R⁶ and R⁷ are hydrogen, R⁴ and R⁵ are methyl, R³ is —C(O)R²⁰, and R²⁰ is optionally substituted aryl. In some embodiments the FXR modulator is a compound of Formula (XIa), wherein R⁶ and R⁷ are hydrogen, R⁴ and R⁵ are methyl, R³ is —C(O)R²⁰, and R²⁰ is optionally substituted heteroaryl.

In some embodiments the FXR modulator is a compound of Formula (XIa), wherein R⁶ and R⁷ are hydrogen, R⁴ and R⁵ are independently optionally substituted C₁-C₆alkyl, R³ is —S(O)₂R²⁰, and R²⁰ is optionally substituted aryl. In some embodiments the FXR modulator is a compound of Formula (XIa), wherein R⁶ and R⁷ are hydrogen, R⁴ and R⁵ are independently optionally substituted C₁-C₆alkyl, R³ is —S(O)₂R²⁰, and R²⁰ is optionally substituted heteroaryl. In some embodiments the FXR modulator is a compound of Formula (XIa), wherein R⁶ and R⁷ are hydrogen, R⁴ and R⁵ are methyl, R³ is —S(O)₂R²⁰, and R²⁰ is optionally substituted aryl. In some embodiments the FXR modulator is a compound of Formula (XIa), wherein R⁶ and R⁷ are hydrogen, R⁴ and R⁵ are methyl, R³ is —S(O)₂R²⁰, and R²⁰ is optionally substituted heteroaryl.

In some embodiments the FXR modulator is a compound of Formula (XIa), wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted aryl. In some embodiments the FXR modulator is a compound of Formula (XIa), wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted heteroaryl. In some embodiments the FXR modulator is a compound of Formula (XIa), wherein R⁶ and R⁷ are hydrogen, R⁴ and R⁵ are methyl, R³ is —C(O)N(R²¹)R²², R²¹ is hydrogen and R²² is optionally substituted aryl. In some embodiments the FXR modulator is a compound of Formula (XIa), wherein R⁶ and R⁷ are hydrogen, R⁴ and R⁵ are methyl, R³ is —C(O)N(R²¹)R²², R²¹ is hydrogen and R²² is optionally substituted heteroaryl.

In some embodiments the FXR modulator is a compound of Formula (XIa), wherein p is 0. In some embodiments the FXR modulator is a compound of Formula (XIa), wherein p is 1. In some embodiments the FXR modulator is a compound of Formula (XIa), wherein p is 2. In some embodiments the FXR modulator is a compound of Formula (XIa), wherein p is 3. In some embodiments the FXR modulator is a compound of Formula (XIa), wherein p is 4.

In some embodiments the FXR modulator is a compound of Formula (XIa), wherein p is 2 and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIa), wherein p is 2 and each R³¹ is independently halogen, or optionally substituted C₁-C₆alkyl. In some embodiments the FXR modulator is a compound of Formula (XIa), wherein p is 2 and each R³¹ is halogen. In some embodiments the FXR modulator is a compound of Formula (XIa), wherein p is 2 and each R³¹ is F.

In some embodiments the FXR modulator is a compound of Formula (XIa), wherein R³⁰ is F, p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIa), wherein R³⁰ is F, p is 2 and each R³¹ is independently halogen, or optionally substituted C₁-C₆alkyl. In some embodiments the FXR modulator is a compound of Formula (XIa), wherein R³⁰ is F, p is 2 and each R³¹ is halogen. In some embodiments the FXR modulator is a compound of Formula (XIa), wherein R³⁰ is F, p is 2 and each R³¹ is F.

In some embodiments the FXR modulator is a compound of Formula (XIa), wherein p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIa), wherein p is 1 and R³¹ is halogen, or optionally substituted C₁-C₆alkyl. In some embodiments the FXR modulator is a compound of Formula (XIa), wherein p is 1 and R³¹ is halogen. In some embodiments the FXR modulator is a compound of Formula (XIa), wherein p is 1 and R³¹ is F.

In some embodiments the FXR modulator is a compound of Formula (XIa), wherein R³⁰ is F, p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIa), wherein R³⁰ is F, p is 1 and R³¹ is halogen, or optionally substituted C₁-C₆alkyl. In some embodiments the FXR modulator is a compound of Formula (XIa), wherein R³⁰ is F, p is 1 and R³¹ is halogen. In some embodiments the FXR modulator is a compound of Formula (XIa), wherein R³⁰ is F, p is 1 and R³¹ is F.

In some embodiments the FXR modulator is a compound of Formula (XIa), wherein R³⁰ is

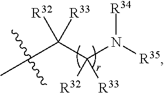

p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIa), wherein R³⁰ is

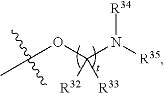

p is 2 and each R³¹ is independently halogen, or optionally substituted C₁-C₆alkyl. In

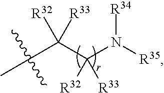

some embodiments the FXR modulator is a compound of Formula (XIa), wherein R³⁰ is p is 2 and each R³¹ is halogen. In some embodiments the FXR modulator is a compound of Formula (XIa), wherein R³⁰ is

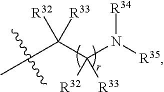

p is 2 and each R³¹ is F.

In some embodiments the FXR modulator is a compound of Formula (XIa), wherein R³⁰ is

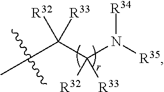

p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIa), wherein R³⁰ is

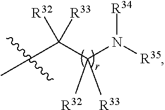

p is 1 and R³¹ is halogen, or optionally substituted C₁-C₆alkyl. In some embodiments the FXR modulator is a compound of Formula (XIa), wherein R³⁰ is

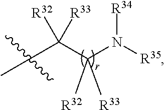

p is 1 and R³¹ is halogen. In some embodiments the FXR modulator is a compound of Formula (XIa), wherein R³⁰ is

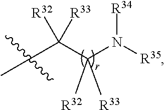

### p is 1 and R³¹ is F.

In some embodiments the FXR modulator is a compound of Formula (XIa), wherein R³⁰ is

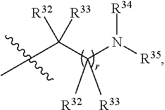

### and p is O.

In some embodiments the FXR modulator is a compound of Formula (XIa), wherein R³⁰ is

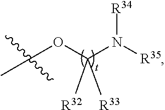

p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIa), wherein R³⁰ is

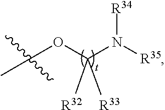

p is 2 and each R³¹ is independently halogen, or optionally substituted C₁-C₆alkyl. In some embodiments the FXR modulator is a compound of Formula (XIa), wherein R³⁰ is

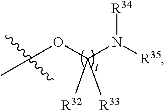

p is 2 and each R³¹ is halogen. In some embodiments the FXR modulator is a compound of Formula (XIa), wherein R³⁰ is

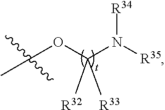

p is 2 and each R³¹ is F.

In some embodiments the FXR modulator is a compound of Formula (XIa), wherein R³⁰ is

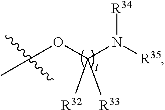

p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIa), wherein R³⁰ is

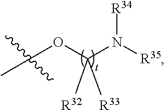

p is 1 and R³¹ is halogen, or optionally substituted C₁-C₆alkyl. In some embodiments the FXR modulator is a compound of Formula (XIa), wherein R³⁰ is

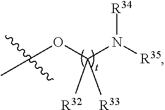

p is 1 and R³¹ is halogen. In some embodiments the FXR modulator is a compound of Formula (XIa), wherein R³⁰ is

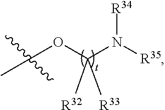

### p is 1 and R³¹ is F.

In some embodiments the FXR modulator is a compound of Formula (XIa), wherein R³⁰ is

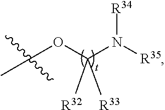

and p is 0.

In a further embodiment of the aforementioned embodiments, the FXR modulator is a compound of Formula (XIa), wherein R² is selected from the group consisting of —CN, —C(O)OR²⁵, —C(O)N(R²⁵)R²⁶.

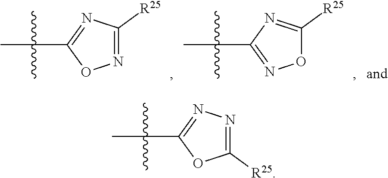

In a further embodiment of the aforementioned embodiments, the FXR modulator is a compound of Formula (XIa), wherein R² is —CN.

In a further embodiment of the aforementioned embodiments, the FXR modulator is a compound of Formula (XIa), wherein R² is —C(O)OR²⁵. In a further embodiment of the aforementioned embodiments, the FXR modulator is a compound of Formula (XIa), wherein R² is —C(O)OR²⁵, and R²⁵ is independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XIa), wherein R² is —C(O)OR²⁵, and R²⁵ is independently selected from the group consisting of hydrogen, and optionally substituted C₁-C₆alkyl. In a further embodiment of the aforementioned embodiments, the FXR modulator is a compound of Formula (XIa), wherein R² is —C(O)OR²⁵, and R²⁵ is hydrogen. In a further embodiment of the aforementioned embodiments, the FXR modulator is a compound of Formula (XIa), wherein R² is —C(O)OR²⁵, and R²⁵ is optionally substituted C₁-C₆alkyl. In a further embodiment of the aforementioned embodiments, the FXR modulator is a compound of Formula (XIa), wherein R² is —C(O)OR²⁵, and R²⁵ is unsubstituted C₁-C₆alkyl. In a further embodiment of the aforementioned embodiments, the FXR modulator is a compound of Formula (XIa), wherein R² is —C(O)OR²⁵, and R²⁵ is methyl. In a further embodiment of the aforementioned embodiments, the FXR modulator is a compound of Formula (XIa), wherein R² is —C(O)OR²⁵, and R²⁵ is ethyl.

In a further embodiment of the aforementioned embodiments, the FXR modulator is a compound of Formula (XIa), wherein R² is —C(O)N(R²⁵)R²⁶. In a further embodiment of the aforementioned embodiments, the FXR modulator is a compound of Formula (XIa), wherein R² is —C(O)N(R²⁵)R²⁶, and R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XIa), wherein R² is —C(O)N(R²⁵)R²⁶, and R²⁵ and R²⁶ are each independently selected from the group consisting of hydrogen, and optionally substituted C₁-C₆alkyl. In a further embodiment of the aforementioned embodiments, the FXR modulator is a compound of Formula (XIa), wherein R² is —C(O)N(R²⁵)R²⁶, and R²⁵ and R²⁶ are hydrogen. In a further embodiment of the aforementioned embodiments, the FXR modulator is a compound of Formula (XIa), wherein R² is —C(O)N(R²⁵)R²⁶, and R²⁵ and R²⁶ are each independently optionally substituted C₁-C₆alkyl. In a further embodiment of the aforementioned embodiments, the FXR modulator is a compound of Formula (XIa), wherein R² is —C(O)N(R²⁵)R²⁶, R²⁵ is hydrogen, and R²⁶ is optionally substituted C₁-C₆alkyl. In a further embodiment of the aforementioned embodiments, the FXR modulator is a compound of Formula (XIa), wherein R² is —C(O)N(R²⁵)R²⁶, and R²⁵ and R²⁶ are each independently unsubstituted C₁-C₆alkyl. In a further embodiment of the aforementioned embodiments, the FXR modulator is a compound of Formula (XIa), wherein R² is —C(O)N(R²⁵)R²⁶, R²⁵ is hydrogen, and R²⁶ are methyl. In a further embodiment of the aforementioned embodiments, the FXR modulator is a compound of Formula (XIa), wherein R² is —C(O)N(R²⁵)R²⁶, and R²⁵ and R²⁶ are methyl. In a further embodiment of the aforementioned embodiments, the FXR modulator is a compound of Formula (XIa), wherein R² is —C(O)N(R²⁵)R²⁶, and R²⁵ and R²⁶ are ethyl.

In a further embodiment of the aforementioned embodiments, the FXR modulator is a compound of Formula (XIa), wherein R² is

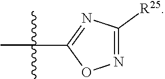

In a further embodiment of the aforementioned embodiments, the FXR modulator is a compound of Formula (XIa), wherein R² is

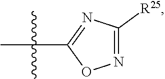

and R²⁵ is optionally substituted C₁-C₆alkyl. In a further embodiment of the aforementioned embodiments, the FXR modulator is a compound of Formula (XIa), wherein R² is

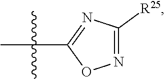

and R²⁵ is methyl. In a further embodiment of the aforementioned embodiments, the FXR modulator is a compound of Formula (XIa), wherein R² is

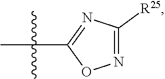

and R²⁵ is ethyl.

In a further embodiment of the aforementioned embodiments, the FXR modulator is a compound of Formula (XIa), wherein R² is

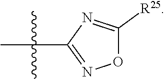

In a further embodiment of the aforementioned embodiments, the FXR modulator is a compound of Formula (XIa), wherein R² is

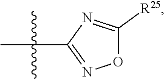

and R²⁵ is optionally substituted C₁-C₆alkyl. In a further embodiment of the aforementioned embodiments, the FXR modulator is a compound of Formula (XIa), wherein R² is

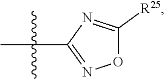

and R²⁵ is methyl. In a further embodiment of the aforementioned embodiments, the FXR modulator is a compound of Formula (XIa), wherein R² is

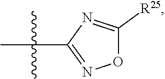

and R²⁵ is ethyl.

In a further embodiment of the aforementioned embodiments, the FXR modulator is a compound of Formula (XIa), wherein R² is

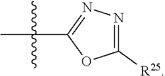

In a further embodiment of the aforementioned embodiments, the FXR modulator is a compound of Formula (XIa), wherein R² is

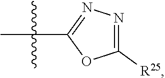

and R²⁵ is optionally substituted C₁-C₆alkyl. In a further embodiment of the aforementioned embodiments, the FXR modulator is a compound of Formula (XIa), wherein R² is

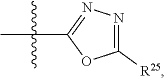

and R²⁵ is methyl. In a further embodiment of the aforementioned embodiments, the FXR modulator is a compound of Formula (XIa), wherein R² is

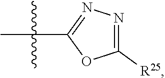

and R²⁵ is ethyl.

In a further embodiment of the aforementioned embodiments, the FXR modulator is a compound of Formula (XIa), wherein R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XIa), wherein R¹ is hydrogen. In a further embodiment of the aforementioned embodiments, the FXR modulator is a compound of Formula (XIa), wherein R¹ is optionally substituted C₁-C₆alkyl. In a further embodiment of the aforementioned embodiments, the FXR modulator is a compound of Formula (XIa), wherein R¹ is methyl. In a further embodiment of the aforementioned embodiments, the FXR modulator is a compound of Formula (XIa), wherein R¹ is optionally substituted C₂-C₆alkenyl. In a further embodiment of the aforementioned embodiments, the FXR modulator is a compound of Formula (XIa), wherein R¹ is optionally substituted C₂-C₆alkynyl.

In a further embodiment of the aforementioned embodiments, the FXR modulator is a compound of Formula (XIa), wherein R¹ and R² together with the carbon atoms to which they are attached, form an optionally substituted C₂-C₉heterocycloalkyl ring or an optionally substituted heteroaryl ring. In a further embodiment of the aforementioned embodiments, the FXR modulator is a compound of Formula (XIa), wherein R¹ and R² together with the carbon atoms to which they are attached, form an optionally substituted C₂-C₉heterocycloalkyl ring. In a further embodiment of the aforementioned embodiments, the FXR modulator is a compound of Formula (XIa), wherein R¹ and R² together with the carbon atoms to which they are attached, form an optionally substituted heteroaryl ring.

In a further embodiment of the aforementioned embodiments, the FXR modulator is a compound of Formula (XIa), wherein R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl). In a further embodiment of the aforementioned embodiments, the FXR modulator is a compound of Formula (XIa), wherein R⁸ is selected from the group consisting of hydrogen, and optionally substituted C₁-C₆alkyl. In a further embodiment of the aforementioned embodiments, the FXR modulator is a compound of Formula (XIa), wherein R⁸ is optionally substituted C₁-C₆alkyl. In a further embodiment of the aforementioned embodiments, the FXR modulator is a compound of Formula (XIa), wherein R⁸ is methyl. In a further embodiment of the aforementioned embodiments, the FXR modulator is a compound of Formula (XIa), wherein R⁸ is optionally substituted C₁-C₆alkyl. In a further embodiment of the aforementioned embodiments, the FXR modulator is a compound of Formula (XIa), wherein R⁸ is ethyl. In a further embodiment of the aforementioned embodiments, the FXR modulator is a compound of Formula (XIa), wherein R⁸ is optionally substituted C₁-C₆alkyl. In a further embodiment of the aforementioned embodiments, the FXR modulator is a compound of Formula (XIa), wherein R⁸ is hydrogen.

In some embodiments, the FXR modulator is a compound of Formula (XII):

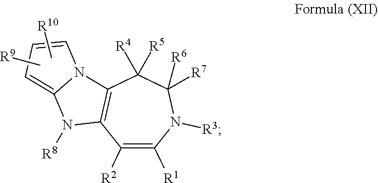

wherein:
R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R² is selected from the group consisting of —CN, —C(O)OR²⁵, —C(O)N(R²⁵)R²⁶,

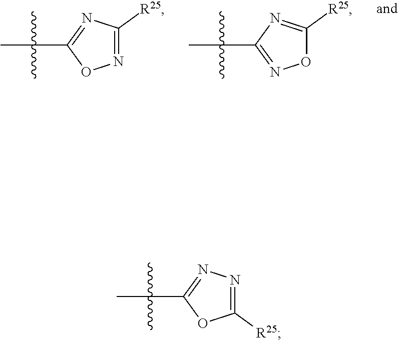

or R¹ and R² together with the carbon atoms to which they are attached, form an optionally substituted C₂-C₉heterocycloalkyl ring or an optionally substituted heteroaryl ring;
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R⁹ and R¹⁰ are each independently selected from the group consisting of hydrogen, halogen, —CN, amino, alkylamino, optionally substituted C₁-C₆alkyl, optionally substituted C₁-C₆alkoxy, optionally substituted C₃-C₈cycloalkyl, optionally substituted C₂-C₉heterocycloalkyl, optionally substituted aryl, and optionally substituted heteroaryl;
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²⁷ and R²⁸ together with the nitrogen atom to which they are attached, form an optionally substituted C₂-C₉heterocycloalkyl ring.

In some embodiments the FXR modulator is a compound of Formula (XII), wherein R⁴ and R⁵ are each independently selected from the group consisting of hydrogen, halogen, and optionally substituted C₁-C₆alkyl. In some embodiments the FXR modulator is a compound of Formula (XII), wherein R⁴ and R⁵ are each independently selected from the group consisting of hydrogen and optionally substituted C₁-C₆alkyl. In some embodiments the FXR modulator is a compound of Formula (XII), wherein R⁴ and R⁵ are each hydrogen. In some embodiments the FXR modulator is a compound of Formula (XII), wherein R⁴ and R⁵ are each independently optionally substituted C₁-C₆alkyl. In some embodiments the FXR modulator is a compound of Formula (XII), wherein R⁴ and R⁵ are each methyl. In some embodiments the FXR modulator is a compound of Formula (XII), wherein R⁴ and R⁵ form an optionally substituted C₃-C₆cycloalkyl ring or an optionally substituted C₂-C₇heterocycloalkyl ring. In some embodiments is a compound of Formula (XII) wherein R⁴ and R^{5 form an optionally substituted C}₃-C₆cycloalkyl ring. In some embodiments is a compound of Formula (XII) wherein R⁴ and R⁵ form an optionally substituted C₂-C₇heterocycloalkyl ring.

In some embodiments the FXR modulator is a compound of Formula (XII), wherein R⁶ and R⁷ are each independently selected from the group consisting of hydrogen, halogen, and optionally substituted C₁-C₆alkyl. In some embodiments the FXR modulator is a compound of Formula (XII), wherein R⁶ and R⁷ are each independently selected from the group consisting of hydrogen and optionally substituted C₁-C₆alkyl. In some embodiments the FXR modulator is a compound of Formula (XII), wherein R⁶ and R⁷ are each independently optionally substituted C₁-C₆alkyl. In some embodiments the FXR modulator is a compound of Formula (XII), wherein R⁶ and R⁷ are each methyl. In some embodiments the FXR modulator is a compound of Formula (XII), wherein R⁶ and R⁷ are each hydrogen.

In some embodiments the FXR modulator is a compound of Formula (XII), wherein R⁶ and R⁷ are hydrogen, R⁴ and R⁵ are independently optionally substituted C₁-C₆alkyl, R³ is —C(O)R²⁰, and R²⁰ is optionally substituted aryl. In some embodiments the FXR modulator is a compound of Formula (XII), wherein R⁶ and R⁷ are hydrogen, R⁴ and R⁵ are independently optionally substituted C₁-C₆alkyl, R³ is —C(O)R²⁰, and R²⁰ is optionally substituted heteroaryl. In some embodiments the FXR modulator is a compound of Formula (XII), wherein R⁶ and R⁷ are hydrogen, R⁴ and R⁵ are methyl, R³ is —C(O)R²⁰, and R²⁰ is optionally substituted aryl. In some embodiments the FXR modulator is a compound of Formula (XII), wherein R⁶ and R⁷ are hydrogen, R⁴ and R⁵ are methyl, R³ is —C(O)R²⁰, and R²⁰ is optionally substituted heteroaryl.

In some embodiments the FXR modulator is a compound of Formula (XII), wherein R⁶ and R⁷ are hydrogen, R⁴ and R⁵ are independently optionally substituted C₁-C₆alkyl, R³ is —S(O)₂R²⁰, and R²⁰ is optionally substituted aryl. In some embodiments the FXR modulator is a compound of Formula (XII), wherein R⁶ and R⁷ are hydrogen, R⁴ and R⁵ are independently optionally substituted C₁-C₆alkyl, R³ is —S(O)₂R²⁰, and R²⁰ is optionally substituted heteroaryl. In some embodiments the FXR modulator is a compound of Formula (XII), wherein R⁶ and R⁷ are hydrogen, R⁴ and R⁵ are methyl, R³ is —S(O)₂R²⁰, and R²⁰ is optionally substituted aryl. In some embodiments the FXR modulator is a compound of Formula (XII), wherein R⁶ and R⁷ are hydrogen, R⁴ and R⁵ are methyl, R³ is —S(O)₂R²⁰, and R²⁰ is optionally substituted heteroaryl.

In some embodiments the FXR modulator is a compound of Formula (XII), wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted aryl. In some embodiments the FXR modulator is a compound of Formula (XII), wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted heteroaryl. In some embodiments the FXR modulator is a compound of Formula (XII), wherein R⁶ and R⁷ are hydrogen, R⁴ and R⁵ are methyl, R³ is —C(O)N(R²¹)R²², R²¹ is hydrogen and R²² is optionally substituted aryl. In some embodiments the FXR modulator is a compound of Formula (XII), wherein R⁶ and R⁷ are hydrogen, R⁴ and R⁵ are methyl, R³ is —C(O)N(R²¹)R²², R²¹ is hydrogen and R²² is optionally substituted heteroaryl.

In a further embodiment of the aforementioned embodiments, the FXR modulator is a compound of Formula (XII), wherein R² is selected from the group consisting of —CN, —C(O)OR²⁵, —C(O)N(R²⁵)R²⁶,

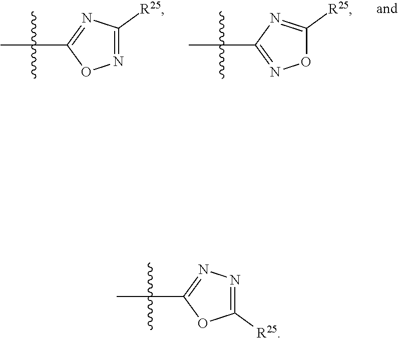

In a further embodiment of the aforementioned embodiments, the FXR modulator is a compound of Formula (XII), wherein R² is —CN.

In a further embodiment of the aforementioned embodiments, the FXR modulator is a compound of Formula (XII), wherein R² is —C(O)OR²⁵. In a further embodiment of the aforementioned embodiments, the FXR modulator is a compound of Formula (XII), wherein R² is —C(O)OR²⁵, and R²⁵ is independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XII), wherein R² is —C(O)OR²⁵, and R²⁵ is independently selected from the group consisting of hydrogen, and optionally substituted C₁-C₆alkyl. In a further embodiment of the aforementioned embodiments, the FXR modulator is a compound of Formula (XII), wherein R² is —C(O)OR²⁵, and R²⁵ is hydrogen. In a further embodiment of the aforementioned embodiments, the FXR modulator is a compound of Formula (XII), wherein R² is —C(O)OR²⁵, and R²⁵ is optionally substituted C₁-C₆alkyl. In a further embodiment of the aforementioned embodiments, the FXR modulator is a compound of Formula (XII), wherein R² is —C(O)OR²⁵, and R²⁵ is unsubstituted C₁-C₆alkyl. In a further embodiment of the aforementioned embodiments, the FXR modulator is a compound of Formula (XII), wherein R² is —C(O)OR²⁵, and R²⁵ is methyl. In a further embodiment of the aforementioned embodiments, the FXR modulator is a compound of Formula (XII), wherein R² is —C(O)OR²⁵, and R²⁵ is ethyl.

In a further embodiment of the aforementioned embodiments, the FXR modulator is a compound of Formula (XII), wherein R² is —C(O)N(R²⁵)R²⁶. In a further embodiment of the aforementioned embodiments, the FXR modulator is a compound of Formula (XII), wherein R² is —C(O)N(R²⁵)R²⁶, and R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XII), wherein R² is —C(O)N(R²⁵)R²⁶, and R²⁵ and R²⁶ are each independently selected from the group consisting of hydrogen, and optionally substituted C₁-C₆alkyl. In a further embodiment of the aforementioned embodiments, the FXR modulator is a compound of Formula (XII), wherein R² is —C(O)N(R²⁵)R²⁶, and R²⁵ and R²⁶ are hydrogen. In a further embodiment of the aforementioned embodiments, the FXR modulator is a compound of Formula (XII), wherein R² is —C(O)N(R²⁵)R²⁶, and R²⁵ and R²⁶ are each independently optionally substituted C₁-C₆alkyl. In a further embodiment of the aforementioned embodiments, the FXR modulator is a compound of Formula (XII), wherein R² is —C(O)N(R²⁵)R²⁶, R²⁵ is hydrogen, and R²⁶ is optionally substituted C₁-C₆alkyl. In a further embodiment of the aforementioned embodiments, the FXR modulator is a compound of Formula (XII), wherein R² is —C(O)N(R²⁵)R²⁶, and R²⁵ and R²⁶ are each independently unsubstituted C₁-C₆alkyl. In a further embodiment of the aforementioned embodiments, the FXR modulator is a compound of Formula (XII), wherein R² is —C(O)N(R²⁵)R²⁶, R²⁵ is hydrogen, and R²⁶ are methyl. In a further embodiment of the aforementioned embodiments, the FXR modulator is a compound of Formula (XII), wherein R² is —C(O)N(R²⁵)R²⁶, and R²⁵ and R²⁶ are methyl. In a further embodiment of the aforementioned embodiments, the FXR modulator is a compound of Formula (XII), wherein R² is —C(O)N(R²⁵)R²⁶, and R²⁵ and R²⁶ are ethyl.

In a further embodiment of the aforementioned embodiments, the FXR modulator is a compound of Formula (XII), wherein R² is

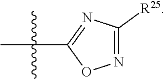

In a further embodiment of the aforementioned embodiments, the FXR modulator is a compound of Formula (XII), wherein R² is

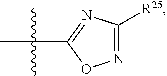

and R²⁵ is optionally substituted C₁-C₆alkyl. In a further embodiment of the aforementioned embodiments, the FXR modulator is a compound of Formula (XII), wherein R² is

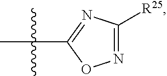

and R²⁵ is methyl. In a further embodiment of the aforementioned embodiments, the FXR modulator is a compound of Formula (XII), wherein R² is

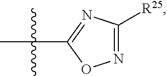

and R²⁵ is ethyl.

In a further embodiment of the aforementioned embodiments, the FXR modulator is a compound of Formula (XII), wherein R² is

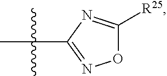

In a further embodiment of the aforementioned embodiments, the FXR modulator is a compound of Formula (XII), wherein R² is

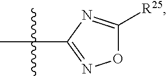

and R²⁵ is optionally substituted C₁-C₆alkyl. In a further embodiment of the aforementioned embodiments, the FXR modulator is a compound of Formula (XII), wherein R² is

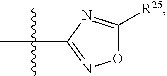

and R²⁵ is methyl. In a further embodiment of the aforementioned embodiments, the FXR modulator is a compound of Formula (XII), wherein R² is

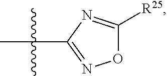

and R²⁵ is ethyl.

In a further embodiment of the aforementioned embodiments, the FXR modulator is a compound of Formula (XII), wherein R² is

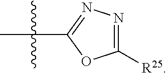

In a further embodiment of the aforementioned embodiments, the FXR modulator is a compound of Formula (XII), wherein R² is

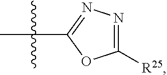

and R²⁵ is optionally substituted C₁-C₆alkyl. In a further embodiment of the aforementioned embodiments, the FXR modulator is a compound of Formula (XII), wherein R² is

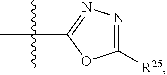

and R²⁵ is methyl. In a further embodiment of the aforementioned embodiments, the FXR modulator is a compound of Formula (XII), wherein R² is

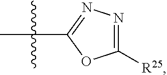

and R²⁵ is ethyl.

In a further embodiment of the aforementioned embodiments, the FXR modulator is a compound of Formula (XII), wherein R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XII), wherein R¹ is hydrogen. In a further embodiment of the aforementioned embodiments, the FXR modulator is a compound of Formula (XII), wherein R¹ is optionally substituted C₁-C₆alkyl. In a further embodiment of the aforementioned embodiments, the FXR modulator is a compound of Formula (XII), wherein R¹ is methyl. In a further embodiment of the aforementioned embodiments, the FXR modulator is a compound of Formula (XII), wherein R¹ is optionally substituted C₂-C₆alkenyl. In a further embodiment of the aforementioned embodiments, the FXR modulator is a compound of Formula (XII), wherein R¹ is optionally substituted C₂-C₆alkynyl.

In a further embodiment of the aforementioned embodiments, the FXR modulator is a compound of Formula (XII), wherein R¹ and R² together with the carbon atoms to which they are attached, form an optionally substituted C₂-C₉heterocycloalkyl ring or an optionally substituted heteroaryl ring. In a further embodiment of the aforementioned embodiments, the FXR modulator is a compound of Formula (XII), wherein R¹ and R² together with the carbon atoms to which they are attached, form an optionally substituted C₂-C₉heterocycloalkyl ring. In a further embodiment of the aforementioned embodiments, the FXR modulator is a compound of Formula (XII), wherein R¹ and R² together with the carbon atoms to which they are attached, form an optionally substituted heteroaryl ring.

In a further embodiment of the aforementioned embodiments, the FXR modulator is a compound of Formula (XII), wherein R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl). In a further embodiment of the aforementioned embodiments, the FXR modulator is a compound of Formula (XII), wherein R⁸ is selected from the group consisting of hydrogen, and optionally substituted C₁-C₆alkyl. In a further embodiment of the aforementioned embodiments, the FXR modulator is a compound of Formula (XII), wherein R⁸ is optionally substituted C₁-C₆alkyl. In a further embodiment of the aforementioned embodiments, the FXR modulator is a compound of Formula (XII), wherein R⁸ is methyl. In a further embodiment of the aforementioned embodiments, the FXR modulator is a compound of Formula (XII), wherein R⁸ is optionally substituted C₁-C₆alkyl. In a further embodiment of the aforementioned embodiments, the FXR modulator is a compound of Formula (XII), wherein R⁸ is ethyl. In a further embodiment of the aforementioned embodiments, the FXR modulator is a compound of Formula (XII), wherein R⁸ is optionally substituted C₁-C₆alkyl. In a further embodiment of the aforementioned embodiments, the FXR modulator is a compound of Formula (XII), wherein R⁸ is hydrogen.

In some embodiments of a compound of Formula (XII), the FXR modulator is a compound of Formula (XIIa), or a pharmaceutically acceptable salt, stereoisomer, or solvate thereof:

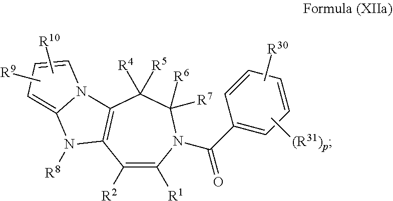

wherein:
R³⁰ is halogen,

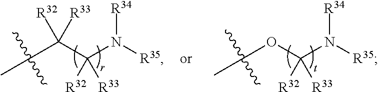

each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring;
p is 0, 1, 2, 3, or 4;
r is 0, 1, 2, 3, or 4; and
t is 2, 3, or 4.

In some embodiments the FXR modulator is a compound of Formula (XIIa), wherein R⁴ and R⁵ are each independently selected from the group consisting of hydrogen, halogen, and optionally substituted C₁-C₆alkyl. In some embodiments the FXR modulator is a compound of Formula (XIIa), wherein R⁴ and R⁵ are each independently selected from the group consisting of hydrogen and optionally substituted C₁-C₆alkyl. In some embodiments the FXR modulator is a compound of Formula (XIIa), wherein R⁴ and R⁵ are each hydrogen. In some embodiments the FXR modulator is a compound of Formula (XIIa), wherein R⁴ and R⁵ are each independently optionally substituted C₁-C₆alkyl. In some embodiments the FXR modulator is a compound of Formula (XIIa), wherein R⁴ and R⁵ are each methyl. In some embodiments the FXR modulator is a compound of Formula (XIIa), wherein R⁴ and R⁵ form an optionally substituted C₃-C₆cycloalkyl ring or an optionally substituted C₂-C₇heterocycloalkyl ring. In some embodiments is a compound of Formula (XIIa) wherein R⁴ and R⁵ form an optionally substituted C₃-C₆cycloalkyl ring. In some embodiments is a compound of Formula (XIIa) wherein R⁴ and R⁵ form an optionally substituted C₂-C₇heterocycloalkyl ring.

In some embodiments the FXR modulator is a compound of Formula (XIIa), wherein R⁶ and R⁷ are each independently selected from the group consisting of hydrogen, halogen, and optionally substituted C₁-C₆alkyl. In some embodiments the FXR modulator is a compound of Formula (XIIa), wherein R⁶ and R⁷ are each independently selected from the group consisting of hydrogen and optionally substituted C₁-C₆alkyl. In some embodiments the FXR modulator is a compound of Formula (XIIa), wherein R⁶ and R⁷ are each independently optionally substituted C₁-C₆alkyl. In some embodiments the FXR modulator is a compound of Formula (XIIa), wherein R⁶ and R⁷ are each methyl. In some embodiments the FXR modulator is a compound of Formula (XIIa), wherein R⁶ and R⁷ are each hydrogen.

In some embodiments the FXR modulator is a compound of Formula (XIIa), wherein R⁶ and R⁷ are hydrogen, R⁴ and R⁵ are independently optionally substituted C₁-C₆alkyl, R³ is —C(O)R²⁰, and R²⁰ is optionally substituted aryl. In some embodiments the FXR modulator is a compound of Formula (XIIa), wherein R⁶ and R⁷ are hydrogen, R⁴ and R⁵ are independently optionally substituted C₁-C₆alkyl, R³ is —C(O)R²⁰, and R²⁰ is optionally substituted heteroaryl. In some embodiments the FXR modulator is a compound of Formula (XIIa), wherein R⁶ and R⁷ are hydrogen, R⁴ and R⁵ are methyl, R³ is —C(O)R²⁰, and R²⁰ is optionally substituted aryl. In some embodiments the FXR modulator is a compound of Formula (XIIa), wherein R⁶ and R⁷ are hydrogen, R⁴ and R⁵ are methyl, R³ is —C(O)R²⁰, and R²⁰ is optionally substituted heteroaryl.

In some embodiments the FXR modulator is a compound of Formula (XIIa), wherein R⁶ and R⁷ are hydrogen, R⁴ and R⁵ are independently optionally substituted C₁-C₆alkyl, R³ is —S(O)₂R²⁰, and R²⁰ is optionally substituted aryl. In some embodiments the FXR modulator is a compound of Formula (XIIa), wherein R⁶ and R⁷ are hydrogen, R⁴ and R⁵ are independently optionally substituted C₁-C₆alkyl, R³ is —S(O)₂R²⁰, and R²⁰ is optionally substituted heteroaryl. In some embodiments the FXR modulator is a compound of Formula (XIIa), wherein R⁶ and R⁷ are hydrogen, R⁴ and R⁵ are methyl, R³ is —S(O)₂R²⁰, and R²⁰ is optionally substituted aryl. In some embodiments the FXR modulator is a compound of Formula (XIIa), wherein R⁶ and R⁷ are hydrogen, R⁴ and R⁵ are methyl, R³ is —S(O)₂R²⁰, and R²⁰ is optionally substituted heteroaryl.

In some embodiments the FXR modulator is a compound of Formula (XIIa), wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted aryl. In some embodiments the FXR modulator is a compound of Formula (XIIa), wherein R⁶ and R⁷ are hydrogen, R⁴ and R⁵ are independently optionally substituted C₁-C₆alkyl, R³ is —C(O)N(R²¹)R²², R²¹ is hydrogen and R²² is optionally substituted heteroaryl. In some embodiments the FXR modulator is a compound of Formula (XIIa), wherein R⁶ and R⁷ are hydrogen, R⁴ and R⁵ are methyl, R³ is —C(O)N(R²¹)R²², R²¹ is hydrogen and R²² is optionally substituted aryl. In some embodiments the FXR modulator is a compound of Formula (XIIa), wherein R⁶ and R⁷ are hydrogen, R⁴ and R⁵ are methyl, R³ is —C(O)N(R²¹)R²², R²¹ is hydrogen and R²² is optionally substituted heteroaryl.

In some embodiments the FXR modulator is a compound of Formula (XIIa), wherein p is 0. In some embodiments the FXR modulator is a compound of Formula (XIIa), wherein p is 1. In some embodiments the FXR modulator is a compound of Formula (XIIa), wherein p is 2. In some embodiments the FXR modulator is a compound of Formula (XIIa), wherein p is 3. In some embodiments the FXR modulator is a compound of Formula (XIIa), wherein p is 4.

In some embodiments the FXR modulator is a compound of Formula (XIIa), wherein p is 2 and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIIa), wherein p is 2 and each R³¹ is independently halogen, or optionally substituted C₁-C₆alkyl. In some embodiments the FXR modulator is a compound of Formula (XIIa), wherein p is 2 and each R³¹ is halogen. In some embodiments the FXR modulator is a compound of Formula (XIIa), wherein p is 2 and each R³¹ is F.

In some embodiments the FXR modulator is a compound of Formula (XIIa), wherein R³⁰ is F, p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIIa), wherein R³⁰ is F, p is 2 and each R³¹ is independently halogen, or optionally substituted C₁-C₆alkyl. In some embodiments the FXR modulator is a compound of Formula (XIIa), wherein R³⁰ is F, p is 2 and each R³¹ is halogen. In some embodiments the FXR modulator is a compound of Formula (XIIa), wherein R³⁰ is F, p is 2 and each R³¹ is F.

In some embodiments the FXR modulator is a compound of Formula (XIIa), wherein p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIIa), wherein p is 1 and R³¹ is halogen, or optionally substituted C₁-C₆alkyl. In some embodiments the FXR modulator is a compound of Formula (XIIa), wherein p is 1 and R³¹ is halogen. In some embodiments the FXR modulator is a compound of Formula (XIIa), wherein p is 1 and R³¹ is F.

In some embodiments the FXR modulator is a compound of Formula (XIIa), wherein R³⁰ is F, p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIIa), wherein R³⁰ is F, p is 1 and R³¹ is halogen, or optionally substituted C₁-C₆alkyl. In some embodiments the FXR modulator is a compound of Formula (XIIa), wherein R³⁰ is F, p is 1 and R³¹ is halogen. In some embodiments the FXR modulator is a compound of Formula (XIIa), wherein R³⁰ is F, p is 1 and R³¹ is F.

In some embodiments the FXR modulator is a compound of Formula (XIIa), wherein R³⁰ is

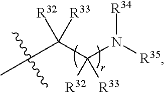

p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIIa), wherein R³⁰ is

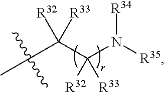

p is 2 and each R³¹ is independently halogen, or optionally substituted C₁-C₆alkyl. In some embodiments the FXR modulator is a compound of Formula (XIIa), wherein R³⁰ is

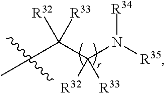

p is 2 and each R³¹ is halogen. In some embodiments the FXR modulator is a compound of Formula (XIIa), wherein R³⁰ is

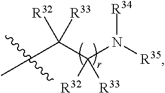

p is 2 and each R³¹ is F.

In some embodiments the FXR modulator is a compound of Formula (XIIa), wherein R³⁰ is

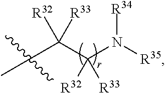

p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIIa), wherein R³⁰ is

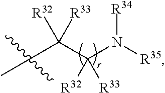

p is 1 and R³¹ is halogen, or optionally substituted C₁-C₆alkyl. In some embodiments the FXR modulator is a compound of Formula (XIIa), wherein R³⁰ is

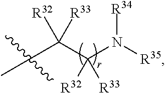

p is 1 and R³¹ is halogen. In some embodiments the FXR modulator is a compound of Formula (XIIa), wherein R³⁰ is

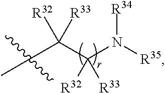

### p is 1 and R³¹ is F.

In some embodiments the FXR modulator is a compound of Formula (XIIa), wherein R³⁰ is

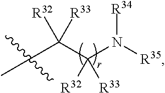

### and p is O.

In some embodiments the FXR modulator is a compound of Formula (XIIa), wherein R³⁰ is

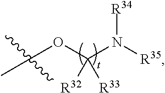

p is 2, and each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIIa), wherein R³⁰ is

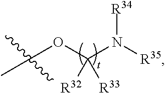

p is 2 and each R³¹ is independently halogen, or optionally substituted C₁-C₆alkyl. In some embodiments the FXR modulator is a compound of Formula (XIIa), wherein R³⁰ is

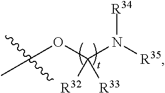

p is 2 and each R³¹ is halogen. In some embodiments the FXR modulator is a compound of Formula (XIIa), wherein R³⁰ is

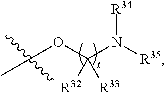

p is 2 and each R³¹ is F.

In some embodiments the FXR modulator is a compound of Formula (XIIa), wherein R³⁰ is

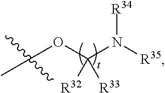

p is 1 and R³¹ is halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl. In some embodiments the FXR modulator is a compound of Formula (XIIa), wherein R³⁰ is

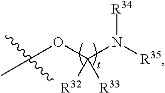

p is 1 and R³¹ is halogen, or optionally substituted C₁-C₆alkyl. In some embodiments the FXR modulator is a compound of Formula (XIIa), wherein R³⁰ is

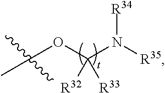

p is 1 and R³¹ is halogen. In some embodiments the FXR modulator is a compound of Formula (XIIa), wherein R³⁰ is

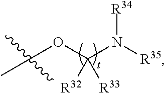

### p is 1 and R³¹ is F.

In some embodiments the FXR modulator is a compound of Formula (XIIa), wherein R³⁰ is

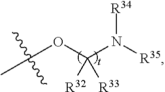

and p is 0.

In a further embodiment of the aforementioned embodiments, the FXR modulator is a compound of Formula (XIIa), wherein R² is selected from the group consisting of —CN, —C(O)OR²⁵, —C(O)N(R²⁵)R²⁶,

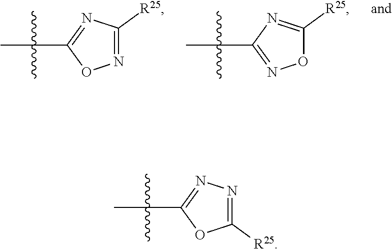

In a further embodiment of the aforementioned embodiments, the FXR modulator is a compound of Formula (XIIa), wherein R² is —CN.

In a further embodiment of the aforementioned embodiments, the FXR modulator is a compound of Formula (XIIa), wherein R² is —C(O)OR²⁵. In a further embodiment of the aforementioned embodiments, the FXR modulator is a compound of Formula (XIIa), wherein R² is —C(O)OR²⁵, and R²⁵ is independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XIIa), wherein R² is —C(O)OR²⁵, and R²⁵ is independently selected from the group consisting of hydrogen, and optionally substituted C₁-C₆alkyl. In a further embodiment of the aforementioned embodiments, the FXR modulator is a compound of Formula (XIIa), wherein R² is —C(O)OR²⁵, and R²⁵ is hydrogen. In a further embodiment of the aforementioned embodiments, the FXR modulator is a compound of Formula (XIIa), wherein R² is —C(O)OR²⁵, and R²⁵ is optionally substituted C₁-C₆alkyl. In a further embodiment of the aforementioned embodiments, the FXR modulator is a compound of Formula (XIIa), wherein R² is —C(O)OR²⁵, and R²⁵ is unsubstituted C₁-C₆alkyl. In a further embodiment of the aforementioned embodiments, the FXR modulator is a compound of Formula (XIIa), wherein R² is —C(O)OR²⁵, and R²⁵ is methyl. In a further embodiment of the aforementioned embodiments, the FXR modulator is a compound of Formula (XIIa), wherein R² is —C(O)OR²⁵, and R²⁵ is ethyl.

In a further embodiment of the aforementioned embodiments, the FXR modulator is a compound of Formula (XIIa), wherein R² is —C(O)N(R²⁵)R²⁶. In a further embodiment of the aforementioned embodiments, the FXR modulator is a compound of Formula (XIIa), wherein R² is —C(O)N(R²⁵)R²⁶, and R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XIIa), wherein R² is —C(O)N(R²⁵)R²⁶, and R²⁵ and R²⁶ are each independently selected from the group consisting of hydrogen, and optionally substituted C₁-C₆alkyl. In a further embodiment of the aforementioned embodiments, the FXR modulator is a compound of Formula (XIIa), wherein R² is —C(O)N(R²⁵)R²⁶, and R²⁵ and R²⁶ are hydrogen. In a further embodiment of the aforementioned embodiments, the FXR modulator is a compound of Formula (XIIa), wherein R² is —C(O)N(R²⁵)R²⁶, and R²⁵ and R²⁶ are each independently optionally substituted C₁-C₆alkyl. In a further embodiment of the aforementioned embodiments, the FXR modulator is a compound of Formula (XIIa), wherein R² is —C(O)N(R²⁵)R²⁶, R²⁵ is hydrogen, and R²⁶ is optionally substituted C₁-C₆alkyl. In a further embodiment of the aforementioned embodiments, the FXR modulator is a compound of Formula (XIIa), wherein R² is —C(O)N(R²⁵)R²⁶, and R²⁵ and R²⁶ are each independently unsubstituted C₁-C₆alkyl. In a further embodiment of the aforementioned embodiments, the FXR modulator is a compound of Formula (XIIa), wherein R² is —C(O)N(R²⁵)R²⁶, R²⁵ is hydrogen, and R²⁶ are methyl. In a further embodiment of the aforementioned embodiments, the FXR modulator is a compound of Formula (XIIa), wherein R² is —C(O)N(R²⁵)R²⁶, and R²⁵ and R²⁶ are methyl. In a further embodiment of the aforementioned embodiments, the FXR modulator is a compound of Formula (XIIa), wherein R² is —C(O)N(R²⁵)R²⁶, and R²⁵ and R²⁶ are ethyl.

In a further embodiment of the aforementioned embodiments, the FXR modulator is a compound of Formula (XIIa), wherein R² is

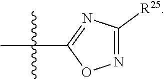

In a further embodiment of the aforementioned embodiments, the FXR modulator is a compound of Formula (XIIa), wherein R² is

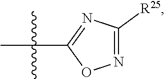

and R²⁵ is optionally substituted C₁-C₆alkyl. In a further embodiment of the aforementioned embodiments, the FXR modulator is a compound of Formula (XIIa), wherein R² is

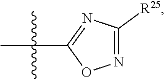

and R²⁵ is methyl. In a further embodiment of the aforementioned embodiments, the FXR modulator is a compound of Formula (XIIa), wherein R² is

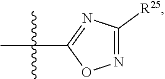

and R²⁵ is ethyl.

In a further embodiment of the aforementioned embodiments, the FXR modulator is a compound of Formula (XIIa), wherein R² is

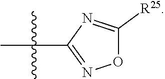

In a further embodiment of the aforementioned embodiments, the FXR modulator is a compound of Formula (XIIa), wherein R² is

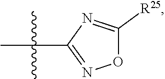

and R²⁵ is optionally substituted C₁-C₆alkyl. In a further embodiment of the aforementioned embodiments, the FXR modulator is a compound of Formula (XIIa), wherein R² is

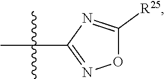

and R²⁵ is methyl. In a further embodiment of the aforementioned embodiments, the FXR modulator is a compound of Formula (XIIa), wherein R² is

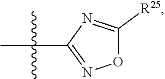

and R²⁵ is ethyl.

In a further embodiment of the aforementioned embodiments, the FXR modulator is a compound of Formula (XIIa), wherein R² is

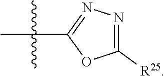

In a further embodiment of the aforementioned embodiments, the FXR modulator is a compound of Formula (XIIa), wherein R² is

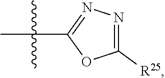

and R²⁵ is optionally substituted C₁-C₆alkyl. In a further embodiment of the aforementioned embodiments, the FXR modulator is a compound of Formula (XIIa), wherein R² is

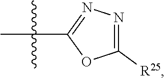

and R²⁵ is methyl. In a further embodiment of the aforementioned embodiments, the FXR modulator is a compound of Formula (XIIa), wherein R² is

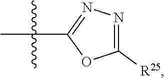

and R²⁵ is ethyl.

In a further embodiment of the aforementioned embodiments, the FXR modulator is a compound of Formula (XIIa), wherein R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl). In a further embodiment of the aforementioned embodiments, the FXR modulator is a compound of Formula (XIIa), wherein R¹ is hydrogen. In a further embodiment of the aforementioned embodiments, the FXR modulator is a compound of Formula (XIIa), wherein R¹ is optionally substituted C₁-C₆alkyl. In a further embodiment of the aforementioned embodiments, the FXR modulator is a compound of Formula (XIIa), wherein R¹ is methyl. In a further embodiment of the aforementioned embodiments, the FXR modulator is a compound of Formula (XIIa), wherein R¹ is optionally substituted C₂-C₆alkenyl. In a further embodiment of the aforementioned embodiments, the FXR modulator is a compound of Formula (XIIa), wherein R¹ is optionally substituted C₂-C₆alkynyl.

In a further embodiment of the aforementioned embodiments, the FXR modulator is a compound of Formula (XIIa), wherein R¹ and R² together with the carbon atoms to which they are attached, form an optionally substituted C₂-C₉heterocycloalkyl ring or an optionally substituted heteroaryl ring. In a further embodiment of the aforementioned embodiments, the FXR modulator is a compound of Formula (XIIa), wherein R¹ and R² together with the carbon atoms to which they are attached, form an optionally substituted C₂-C₉heterocycloalkyl ring. In a further embodiment of the aforementioned embodiments, the FXR modulator is a compound of Formula (XIIa), wherein R¹ and R² together with the carbon atoms to which they are attached, form an optionally substituted heteroaryl ring.

In a further embodiment of the aforementioned embodiments, the FXR modulator is a compound of Formula (XIIa), wherein R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl). In a further embodiment of the aforementioned embodiments, the FXR modulator is a compound of Formula (XIIa), wherein R⁸ is selected from the group consisting of hydrogen, and optionally substituted C₁-C₆alkyl. In a further embodiment of the aforementioned embodiments, the FXR modulator is a compound of Formula (XIIa), wherein R⁸ is optionally substituted C₁-C₆alkyl. In a further embodiment of the aforementioned embodiments, the FXR modulator is a compound of Formula (XIIa), wherein R⁸ is methyl. In a further embodiment of the aforementioned embodiments, the FXR modulator is a compound of Formula (XIIa), wherein R⁸ is optionally substituted C₁-C₆alkyl. In a further embodiment of the aforementioned embodiments, the FXR modulator is a compound of Formula (XIIa), wherein R⁸ is ethyl. In a further embodiment of the aforementioned embodiments, the FXR modulator is a compound of Formula (XIIa), wherein R⁸ is optionally substituted C₁-C₆alkyl. In a further embodiment of the aforementioned embodiments, the FXR modulator is a compound of Formula (XIIa), wherein R⁸ is hydrogen.

Any combination of the groups described above for the various variables is contemplated herein. Throughout the specification, groups and substituents thereof can be chosen by one skilled in the field to provide stable moieties and compounds.

In some embodiments of the methods described herein, the FXR modulator is (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof.

In some embodiments is the administration of an FXR modulator described herein to a mammal in the treatment of diseases, disorders or conditions that would benefit from FXR modulation, wherein the FXR modulator is (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, or obesity. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is nonalcoholic steatohepatitis (NASH). In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is hyperlipidemia. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is hypercholesterolemia. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is hypertriglyceridemia. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is dyslipidemia. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is lipodystrophy. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is atherosclerosis. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is atherosclerotic disease. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is atherosclerotic disease events. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is atherosclerotic cardiovascular disease. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is Syndrome X. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is diabetes mellitus. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is type II diabetes. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is insulin insensitivity. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is hyperglycemia. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is cholestasis. In some embodiments is a method of treating a metabolic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the metabolic disorder is obesity.

Further disclosed herein, is a method of treating a choleastatic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof. In some embodiments is a method of treating a choleastatic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the cholestatic disorder is primary biliary cirrhosis. In some embodiments is a method of treating a cholestatic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the cholestatic disorder is primary sclerosing cholangitis. In some embodiments is a method of treating a cholestatic disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the cholestatic disorder is biliary atresia.

Further disclosed herein, is a method of treating fibrosis associated with nonalcoholic steatohepatitis (NASH), chronic viral hepatitis, or autoimmune hepatitis, in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof. In some embodiments is a method of treating fibrosis associated with nonalcoholic steatohepatitis (NASH), in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof. In some embodiments is a method of treating fibrosis associated with chronic viral hepatitis, in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof. In some embodiments is a method of treating fibrosis associated with autoimmune hepatitis, in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof.

Further disclosed herein, is a method of treating cholesterol gallstone disease, in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof.

Further disclosed herein, is a method of treating portal hypertension, in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof.

Further disclosed herein, is a method of treating a gastrointestinal disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof. In some embodiments is a method of treating a gastrointestinal disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the gastrointestinal disorder is inflammatory bowel disease. In some embodiments is a method of treating a gastrointestinal disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the gastrointestinal disorder is irritable bowel syndrome. In some embodiments is a method of treating a gastrointestinal disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the gastrointestinal disorder is bile acid diarrhea.

Further disclosed herein, is a method of treating a kidney disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof. In some embodiments is a method of treating a kidney disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the kidney disorder is diabetic nephropathy. In some embodiments is a method of treating a kidney disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the kidney disorder is renal fibrosis. In some embodiments is a method of treating a kidney disorder in a subject in need thereof, comprising administering to the subject in need thereof an effective dose of (E)-6-(3,4-difluorobenzoyl)-N-isopropyl-4,4-dimethyl-3-(trifluoromethyl)-1,4,5,6-tetrahydropyrazolo[3,4-d]azepine-8-carboxamide (Compound 1), or a pharmaceutically acceptable salt thereof, wherein the kidney disorder is focal segmental glomerulosclerosis.

In some embodiments of the methods described herein, the FXR modulator is (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof.

In some embodiments is the administration of an FXR modulator described herein to a mammal in the treatment of diseases, disorders or conditions that would benefit from FXR modulation, wherein the FXR modulator is (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, or obesity. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is nonalcoholic steatohepatitis (NASH). In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is hyperlipidemia. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is hypercholesterolemia. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is hypertriglyceridemia. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is dyslipidemia. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is lipodystrophy. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is atherosclerosis. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is atherosclerotic disease. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is atherosclerotic disease events. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is atherosclerotic cardiovascular disease. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is Syndrome X. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is diabetes mellitus. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is type II diabetes. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is insulin insensitivity. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is hyperglycemia. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is cholestasis. In some embodiments is a method of treating a metabolic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the metabolic disorder is obesity.

Further disclosed herein, is a method of treating a choleastatic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof. In some embodiments is a method of treating a choleastatic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the cholestatic disorder is primary biliary cirrhosis. In some embodiments is a method of treating a cholestatic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the cholestatic disorder is primary sclerosing cholangitis. In some embodiments is a method of treating a cholestatic disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the cholestatic disorder is biliary atresia.

Further disclosed herein, is a method of treating fibrosis associated with nonalcoholic steatohepatitis (NASH), chronic viral hepatitis, or autoimmune hepatitis, in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof. In some embodiments is a method of treating fibrosis associated with nonalcoholic steatohepatitis (NASH), in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof. In some embodiments is a method of treating fibrosis associated with chronic viral hepatitis, in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof. In some embodiments is a method of treating fibrosis associated with autoimmune hepatitis, in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof.

Further disclosed herein, is a method of treating cholesterol gallstone disease, in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof.

Further disclosed herein, is a method of treating portal hypertension, in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof.

Further disclosed herein, is a method of treating a gastrointestinal disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof. In some embodiments is a method of treating a gastrointestinal disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the gastrointestinal disorder is inflammatory bowel disease. In some embodiments is a method of treating a gastrointestinal disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the gastrointestinal disorder is irritable bowel syndrome. In some embodiments is a method of treating a gastrointestinal disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the gastrointestinal disorder is bile acid diarrhea.

Further disclosed herein, is a method of treating a kidney disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof. In some embodiments is a method of treating a kidney disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the kidney disorder is diabetic nephropathy. In some embodiments is a method of treating a kidney disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the kidney disorder is renal fibrosis. In some embodiments is a method of treating a kidney disorder in a subject in need thereof, comprising administering to the subject in need thereof an effective dose of (E)-isopropyl 4,4-dimethyl-6-(4-(2-morpholinoethoxy)benzoyl)-3-(trifluoromethyl)-1,4,5,6-tetrahydropyrazolo[3,4-d]azepine-8-carboxylate (Compound 2), or a pharmaceutically acceptable salt thereof, wherein the kidney disorder is focal segmental glomerulosclerosis.

In some embodiments of the methods described herein, the FXR modulator is (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

In some embodiments is the administration of an FXR modulator described herein to a mammal in the treatment of diseases, disorders or conditions that would benefit from FXR modulation, wherein the FXR modulator is (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, or obesity. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is nonalcoholic steatohepatitis (NASH). In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is hyperlipidemia. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is hypercholesterolemia. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is hypertriglyceridemia. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is dyslipidemia. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is lipodystrophy. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is atherosclerosis. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is atherosclerotic disease. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is atherosclerotic disease events. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is atherosclerotic cardiovascular disease. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is Syndrome X. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is diabetes mellitus. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is type II diabetes. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is insulin insensitivity. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is hyperglycemia. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is cholestasis. In some embodiments is a method of treating a metabolic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the metabolic disorder is obesity.

Further disclosed herein, is a method of treating a choleastatic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof. In some embodiments is a method of treating a choleastatic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the cholestatic disorder is primary biliary cirrhosis. In some embodiments is a method of treating a cholestatic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the cholestatic disorder is primary sclerosing cholangitis. In some embodiments is a method of treating a cholestatic disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the cholestatic disorder is biliary atresia.

Further disclosed herein, is a method of treating fibrosis associated with nonalcoholic steatohepatitis (NASH), chronic viral hepatitis, or autoimmune hepatitis, in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof. In some embodiments is a method of treating fibrosis associated with nonalcoholic steatohepatitis (NASH), in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof. In some embodiments is a method of treating fibrosis associated with chronic viral hepatitis, in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof. In some embodiments is a method of treating fibrosis associated with autoimmune hepatitis, in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

Further disclosed herein, is a method of treating cholesterol gallstone disease, in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

Further disclosed herein, is a method of treating portal hypertension, in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof.

Further disclosed herein, is a method of treating a gastrointestinal disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof. In some embodiments is a method of treating a gastrointestinal disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the gastrointestinal disorder is inflammatory bowel disease. In some embodiments is a method of treating a gastrointestinal disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the gastrointestinal disorder is irritable bowel syndrome. In some embodiments is a method of treating a gastrointestinal disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the gastrointestinal disorder is bile acid diarrhea.

Further disclosed herein, is a method of treating a kidney disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof. In some embodiments is a method of treating a kidney disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the kidney disorder is diabetic nephropathy. In some embodiments is a method of treating a kidney disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the kidney disorder is renal fibrosis. In some embodiments is a method of treating a kidney disorder in a subject in need thereof, comprising administering to the subject in need thereof an effective dose of (E)-isopropyl 6-(3-fluoro-4-(2-morpholinoethoxy)benzoyl)-4,4-dimethyl-3-(trifluoromethyl)-1,4,5,6-tetrahydropyrazolo[3,4-d]azepine-8-carboxylate (Compound 3), or a pharmaceutically acceptable salt thereof, wherein the kidney disorder is focal segmental glomerulosclerosis.

In some embodiments is a method of treating a metabolic disorder in an individual in need thereof, comprising co-administering to the individual a therapeutically effective amount of: (a) a first agent that is an FXR modulator; and (b) at least one second agent that is an CC2/CCR5 antagonist, ASK1 inhibitor, DPP-IV inhibitor, caspase preotease inhibitor, an SGLT2 inhibitor, acetyl-CoA carboxylase inhibitor, sodium-bile acide cotransporter-inhibitor, TLR-4 antagonist, PPAR alpha/delta agonist, a GLP-1 agonist, or a combination thereof; wherein the FXR modulator is a compound having the structure:

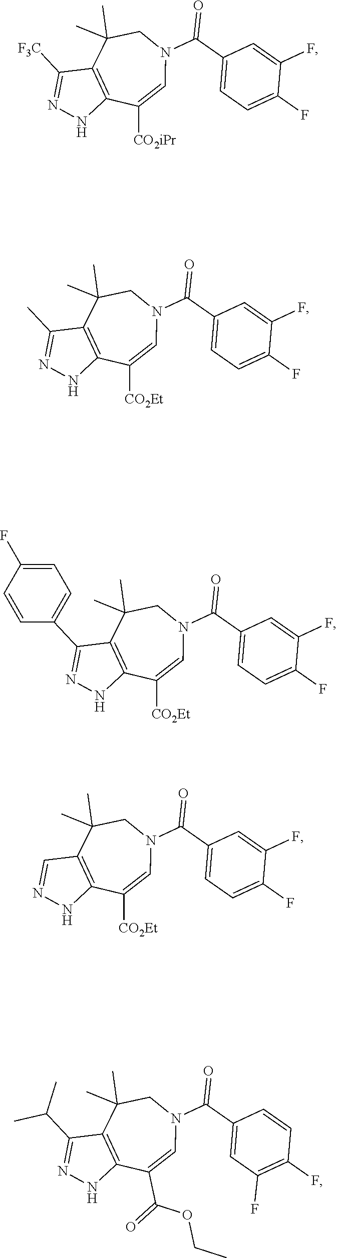

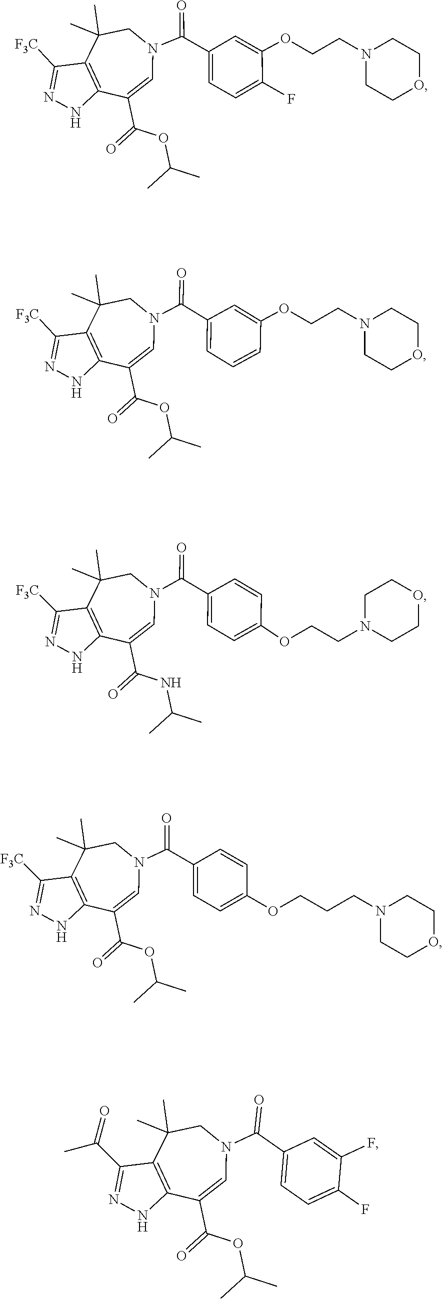 [SMILES: CC(C)OC(=O)C1=CN(C(=O)c2ccc(F)c(OCCN3CCOCC3)c2)CC(C)(C)c2c(C(F)(F)F)n[nH]c21 ; CC(C)OC(=O)C1=CN(C(=O)c2cccc(OCCN3CCOCC3)c2)CC(C)(C)c2c(C(F)(F)F)n[nH]c21 ; CC(C)NC(=O)C1=CN(C(=O)c2ccc(OCCN3CCOCC3)cc2)CC(C)(C)c2c(C(F)(F)F)n[nH]c21 ; CC(C)OC(=O)C1=CN(C(=O)c2ccc(OCCCN3CCOCC3)cc2)CC(C)(C)c2c(C(F)(F)F)n[nH]c21 ; CC(=O)c1n[nH]c2c1C(C)(C)CN(C(=O)c1ccc(F)c(F)c1)C=C2C(=O)OC(C)C]

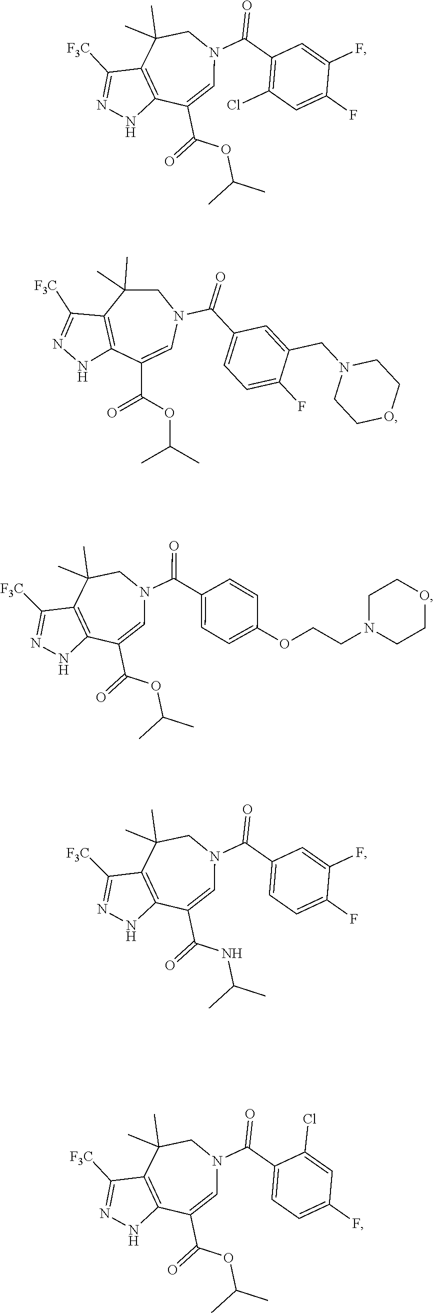 [SMILES: CC(C)OC(=O)C1=CN(C(=O)c2cc(F)c(F)cc2Cl)CC(C)(C)c2c(C(F)(F)F)n[nH]c21 ; CC(C)OC(=O)C1=CN(C(=O)c2ccc(F)c(CN3CCOCC3)c2)CC(C)(C)c2c(C(F)(F)F)n[nH]c21 ; CC(C)OC(=O)C1=CN(C(=O)c2ccc(OCCN3CCOCC3)cc2)CC(C)(C)c2c(C(F)(F)F)n[nH]c21 ; CC(C)NC(=O)C1=CN(C(=O)c2ccc(F)c(F)c2)CC(C)(C)c2c(C(F)(F)F)n[nH]c21 ; CC(C)OC(=O)C1=CN(C(=O)c2ccc(F)cc2Cl)CC(C)(C)c2c(C(F)(F)F)n[nH]c21]

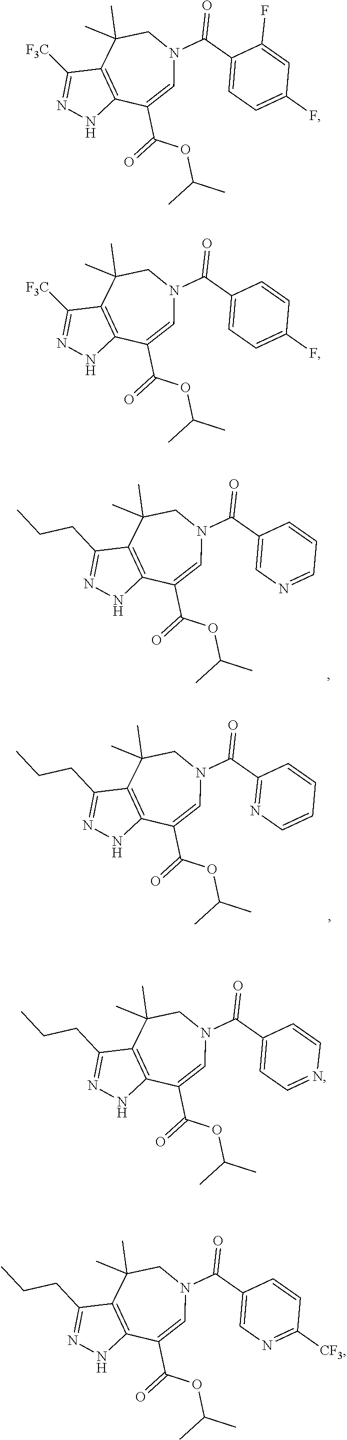 [SMILES: CC(C)OC(=O)C1=CN(C(=O)c2ccc(F)cc2F)CC(C)(C)c2c(C(F)(F)F)n[nH]c21 ; CC(C)OC(=O)C1=CN(C(=O)c2ccc(F)cc2)CC(C)(C)c2c(C(F)(F)F)n[nH]c21 ; CCCc1n[nH]c2c1C(C)(C)CN(C(=O)c1cccnc1)C=C2C(=O)OC(C)C ; CCCc1n[nH]c2c1C(C)(C)CN(C(=O)c1ccccn1)C=C2C(=O)OC(C)C ; CCCc1n[nH]c2c1C(C)(C)CN(C(=O)c1ccncc1)C=C2C(=O)OC(C)C ; CCCc1n[nH]c2c1C(C)(C)CN(C(=O)c1ccc(C(F)(F)F)nc1)C=C2C(=O)OC(C)C]

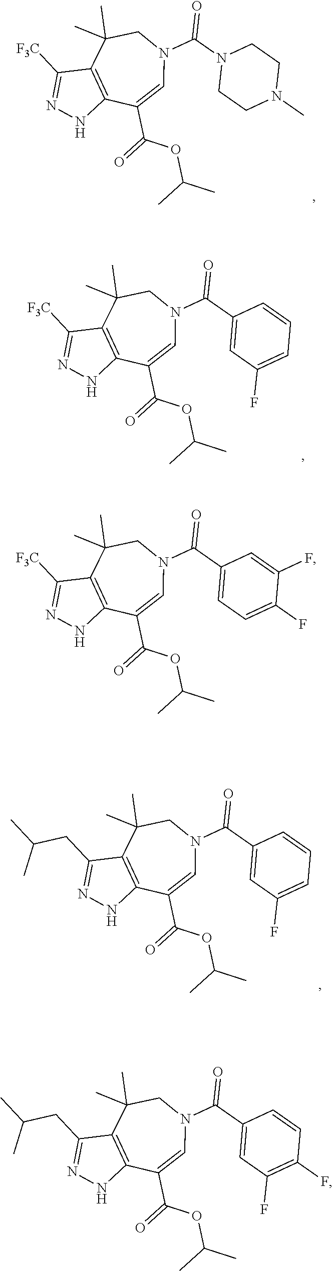

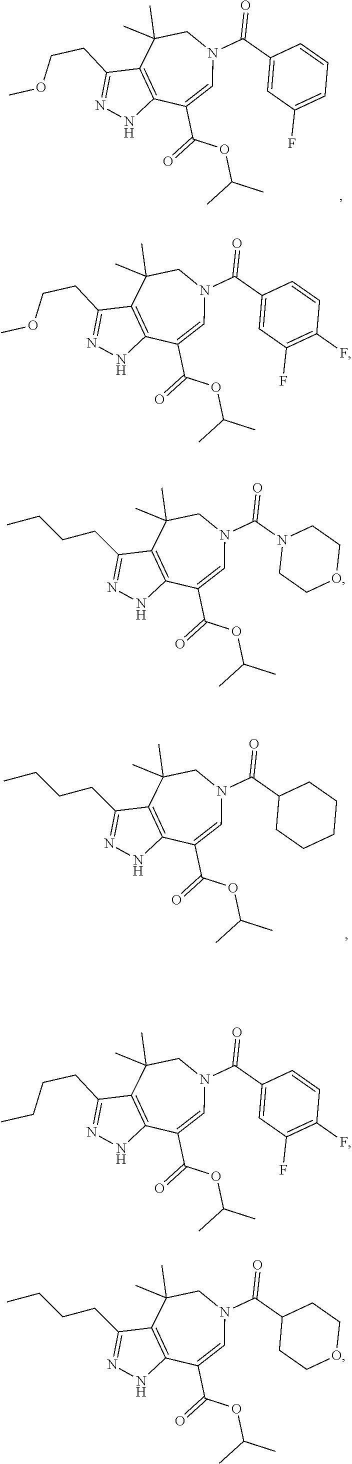

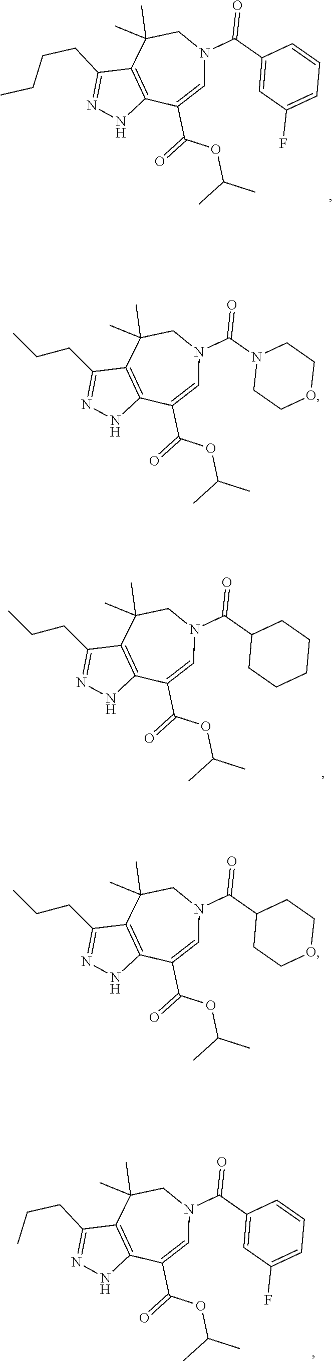 [SMILES: CCCCc1n[nH]c2c1C(C)(C)CN(C(=O)c1cccc(F)c1)C=C2C(=O)OC(C)C ; CCCc1n[nH]c2c1C(C)(C)CN(C(=O)N1CCOCC1)C=C2C(=O)OC(C)C ; CCCc1n[nH]c2c1C(C)(C)CN(C(=O)C1CCCCC1)C=C2C(=O)OC(C)C ; CCCc1n[nH]c2c1C(C)(C)CN(C(=O)C1CCOCC1)C=C2C(=O)OC(C)C ; CCCc1n[nH]c2c1C(C)(C)CN(C(=O)c1cccc(F)c1)C=C2C(=O)OC(C)C]

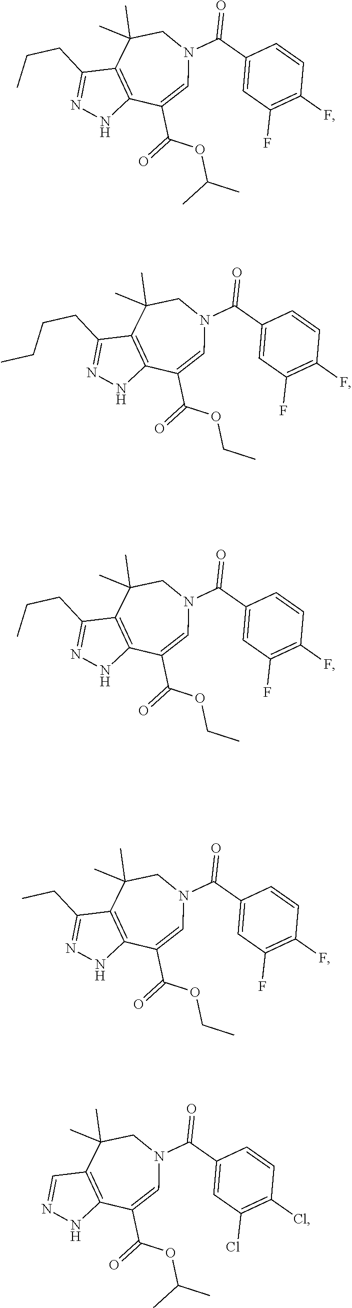 [SMILES: CCCc1n[nH]c2c1C(C)(C)CN(C(=O)c1ccc(F)c(F)c1)C=C2C(=O)OC(C)C ; CCCCc1n[nH]c2c1C(C)(C)CN(C(=O)c1ccc(F)c(F)c1)C=C2C(=O)OCC ; CCCc1n[nH]c2c1C(C)(C)CN(C(=O)c1ccc(F)c(F)c1)C=C2C(=O)OCC ; CCOC(=O)C1=CN(C(=O)c2ccc(F)c(F)c2)CC(C)(C)c2c(CC)n[nH]c21 ; CC(C)OC(=O)C1=CN(C(=O)c2ccc(Cl)c(Cl)c2)CC(C)(C)c2cn[nH]c21]

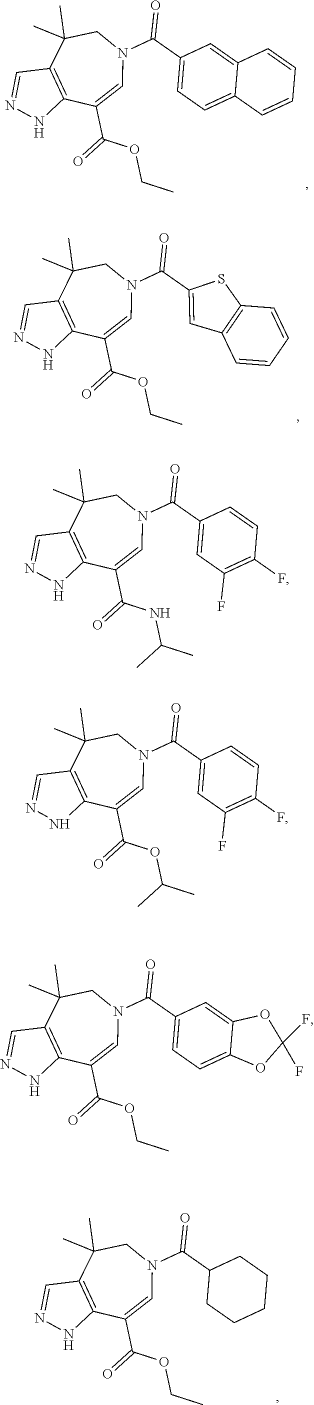

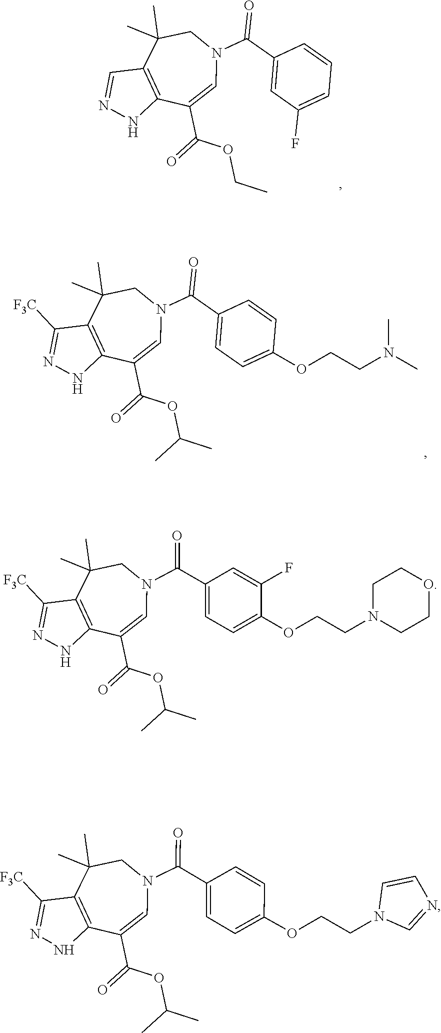 [SMILES: CCOC(=O)C1=CN(C(=O)c2cccc(F)c2)CC(C)(C)c2cn[nH]c21 ; CC(C)OC(=O)C1=CN(C(=O)c2ccc(OCCN(C)C)cc2)CC(C)(C)c2c(C(F)(F)F)n[nH]c21 ; CC(C)OC(=O)C1=CN(C(=O)c2ccc(OCCN3CCOCC3)c(F)c2)CC(C)(C)c2c(C(F)(F)F)n[nH]c21 ; CC(C)OC(=O)C1=CN(C(=O)c2ccc(OCCn3ccnc3)cc2)CC(C)(C)c2c(C(F)(F)F)n[nH]c21]

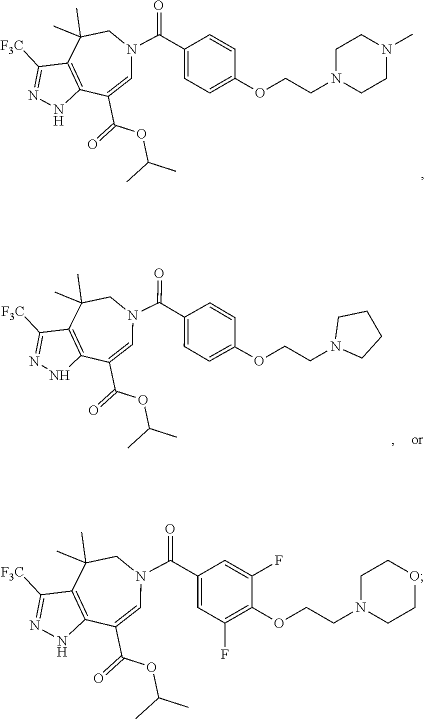 [SMILES: CC(C)OC(=O)C1=CN(C(=O)c2ccc(OCCN3CCN(C)CC3)cc2)CC(C)(C)c2c(C(F)(F)F)n[nH]c21 ; CC(C)OC(=O)C1=CN(C(=O)c2ccc(OCCN3CCCC3)cc2)CC(C)(C)c2c(C(F)(F)F)n[nH]c21 ; CC(C)OC(=O)C1=CN(C(=O)c2cc(F)c(OCCN3CCOCC3)c(F)c2)CC(C)(C)c2c(C(F)(F)F)n[nH]c21]

or a pharmaceutically acceptable salt, stereoisomer, or solvate thereof.

In some embodiments is a method of treating a metabolic disorder in an individual in need thereof, comprising co-administering to the individual a therapeutically effective amount of: (a) a first agent that is an FXR modulator; and (b) at least one second agent that is an CC2/CCR5 antagonist, ASK1 inhibitor, DPP-IV inhibitor, caspase preotease inhibitor, an SGLT2 inhibitor, acetyl-CoA carboxylase inhibitor, sodium-bile acide cotransporter-inhibitor, TLR-4 antagonist, PPAR alpha/delta agonist, a GLP-1 agonist, or a combination thereof; wherein the FXR modulator is a compound having the structure:

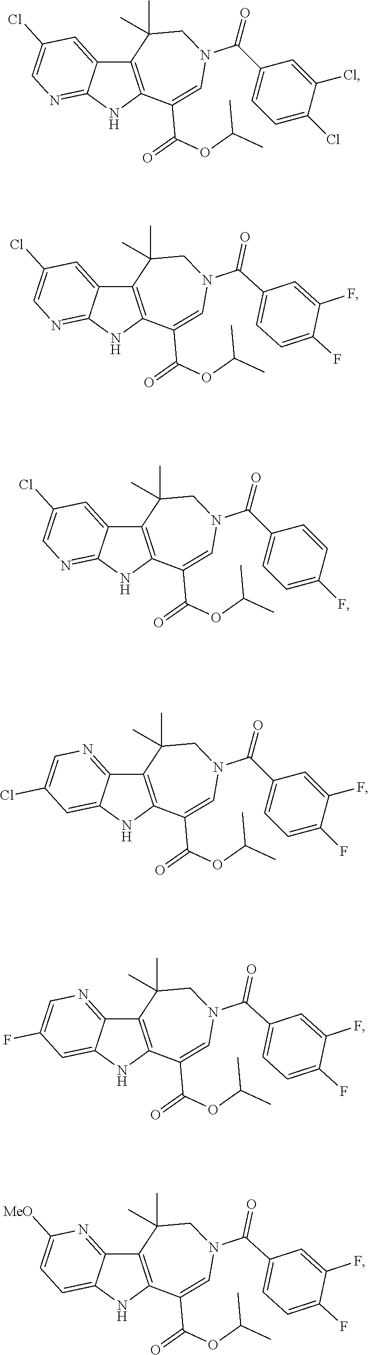 [SMILES: CC(C)OC(=O)C1=CN(C(=O)c2ccc(Cl)c(Cl)c2)CC(C)(C)c2c1[nH]c1ncc(Cl)cc21 ; CC(C)OC(=O)C1=CN(C(=O)c2ccc(F)c(F)c2)CC(C)(C)c2c1[nH]c1ncc(Cl)cc21 ; CC(C)OC(=O)C1=CN(C(=O)c2ccc(F)cc2)CC(C)(C)c2c1[nH]c1ncc(Cl)cc21 ; CC(C)OC(=O)C1=CN(C(=O)c2ccc(F)c(F)c2)CC(C)(C)c2c1[nH]c1cc(Cl)cnc21 ; CC(C)OC(=O)C1=CN(C(=O)c2ccc(F)c(F)c2)CC(C)(C)c2c1[nH]c1cc(F)cnc21 ; COc1ccc2[nH]c3c(c2n1)C(C)(C)CN(C(=O)c1ccc(F)c(F)c1)C=C3C(=O)OC(C)C]

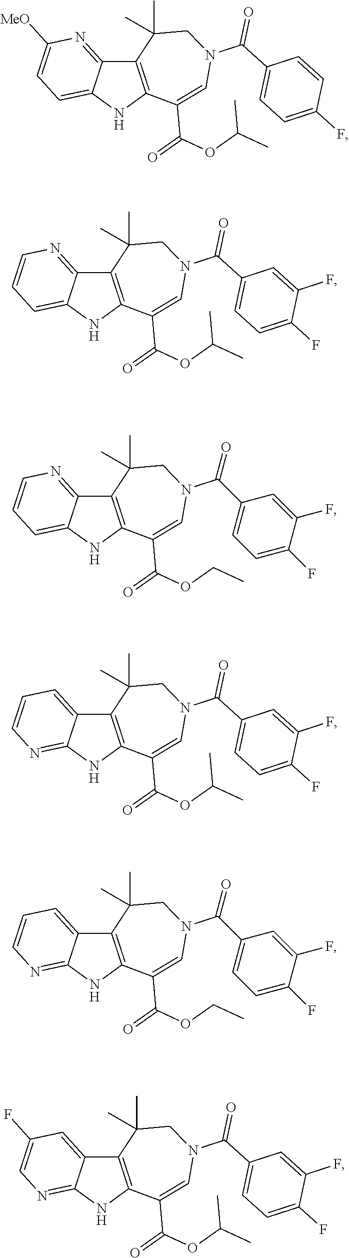 [SMILES: COc1ccc2[nH]c3c(c2n1)C(C)(C)CN(C(=O)c1ccc(F)cc1)C=C3C(=O)OC(C)C ; CC(C)OC(=O)C1=CN(C(=O)c2ccc(F)c(F)c2)CC(C)(C)c2c1[nH]c1cccnc21 ; CCOC(=O)C1=CN(C(=O)c2ccc(F)c(F)c2)CC(C)(C)c2c1[nH]c1cccnc21 ; CC(C)OC(=O)C1=CN(C(=O)c2ccc(F)c(F)c2)CC(C)(C)c2c1[nH]c1ncccc21 ; CCOC(=O)C1=CN(C(=O)c2ccc(F)c(F)c2)CC(C)(C)c2c1[nH]c1ncccc21 ; CC(C)OC(=O)C1=CN(C(=O)c2ccc(F)c(F)c2)CC(C)(C)c2c1[nH]c1ncc(F)cc21]

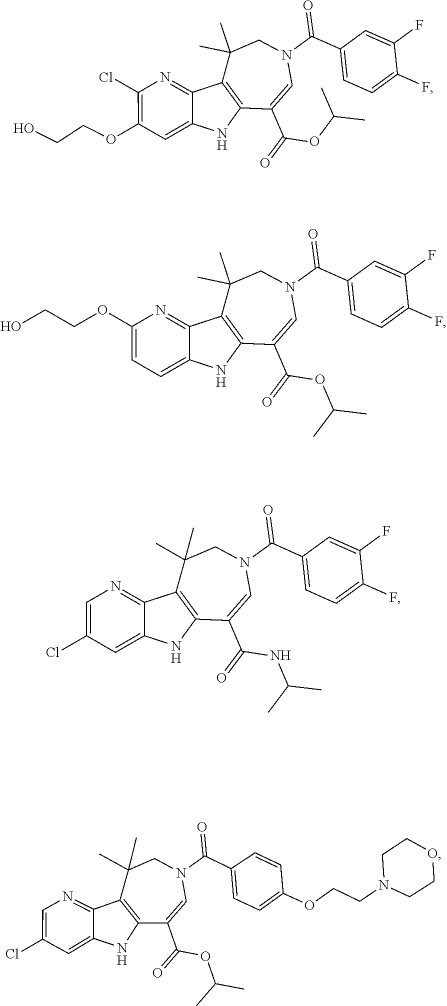 [SMILES: CC(C)OC(=O)C1=CN(C(=O)c2ccc(F)c(F)c2)CC(C)(C)c2c1[nH]c1cc(OCCO)c(Cl)nc21 ; CC(C)OC(=O)C1=CN(C(=O)c2ccc(F)c(F)c2)CC(C)(C)c2c1[nH]c1ccc(OCCO)nc21 ; CC(C)NC(=O)C1=CN(C(=O)c2ccc(F)c(F)c2)CC(C)(C)c2c1[nH]c1cc(Cl)cnc21 ; CC(C)OC(=O)C1=CN(C(=O)c2ccc(OCCN3CCOCC3)cc2)CC(C)(C)c2c1[nH]c1cc(Cl)cnc21]

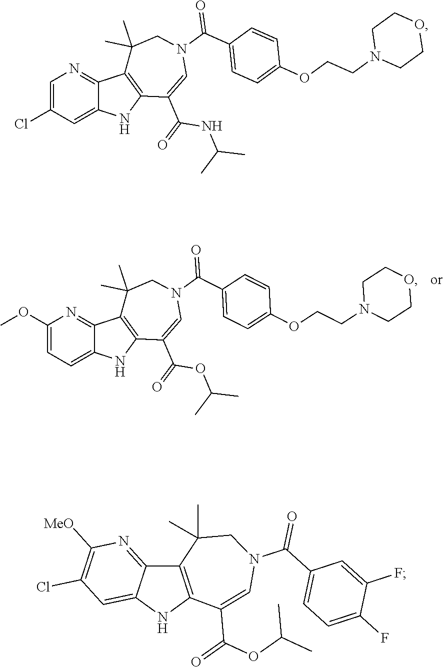 [SMILES: CC(C)NC(=O)C1=CN(C(=O)c2ccc(OCCN3CCOCC3)cc2)CC(C)(C)c2c1[nH]c1cc(Cl)cnc21 ; COc1ccc2[nH]c3c(c2n1)C(C)(C)CN(C(=O)c1ccc(OCCN2CCOCC2)cc1)C=C3C(=O)OC(C)C ; COc1nc2c3c([nH]c2cc1Cl)C(C(=O)OC(C)C)=CN(C(=O)c1ccc(F)c(F)c1)CC3(C)C]

or a pharmaceutically acceptable salt, stereoisomer, or solvate thereof.

### CCR2/CCR5 Antagonists:

Embodiments of CCR2/CCR5 antagonists used in combination with a first agent that is an FXR inhibitor includes cenicriviroc (CVC), aplaviroc, vicriviroc 5-({4-[(3S)-4-{2-methoxy-1-[4-(trifluoromethyl)phenyl]ethyl}-3-methylpiperazin-1-yl]-4-methylpiperidin-1-yl}carbonyl)-4,6-dimethylpyrimidine), maraviroc (e.g., 4,4′-difluorocyclohexylamide) and cochilioquinone A Caspase protease inhibitors:

Embodiments of caspase protease inhibitors used in combination with a first agent that is an FXR inhibitor includes emricasan, Q-VD-Oph, DEVD-CHO, zVAD-FMK, Pralnacasan, and M867.

### Acetyl-CoA Carboxylase Inhibitors:

Embodiments of acetyl-CoA carboxylase inhibitor used in combination with a first agent that is an FXR inhibitor includes (R)-2-(1-(2-(2-methoxyphenyl)-2-((tetrahydro-2H-pyran-4-yl)oxy)ethyl)-5-methyl-6-(oxazol-2-yl)-2,4-dioxo-1,4-dihydrothieno[2,3-d]pyrimidin-3(2H)-yl)-2-methylpropanoicacid, 5-(tetradecyloxy)-2-furoic acid, Medica 16, and (3R)-1′-(9-anthracenylcarbonyl) [1,4′-bipiperidin]-3-yl]-4-morpholinyl-methanone.

### Diacylglycerol Acyltransferase-1 Inhibitors:

Embodiments of diacylglycerol acyltransferase-1 inhibitors used in combination with a first agent that is an FXR inhibitor includes pradigastat, VK5211, A 922500, amidepsine A, and amidepisne D.

### Sodium-Bile Cotransporter Inhibitors:

Embodiments of sodium-bile cotransporter inhibitors used in combination with a first agent that is an FXR inhibitor includes volixibat, LJN 452, GSK2330672, AZD-7806, S-8921, AK-105, BARI-1741, SC-435 and SC-635.

### TLR-4 Antagonist:

Embodiments of TLR-4 antagonists used in combination with a first agent that is an FXR inhibitor includes JKB-121, amitriptyline, imipramine, naloxone, LPS-RS, cyclbenzprine, mianserin, naltrexone, propentofylline, ketotitfen, ibudilast, (+)-naltrexone, tapentradol, and eritoran.

### PPAR Alpha/Delta Agonist:

Embodiments of PPAR alpha/delta agonists used in combination with a first agent that is an FXR inhibitor includes GFT505, clofibrate, gemfibrozil, ciprofibrate, bezafibrate, and fenofibrate, GW501516, aleglitzar, muraglitazar, tesaglitazar, and saroglitazar.

### DPP-IV Inhibitors:

Disclosed herein are DPP-IV inhibitors used in combination with a first agent that is an FXR inhibitor. In some embodiments, the DPP-IV inhibitor is selected from sitagliptin, saxagliptin, linagliptin, alogliptin, vildagliptin, gemigliptin, anagliptin, teneligliptin, trelagliptin, dutogliptin, and omarigliptin.

### SGLT2 Inhibitors:

Disclosed herein are SGLT2 inhibitors used in combination with a first agent that is an FXR inhibitor. In some embodiments, the SGLT2 inhibitor is selected from canagliflozin, empagliflozin, dapagliflozin, ipragliflozin, tofogliflozin, sergliflozin etabonate, remogliflozin etabonate, and ertugliflozin.

### ASK1 Inhibitors:

Disclosed herein are ASK1 inhibitors used in combination with a first agent that is an FXR inhibitor. In some embodiments, the ASK1 inhibitor is selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyri dinecarboxamide), and TC ASK 10 (4-(1,1-dimethylethyl)-N-[6-(1H-imidazol-1-yl)imidazo[1,2-a]pyridin-2-yl]benzamide dihydrochloride).

In some embodiments, the ASK1 inhibitor is selected from a compound disclosed in any of the following publications: WO2008/016131; EP2058309, US2010/0029619, WO2016/49069, US2011/0009410, US2013/0197037, US2013/0197037, US201410179663, and US201410018370; each of which is incorporated by reference in their entirety.

### GLP-1 Agonists:

Disclosed herein are GLP-1 agonists used in combination with a first agent that is an FXR inhibitor. In some embodiments, the GLP-1 agonist is selected from exenatide, liraglutide, lixisenatide, albiglutide, dulaglutide, taspoglutide, and semaglutide.

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein.

In some situations, the compounds described herein possess one or more chiral centers and each center exists in the R configuration, or S configuration. In some embodiments, the compounds described herein possess three chiral centers and each center exists in the R configuration, or S configuration. In some embodiments, the compounds described herein possess four chiral centers and each center exists in the R configuration, or S configuration. In some embodiments, the compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran or methanol. In addition, the compounds provided herein exist in unsolvated as well as solvated forms. In general, the solvated forms are considered equivalent to the unsolvated forms for the purposes of the compounds and methods provided herein.

### Combination Treatment

In some embodiments, the compounds disclosed herein are used in combination for the treatment of a disease, disorder or condition in a mammal that would benefit from combined FXR modulation and DPP-IV inhibition. In some embodiments, the compounds disclosed herein are used in combination for the treatment of a disease, disorder or condition in a mammal that would benefit from combined FXR modulation and SGLT2 inhibition. In some embodiments, the compounds disclosed herein are used in combination for the treatment of a disease, disorder or condition in a mammal that would benefit from combined FXR modulation and ASK1 inhibition. In some embodiments, the compounds disclosed herein are used in combination for the treatment of a disease, disorder or condition in a mammal that would benefit from combined FXR modulation and GLP-1 modulation.

Disclosed herein, are methods of treating a metabolic disorder in an individual in need thereof, comprising co-administering to the individual a therapeutically effective amount of: (a) a first agent that is an FXR modulator; and (b) a second agent that is a CCR2/CCR5 antagonist.

Disclosed herein, are methods of treating a metabolic disorder in an individual in need thereof, comprising co-administering to the individual a therapeutically effective amount of: (a) a first agent that is an FXR modulator; and (b) a second agent that is an ASK1 inhibitor.

Disclosed herein, are methods of treating a metabolic disorder in an individual in need thereof, comprising co-administering to the individual a therapeutically effective amount of: (a) a first agent that is an FXR modulator; and (b) a second agent that is a DPP-IV inhibitor.

Disclosed herein, are methods of treating a metabolic disorder in an individual in need thereof, comprising co-administering to the individual a therapeutically effective amount of: (a) a first agent that is an FXR modulator; and (b) a second agent that is a caspase protease inhibitor.

Disclosed herein, are methods of treating a metabolic disorder in an individual in need thereof, comprising co-administering to the individual a therapeutically effective amount of: (a) a first agent that is an FXR modulator; and (b) a second agent that is an SGLT2 inhibitor.

Disclosed herein, are methods of treating a metabolic disorder in an individual in need thereof, comprising co-administering to the individual a therapeutically effective amount of: (a) a first agent that is an FXR modulator; and (b) a second agent that is an acetyl-CoA carboxylase inhibitor.

Disclosed herein, are methods of treating a metabolic disorder in an individual in need thereof, comprising co-administering to the individual a therapeutically effective amount of: (a) a first agent that is an FXR modulator; and (b) a second agent that is a diacylglycerol acyltransferase-1 inhibitor.

Disclosed herein are methods of treating a metabolic disorder in an individual in need thereof, comprising co-administering to the individual a therapeutically effective amount of: (a) a first agent that is an FXR modulator; and (b) a second agent that is a CCR2/CCR5 antagonist selected from the group consisting of cenicriviroc (CVC), aplaviroc, vicriviroc 5-({4-[(3S)-4-{2-methoxy-1-[4-(trifluoromethyl)phenyl]ethyl}-3-methylpiperazin-1-yl]-4-methylpiperidin-1-yl}carbonyl)-4,6-dimethylpyrimidine), maraviroc (e.g., 4,4′-difluorocyclohexylamide), and cochilioquinone A.

Disclosed herein, are methods of treating a metabolic disorder in an individual in need thereof, comprising co-administering to the individual a therapeutically effective amount of: (a) a first agent that is an FXR modulator; and (b) a second agent that is a GLP-1 agonist.

In some embodiments, two second agents are co-administered with the first agent that is an FXR modulator. In some embodiments, the two second agents co-administered with a first agent that is an FXR modulator are a DPP-IV inhibitor and an SGLT2 inhibitor. In some embodiments, the two second agents co-administered with a first agent that is an FXR modulator are a DPP-IV inhibitor and an ASK1 inhibitor. In some embodiments, the two second agents co-administered with a first agent that is an FXR modulator are a DPP-IV inhibitor and a GLP-1 agonist. In some embodiments, the two second agents co-administered with a first agent that is an FXR modulator are an SGLT2 inhibitor and an ASK1 inhibitor. In some embodiments, the two second agents co-administered with a first agent that is an FXR modulator are an SGLT2 inhibitor and a GLP-1 agonist. In some embodiments, the two second agents co-administered with a first agent that is an FXR modulator are an ASK1 inhibitor and a GLP-1 agonist.

In some embodiments, is a method of treating a disease, disorder or condition in a mammal that would benefit from combined FXR modulation and DPP-IV inhibition comprising administering compounds disclosed herein, wherein the disease, disorder or condition in a mammal is selected from nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, diabetic nephropathy and nephrotic syndrome. In some embodiments, is a method of treating a disease, disorder or condition in a mammal that would benefit from combined FXR modulation and DPP-IV inhibition comprising administering compounds disclosed herein, wherein the disease, disorder or condition in a mammal is nonalcoholic steatohepatitis (NASH). In some embodiments, is a method of (a) modulating FXR activity comprising contacting FXR, or portion thereof, with an FXR modulator disclosed herein and (b) inhibiting DPP-IV activity comprising contacting DPP-IV, or a portion thereof, with a DPP-IV inhibitor disclosed herein. In some embodiments, the compound of FXR modulator is an FXR agonist. In some embodiments, the FXR modulator is an FXR partial agonist. In some embodiments, the FXR modulator is an FXR antagonist. In some embodiments, the disease, disorder or condition in a mammal that would benefit from (a) FXR modulation and (b) DPP-IV inhibition is selected from nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, diabetic nephropathy and nephrotic syndrome.

In some embodiments, is a method of treating a disease, disorder or condition in a mammal that would benefit from combined FXR modulation and SGLT2 inhibition comprising administering compounds disclosed herein, wherein the disease, disorder or condition in a mammal is selected from nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, diabetic nephropathy and nephrotic syndrome. In some embodiments, is a method of treating a disease, disorder or condition in a mammal that would benefit from combined FXR modulation and SGLT2 inhibition comprising administering compounds disclosed herein, wherein the disease, disorder or condition in a mammal is nonalcoholic steatohepatitis (NASH). In some embodiments, is a method of (a) modulating FXR activity comprising contacting FXR, or portion thereof, with an FXR modulator disclosed herein and (b) inhibiting SGLT2 activity comprising contacting SGLT2, or a portion thereof, with an SGLT2 inhibitor disclosed herein. In some embodiments, the compound of FXR modulator is an FXR agonist. In some embodiments, the FXR modulator is an FXR partial agonist. In some embodiments, the FXR modulator is an FXR antagonist. In some embodiments, the disease, disorder or condition in a mammal that would benefit from (a) FXR modulation and (b) SGLT2 inhibition is selected from nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, diabetic nephropathy and nephrotic syndrome.

In some embodiments, is a method of treating a disease, disorder or condition in a mammal that would benefit from combined FXR modulation and ASK1 inhibition comprising administering compounds disclosed herein, wherein the disease, disorder or condition in a mammal is selected from nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, diabetic nephropathy and nephrotic syndrome. In some embodiments, is a method of treating a disease, disorder or condition in a mammal that would benefit from combined FXR modulation and ASK1 inhibition comprising administering compounds disclosed herein, wherein the disease, disorder or condition in a mammal is nonalcoholic steatohepatitis (NASH). In some embodiments, is a method of (a) modulating FXR activity comprising contacting FXR, or portion thereof, with an FXR modulator disclosed herein and (b) inhibiting ASK1 activity comprising contacting ASK1, or a portion thereof, with a ASK1 inhibitor disclosed herein. In some embodiments, the compound of FXR modulator is an FXR agonist. In some embodiments, the FXR modulator is an FXR partial agonist. In some embodiments, the FXR modulator is an FXR antagonist. In some embodiments, the disease, disorder or condition in a mammal that would benefit from (a) FXR modulation and (b) ASK1 inhibition is selected from nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, diabetic nephropathy and nephrotic syndrome.

In some embodiments, is a method of treating a disease, disorder or condition in a mammal that would benefit from combined FXR modulation and GLP-1 agonism comprising administering compounds disclosed herein, wherein the disease, disorder or condition in a mammal is selected from nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, diabetic nephropathy and nephrotic syndrome. In some embodiments, is a method of treating a disease, disorder or condition in a mammal that would benefit from combined FXR modulation and GLP-1 agonism comprising administering compounds disclosed herein, wherein the disease, disorder or condition in a mammal is nonalcoholic steatohepatitis (NASH). In some embodiments, is a method of (a) modulating FXR activity comprising contacting FXR, or portion thereof, with an FXR modulator disclosed herein and (b) inhibiting GLP-1 activity comprising contacting GLP-1, or a portion thereof, with a GLP-1 agonist disclosed herein. In some embodiments, the compound of FXR modulator is an FXR agonist. In some embodiments, the FXR modulator is an FXR partial agonist. In some embodiments, the FXR modulator is an FXR antagonist. In some embodiments, the disease, disorder or condition in a mammal that would benefit from (a) FXR modulation and (b) GLP-1 agonism is selected from nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, diabetic nephropathy and nephrotic syndrome.

Combination with DPP-IV Inhibitors:

Disclosed herein are methods of treating a metabolic disorder in an individual in need thereof, comprising co-administering to the individual a therapeutically effective amount of: (a) a first agent that is an FXR modulator; and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, and omarigliptin.

In some embodiments, is a method of treating a metabolic disorder in an individual in need thereof, comprising co-administering to the individual a therapeutically effective amount of: (a) a first agent that is an FXR modulator of Formula (I) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (I) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (I) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (I) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (I) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (I) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (I) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (I) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (I) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (I) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (I) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (I) and (b) a second agent that is omarigliptin.

In some embodiments, is a method of treating a metabolic disorder in an individual in need thereof, comprising co-administering to the individual a therapeutically effective amount of: (a) a first agent that is an FXR modulator of Formula (II) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (II) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (II) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (II) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (II) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (II) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (II) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (II) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (II) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (II) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (II) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (II) and (b) a second agent that is omarigliptin.

In some embodiments, is a method of treating a metabolic disorder in an individual in need thereof, comprising co-administering to the individual a therapeutically effective amount of: (a) a first agent that is an FXR modulator of Formula (III) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (III) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (III) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (III) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (III) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (III) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (III) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (III) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (III) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (III) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (III) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (III) and (b) a second agent that is omarigliptin.

In some embodiments, is a method of treating a metabolic disorder in an individual in need thereof, comprising co-administering to the individual a therapeutically effective amount of: (a) a first agent that is an FXR modulator of Formula (IV) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is omarigliptin.

In some embodiments, is a method of treating a metabolic disorder in an individual in need thereof, comprising co-administering to the individual a therapeutically effective amount of: (a) a first agent that is an FXR modulator of Formula (V) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (V) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (V) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (V) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (V) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (V) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (V) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (V) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (V) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (V) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (V) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (V) and (b) a second agent that is omarigliptin.

In some embodiments, is a method of treating a metabolic disorder in an individual in need thereof, comprising co-administering to the individual a therapeutically effective amount of: (a) a first agent that is an FXR modulator of Formula (VI) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is omarigliptin.

In some embodiments, is a method of treating a metabolic disorder in an individual in need thereof, comprising co-administering to the individual a therapeutically effective amount of: (a) a first agent that is an FXR modulator of Formula (VII) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is omarigliptin.

In some embodiments, is a method of treating a metabolic disorder in an individual in need thereof, comprising co-administering to the individual a therapeutically effective amount of: (a) a first agent that is an FXR modulator of Formula (VIII) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is omarigliptin.

In some embodiments, is a method of treating a metabolic disorder in an individual in need thereof, comprising co-administering to the individual a therapeutically effective amount of: (a) a first agent that is an FXR modulator of Formula (IX) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is omarigliptin.

In some embodiments, is a method of treating a metabolic disorder in an individual in need thereof, comprising co-administering to the individual a therapeutically effective amount of: (a) a first agent that is an FXR modulator of Formula (X) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (X) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (X) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (X) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (X) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (X) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (X) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (X) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (X) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (X) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (X) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (X) and (b) a second agent that is omarigliptin.

In some embodiments, is a method of treating a metabolic disorder in an individual in need thereof, comprising co-administering to the individual a therapeutically effective amount of: (a) a first agent that is an FXR modulator of Formula (XI) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is omarigliptin.

In some embodiments, is a method of treating a metabolic disorder in an individual in need thereof, comprising co-administering to the individual a therapeutically effective amount of: (a) a first agent that is an FXR modulator of Formula (XII) and (b) a second agent that is a DPP-IV inhibitor selected from sitagliptin, saxagliptin, linagliptin, alogliptin, vildagliptin, gemigliptin, anagliptin, teneligliptin, trelagliptin, dutogliptin, or omarigliptin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is sitagliptin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is saxagliptin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is linagliptin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is alogliptin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is vildagliptin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is gemigliptin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is anagliptin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is teneligliptin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is trelagliptin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is dutogliptin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is omarigliptin.

Combination with SGLT2 Inhibitors:

Disclosed herein are methods of treating a metabolic disorder in an individual in need thereof, comprising co-administering to the individual a therapeutically effective amount of: (a) a first agent that is an FXR modulator; and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin.

In some embodiments, is a method of treating a metabolic disorder in an individual in need thereof, comprising co-administering to the individual a therapeutically effective amount of: (a) a first agent that is an FXR modulator of Formula (I) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (I) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (I) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (I) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (I) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (I) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (I) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (I) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (I) and (b) a second agent that is ertugliflozin.

In some embodiments, is a method of treating a metabolic disorder in an individual in need thereof, comprising co-administering to the individual a therapeutically effective amount of: (a) a first agent that is an FXR modulator of Formula (II) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (II) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (II) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (II) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (II) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (II) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (II) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (II) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (II) and (b) a second agent that is ertugliflozin.

In some embodiments, is a method of treating a metabolic disorder in an individual in need thereof, comprising co-administering to the individual a therapeutically effective amount of: (a) a first agent that is an FXR modulator of Formula (III) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (III) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (III) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (III) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (III) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (III) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (III) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (III) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (III) and (b) a second agent that is ertugliflozin.

In some embodiments, is a method of treating a metabolic disorder in an individual in need thereof, comprising co-administering to the individual a therapeutically effective amount of: (a) a first agent that is an FXR modulator of Formula (IV) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (IV) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (IV) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (IV) and (b) a second agent that is ertugliflozin.

In some embodiments, is a method of treating a metabolic disorder in an individual in need thereof, comprising co-administering to the individual a therapeutically effective amount of: (a) a first agent that is an FXR modulator of Formula (V) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (V) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (V) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (V) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (V) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (V) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (V) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (V) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (V) and (b) a second agent that is ertugliflozin.

In some embodiments, is a method of treating a metabolic disorder in an individual in need thereof, comprising co-administering to the individual a therapeutically effective amount of: (a) a first agent that is an FXR modulator of Formula (VI) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (VI) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (VI) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (VI) and (b) a second agent that is ertugliflozin.

In some embodiments, is a method of treating a metabolic disorder in an individual in need thereof, comprising co-administering to the individual a therapeutically effective amount of: (a) a first agent that is an FXR modulator of Formula (VII) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (VII) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (VII) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (VII) and (b) a second agent that is ertugliflozin.

In some embodiments, is a method of treating a metabolic disorder in an individual in need thereof, comprising co-administering to the individual a therapeutically effective amount of: (a) a first agent that is an FXR modulator of Formula (VIII) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is ertugliflozin.

In some embodiments, is a method of treating a metabolic disorder in an individual in need thereof, comprising co-administering to the individual a therapeutically effective amount of: (a) a first agent that is an FXR modulator of Formula (IX) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (IX) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (IX) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (IX) and (b) a second agent that is ertugliflozin.

In some embodiments, is a method of treating a metabolic disorder in an individual in need thereof, comprising co-administering to the individual a therapeutically effective amount of: (a) a first agent that is an FXR modulator of Formula (X) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (X) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (X) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (X) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (X) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (X) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (X) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (X) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (X) and (b) a second agent that is ertugliflozin.

In some embodiments, is a method of treating a metabolic disorder in an individual in need thereof, comprising co-administering to the individual a therapeutically effective amount of: (a) a first agent that is an FXR modulator of Formula (XI) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (XI) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (XI) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (XI) and (b) a second agent that is ertugliflozin.

In some embodiments, is a method of treating a metabolic disorder in an individual in need thereof, comprising co-administering to the individual a therapeutically effective amount of: (a) a first agent that is an FXR modulator of Formula (XII) and (b) a second agent that is an SGLT2 inhibitor selected from canagliflozin, empagliflozin, dapagliflozin, ipragliflozin, tofogliflozin, sergliflozin etabonate, remogliflozin etabonate, or ertugliflozin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is canagliflozin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is empagliflozin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is dapagliflozin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is ipragliflozin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is tofogliflozin. In some embodiments, is a method of treating a metabolic disorder in an individual in need thereof, comprising co-administering to the individual a therapeutically effective amount of: (a) a first agent that is an FXR modulator of Formula (XII) and (b) a second agent that is sergliflozin etabonate. In some embodiments, is a method of treating a metabolic disorder in an individual in need thereof, comprising co-administering to the individual a therapeutically effective amount of: (a) a first agent that is an FXR modulator of Formula (XII) and (b) a second agent that is remogliflozin etabonate. In some embodiments, is a method of treating a metabolic disorder in an individual in need thereof, comprising co-administering to the individual a therapeutically effective amount of: (a) a first agent that is an FXR modulator of Formula (XII) and (b) a second agent that is ertugliflozin.

Combination with ASK1 Inhibitors:

Disclosed herein are methods of treating a metabolic disorder in an individual in need thereof, comprising co-administering to the individual a therapeutically effective amount of: (a) a first agent that is an FXR modulator; and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride).

In some embodiments, is a method of treating a metabolic disorder in an individual in need thereof, comprising co-administering to the individual a therapeutically effective amount of: (a) a first agent that is an FXR modulator of Formula (I) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (I) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (I) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (I) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (I) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (I) and (b) a second agent that is TC ASK 10.

In some embodiments, is a method of treating a metabolic disorder in an individual in need thereof, comprising co-administering to the individual a therapeutically effective amount of: (a) a first agent that is an FXR modulator of Formula (II) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (II) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (II) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (II) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (II) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (II) and (b) a second agent that is TC ASK 10.

In some embodiments, is a method of treating a metabolic disorder in an individual in need thereof, comprising co-administering to the individual a therapeutically effective amount of: (a) a first agent that is an FXR modulator of Formula (III) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (III) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (III) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (III) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (III) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (III) and (b) a second agent that is TC ASK 10.

In some embodiments, is a method of treating a metabolic disorder in an individual in need thereof, comprising co-administering to the individual a therapeutically effective amount of: (a) a first agent that is an FXR modulator of Formula (IV) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (IV) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (IV) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (IV) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (IV) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (IV) and (b) a second agent that is TC ASK 10.

In some embodiments, is a method of treating a metabolic disorder in an individual in need thereof, comprising co-administering to the individual a therapeutically effective amount of: (a) a first agent that is an FXR modulator of Formula (V) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (V) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (V) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (V) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (V) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (V) and (b) a second agent that is TC ASK 10.

In some embodiments, is a method of treating a metabolic disorder in an individual in need thereof, comprising co-administering to the individual a therapeutically effective amount of: (a) a first agent that is an FXR modulator of Formula (VI) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (VI) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (VI) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (VI) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (VI) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (VI) and (b) a second agent that is TC ASK 10.

In some embodiments, is a method of treating a metabolic disorder in an individual in need thereof, comprising co-administering to the individual a therapeutically effective amount of: (a) a first agent that is an FXR modulator of Formula (VII) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (VII) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (VII) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (VII) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (VII) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (VII) and (b) a second agent that is TC ASK 10.

In some embodiments, is a method of treating a metabolic disorder in an individual in need thereof, comprising co-administering to the individual a therapeutically effective amount of: (a) a first agent that is an FXR modulator of Formula (VIII) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (VIII) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is TC ASK 10.

In some embodiments, is a method of treating a metabolic disorder in an individual in need thereof, comprising co-administering to the individual a therapeutically effective amount of: (a) a first agent that is an FXR modulator of Formula (IX) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (IX) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (IX) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (IX) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (IX) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (IX) and (b) a second agent that is TC ASK 10.

In some embodiments, is a method of treating a metabolic disorder in an individual in need thereof, comprising co-administering to the individual a therapeutically effective amount of: (a) a first agent that is an FXR modulator of Formula (X) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (X) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (X) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (X) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (X) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (X) and (b) a second agent that is TC ASK 10.

In some embodiments, is a method of treating a metabolic disorder in an individual in need thereof, comprising co-administering to the individual a therapeutically effective amount of: (a) a first agent that is an FXR modulator of Formula (XI) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (XI) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (XI) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (XI) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (XI) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (XI) and (b) a second agent that is TC ASK 10.

In some embodiments, is a method of treating a metabolic disorder in an individual in need thereof, comprising co-administering to the individual a therapeutically effective amount of: (a) a first agent that is an FXR modulator of Formula (XII) and (b) a second agent that is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyridinecarboxamide), and TC ASK 10 (4-(1,1-dimethylethyl)-N-[6-(1H-imidazol-1-yl)imidazo[1,2-a]pyridin-2-yl]benzamide dihydrochloride). In some embodiments, is a method of treating a metabolic disorder in an individual in need thereof, comprising co-administering to the individual a therapeutically effective amount of: (a) a first agent that is an FXR modulator of Formula (XII) and (b) a second agent that is selonsertib. In some embodiments, is a method of treating a metabolic disorder in an individual in need thereof, comprising co-administering to the individual a therapeutically effective amount of: (a) a first agent that is an FXR modulator of Formula (XII) and (b) a second agent that is NQDI-1. In some embodiments, is a method of treating a metabolic disorder in an individual in need thereof, comprising co-administering to the individual a therapeutically effective amount of: (a) a first agent that is an FXR modulator of Formula (XII) and (b) a second agent that is ML365. In some embodiments, is a method of treating a metabolic disorder in an individual in need thereof, comprising co-administering to the individual a therapeutically effective amount of: (a) a first agent that is an FXR modulator of Formula (XII) and (b) a second agent that is MSC 2032964A. In some embodiments, is a method of treating a metabolic disorder in an individual in need thereof, comprising co-administering to the individual a therapeutically effective amount of: (a) a first agent that is an FXR modulator of Formula (XII) and (b) a second agent that is TC ASK 10.

Disclosed herein are methods of treating a metabolic disorder in an individual in need thereof, comprising co-administering to the individual a therapeutically effective amount of: (a) a first agent that is an FXR modulator; and (b) a second agent that is an ASK1 inhibitor selected from a compound disclosed in any of the following publications: WO2008/016131; EP2058309, US2010/0029619, WO2016/49069, U S2011/0009410, US 2013/0197037, US 2013/0197037 US 2014/0179663, and US2014/0018370.

Disclosed herein are methods of treating a metabolic disorder in an individual in need thereof, comprising co-administering to the individual a therapeutically effective amount of: (a) a first agent that is an FXR modulator of Formula (I), (II), (III), (IV), (V), (VI), (VII), (VIII), (IX), (X), (XI), or (XII); and (b) a second agent that is an ASK1 inhibitor selected from a compound disclosed in any of the following publications: WO2008/016131; EP2058309, US2010/0029619, WO2016/49069, US2011/0009410, US2013/0197037, US2013/0197037, US2014/0179663, and US2014/0018370.

Combination with GLP-1 Agonists:

Disclosed herein are methods of treating a metabolic disorder in an individual in need thereof, comprising co-administering to the individual a therapeutically effective amount of: (a) a first agent that is an FXR modulator; and (b) a second agent that is a GLP-1 agonist selected from exenatide, liraglutide, lixisenatide, albiglutide, dulaglutide, taspoglutide, semaglutide.

In some embodiments, is a method of treating a metabolic disorder in an individual in need thereof, comprising co-administering to the individual a therapeutically effective amount of: (a) a first agent that is an FXR modulator of Formula (I) and (b) a second agent that is a GLP-1 agonist selected from exenatide, liraglutide, lixisenatide, albiglutide, dulaglutide, taspoglutide, semaglutide. In some embodiments, is a method of treating a metabolic disorder in an individual in need thereof, comprising co-administering to the individual a therapeutically effective amount of: (a) a first agent that is an FXR modulator of Formula (I) and (b) a second agent that is exenatide. In some embodiments, is a method of treating a metabolic disorder in an individual in need thereof, comprising co-administering to the individual a therapeutically effective amount of: (a) a first agent that is an FXR modulator of Formula (I) and (b) a second agent that is liraglutide. In some embodiments, is a method of treating a metabolic disorder in an individual in need thereof, comprising co-administering to the individual a therapeutically effective amount of: (a) a first agent that is an FXR modulator of Formula (I) and (b) a second agent that is lixisenatide. In some embodiments, is a method of treating a metabolic disorder in an individual in need thereof, comprising co-administering to the individual a therapeutically effective amount of: (a) a first agent that is an FXR modulator of Formula (I) and (b) a second agent that is albiglutide. In some embodiments, is a method of treating a metabolic disorder in an individual in need thereof, comprising co-administering to the individual a therapeutically effective amount of: (a) a first agent that is an FXR modulator of Formula (I) and (b) a second agent that is dulaglutide. In some embodiments, is a method of treating a metabolic disorder in an individual in need thereof, comprising co-administering to the individual a therapeutically effective amount of: (a) a first agent that is an FXR modulator of Formula (I) and (b) a second agent that is taspoglutide. In some embodiments, is a method of treating a metabolic disorder in an individual in need thereof, comprising co-administering to the individual a therapeutically effective amount of: (a) a first agent that is an FXR modulator of Formula (I) and (b) a second agent that is semaglutide.

In some embodiments, is a method of treating a metabolic disorder in an individual in need thereof, comprising co-administering to the individual a therapeutically effective amount of: (a) a first agent that is an FXR modulator of Formula (II) and (b) a second agent that is a GLP-1 agonist selected from exenatide, liraglutide, lixisenatide, albiglutide, dulaglutide, taspoglutide, semaglutide. In some embodiments, is a method of treating a metabolic disorder in an individual in need thereof, comprising co-administering to the individual a therapeutically effective amount of: (a) a first agent that is an FXR modulator of Formula (II) and (b) a second agent that is exenatide. In some embodiments, is a method of treating a metabolic disorder in an individual in need thereof, comprising co-administering to the individual a therapeutically effective amount of: (a) a first agent that is an FXR modulator of Formula (II) and (b) a second agent that is liraglutide. In some embodiments, is a method of treating a metabolic disorder in an individual in need thereof, comprising co-administering to the individual a therapeutically effective amount of: (a) a first agent that is an FXR modulator of Formula (II) and (b) a second agent that is lixisenatide. In some embodiments, is a method of treating a metabolic disorder in an individual in need thereof, comprising co-administering to the individual a therapeutically effective amount of: (a) a first agent that is an FXR modulator of Formula (II) and (b) a second agent that is albiglutide. In some embodiments, is a method of treating a metabolic disorder in an individual in need thereof, comprising co-administering to the individual a therapeutically effective amount of: (a) a first agent that is an FXR modulator of Formula (II) and (b) a second agent that is dulaglutide. In some embodiments, is a method of treating a metabolic disorder in an individual in need thereof, comprising co-administering to the individual a therapeutically effective amount of: (a) a first agent that is an FXR modulator of Formula (II) and (b) a second agent that is taspoglutide. In some embodiments, is a method of treating a metabolic disorder in an individual in need thereof, comprising co-administering to the individual a therapeutically effective amount of: (a) a first agent that is an FXR modulator of Formula (II) and (b) a second agent that is semaglutide.

In some embodiments, is a method of treating a metabolic disorder in an individual in need thereof, comprising co-administering to the individual a therapeutically effective amount of: (a) a first agent that is an FXR modulator of Formula (III) and (b) a second agent that is a GLP-1 agonist selected from exenatide, liraglutide, lixisenatide, albiglutide, dulaglutide, taspoglutide, semaglutide. In some embodiments, is a method of treating a metabolic disorder in an individual in need thereof, comprising co-administering to the individual a therapeutically effective amount of: (a) a first agent that is an FXR modulator of Formula (III) and (b) a second agent that is exenatide. In some embodiments, is a method of treating a metabolic disorder in an individual in need thereof, comprising co-administering to the individual a therapeutically effective amount of: (a) a first agent that is an FXR modulator of Formula (III) and (b) a second agent that is liraglutide. In some embodiments, is a method of treating a metabolic disorder in an individual in need thereof, comprising co-administering to the individual a therapeutically effective amount of: (a) a first agent that is an FXR modulator of Formula (III) and (b) a second agent that is lixisenatide. In some embodiments, is a method of treating a metabolic disorder in an individual in need thereof, comprising co-administering to the individual a therapeutically effective amount of: (a) a first agent that is an FXR modulator of Formula (III) and (b) a second agent that is albiglutide. In some embodiments, is a method of treating a metabolic disorder in an individual in need thereof, comprising co-administering to the individual a therapeutically effective amount of: (a) a first agent that is an FXR modulator of Formula (III) and (b) a second agent that is dulaglutide. In some embodiments, is a method of treating a metabolic disorder in an individual in need thereof, comprising co-administering to the individual a therapeutically effective amount of: (a) a first agent that is an FXR modulator of Formula (III) and (b) a second agent that is taspoglutide. In some embodiments, is a method of treating a metabolic disorder in an individual in need thereof, comprising co-administering to the individual a therapeutically effective amount of: (a) a first agent that is an FXR modulator of Formula (III) and (b) a second agent that is semaglutide.

In some embodiments, is a method of treating a metabolic disorder in an individual in need thereof, comprising co-administering to the individual a therapeutically effective amount of: (a) a first agent that is an FXR modulator of Formula (IV) and (b) a second agent that is a GLP-1 agonist selected from exenatide, liraglutide, lixisenatide, albiglutide, dulaglutide, taspoglutide, semaglutide. In some embodiments, is a method of treating a metabolic disorder in an individual in need thereof, comprising co-administering to the individual a therapeutically effective amount of: (a) a first agent that is an FXR modulator of Formula (IV) and (b) a second agent that is exenatide. In some embodiments, is a method of treating a metabolic disorder in an individual in need thereof, comprising co-administering to the individual a therapeutically effective amount of: (a) a first agent that is an FXR modulator of Formula (IV) and (b) a second agent that is liraglutide. In some embodiments, is a method of treating a metabolic disorder in an individual in need thereof, comprising co-administering to the individual a therapeutically effective amount of: (a) a first agent that is an FXR modulator of Formula (IV) and (b) a second agent that is lixisenatide. In some embodiments, is a method of treating a metabolic disorder in an individual in need thereof, comprising co-administering to the individual a therapeutically effective amount of: (a) a first agent that is an FXR modulator of Formula (IV) and (b) a second agent that is albiglutide. In some embodiments, is a method of treating a metabolic disorder in an individual in need thereof, comprising co-administering to the individual a therapeutically effective amount of: (a) a first agent that is an FXR modulator of Formula (IV) and (b) a second agent that is dulaglutide. In some embodiments, is a method of treating a metabolic disorder in an individual in need thereof, comprising co-administering to the individual a therapeutically effective amount of: (a) a first agent that is an FXR modulator of Formula (IV) and (b) a second agent that is taspoglutide. In some embodiments, is a method of treating a metabolic disorder in an individual in need thereof, comprising co-administering to the individual a therapeutically effective amount of: (a) a first agent that is an FXR modulator of Formula (IV) and (b) a second agent that is semaglutide.

In some embodiments, is a method of treating a metabolic disorder in an individual in need thereof, comprising co-administering to the individual a therapeutically effective amount of: (a) a first agent that is an FXR modulator of Formula (V) and (b) a second agent that is a GLP-1 agonist selected from exenatide, liraglutide, lixisenatide, albiglutide, dulaglutide, taspoglutide, semaglutide. In some embodiments, is a method of treating a metabolic disorder in an individual in need thereof, comprising co-administering to the individual a therapeutically effective amount of: (a) a first agent that is an FXR modulator of Formula (V) and (b) a second agent that is exenatide. In some embodiments, is a method of treating a metabolic disorder in an individual in need thereof, comprising co-administering to the individual a therapeutically effective amount of: (a) a first agent that is an FXR modulator of Formula (V) and (b) a second agent that is liraglutide. In some embodiments, is a method of treating a metabolic disorder in an individual in need thereof, comprising co-administering to the individual a therapeutically effective amount of: (a) a first agent that is an FXR modulator of Formula (V) and (b) a second agent that is lixisenatide. In some embodiments, is a method of treating a metabolic disorder in an individual in need thereof, comprising co-administering to the individual a therapeutically effective amount of: (a) a first agent that is an FXR modulator of Formula (V) and (b) a second agent that is albiglutide. In some embodiments, is a method of treating a metabolic disorder in an individual in need thereof, comprising co-administering to the individual a therapeutically effective amount of: (a) a first agent that is an FXR modulator of Formula (V) and (b) a second agent that is dulaglutide. In some embodiments, is a method of treating a metabolic disorder in an individual in need thereof, comprising co-administering to the individual a therapeutically effective amount of: (a) a first agent that is an FXR modulator of Formula (V) and (b) a second agent that is taspoglutide. In some embodiments, is a method of treating a metabolic disorder in an individual in need thereof, comprising co-administering to the individual a therapeutically effective amount of: (a) a first agent that is an FXR modulator of Formula (V) and (b) a second agent that is semaglutide.

In some embodiments, is a method of treating a metabolic disorder in an individual in need thereof, comprising co-administering to the individual a therapeutically effective amount of: (a) a first agent that is an FXR modulator of Formula (VI) and (b) a second agent that is a GLP-1 agonist selected from exenatide, liraglutide, lixisenatide, albiglutide, dulaglutide, taspoglutide, semaglutide. In some embodiments, is a method of treating a metabolic disorder in an individual in need thereof, comprising co-administering to the individual a therapeutically effective amount of: (a) a first agent that is an FXR modulator of Formula (VI) and (b) a second agent that is exenatide. In some embodiments, is a method of treating a metabolic disorder in an individual in need thereof, comprising co-administering to the individual a therapeutically effective amount of: (a) a first agent that is an FXR modulator of Formula (VI) and (b) a second agent that is liraglutide. In some embodiments, is a method of treating a metabolic disorder in an individual in need thereof, comprising co-administering to the individual a therapeutically effective amount of: (a) a first agent that is an FXR modulator of Formula (VI) and (b) a second agent that is lixisenatide. In some embodiments, is a method of treating a metabolic disorder in an individual in need thereof, comprising co-administering to the individual a therapeutically effective amount of: (a) a first agent that is an FXR modulator of Formula (VI) and (b) a second agent that is albiglutide. In some embodiments, is a method of treating a metabolic disorder in an individual in need thereof, comprising co-administering to the individual a therapeutically effective amount of: (a) a first agent that is an FXR modulator of Formula (VI) and (b) a second agent that is dulaglutide. In some embodiments, is a method of treating a metabolic disorder in an individual in need thereof, comprising co-administering to the individual a therapeutically effective amount of: (a) a first agent that is an FXR modulator of Formula (VI) and (b) a second agent that is taspoglutide. In some embodiments, is a method of treating a metabolic disorder in an individual in need thereof, comprising co-administering to the individual a therapeutically effective amount of: (a) a first agent that is an FXR modulator of Formula (VI) and (b) a second agent that is semaglutide.

In some embodiments, is a method of treating a metabolic disorder in an individual in need thereof, comprising co-administering to the individual a therapeutically effective amount of: (a) a first agent that is an FXR modulator of Formula (VII) and (b) a second agent that is a GLP-1 agonist selected from exenatide, liraglutide, lixisenatide, albiglutide, dulaglutide, taspoglutide, semaglutide. In some embodiments, is a method of treating a metabolic disorder in an individual in need thereof, comprising co-administering to the individual a therapeutically effective amount of: (a) a first agent that is an FXR modulator of Formula (VII) and (b) a second agent that is exenatide. In some embodiments, is a method of treating a metabolic disorder in an individual in need thereof, comprising co-administering to the individual a therapeutically effective amount of: (a) a first agent that is an FXR modulator of Formula (VII) and (b) a second agent that is liraglutide. In some embodiments, is a method of treating a metabolic disorder in an individual in need thereof, comprising co-administering to the individual a therapeutically effective amount of: (a) a first agent that is an FXR modulator of Formula (VII) and (b) a second agent that is lixisenatide. In some embodiments, is a method of treating a metabolic disorder in an individual in need thereof, comprising co-administering to the individual a therapeutically effective amount of: (a) a first agent that is an FXR modulator of Formula (VII) and (b) a second agent that is albiglutide. In some embodiments, is a method of treating a metabolic disorder in an individual in need thereof, comprising co-administering to the individual a therapeutically effective amount of: (a) a first agent that is an FXR modulator of Formula (VII) and (b) a second agent that is dulaglutide. In some embodiments, is a method of treating a metabolic disorder in an individual in need thereof, comprising co-administering to the individual a therapeutically effective amount of: (a) a first agent that is an FXR modulator of Formula (VII) and (b) a second agent that is taspoglutide. In some embodiments, is a method of treating a metabolic disorder in an individual in need thereof, comprising co-administering to the individual a therapeutically effective amount of: (a) a first agent that is an FXR modulator of Formula (VII) and (b) a second agent that is semaglutide.

In some embodiments, is a method of treating a metabolic disorder in an individual in need thereof, comprising co-administering to the individual a therapeutically effective amount of: (a) a first agent that is an FXR modulator of Formula (VIII) and (b) a second agent that is a GLP-1 agonist selected from exenatide, liraglutide, lixisenatide, albiglutide, dulaglutide, taspoglutide, semaglutide. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is exenatide. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is liraglutide. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is lixisenatide. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is albiglutide. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is dulaglutide. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is taspoglutide. In some embodiments, is a method of treating a metabolic disorder in an individual in need thereof, comprising co-administering to the individual a therapeutically effective amount of: (a) a first agent that is an FXR modulator of Formula (VIII) and (b) a second agent that is semaglutide.

In some embodiments, is a method of treating a metabolic disorder in an individual in need thereof, comprising co-administering to the individual a therapeutically effective amount of: (a) a first agent that is an FXR modulator of Formula (IX) and (b) a second agent that is a GLP-1 agonist selected from exenatide, liraglutide, lixisenatide, albiglutide, dulaglutide, taspoglutide, semaglutide. In some embodiments, is a method of treating a metabolic disorder in an individual in need thereof, comprising co-administering to the individual a therapeutically effective amount of: (a) a first agent that is an FXR modulator of Formula (IX) and (b) a second agent that is exenatide. In some embodiments, is a method of treating a metabolic disorder in an individual in need thereof, comprising co-administering to the individual a therapeutically effective amount of: (a) a first agent that is an FXR modulator of Formula (IX) and (b) a second agent that is liraglutide. In some embodiments, is a method of treating a metabolic disorder in an individual in need thereof, comprising co-administering to the individual a therapeutically effective amount of: (a) a first agent that is an FXR modulator of Formula (IX) and (b) a second agent that is lixisenatide. In some embodiments, is a method of treating a metabolic disorder in an individual in need thereof, comprising co-administering to the individual a therapeutically effective amount of: (a) a first agent that is an FXR modulator of Formula (IX) and (b) a second agent that is albiglutide. In some embodiments, is a method of treating a metabolic disorder in an individual in need thereof, comprising co-administering to the individual a therapeutically effective amount of: (a) a first agent that is an FXR modulator of Formula (IX) and (b) a second agent that is dulaglutide. In some embodiments, is a method of treating a metabolic disorder in an individual in need thereof, comprising co-administering to the individual a therapeutically effective amount of: (a) a first agent that is an FXR modulator of Formula (IX) and (b) a second agent that is taspoglutide. In some embodiments, is a method of treating a metabolic disorder in an individual in need thereof, comprising co-administering to the individual a therapeutically effective amount of: (a) a first agent that is an FXR modulator of Formula (IX) and (b) a second agent that is semaglutide.

In some embodiments, is a method of treating a metabolic disorder in an individual in need thereof, comprising co-administering to the individual a therapeutically effective amount of: (a) a first agent that is an FXR modulator of Formula (X) and (b) a second agent that is a GLP-1 agonist selected from exenatide, liraglutide, lixisenatide, albiglutide, dulaglutide, taspoglutide, semaglutide. In some embodiments, is a method of treating a metabolic disorder in an individual in need thereof, comprising co-administering to the individual a therapeutically effective amount of: (a) a first agent that is an FXR modulator of Formula (X) and (b) a second agent that is exenatide. In some embodiments, is a method of treating a metabolic disorder in an individual in need thereof, comprising co-administering to the individual a therapeutically effective amount of: (a) a first agent that is an FXR modulator of Formula (X) and (b) a second agent that is liraglutide. In some embodiments, is a method of treating a metabolic disorder in an individual in need thereof, comprising co-administering to the individual a therapeutically effective amount of: (a) a first agent that is an FXR modulator of Formula (X) and (b) a second agent that is lixisenatide. In some embodiments, is a method of treating a metabolic disorder in an individual in need thereof, comprising co-administering to the individual a therapeutically effective amount of: (a) a first agent that is an FXR modulator of Formula (X) and (b) a second agent that is albiglutide. In some embodiments, is a method of treating a metabolic disorder in an individual in need thereof, comprising co-administering to the individual a therapeutically effective amount of: (a) a first agent that is an FXR modulator of Formula (X) and (b) a second agent that is dulaglutide. In some embodiments, is a method of treating a metabolic disorder in an individual in need thereof, comprising co-administering to the individual a therapeutically effective amount of: (a) a first agent that is an FXR modulator of Formula (X) and (b) a second agent that is taspoglutide. In some embodiments, is a method of treating a metabolic disorder in an individual in need thereof, comprising co-administering to the individual a therapeutically effective amount of: (a) a first agent that is an FXR modulator of Formula (X) and (b) a second agent that is semaglutide.

### Pharmaceutical Compositions and Methods of Administration

Administration of FXR modulators as described herein can be in any pharmacological form including a therapeutically effective amount of an FXR modulator alone or in combination with a pharmaceutically acceptable carrier.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. Additional details about suitable excipients for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of Compound 1, Compound 2, or Compound 3 described herein, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. Compound 1, Compound 2, or Compound 3 can be used singly or in combination with one or more therapeutic agents as components of mixtures (as in combination therapy).

The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. Moreover, the pharmaceutical compositions described herein, which include Compound 1, Compound 2, or Compound 3 described herein, can be formulated into any suitable dosage form, including but not limited to, aqueous oral dispersions, liquids, gels, syrups, elixirs, slurries, suspensions, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

Dose administration can be repeated depending upon the pharmacokinetic parameters of the dosage formulation and the route of administration used.

It is especially advantageous to formulate compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of Compound 1, Compound 2, or Compound 3 and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the Compound 1, Compound 2, or Compound 3 activities disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Disclosed herein, in certain embodiments, are compositions for treating a metabolic disorder in an individual in need thereof comprising a first agent that is an FXR modulator and at least one second agent that is an CC2/CCR5 antagonist, ASK1 inhibitor, DPP-IV inhibitor, caspase preotease inhibitor, an SGLT2 inhibitor, acetyl-CoA carboxylase inhibitor, sodium-bile acide cotransporter-inhibitor, TLR-4 antagonist, PPAR alpha/delta agonist, a GLP-1 agonist, or a combination thereof. Disclosed herein, in certain embodiments, are compositions for treating a metabolic disorder in an individual in need thereof comprising a first agent that is an FXR modulator and a second agent that is a DPP-IV inhibitor. Disclosed herein, in certain embodiments, are compositions for treating a metabolic disorder in an individual in need thereof comprising a first agent that is an FXR modulator and a second agent that is an SGLT2 inhibitor. Disclosed herein, in certain embodiments, are compositions for treating a metabolic disorder in an individual in need thereof comprising a first agent that is an FXR modulator and a second agent that is an ASK1 inhibitor. Disclosed herein, in certain embodiments, are compositions for treating a metabolic disorder in an individual in need thereof comprising a first agent that is an FXR modulator and a second agent that is a GLP-1 inhibitor.

Pharmaceutical compositions of a first agent that is an FXR modulator and at least one second agent that is a DPP-IV inhibitor, an SGLT2 inhibitor, an ASK1 inhibitor, a GLP-1 agonist, or a combination thereof are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Tweentysecond Ed (Pharmaceutical Press, 2012); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some embodiments, the first agent that is an FXR modulator and second agent that is a DPP-IV inhibitor are administered concurrently (simultaneously, essentially simultaneously, or withing the same treatment protocol) or sequentially, depending upon the nature of the diseases, the condition of the patient, and the actual choice of compounds used.

### Methods of Dosing and Treatment Regimens

In certain embodiments, the determination of the order of administration, and the number of repetitions of administration of each therapeutic agent during a treatment protocol, is based upon evaluation of the disease being treated and the condition of the patient.

The compounds described herein can be used in the preparation of medicaments for the modulation of FXR, or for the treatment of diseases or conditions that would benefit, at least in part, from modulation of FXR. In addition, a method for treating any of the diseases or conditions described herein in a subject in need of such treatment, involves administration of pharmaceutical compositions containing at least one compound described herein, or a pharmaceutically acceptable salt, or pharmaceutically acceptable solvate or hydrate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician. In certain embodiments, the first agent that is an FXR modulator and the second agent are part of the same composition (fixed combination). In some embodiments, the FXR modulator and the second agent are administered as different compositions (non-fixed combinations). In another embodiment, the FXR modulator is administered prior to the second agent. In some embodiments, the DPP-IV inhibitor is administered prior to the FXR modulator. As many of the disorders for which the compounds and compositions of the invention are useful in treating are chronic disorders, in one embodiment combination therapy involves alternating between administering an FXR modulator and a second agent, e.g., to minimize the toxicity associated with a particular drug. The duration of administration of each drug or therapeutic agent can be one day, one week, one month, three months, six months, or a year.

In some embodiments, the initial administration of FXR modulator and the second agent are via any route practical, such as, for example, an intravenous injection, a bolus injection, infusion over 5 minutes to about 5 hours, a pill, a capsule, transdermal patch, buccal delivery, and the like, or combination thereof.

The FXR modulator and the second agent should be administered as soon as is practicable after the onset of a disorder is detected or suspected, and for a length of time necessary for the treatment of the disease, such as, for example, from about 1 month to about 3 months, or continuously throughout the individual's life. The length of treatment can vary for each subject, and the length can be determined using the known criteria. In some embodiments, the FXR modulator and the second agent are administered for at least 2 weeks, between about 1 month to about 5 years, or from about 1 month to about 3 years. In some embodiments, the FXR modulator and the second agent are administered throughout the individual's life.

Therapeutically effective amounts will depend on the severity and course of the disorder, previous therapy, the patient's health status, weight, and response to the drugs, and the judgment of the treating physician. Prophylactically effective amounts depend on the patient's state of health, weight, the severity and course of the disease, previous therapy, response to the drugs, and the judgment of the treating physician.

In some embodiments, the FXR modulator and the second agent are administered to the patient on a regular basis, e.g., three times a day, two times a day, once a day, every other day or every 3 days. In other embodiments, the FXR modulator and the second agent are administered to the patient on an intermittent basis, e.g., twice a day followed by once a day followed by three times a day; or the first two days of every week; or the first, second and third day of a week. In some embodiments, intermittent dosing is as effective as regular dosing. In further or alternative embodiments, the FXR modulator and the second agent are administered only when the patient exhibits a particular symptom, e.g., the onset of pain, or the onset of a fever, or the onset of an inflammation, or the onset of a skin disorder. Dosing schedules of each compound may depend on the other or may be independent of the other.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance regimen is administered if necessary. Subsequently, the dosage or the frequency of administration, or both, of The FXR modulator and the second agent can be reduced, as a function of the symptoms, to a level at which the individual's improved condition is retained. Individuals can, however, require intermittent treatment on a long-term basis upon any recurrence of symptoms.

The amount of the FXR modulator and the second agent are will vary depending upon factors such as the particular compound, disorder and its severity, the identity (e.g., weight) of the subject or host in need of treatment, and is determined according to the particular circumstances surrounding the case, including, e.g., the specific agents being administered, the routes of administration, and the subject or host being treated. In general, however, doses employed for adult human treatment will typically be in the range of 0.02-5000 mg per day.

In some embodiments, the effective dose of the FXR modulator is about 1 mg to about 1500 mg, about 1 mg to about 1400 mg, about 1 mg to about 1300 mg, about 1 mg to about 1200 mg, about 1 mg to about 1100 mg, about 1 mg to about 1000 mg, 1 mg to about 900 mg, about 1 mg to about 800 mg, about 1 mg to about 700 mg. In some embodiments, the effective dose of the FXR modulator is about 1 mg to about 600 mg, about 1 mg to about 500 mg, about 1 mg to about 400 mg, about 1 mg to about 300 mg, about 1 mg to about 200 mg, about 1 mg to about 100 mg, about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 10 mg, or about 1 mg to about 5 mg.

In some embodiments, the effective dose of the FXR modulator is about 10 mg to about 1500 mg, about 20 mg to about 1500 mg, about 30 mg to about 1500 mg, about 40 mg to about 1500 mg, about 50 mg to about 1500 mg, about 60 mg to about 1500 mg, about 70 mg to about 1500 mg, about 80 mg to about 1500 mg, about 90 mg to about 1500 mg, about 100 mg to about 1500 mg, about 200 mg to about 1500 mg, about 300 mg to about 1500 mg, about 400 mg to about 1500 mg, about 500 mg to about 1500 mg, about 600 mg to about 1500 mg, about 700 mg to about 1500 mg, about 800 mg to about 1500 mg, about 900 mg to about 1500 mg, about 1000 mg to about 1500 mg, about 1100 mg to about 1500 mg, about 1200 mg to about 1500 mg, about 1300 mg to about 1500 mg, or about 1400 mg to about 1500 mg. In some embodiments, the effective dose of the FXR modulator of Formula (I), (II), (III), (IV), (V), (VI), (VII), (VIII), (IX), (X), (XI), or (XII) is about 10 mg to about 450 mg, about 10 mg to about 50 mg, about 50 mg to about 100 mg, about 100 mg to about 200 mg, about 200 mg to about 300 mg, or about 300 mg to about 450 mg. In some embodiments, the effective dose of the FXR modulator of Formula (I), (II), (III), (IV), (V), (VI), (VII), (VIII), (IX), (X), (XI), or (XII) is about 1 mg to about 100 mg, about 1 mg to about 5 mg, about 5 mg to about 10 mg, about 10 mg to about 25 mg, about 25 mg to about 50 mg, about 50 mg to about 75 mg, or about 75 mg to about 100 mg. In some embodiments, the effective dose of the FXR modulator of Formula (I), (II), (III), (IV), (V), (VI), (VII), (VIII), (IX), (X), (XI), or (XII) is about 10 mg to about 250 mg, about 10 mg to about 50 mg, about 50 mg to about 100 mg, about 100 mg to about 150 mg, about 150 mg to about 200 mg, or about 200 mg to about 250 mg. In some embodiments, the effective dose of the FXR modulator of Formula (I), (II), (III), (IV), (V), (VI), (VII), (VIII), (IX), (X), (XI), or (XII) is about 500 mg to about 1500 mg, about 500 mg to about 600 mg, about 600 mg to about 700 mg, about 700 mg to about 800 mg, about 800 mg to about 900 mg, about 900 mg to about 1000 mg, about 1000 mg to about 1200 mg, or about 1200 mg to about 1500 mg.

In some embodiments, the effective dose of the DPP-IV inhibitor is about 1 mg to about 1000 mg. In some embodiments, the effective dose of the DPP-IV inhibitor is about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5 mg, about 24 mg, about 24.5 mg, about 25 mg, about 25.5 mg, about 26 mg, about 26.5 mg, about 27 mg, about 27.5 mg, about 28 mg, about 28.5 mg, about 29 mg, about 29.5 mg, about 30 mg, about 30.5 mg, about 31 mg, about 31.5 mg, about 32 mg, about 32.5 mg, about 33 mg, about 33.5 mg, about 34 mg, about 34.5 mg, about 35 mg, about 35.5 mg, about 36 mg, about 36.5 mg, about 37 mg, about 37.5 mg, about 38 mg, about 38.5 mg, about 39 mg, about 39.5 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, or about 950 mg, about 1000 mg. In certain embodiments, any two of the doses in this paragraph may be combined to form a range of dosages included within the disclosure, e.g., the topical composition comprises from about 2 mg to about 100 mg, from about 10 mg to about 150 mg, from about 50 mg to about 200 mg, or from about 100 mg to about 300 mg.

In some embodiments, the effective dose of the SGLT2 inhibitor is about 1 mg to about 1000 mg. In some embodiments, the effective dose of the SGLT2 inhibitor is about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5 mg, about 24 mg, about 24.5 mg, about 25 mg, about 25.5 mg, about 26 mg, about 26.5 mg, about 27 mg, about 27.5 mg, about 28 mg, about 28.5 mg, about 29 mg, about 29.5 mg, about 30 mg, about 30.5 mg, about 31 mg, about 31.5 mg, about 32 mg, about 32.5 mg, about 33 mg, about 33.5 mg, about 34 mg, about 34.5 mg, about 35 mg, about 35.5 mg, about 36 mg, about 36.5 mg, about 37 mg, about 37.5 mg, about 38 mg, about 38.5 mg, about 39 mg, about 39.5 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, or about 950 mg, about 1000 mg. In certain embodiments, any two of the doses in this paragraph may be combined to form a range of dosages included within the disclosure, e.g., the topical composition comprises from about 2 mg to about 100 mg, from about 10 mg to about 150 mg, from about 50 mg to about 200 mg, or from about 100 mg to about 300 mg.

In some embodiments, the effective dose of the ASK1 inhibitor is about 1 mg to about 1000 mg. In some embodiments, the effective dose of the ASK1 inhibitor is about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5 mg, about 24 mg, about 24.5 mg, about 25 mg, about 25.5 mg, about 26 mg, about 26.5 mg, about 27 mg, about 27.5 mg, about 28 mg, about 28.5 mg, about 29 mg, about 29.5 mg, about 30 mg, about 30.5 mg, about 31 mg, about 31.5 mg, about 32 mg, about 32.5 mg, about 33 mg, about 33.5 mg, about 34 mg, about 34.5 mg, about 35 mg, about 35.5 mg, about 36 mg, about 36.5 mg, about 37 mg, about 37.5 mg, about 38 mg, about 38.5 mg, about 39 mg, about 39.5 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, or about 950 mg, about 1000 mg. In certain embodiments, any two of the doses in this paragraph may be combined to form a range of dosages included within the disclosure, e.g., the topical composition comprises from about 2 mg to about 100 mg, from about 10 mg to about 150 mg, from about 50 mg to about 200 mg, or from about 100 mg to about 300 mg.

In some embodiments, the effective dose of the GLP-1 agonist is about 1 mg to about 1000 mg. In some embodiments, the effective dose of the GLP-1 agonist is about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 20.5 mg, about 21 mg, about 21.5 mg, about 22 mg, about 22.5 mg, about 23 mg, about 23.5 mg, about 24 mg, about 24.5 mg, about 25 mg, about 25.5 mg, about 26 mg, about 26.5 mg, about 27 mg, about 27.5 mg, about 28 mg, about 28.5 mg, about 29 mg, about 29.5 mg, about 30 mg, about 30.5 mg, about 31 mg, about 31.5 mg, about 32 mg, about 32.5 mg, about 33 mg, about 33.5 mg, about 34 mg, about 34.5 mg, about 35 mg, about 35.5 mg, about 36 mg, about 36.5 mg, about 37 mg, about 37.5 mg, about 38 mg, about 38.5 mg, about 39 mg, about 39.5 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, or about 950 mg, about 1000 mg. In certain embodiments, any two of the doses in this paragraph may be combined to form a range of dosages included within the disclosure, e.g., the topical composition comprises from about 2 mg to about 100 mg, from about 10 mg to about 150 mg, from about 50 mg to about 200 mg, or from about 100 mg to about 300 mg.

It is understood that a medical professional will determine the dosage regimen in accordance with a variety of factors. These factors include the age, weight, sex, diet, and medical condition of the subject. The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.01 mg per day to about 5000 mg per day, in some embodiments, about 1 mg per day to about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

In some embodiments, a pharmaceutical composition, refers to a mixture of an FXR modulator and a second agent with other chemical components, such as carriers, stabilizers, diluents, dispersing agents, suspending agents, thickening agents, and/or excipients. In some embodiments, a pharmaceutical composition refers to an FXR modulator with other chemical components, such as carriers, stabilizers, diluents, dispersing agents, suspending agents, thickening agents, and/or excipients. In some embodiments, a pharmaceutical composition refers to a DPP-IV inhibitor with other chemical components, such as carriers, stabilizers, diluents, dispersing agents, suspending agents, thickening agents, and/or excipients. In some embodiments, a pharmaceutical composition refers to an SGLT2 inhibitor with other chemical components, such as carriers, stabilizers, diluents, dispersing agents, suspending agents, thickening agents, and/or excipients. In some embodiments, a pharmaceutical composition refers to a ASK1 inhibitor with other chemical components, such as carriers, stabilizers, diluents, dispersing agents, suspending agents, thickening agents, and/or excipients. In some embodiments, a pharmaceutical composition refers to a GLP-1 agonist with other chemical components, such as carriers, stabilizers, diluents, dispersing agents, suspending agents, thickening agents, and/or excipients.

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical formulations described herein are administered by any suitable administration route, including but not limited to, oral, parenteral (e.g., intravenous, subcutaneous, intramuscular), intranasal, buccal, topical, rectal, or transdermal administration routes.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the compositions are formulated into capsules. In some embodiments, the compositions are formulated into solutions (for example, for IV administration).

The pharmaceutical compositions described herein are formulated into unit dosage forms suitable for single administration of precise dosages. In unit dosage form, the formulation is divided into unit doses containing appropriate quantities of one or both compounds. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v, polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

The term “disintegrate” includes both the dissolution and dispersion of the dosage form when contacted with gastrointestinal fluid. “Disintegration agents or disintegrants” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

An “enteric coating” is a substance that remains substantially intact in the stomach but dissolves and releases the drug in the small intestine or colon. Generally, the enteric coating comprises a polymeric material that prevents release in the low pH environment of the stomach but that ionizes at a higher pH, typically a pH of 6 to 7, and thus dissolves sufficiently in the small intestine or colon to release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids. Combinations of one or more erosion facilitator with one or more diffusion facilitator can also be used in the present compositions.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” and/or “sweeteners” useful in the formulations described herein, include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Steady state,” as used herein, is when the amount of drug administered is equal to the amount of drug eliminated within one dosing interval resulting in a plateau or constant plasma drug exposure.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

It should be appreciated that there is considerable overlap between additives used in the solid dosage forms described herein. Thus, the above-listed additives should be taken as merely exemplary, and not limiting, of the types of additives that can be included in solid dosage forms described herein. The amounts of such additives can be readily determined by one skilled in the art, according to the particular properties desired.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

Compressed tablets are solid dosage forms prepared by compacting the bulk blend of the formulations described above. In various embodiments, compressed tablets which are designed to dissolve in the mouth will include one or more flavoring agents. In other embodiments, the compressed tablets will include a film surrounding the final compressed tablet. In some embodiments, the film coating can provide a delayed release of the FXR modulator in combination with or separately from a second agent, from the formulation. In other embodiments, the film coating aids in patient compliance (e.g., Opadry® coatings or sugar coating). Film coatings including Opadry® typically range from about 1% to about 3% of the tablet weight. In other embodiments, the compressed tablets include one or more excipients.

A capsule may be prepared, for example, by placing the bulk blend of the formulation of the compounds described herein, inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule may be swallowed whole or the capsule may be opened and the contents sprinkled on food prior to eating. In some embodiments, the therapeutic dose is split into multiple (e.g., two, three, or four) capsules. In some embodiments, the entire dose of the formulation is delivered in a capsule form.

In various embodiments, the particles of the compounds described herein and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with the compounds described herein, which sufficiently isolate the compounds from other non-compatible excipients. Materials compatible with compounds of any of the FXR modulators and the second agent, are those that delay the release of the compounds of any of the FXR modulator and the second agent, in vivo.

Exemplary microencapsulation materials useful for delaying the release of the formulations including compounds described herein, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated compounds of the compounds described herein may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

In one embodiment, the particles of the compounds described herein are microencapsulated prior to being formulated into one of the above forms. In still another embodiment, some or most of the particles are coated prior to being further formulated by using standard coating procedures, such as those described in Remington's Pharmaceutical Sciences, 20th Edition (2000).

In still other embodiments, effervescent powders are also prepared in accordance with the present disclosure. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the compositions described herein are added to water, the acids and the base react to liberate carbon dioxide gas, thereby causing “effervescence.” Examples of effervescent salts include, e.g., the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions described herein to achieve delivery to the lower gastrointestinal tract. In some embodiments the polymers described herein are anionic carboxylic polymers. In other embodiments, the polymers and compatible mixtures thereof, and some of their properties, include, but are not limited to:

Shellac, also called purified lac, a refined product obtained from the resinous secretion of an insect. This coating dissolves in media of pH >7;

Acrylic polymers. The performance of acrylic polymers (primarily their solubility in biological fluids) can vary based on the degree and type of substitution. Examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH >6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; Poly Vinyl Acetate Phthalate (PVAP). PVAP dissolves in pH >5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

In other embodiments, the formulations described herein, which include the compounds described herein, are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

In some embodiments, pharmaceutical formulations are provided that include particles of the compounds described herein and at least one dispersing agent or suspending agent for oral administration to a subject. The formulations may be a powder and/or granules for suspension, and upon admixture with water, a substantially uniform suspension is obtained.

Liquid formulation dosage forms for oral administration can be aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2^{nd} Ed., pp. 754-757 (2002). In addition the liquid dosage forms may include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

The aqueous suspensions and dispersions described herein can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

### Intranasal Formulations

Intranasal formulations are known in the art and are described in, for example, U.S. Pat. Nos. 4,476,116, 5,116,817 and 6,391,452, each of which is specifically incorporated by reference. Formulations that include an FXR modulator in combination with or separately from the second agent, which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 22st edition, 2012, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. The nasal dosage form should be isotonic with nasal secretions.

For administration by inhalation described herein may be in a form as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

### Buccal Formulations

Buccal formulations may be administered using a variety of formulations known in the art. For example, such formulations include, but are not limited to, U.S. Pat. Nos. 4,229,447, 4,596,795, 4,755,386, and 5,739,136, each of which is specifically incorporated by reference. In addition, the buccal dosage forms described herein can further include a bioerodible (hydrolysable) polymeric carrier that also serves to adhere the dosage form to the buccal mucosa. The buccal dosage form is fabricated so as to erode gradually over a predetermined time period, wherein the delivery is provided essentially throughout. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not compromised, and the carrier is compatible with an FXR modulator and a second agent, and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like. For buccal or sublingual administration, the compositions may take the form of tablets, lozenges, or gels formulated in a conventional manner.

### Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety.

The transdermal dosage forms described herein may incorporate certain pharmaceutically acceptable excipients which are conventional in the art. In one embodiments, the transdermal formulations described herein include at least three components: (1) a formulation of a compound of the compounds described herein; (2) a penetration enhancer; and (3) an aqueous adjuvant. In addition, transdermal formulations can include additional components such as, but not limited to, gelling agents, creams and ointment bases, and the like. In some embodiments, the transdermal formulation can further include a woven or non-woven backing material to enhance absorption and prevent the removal of the transdermal formulation from the skin. In other embodiments, the transdermal formulations described herein can maintain a saturated or supersaturated state to promote diffusion into the skin.

Formulations suitable for transdermal administration of compounds described herein may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds described herein can be accomplished by means of iontophoretic patches and the like. Additionally, transdermal patches can provide controlled delivery of the compounds described herein. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

### Injectable Formulations

Formulations that include the compounds described herein, suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds described herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

### Other Formulations

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the compounds described herein may be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

The compounds described herein may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In some embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compound as disclosed herein may be derived from reactions and the reactions may be modified by the use of appropriate reagents and conditions, for the introduction of the various moieties found in the formulae as provided herein.

In some embodiments, the compounds described herein are prepared as outlined in the following schemes.

Scheme 1 provides a synthetic procedure generally applicable to the synthesis of compounds of Formulae (I) and (III) according to procedures found here and elsewhere, e.g., PCT/US2015/062017 herein incorporated by reference in its entirety.

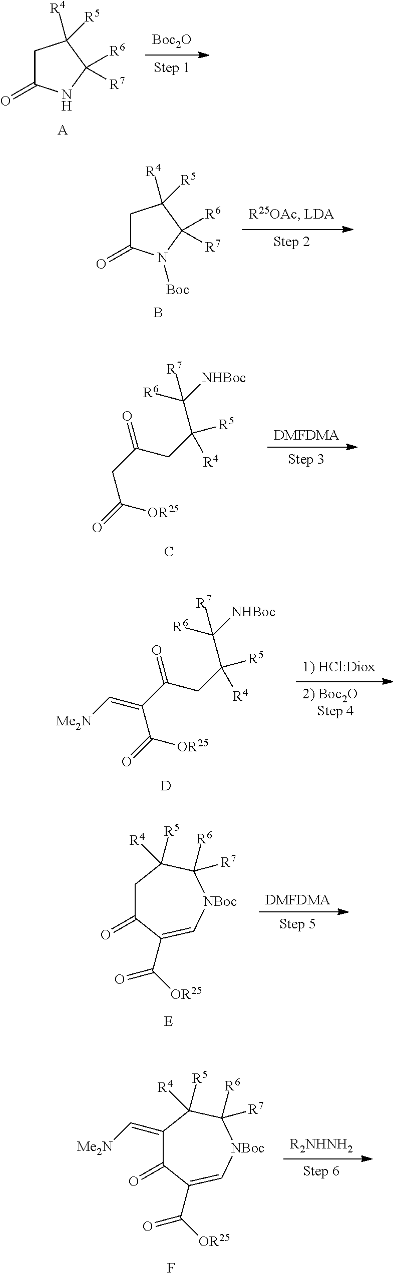

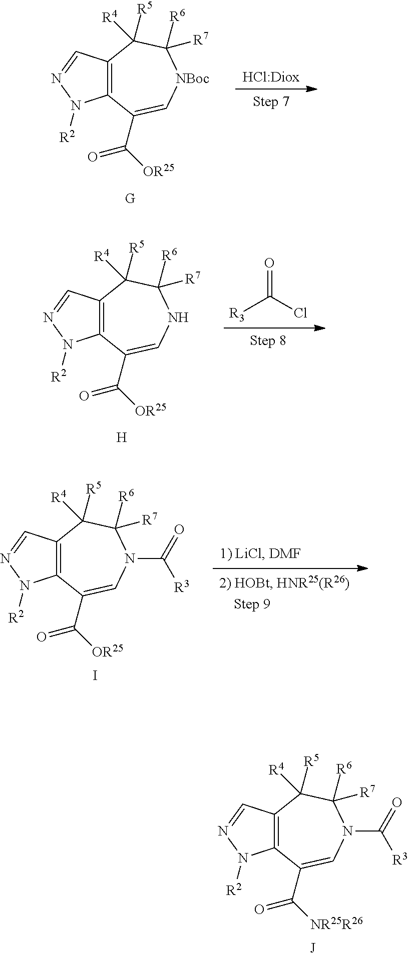

Scheme 2 provides a synthetic procedure generally applicable to the synthesis of compounds of Formulae (II) and (IV) according to procedures found here and elsewhere, e.g., PCT/US2015/062017 herein incorporated by reference in its entirety.

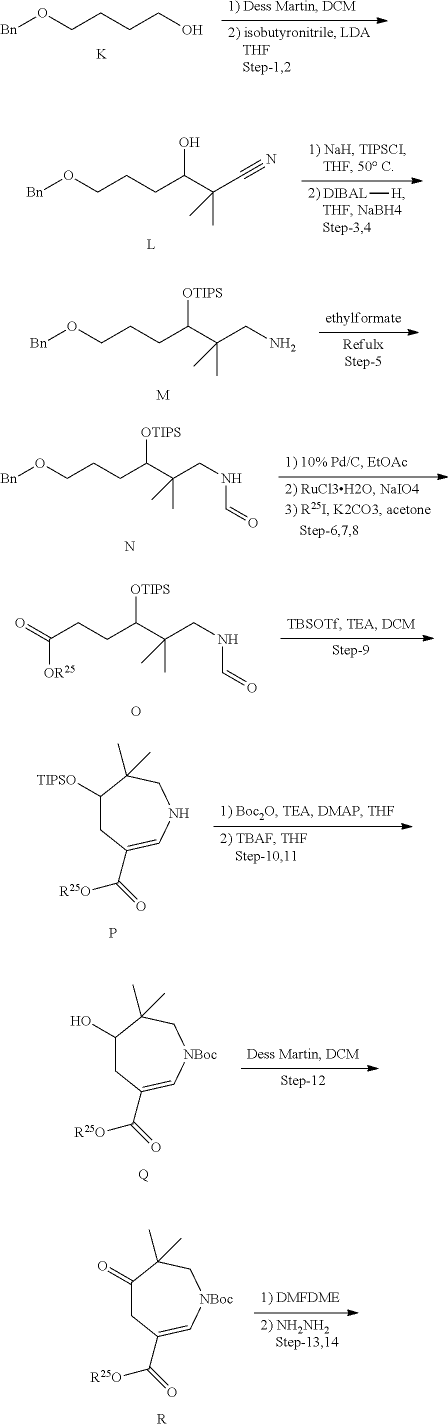

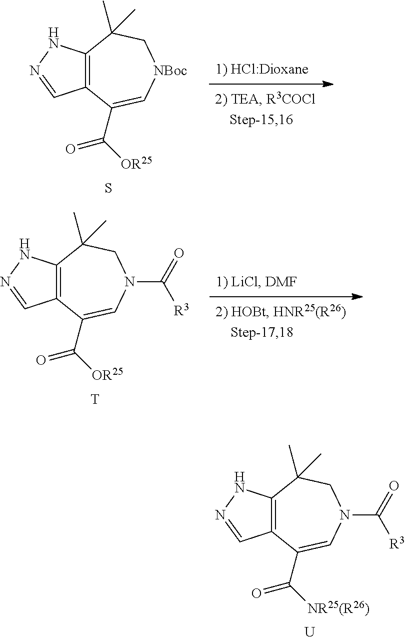

The following examples are offered for purposes of illustration, and are not intended to limit the scope of the claims provided herein. All literature citations in these examples and throughout this specification are incorporated herein by references for all legal purposes to be served thereby. The starting materials and reagents used for the synthesis of the compounds described herein may be synthesized or can be obtained from commercial sources, such as, but not limited to, Sigma-Aldrich, Acros Organics, Fluka, and Fischer Scientific.

### Example 3: Synthesis of (E)-isopropyl 6-(3,4-difluorobenzoyl)-4,4-dimethyl-3-(trifluoromethyl)-1,4,5,6-tetrahydropyrazolo[3,4-d]azepine-8-carboxylate (see also PCT/US2015/062017). Generally Applicable to the Synthesis of Compounds of Formulae (I), (III), and (IIIa)

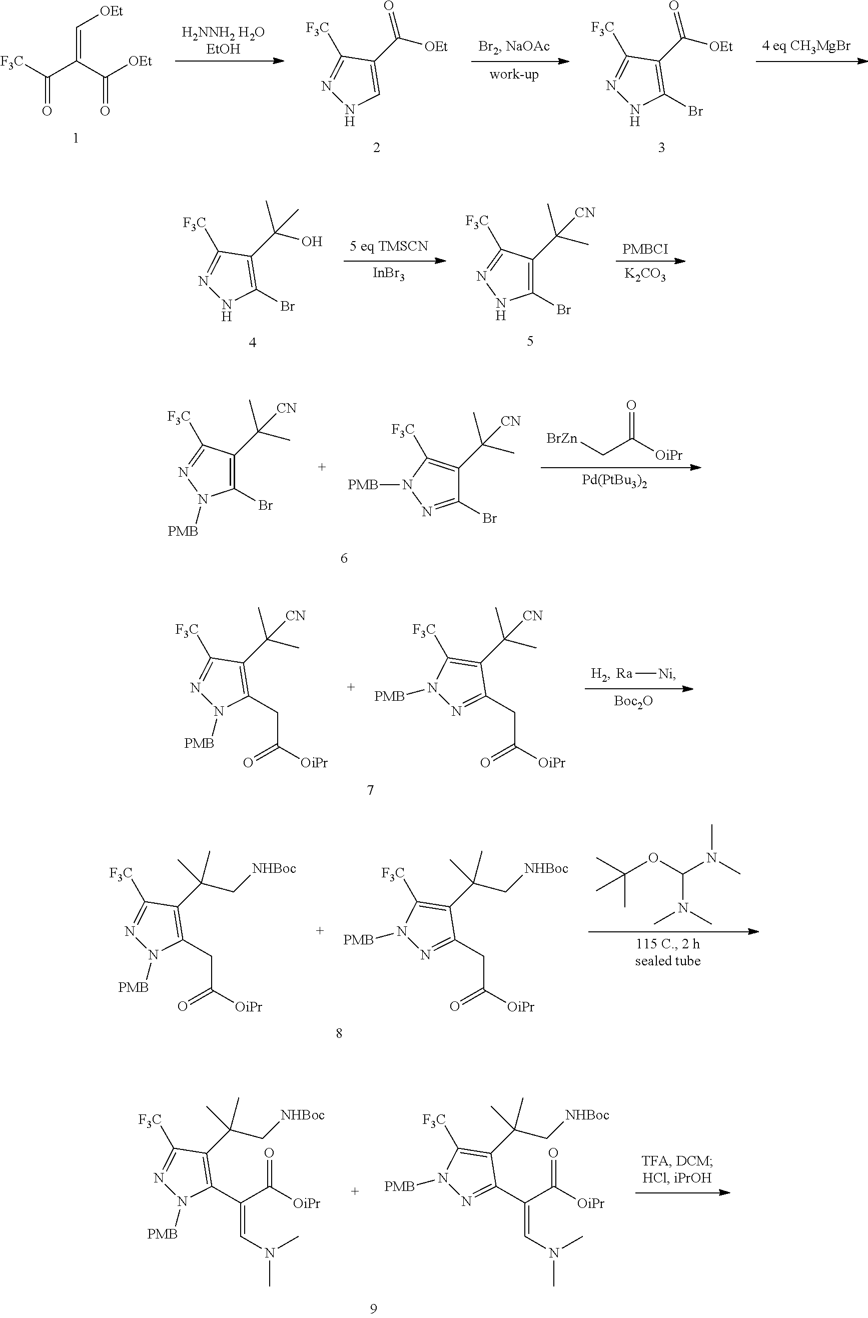

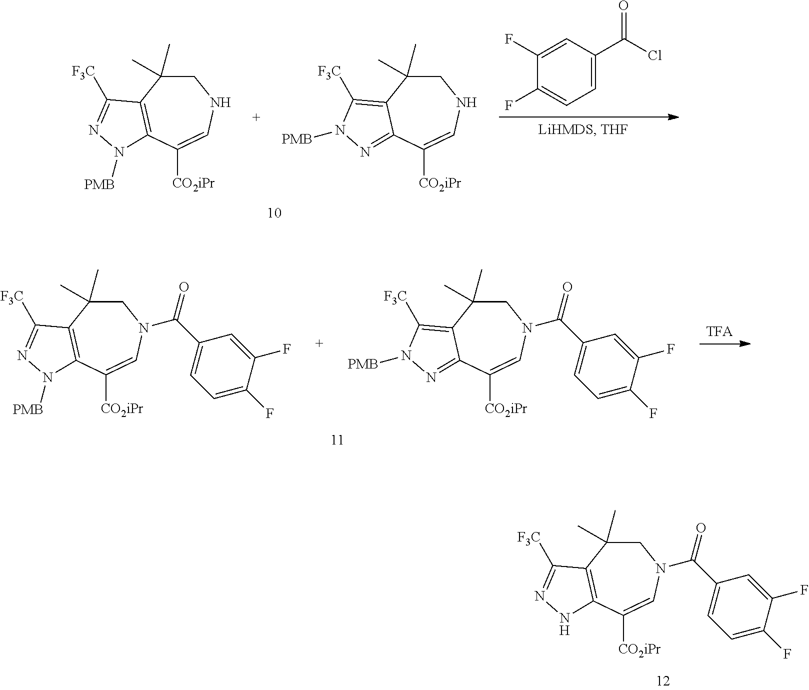

Step 1: A solution of hydrazine hydrate (34.4 g, 0.687 mol, 1.1 eq) in ethanol (400 mL) was added to a solution of compound 1 (150 g, 0.62 mol) in ethanol (1000 mL) at 0° C. The reaction was allowed to warm to room temperature and stirred for 24 hr. The reaction was concentrated in vacuo, dissolved in ethyl acetate (2000 mL), washed with 5% citric acid (2000 mL), sat′d NaHCO₃ (2000 mL) and brine, dried (MgSO₄), and concentrated in vacuo to afford a light yellow solid, compound 2 (113 g, 88%).

Step 2: To a solution of compound 2 (20.0 g, 96.1 mmol) in acetic acid (200 mL) was added sodium acetate (23.6 g, 288.3 mmol, 3.0 eq.). To the suspended solution was added Br₂ (14.7 mL, 288.3 mmol, 3.0 eq.) dropwise. The resulting mixture was stirred at room temperature for 10 minutes, and then heated at 100° C. in a sealed-tube for 5 hr. The solvent and Br₂ was removed in vacuo. The residue was diluted with ethyl acetate (600 mL), washed with water (2×600 mL), saturated NaHCO₃ (600 mL), and brine. The organic phase was dried over MgSO₄, and concentrated in vacuo. The crude product was purified by column chromatography (SiO₂, DCM/EA=9/1) to afford an ivory solid 3 (20 g×2 batch; 51.4 g, 188.3 mol, 98%).

Step 3: A solution of compound 3 (96.5 g, 353.4 mmol, 1.0 eq.) in dry THF (1.2 L), and was cooled in an ice-water bath. MeMgBr (471 mL, 3M in ether solution, 1.41 mol, 4.0 eq.) was added dropwise. The resulting mixture was stirred at 0° C. for 30 minutes, then room temperature overnight. The reaction was cooled to 0° C., then quenched with saturated NH₄Cl solution (1.6 L). The organic phase was washed with brine, and dried over MgSO₄, filtered and concentrated. The crude product was purified by column chromatography (SiO₂, DCM/EA=9/1) to afford an ivory solid 4 (69.1 g, 253.2 mmol, 72%).

Step 4: To a suspension of indium( [symbol] ) bromide (6.5 g, 18.3 mmol, 0.1 eq.) in dichloromethane (500 mL) was added trimethylsilyl cyanide (69 mL, 549.4 mmol, 3.0 eq.). To this mixture, at room temperature, was added dropwise compound 4 (50.0 g, 183.1 mmol, 1.0 eq.) in dichloromethane (1500 mL). The resulting mixture was stirred at room temperature overnight. Saturated NaHCO₃was added and the mixture was filtered through a celite pad. The filtrate was partitioned between saturated NaHCO₃and dichloromethane and the aqueous layer was extracted one more time with ethyl acetate. The combined organic layers were dried over MgSO₄, filtered and concentrated. The crude product was purified by column chromatography (SiO₂, DCM to DCM/MeOH=30/1) to afford a brown oil 5 (50 g×2 batch; 107.1 g).

Step 5: To a solution of compound 5 (56.3 g, 199.7 mmol, 1.0 eq.) in CH₃CN (1600 mL), was added K₂CO₃ (82.8 g, 599.1 mmol, 3.0 eq.) and PMBCl (32.5 mL, 239.6 mmol, 1.2 eq.). The mixture was heated at reflux for 2 hr. The reaction was cooled to room temperature. The inorganic solid was removed by filtration, and the mother liquid was concentrated in vacuo. The crude product was purified by column chromatography (SiO₂, Hex/EA=9/1) to afford a yellow oil 6 (56.3 g, 50.8 g×2 batch, 133.5 g, 332.0 mmol, 91%).

Step 6A: To a suspension of zinc dust (4.1 g, 31.0 mmol, 2.0 eq.) in dry ether (40 mL) was added dropwise HCl (2M solution in ether; 2 mL, 0.13 eq.). The suspension was heated to reflux, and isopropyl bromoacetate (4 mL, 31.0 mmol, 2.5 eq.) was added dropwise. The solution was stirred at this temperature for 4 hr and cooled to room temperature.

Step 6B: To a solution of 6 ((5.0 g, 12.4 mmol, 1.0 eq.) in anhydrous THF (100 mL) was added Pd(P(tBu)₃)₂ (5.1 g, 9.94 mmol, 0.8 eq.) under argon. The solution of (2-isopropoxy-2-oxoethyl) zinc bromide from step 6A was added drop-wise. The resulting mixture was stirred in an oil bath with heating from room temperature to 75° C. within 10 minutes. The reaction mixture was heated at 75° C. for 2 hr. The reaction mixture was cooled to room temperature and quenched with saturated NH₄Cl (200 mL). After extraction of the product with ethyl acetate, the crude product was purified by column chromatography (SiO₂, Hex/EA=9/1→Hex/EA=6/1) to afford an ivory oil 7 (2.4 g, 5.7 mmol, 46%).

Step 7: To a solution of compound 7 (7.8 g, 18.42 mmol, 1.0 eq) in THF (80 mL) and iPrOH (160 mL) was added Boc anhydride (8.04 g, 36.84 mmol, 2.0 eq) and a Ra—Ni slurry in water (40 mL). The resulting mixture was hydrogenated at H₂ 40 psi for 4 h. The catalyst was carefully removed by filtration. The filtrate was concentrated in vacuo. The crude product was purified by column chromatography (SiO₂, HX/EA=5/1) to afford a sticky oil 8 (6.9 g, 71%).

Step 8: Compound 8 (6.9 g, 13.08 mmol) was dissolved in Bredereck's reagent (55 mL). The solution was flushed with nitrogen, and then heated at 115° C. in a sealed tube for 3 h. The mixture was diluted with CH₂Cl₂ (500 mL). The organic phase was washed with water and brine, dried over MgSO₄, filtered and concentrated. The crude mixture was purified by column chromatography (SiO₂, Hx/EA=2/1) to afford a sticky oil 9 (6.8 g, 89%).

Step 9A: To a solution of compound 9 (6.8 g, 11.67 mmol) in dry CH₂Cl₂ (50 mL) was added TFA (30 mL). The solution was stirred at room temperature for 15 minutes. The solvent was removed in vacuo. The residue was diluted with CH₂Cl₂ (500 mL), washed with saturated NaHCO₃and brine, dried over MgSO₄, filtered and concentrated to afford the free amine intermediate.

Step 9B: To a solution of the intermediate from step 9A in iPrOH (100 mL) was added concentrated HCl in water (3.4 mL). The resulting mixture was heated at 100° C. in a sealed tube for 18 h. The solvent was removed in vacuo. The residue was dissolved in CH₂Cl₂ (500 mL), washed with saturated NaHCO₃and brine, dried over MgSO₄, filtered and concentrated. The crude product was purified by column chromatography (SiO₂, Hx/EA=2/1) to afford solid 10 (3.7 g, 72%).

Step 10: To a solution of 10 (2 g, 4.57 mmol) in dry THF (50 mL) was added LiHMDS (1M in hexane, 6.85 mL, 1.5 eq) dropwise at 0° C. 3,4-difluorobenzoyl chloride (1.15 mL, 2.0 eq) was then added dropwise. The resulting mixture was stirred at room temperature for 2 h. The mixture was quenched with saturated NH₄Cl and extracted with ethyl acetate. The organic solution was dried over MgSO₄, filtered and concentrated. The crude product was purified by column chromatography (SiO₂, Hx/EA=5/1) to afford solid 11 (2 g, 75%).

Step 11: A solution of compound 11 (2 g, 3.46 mmol) in TFA (20 mL) was heated at 90° C. in a sealed tube for 10 minutes. The TFA was removed in vacuo and the crude product was purified by column chromatography (SiO₂, DCM/Hx/EA=10/20/0.5) to afford the title compound 12 (1.3 g, 82%). LCMS m/z: 444.1 [M+H]+.

### Example 4: Synthesis of (E)-ethyl 6-(3,4-difluorobenzoyl)-3,4,4-trimethyl-1,4,5,6-tetrahydropyrazolo[3,4-d]azepine-8-carboxylate (23) (see also PCT/US2015/062017). Generally Applicable to the Synthesis of Compounds of Formulae (I), (III), and (IIIa)

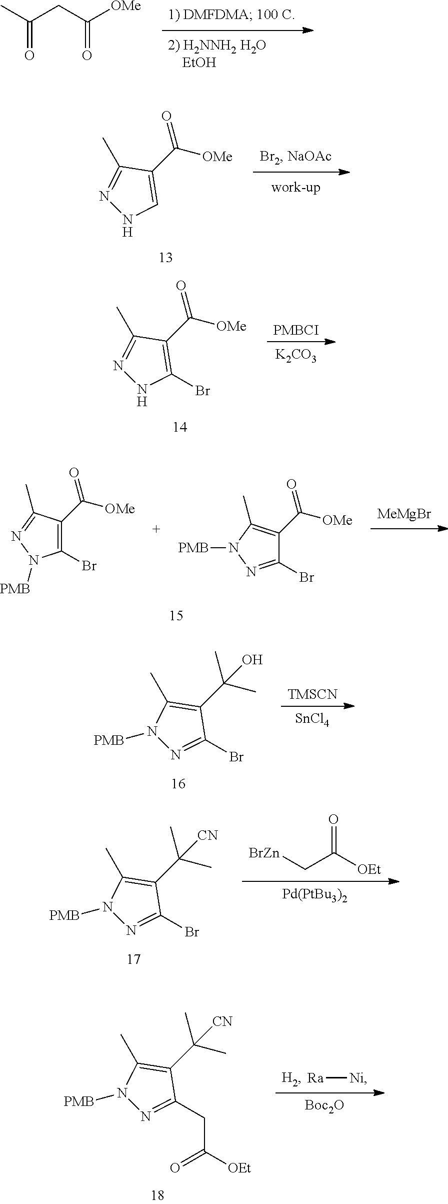

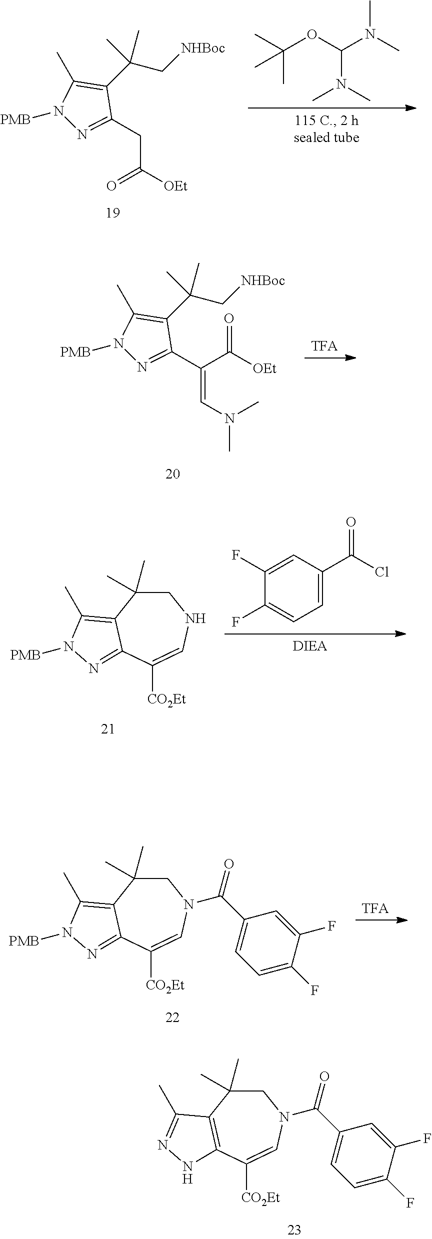

In some embodiments, the compounds described herein are prepared as outlined in the following schemes:

Scheme 3 as applicable to compounds of the Formulae (VII), (VIIa)-(VIIj), (VIII), (VIIIa)-(VIIIj), see also PCT/IB2015/002549 herein incorporated by reference in its entirety, Published as WO 2016/103037:

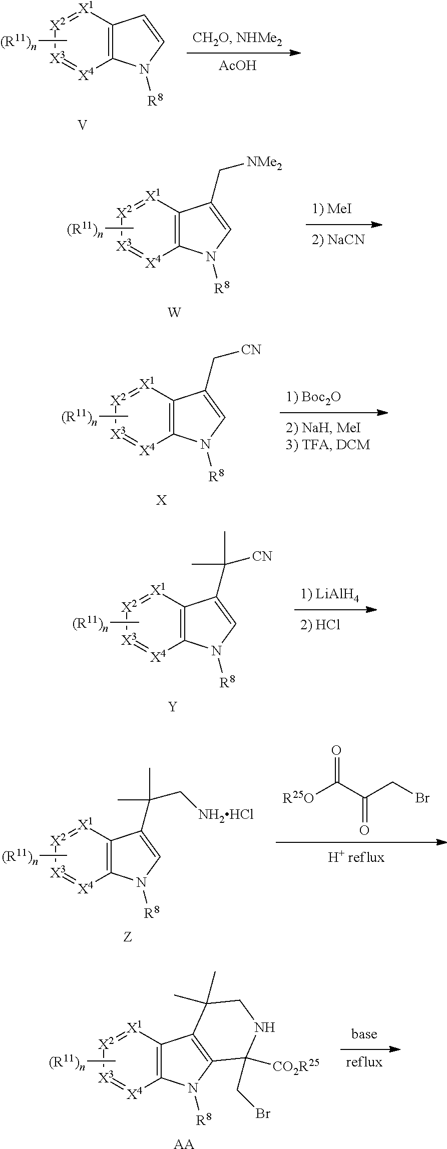

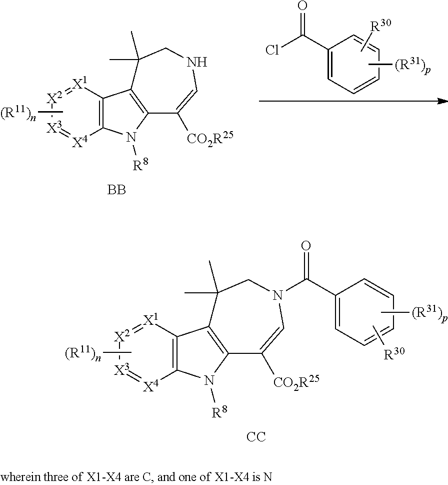

Scheme 4 as applicable to compounds of the Formulae (VII), (VIIa)-(VIIj), (VIII), (VIIIa)-(VIIIj), see also PCT/IB2015/002549 herein incorporated by reference in its entirety, Published as WO 2016/103037

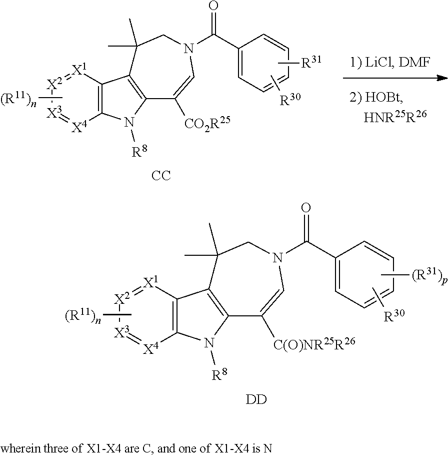

### Example 5: Synthesis of propan-2-yl 4-chloro-12-(3,4-dichlorobenzoyl)-14,14-dimethyl-6,8,12-triazatricyclo[7.5.0.02,7]tetradeca-1(9),2,4,6,10-pentaene-10-carboxylate (31) See Also PCT/IB2015/002549 Herein Incorporated by Reference in its Entirety, Published as WO 2016/103037

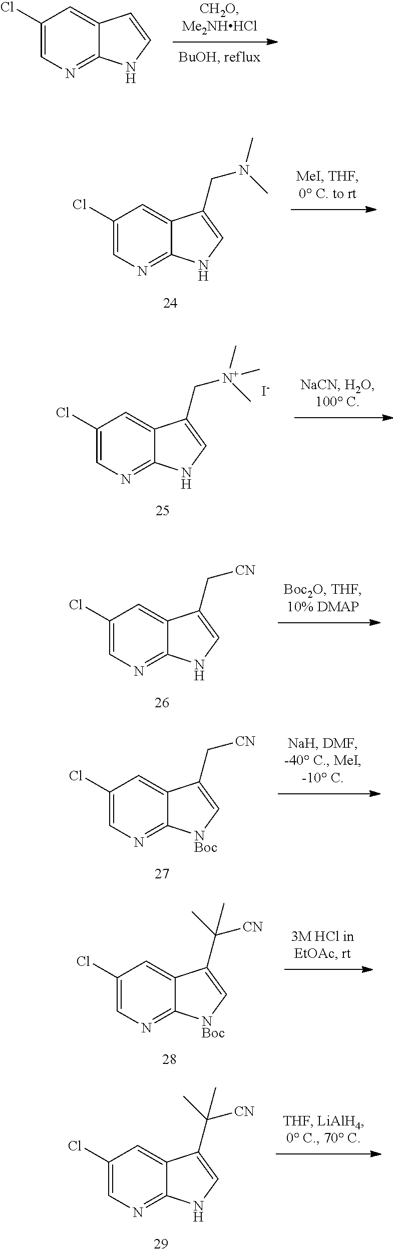

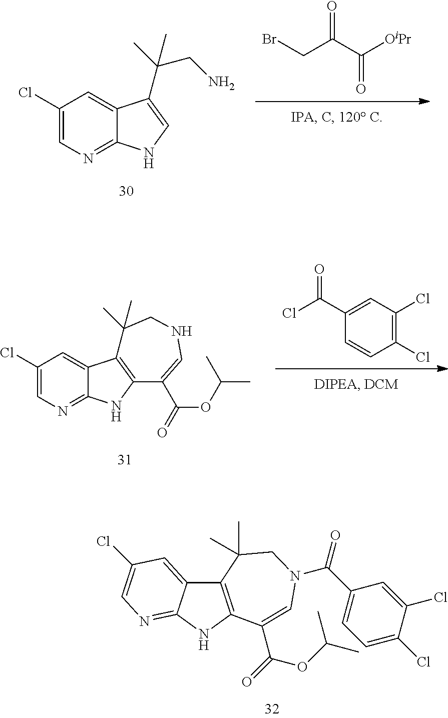 [SMILES: CC(C)(CN)c1c[nH]c2ncc(Cl)cc12 ; CC(C)OC(=O)C(=O)CBr ; CC(C)OC(=O)C1=CNCC(C)(C)c2c1[nH]c1ncc(Cl)cc21 ; O=C(Cl)c1ccc(Cl)c(Cl)c1 ; CC(C)OC(=O)C1=CN(C(=O)c2ccc(Cl)c(Cl)c2)CC(C)(C)c2c1[nH]c1ncc(Cl)cc21]

### Example 6: Synthesis of propan-2-yl 4-chloro-12-(3,4-difluorobenzoyl)-14,14-dimethyl-6,8,12-triazatricyclo[7.5.0.02,7]tetradeca-1(9),2,4,6,10-pentaene-10-carboxylate (33)

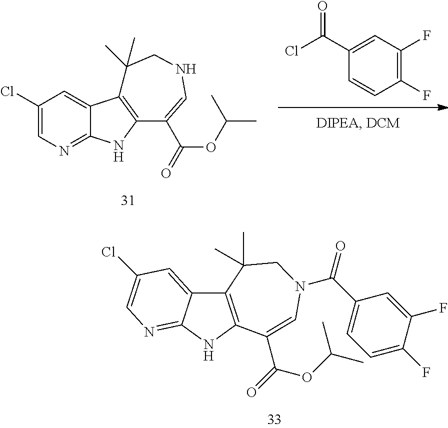 [SMILES: CC(C)OC(=O)C1=CNCC(C)(C)c2c1[nH]c1ncc(Cl)cc21 ; O=C(Cl)c1ccc(F)c(F)c1 ; CC(C)OC(=O)C1=CN(C(=O)c2ccc(F)c(F)c2)CC(C)(C)c2c1[nH]c1ncc(Cl)cc21]

To a stirred solution of compound 31 (52 mg, 0.15 mmol) and DIPEA (0.13 mL, 0.75 mmol) in dichloromethane (4 mL), 3,4-difluorobenzoyl chloride (0.09 mL, 0.75 mmol) in DCM (1 mL) was added. The reaction mixture was stirred at rt for 10 mins and it was then diluted with DCM. The mixture was washed with aqueous NaHCO₃and brine. The organics were dried over anhydrous sodium sulfate and concentrated in vacuo. The residue was purified by silica gel column chromatography (PE/EA, from 20/1 to 10/1) to afford the title compound (33 mg, 43%). LC-MS: 474.30 (M+H), C₂₄H₂₂C₁F₂N₃O₃. ¹H NMR (CDCl₃, 400 MHz) δ 11.10 (br, 1H), 8.23 (s, 1H), 8.04 (s, 1H), 7.90 (s, 1H), 7.54-7.49 (m, 1H), 7.38-7.34 (m, 1H), 7.29-7.22 (m, 1H), 5.19-5.13 (m, 1H), 4.06 (br, 1H), 1.56 (s, 6H), 1.22 (d, J=6.4 Hz, 6H).

### Example 7: Synthesis of propan-2-yl 5-chloro-12-(3,4-difluorobenzoyl)-14,14-dimethyl-3,8,12-triazatricyclo[7.5.0.02,7]tetradeca-1(9),2,4,6,10-pentaene-10-carboxylate (44) See Also PCT/IB2015/002549 Herein Incorporated by Reference in its Entirety, Published as WO 2016/103037

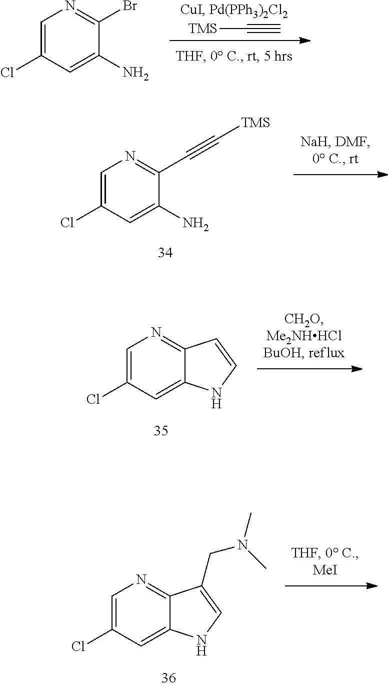

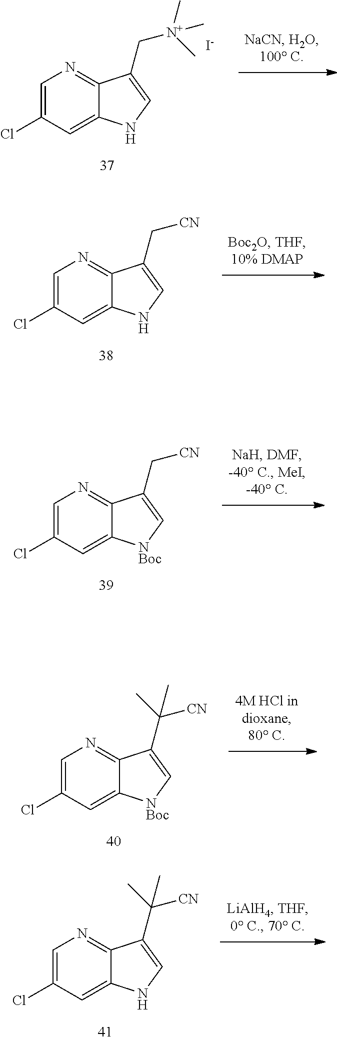 [SMILES: C[N+](C)(C)Cc1c[nH]c2cc(Cl)cnc12 ; [I-] ; N#CCc1c[nH]c2cc(Cl)cnc12 ; CC(C)(C)OC(=O)n1cc(CC#N)c2ncc(Cl)cc21 ; CC(C)(C)OC(=O)n1cc(C(C)(C)C#N)c2ncc(Cl)cc21 ; CC(C)(C#N)c1c[nH]c2cc(Cl)cnc12]

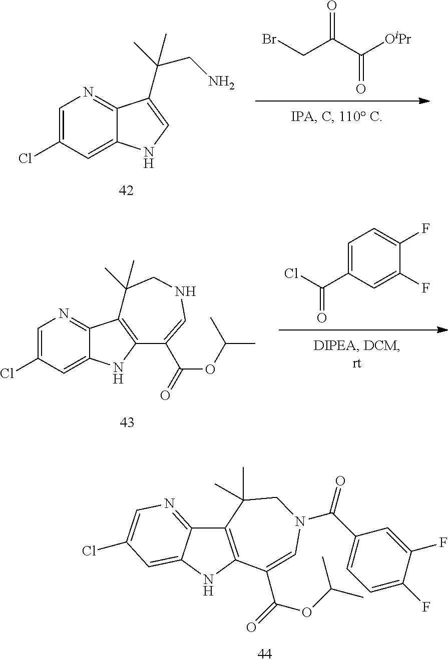 [SMILES: CC(C)(CN)c1c[nH]c2cc(Cl)cnc12 ; CC(C)OC(=O)C(=O)CBr ; CC(C)OC(=O)C1=CNCC(C)(C)c2c1[nH]c1cc(Cl)cnc21 ; O=C(Cl)c1ccc(F)c(F)c1 ; CC(C)OC(=O)C1=CN(C(=O)c2ccc(F)c(F)c2)CC(C)(C)c2c1[nH]c1cc(Cl)cnc21]

### Synthesis of 3-chloro-8-(3,4-difluorobenzoyl)-N-isopropyl-10,10-dimethyl-5,8,9,10-tetrahydropyrido[2′,3′:4,5]pyrrolo[2,3-d]azepine-6-carboxamide (85). General Procedure Applicable to Compounds of the Formulae (VII), (VIId), (VIII), and (VIIId)

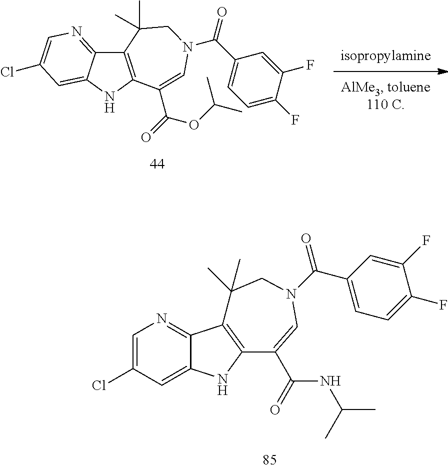 [SMILES: CC(C)OC(=O)C1=CN(C(=O)c2ccc(F)c(F)c2)CC(C)(C)c2c1[nH]c1cc(Cl)cnc21 ; CC(C)NC(=O)C1=CN(C(=O)c2ccc(F)c(F)c2)CC(C)(C)c2c1[nH]c1cc(Cl)cnc21]

To a solution of isopropyl amine (70 mg, 1.10 mol) in toluene, trimethyl aluminium [0.4 mL (2 M solution in toluene), 0.90 mol] was added at 0° C. After 15 minutes, compound 44 (110 mg, 0.23 mol) was added and reaction mixture was heated at 110° C. in a seal tube for 16 h. The reaction was quenched with ice water and extracted with ethyl acetate. The combined ethyl acetate layers were washed with water and brine, dried over sodium sulfate and concentrated under reduced pressure. The residue was purified on silica gel using 30% ethyl acetate in hexane to afford compound 85 (40 mg, 51%) as a yellow solid. LC-MS: 473.1.

Scheme 5 is generally applicable to compounds of Formula (XI)

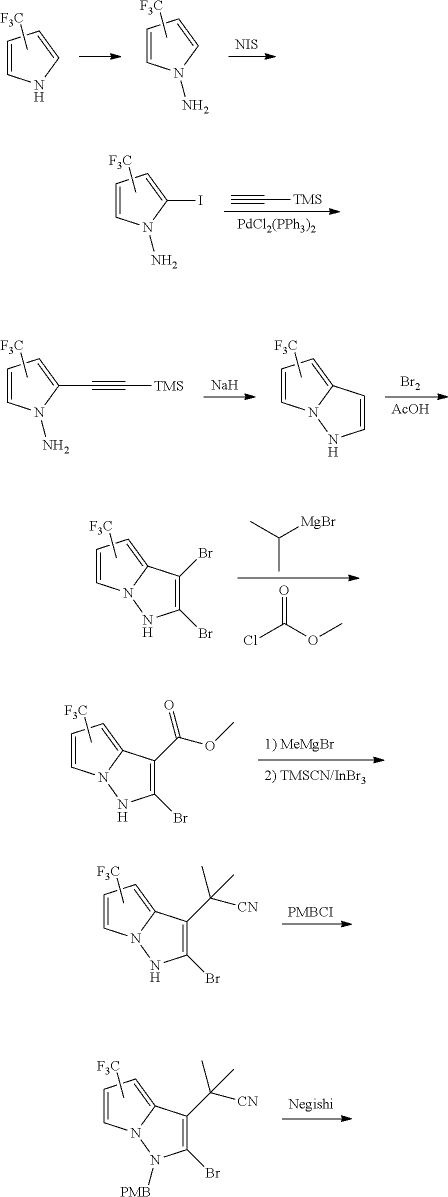

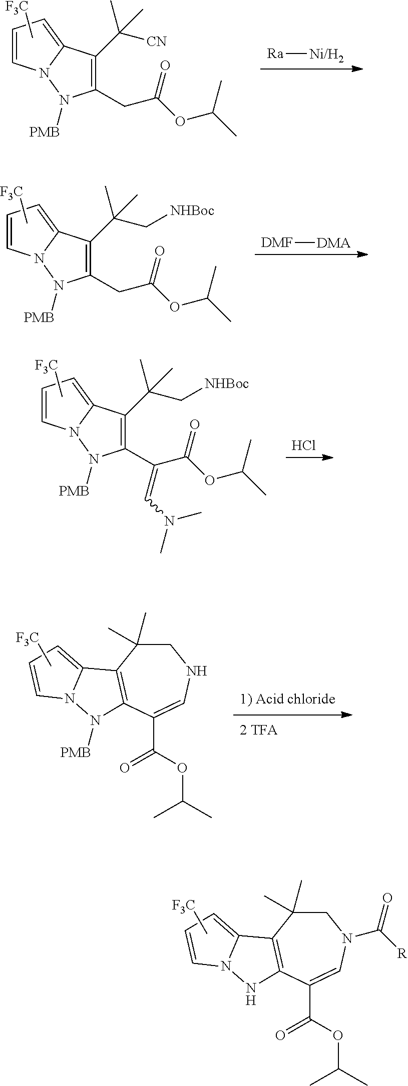

Scheme 6 is generally applicable to compounds of Formula (XII)

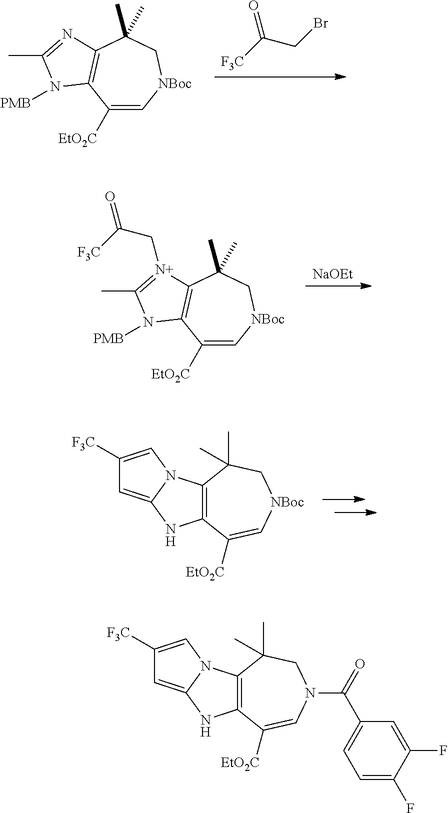

### Example 8: Phase 1 Study to Evaluate Safety of a Combination of an FXR Modulator and a DPP-IV Inhibitor in Subjects with Non-Alcoholic Steatohepatitis (NASH) and Advanced Fibrosis

The primary objective of this study is to characterize the safety, tolerability and dose-limiting toxicities (DLTs) for a combination of an FXR modulator and a DPP-IV inhibitor when administered orally to subjects with biopsy-proven NASH with advanced liver fibrosis.
The safety and tolerability of multiple doses of an FXR modulator and a DPP-IV inhibitor;
The effects of multiple doses of a combination of an FXR modulator and a DPP-IV inhibitor on insulin resistance and glucose homeostasis; and
Effects of a combination of an FXR modulator and a DPP-IV inhibitor on hepatocellular function as measured by assessment of liver enzymes and biochemical markers of hepatic and metabolic function and inflammation.

Patients: Eligible subjects will be men and women 18 years to 75 years of age.

Criteria:

Inclusion Criteria:
Institutional Review Board (IRB approved written Informed Consent and privacy language as per national regulation (eg, Health Insurance Portability and Accountability Act [HIPAA] Authorization for US sites) must be obtained from the subject or legally authorized representative prior to any study related procedures, including screening evaluations and tests
Subject is ≥18 years of age and <76 years old at the time of consent
Subject has had a percutaneous liver biopsy within 12 months from Screening that shows a definitive diagnosis of NASH with advanced (Brunt stage 3) hepatic fibrosis

Exclusion Criteria:
Subject is a pregnant or lactating female
Subject with current, significant alcohol consumption or a history of significant alcohol consumption for a period of more than 3 consecutive months any time within 1 year prior to screening. Significant alcohol consumption is defined as more than 20 gram per day in females and more than 30 grams per day in males, on average (a standard drink in the US is considered to be 14 grams of alcohol).
Subject is unable to reliably quantify alcohol consumption based upon local study physician judgment.
Subject uses drugs historically associated with nonalcoholic fatty liver disease (NAFLD) (amiodarone, methotrexate, systemic glucocorticoids, tetracyclines, tamoxifen, estrogens at doses greater than those used for hormone replacement, anabolic steroids, valproic acid, and other known hepatotoxins) for more than 2 weeks in the year prior to Screening.
Subject requires use of drugs with a narrow therapeutic window metabolized by CYP3A4 such as fast acting opioids (alfentanil and fentanyl), immunosuppressive drugs (cyclosporine, sirolimus, and tacrolimus), some cardiovascular agents (ergotamine, quinidine and dihydroergotamine), and select psychotropic agents (pimozide).
Subject has prior or has planned (during the study period) bariatric surgery (eg, gastroplasty, Roux-en-Y gastric bypass).
Subject has concurrent infection including diagnoses of fever of unknown origin and evidence of possible central line sepsis (subjects must be afebrile at the start of therapy).
Subject with a platelet count below 100,000/mm3 at Screening.
Subject with clinical evidence of hepatic decompensation as defined by the presence of any of the following abnormalities at Screening:
Serum albumin less than 3.5 grams/deciliter (g/dL).
An INR greater than 1.1.
Direct bilirubin greater than 1.3 milligrams per deciliter (mg/dL).
Subject has a history of bleeding esophageal varices, ascites or hepatic encephalopathy
Subject has a history of hepatitis C. Patients found on screening to have hepatitis C antibody, even if PCR negative for HCV RNA, are excluded from this study.
Subject has evidence of other forms of chronic liver disease:
Hepatitis B as defined by presence of hepatitis B surface antigen.
Evidence of ongoing autoimmune liver disease as defined by compatible liver histology.
Primary biliary cirrhosis as defined by the presence of at least 2 of these criteria (i) Biochemical evidence of cholestasis based mainly on alkaline phosphatase elevation (ii) Presence of anti-mitochondrial antibody (iii) Histologic evidence of nonsuppurative destructive cholangitis and destruction of interlobular bile ducts.
Primary sclerosing cholangitis.
Wilson's disease as defined by ceruloplasmin below the limits of normal and compatible liver histology.
Alpha-1-antitrypsin deficiency as defined by diagnostic features in liver histology (confirmed by alpha-1 antitrypsin level less than normal; exclusion at the discretion of the study physician).
History of hemochromatosis or iron overload as defined by presence of 3+ or 4+ stainable iron on liver biopsy.
Drug-induced liver disease as defined on the basis of typical exposure and history.
Known bile duct obstruction.
Suspected or proven liver cancer.
Any other type of liver disease other than NASH.
Subject with serum ALT greater than 300 units per liter (U/L) at Screening.
Subject with serum creatinine of 1.5 mg/dL or greater at Screening.
Subject using of any prescription or over-the-counter medication or herbal remedy that are believed to improve or treat NASH or liver disease or obesity during the period beginning 30 days prior to randomization. Subjects who are using Vitamin E or omega-3 fatty acids may continue their use.
Subject had major surgery within 8 weeks prior to Day 0, significant traumatic injury, or anticipation of need for major surgical procedure during the course of the study.
Subject with a history of biliary diversion.
Subject with known positivity for Human Immunodeficiency Virus infection.
Subject with an active, serious medical disease with likely life expectancy of less than 5 years.
Subject with active substance abuse, including inhaled or injection drugs, in the year prior to Screening.
Subject who has clinically significant and uncontrolled cardiovascular disease (eg, uncontrolled hypertension, myocardial infarction, unstable angina), New York Heart Association Grade II or greater congestive heart failure, serious cardiac arrhythmia requiring medication, or Grade II or greater peripheral vascular disease within 12 months prior to Day 0.
Subject has participated in an investigational new drug (IND) trial in the 30 days before randomization.
Subject has a clinically significant medical or psychiatric condition considered a high risk for participation in an investigational study.
Subject has any other condition which, in the opinion of the Investigator, would impede compliance or hinder completion of the study.
Subject has been previously exposed to GR MD 02.
Subject with known allergies to the study drug or any of its excipients.
Subject with malignant disease (other than basal and squamous cell carcinoma of the skin and in situ carcinoma of the cervix) with at least 5 years of follow-up showing no recurrence.
Subject has an abnormal chest x-ray indicative of acute or chronic lung disease on screening examination.

Study Design:
Allocation: Randomized
Endpoint Classification: Safety/Efficacy Study
Intervention Model: Parallel Assignment
Masking: Double Blind (Subject, Investigator)
Primary Purpose: Treatment

Primary Outcome Measures:
The primary objective of this study is to characterize the safety, which includes the tolerability and dose-limiting toxicity (DLT), for a combination of an FXR modulator and a DPP-IV inhibitor when administered to subjects with biopsy-proven NASH with advanced liver fibrosis. Specifically, this measure will be assessed by number of subjects experiencing treatment emergent adverse events indicative of DLT.

Secondary Outcome Measures:
A secondary objective is to evaluate change in serum alanine aminotransferase (ALT), aspartate aminotransferase (AST), ratio of AST:ALT, alkaline phosphatase, and gamma glutamyl transpeptidase (GGTP); change in AST/platelet ratio index. [Time Frame: Baseline; Week 7 (End of Study)] [Designated as safety issue: No]
A secondary objective for this study is to evaluate change in serum alanine aminotransferase (ALT), aspartate aminotransferase (AST), ratio of AST:ALT, alkaline phosphatase, and gamma glutamyl transpeptidase (GGTP) levels; and change in AST/platelet ratio index.
A secondary objective for this study is to evaluate changes in exploratory pharmacodynamic biomarkers in serum [Time Frame: Baseline; Week 7 (End of Study)] [Designated as safety issue: No]
A secondary objective for this study is to evaluate levels of exploratory pharmacodynamic biomarkers in serum including galectin-3, inflammatory, cell-death, and fibrosis markers.
Hepatocellular function as measured by assessment of liver enzymes and biochemical markers of hepatic and metabolic function.

| Arms | Assigned Interventions |
|---|---|
| Active Comparator: Cohort 1 | Drug: Compound 1, Compound 2, |
| Patient receives dose of | or Compound 3 |
| Compound 1, Compound 2, or | Drug: Placebo |
| Compound 3 or Placebo | |
| Active Comparator: Cohort 2 | Drug: Compound 1, Compound 2, |
| Patient receives dose of | or Compound 3 |
| Compound 1, Compound 2, or | Drug: Placebo |
| Compound 3 or Placebo | |
| Active Comparator: Cohort 3 | Drug: Compound 1, Compound 2, |
| Patient receives dose of | or Compound 3 |
| Compound 1, Compound 2, or | Drug: Placebo |
| Compound 3 or Placebo | |

This study is a dose ranging study to assess in sequential fashion, the safety, tolerability, and dose limiting toxicities (DLTs) of a combination of an FXR modulator and a DPP-IV inhibitor in subjects with biopsy-proven NASH with advanced fibrosis.

The examples and embodiments described herein are for illustrative purposes only and in some embodiments, various modifications or changes are to be included within the purview of disclosure and scope of the appended claims.

### Example 9: Phase 1 Study to Evaluate Safety of a Combination of an FXR Modulator and an SGL2 Inhibitor in Subjects with Non-Alcoholic Steatohepatitis (NASH) and Advanced Fibrosis

The primary objective of this study is to characterize the safety, tolerability and dose-limiting toxicities (DLTs) for a combination of an FXR modulator and an SGLT2 inhibitor when administered orally to subjects with biopsy-proven NASH with advanced liver fibrosis.
The safety and tolerability of multiple doses of an FXR modulator and an SGLT2 inhibitor;
The effects of multiple doses of a combination of an FXR modulator and an SGLT2 inhibitor on insulin resistance and glucose homeostasis; and
Effects of a combination of an FXR modulator and an SGLT2 inhibitor on hepatocellular function as measured by assessment of liver enzymes and biochemical markers of hepatic and metabolic function and inflammation.

Patients: Eligible subjects will be men and women 18 years to 75 years of age.

Criteria:

Inclusion Criteria:
Institutional Review Board (IRB approved written Informed Consent and privacy language as per national regulation (eg, Health Insurance Portability and Accountability Act [HIPAA] Authorization for US sites) must be obtained from the subject or legally authorized representative prior to any study related procedures, including screening evaluations and tests
Subject is ≥18 years of age and <76 years old at the time of consent
Subject has had a percutaneous liver biopsy within 12 months from Screening that shows a definitive diagnosis of NASH with advanced (Brunt stage 3) hepatic fibrosis

Exclusion Criteria:
Subject is a pregnant or lactating female
Subject with current, significant alcohol consumption or a history of significant alcohol consumption for a period of more than 3 consecutive months any time within 1 year prior to screening. Significant alcohol consumption is defined as more than 20 gram per day in females and more than 30 grams per day in males, on average (a standard drink in the US is considered to be 14 grams of alcohol).
Subject is unable to reliably quantify alcohol consumption based upon local study physician judgment.
Subject uses drugs historically associated with nonalcoholic fatty liver disease (NAFLD) (amiodarone, methotrexate, systemic glucocorticoids, tetracyclines, tamoxifen, estrogens at doses greater than those used for hormone replacement, anabolic steroids, valproic acid, and other known hepatotoxins) for more than 2 weeks in the year prior to Screening.
Subject requires use of drugs with a narrow therapeutic window metabolized by CYP3A4 such as fast acting opioids (alfentanil and fentanyl), immunosuppressive drugs (cyclosporine, sirolimus, and tacrolimus), some cardiovascular agents (ergotamine, quinidine and dihydroergotamine), and select psychotropic agents (pimozide).
Subject has prior or has planned (during the study period) bariatric surgery (eg, gastroplasty, Roux-en-Y gastric bypass).
Subject has concurrent infection including diagnoses of fever of unknown origin and evidence of possible central line sepsis (subjects must be afebrile at the start of therapy).
Subject with a platelet count below 100,000/mm3 at Screening.
Subject with clinical evidence of hepatic decompensation as defined by the presence of any of the following abnormalities at Screening:
Serum albumin less than 3.5 grams/deciliter (g/dL).
An INR greater than 1.1.
Direct bilirubin greater than 1.3 milligrams per deciliter (mg/dL).
Subject has a history of bleeding esophageal varices, ascites or hepatic encephalopathy
Subject has a history of hepatitis C. Patients found on screening to have hepatitis C antibody, even if PCR negative for HCV RNA, are excluded from this study.
Subject has evidence of other forms of chronic liver disease:
Hepatitis B as defined by presence of hepatitis B surface antigen.
Evidence of ongoing autoimmune liver disease as defined by compatible liver histology.
Primary biliary cirrhosis as defined by the presence of at least 2 of these criteria (i) Biochemical evidence of cholestasis based mainly on alkaline phosphatase elevation (ii) Presence of anti-mitochondrial antibody (iii) Histologic evidence of nonsuppurative destructive cholangitis and destruction of interlobular bile ducts.
Primary sclerosing cholangitis.
Wilson's disease as defined by ceruloplasmin below the limits of normal and compatible liver histology.
Alpha-1-antitrypsin deficiency as defined by diagnostic features in liver histology (confirmed by alpha-1 antitrypsin level less than normal; exclusion at the discretion of the study physician).
History of hemochromatosis or iron overload as defined by presence of 3+ or 4+ stainable iron on liver biopsy.
Drug-induced liver disease as defined on the basis of typical exposure and history.
Known bile duct obstruction.
Suspected or proven liver cancer.
Any other type of liver disease other than NASH.
Subject with serum ALT greater than 300 units per liter (U/L) at Screening.
Subject with serum creatinine of 1.5 mg/dL or greater at Screening.
Subject using of any prescription or over-the-counter medication or herbal remedy that are believed to improve or treat NASH or liver disease or obesity during the period beginning 30 days prior to randomization. Subjects who are using Vitamin E or omega-3 fatty acids may continue their use.
Subject had major surgery within 8 weeks prior to Day 0, significant traumatic injury, or anticipation of need for major surgical procedure during the course of the study.
Subject with a history of biliary diversion.
Subject with known positivity for Human Immunodeficiency Virus infection.
Subject with an active, serious medical disease with likely life expectancy of less than 5 years.
Subject with active substance abuse, including inhaled or injection drugs, in the year prior to Screening.
Subject who has clinically significant and uncontrolled cardiovascular disease (eg, uncontrolled hypertension, myocardial infarction, unstable angina), New York Heart Association Grade II or greater congestive heart failure, serious cardiac arrhythmia requiring medication, or Grade II or greater peripheral vascular disease within 12 months prior to Day 0.
Subject has participated in an investigational new drug (IND) trial in the 30 days before randomization.
Subject has a clinically significant medical or psychiatric condition considered a high risk for participation in an investigational study.
Subject has any other condition which, in the opinion of the Investigator, would impede compliance or hinder completion of the study.
Subject has been previously exposed to GR MD 02.
Subject with known allergies to the study drug or any of its excipients.
Subject with malignant disease (other than basal and squamous cell carcinoma of the skin and in situ carcinoma of the cervix) with at least 5 years of follow-up showing no recurrence.
Subject has an abnormal chest x-ray indicative of acute or chronic lung disease on screening examination.

Study Design:
Allocation: Randomized
Endpoint Classification: Safety/Efficacy Study
Intervention Model: Parallel Assignment
Masking: Double Blind (Subject, Investigator)
Primary Purpose: Treatment

Primary Outcome Measures:
The primary objective of this study is to characterize the safety, which includes the tolerability and dose-limiting toxicity (DLT), for a combination of an FXR modulator and an SGLT2 inhibitor when administered to subjects with biopsy-proven NASH with advanced liver fibrosis. Specifically, this measure will be assessed by number of subjects experiencing treatment emergent adverse events indicative of DLT.

Secondary Outcome Measures:
A secondary objective is to evaluate change in serum alanine aminotransferase (ALT), aspartate aminotransferase (AST), ratio of AST:ALT, alkaline phosphatase, and gamma glutamyl transpeptidase (GGTP); change in AST/platelet ratio index. [Time Frame: Baseline; Week 7 (End of Study)] [Designated as safety issue: No]
A secondary objective for this study is to evaluate change in serum alanine aminotransferase (ALT), aspartate aminotransferase (AST), ratio of AST:ALT, alkaline phosphatase, and gamma glutamyl transpeptidase (GGTP) levels; and change in AST/platelet ratio index.
A secondary objective for this study is to evaluate changes in exploratory pharmacodynamic biomarkers in serum [Time Frame: Baseline; Week 7 (End of Study)] [Designated as safety issue: No]
A secondary objective for this study is to evaluate levels of exploratory pharmacodynamic biomarkers in serum including galectin-3, inflammatory, cell-death, and fibrosis markers.
Hepatocellular function as measured by assessment of liver enzymes and biochemical markers of hepatic and metabolic function.

This study is a dose ranging study to assess in sequential fashion, the safety, tolerability, and dose limiting toxicities (DLTs) of a combination of an FXR modulator and an SGLT2 inhibitor in subjects with biopsy-proven NASH with advanced fibrosis.

### Example 10: Phase 1 Study to Evaluate Safety of a Combination of an FXR Modulator and an ASK1 Inhibitor in Subjects with Non-Alcoholic Steatohepatitis (NASH) and Advanced Fibrosis

The primary objective of this study is to characterize the safety, tolerability and dose-limiting toxicities (DLTs) for a combination of an FXR modulator and an ASK1 inhibitor when administered orally to subjects with biopsy-proven NASH with advanced liver fibrosis.
The safety and tolerability of multiple doses of an FXR modulator and an ASK1 inhibitor;
The effects of multiple doses of a combination of an FXR modulator and an ASK1 inhibitor on insulin resistance and glucose homeostasis; and
Effects of a combination of an FXR modulator and an ASK1 inhibitor on hepatocellular function as measured by assessment of liver enzymes and biochemical markers of hepatic and metabolic function and inflammation.

Patients: Eligible subjects will be men and women 18 years to 75 years of age.

Criteria:

Inclusion Criteria:
Institutional Review Board (IRB approved written Informed Consent and privacy language as per national regulation (eg, Health Insurance Portability and Accountability Act [HIPAA] Authorization for US sites) must be obtained from the subject or legally authorized representative prior to any study related procedures, including screening evaluations and tests
Subject is ≥18 years of age and <76 years old at the time of consent
Subject has had a percutaneous liver biopsy within 12 months from Screening that shows a definitive diagnosis of NASH with advanced (Brunt stage 3) hepatic fibrosis

Exclusion Criteria:
Subject is a pregnant or lactating female
Subject with current, significant alcohol consumption or a history of significant alcohol consumption for a period of more than 3 consecutive months any time within 1 year prior to screening. Significant alcohol consumption is defined as more than 20 gram per day in females and more than 30 grams per day in males, on average (a standard drink in the US is considered to be 14 grams of alcohol).
Subject is unable to reliably quantify alcohol consumption based upon local study physician judgment.
Subject uses drugs historically associated with nonalcoholic fatty liver disease (NAFLD) (amiodarone, methotrexate, systemic glucocorticoids, tetracyclines, tamoxifen, estrogens at doses greater than those used for hormone replacement, anabolic steroids, valproic acid, and other known hepatotoxins) for more than 2 weeks in the year prior to Screening.
Subject requires use of drugs with a narrow therapeutic window metabolized by CYP3A4 such as fast acting opioids (alfentanil and fentanyl), immunosuppressive drugs (cyclosporine, sirolimus, and tacrolimus), some cardiovascular agents (ergotamine, quinidine and dihydroergotamine), and select psychotropic agents (pimozide).
Subject has prior or has planned (during the study period) bariatric surgery (eg, gastroplasty, Roux-en-Y gastric bypass).
Subject has concurrent infection including diagnoses of fever of unknown origin and evidence of possible central line sepsis (subjects must be afebrile at the start of therapy).
Subject with a platelet count below 100,000/mm3 at Screening.
Subject with clinical evidence of hepatic decompensation as defined by the presence of any of the following abnormalities at Screening:
Serum albumin less than 3.5 grams/deciliter (g/dL).
An INR greater than 1.1.
Direct bilirubin greater than 1.3 milligrams per deciliter (mg/dL).
Subject has a history of bleeding esophageal varices, ascites or hepatic encephalopathy
Subject has a history of hepatitis C. Patients found on screening to have hepatitis C antibody, even if PCR negative for HCV RNA, are excluded from this study.
Subject has evidence of other forms of chronic liver disease:
Hepatitis B as defined by presence of hepatitis B surface antigen.
Evidence of ongoing autoimmune liver disease as defined by compatible liver histology.
Primary biliary cirrhosis as defined by the presence of at least 2 of these criteria (i) Biochemical evidence of cholestasis based mainly on alkaline phosphatase elevation (ii) Presence of anti-mitochondrial antibody (iii) Histologic evidence of nonsuppurative destructive cholangitis and destruction of interlobular bile ducts.
Primary sclerosing cholangitis.
Wilson's disease as defined by ceruloplasmin below the limits of normal and compatible liver histology.
Alpha-1-antitrypsin deficiency as defined by diagnostic features in liver histology (confirmed by alpha-1 antitrypsin level less than normal; exclusion at the discretion of the study physician).
History of hemochromatosis or iron overload as defined by presence of 3+ or 4+ stainable iron on liver biopsy.
Drug-induced liver disease as defined on the basis of typical exposure and history.
Known bile duct obstruction.
Suspected or proven liver cancer.
Any other type of liver disease other than NASH.
Subject with serum ALT greater than 300 units per liter (U/L) at Screening.
Subject with serum creatinine of 1.5 mg/dL or greater at Screening.
Subject using of any prescription or over-the-counter medication or herbal remedy that are believed to improve or treat NASH or liver disease or obesity during the period beginning 30 days prior to randomization. Subjects who are using Vitamin E or omega-3 fatty acids may continue their use.
Subject had major surgery within 8 weeks prior to Day 0, significant traumatic injury, or anticipation of need for major surgical procedure during the course of the study.
Subject with a history of biliary diversion.
Subject with known positivity for Human Immunodeficiency Virus infection.
Subject with an active, serious medical disease with likely life expectancy of less than 5 years.
Subject with active substance abuse, including inhaled or injection drugs, in the year prior to Screening.
Subject who has clinically significant and uncontrolled cardiovascular disease (eg, uncontrolled hypertension, myocardial infarction, unstable angina), New York Heart Association Grade II or greater congestive heart failure, serious cardiac arrhythmia requiring medication, or Grade II or greater peripheral vascular disease within 12 months prior to Day 0.
Subject has participated in an investigational new drug (IND) trial in the 30 days before randomization.
Subject has a clinically significant medical or psychiatric condition considered a high risk for participation in an investigational study.
Subject has any other condition which, in the opinion of the Investigator, would impede compliance or hinder completion of the study.
Subject has been previously exposed to GR MD 02.
Subject with known allergies to the study drug or any of its excipients.
Subject with malignant disease (other than basal and squamous cell carcinoma of the skin and in situ carcinoma of the cervix) with at least 5 years of follow-up showing no recurrence.
Subject has an abnormal chest x-ray indicative of acute or chronic lung disease on screening examination.

Study Design:
Allocation: Randomized
Endpoint Classification: Safety/Efficacy Study
Intervention Model: Parallel Assignment
Masking: Double Blind (Subject, Investigator)
Primary Purpose: Treatment

Primary Outcome Measures:
The primary objective of this study is to characterize the safety, which includes the tolerability and dose-limiting toxicity (DLT), for a combination of an FXR modulator and an ASK1 inhibitor when administered to subjects with biopsy-proven NASH with advanced liver fibrosis. Specifically, this measure will be assessed by number of subjects experiencing treatment emergent adverse events indicative of DLT.

Secondary Outcome Measures:
A secondary objective is to evaluate change in serum alanine aminotransferase (ALT), aspartate aminotransferase (AST), ratio of AST:ALT, alkaline phosphatase, and gamma glutamyl transpeptidase (GGTP); change in AST/platelet ratio index. [Time Frame: Baseline; Week 7 (End of Study)] [Designated as safety issue: No]
A secondary objective for this study is to evaluate change in serum alanine aminotransferase (ALT), aspartate aminotransferase (AST), ratio of AST:ALT, alkaline phosphatase, and gamma glutamyl transpeptidase (GGTP) levels; and change in AST/platelet ratio index.
A secondary objective for this study is to evaluate changes in exploratory pharmacodynamic biomarkers in serum [Time Frame: Baseline; Week 7 (End of Study)] [Designated as safety issue: No]
A secondary objective for this study is to evaluate levels of exploratory pharmacodynamic biomarkers in serum including galectin-3, inflammatory, cell-death, and fibrosis markers.
Hepatocellular function as measured by assessment of liver enzymes and biochemical markers of hepatic and metabolic function.

This study is a dose ranging study to assess in sequential fashion, the safety, tolerability, and dose limiting toxicities (DLTs) of a combination of an FXR modulator and an ASK1 inhibitor in subjects with biopsy-proven NASH with advanced fibrosis.

### Example 11: Phase 1 Study to Evaluate Safety of a Combination of an FXR Modulator and an GLP-1 Agonist in Subjects with Non-Alcoholic Steatohepatitis (NASH) and Advanced Fibrosis

The primary objective of this study is to characterize the safety, tolerability and dose-limiting toxicities (DLTs) for a combination of an FXR modulator and a GLP-1 agonist when administered orally to subjects with biopsy-proven NASH with advanced liver fibrosis.
The safety and tolerability of multiple doses of an FXR modulator and a GLP-1 agonist;
The effects of multiple doses of a combination of an FXR modulator and a GLP-1 agonist on insulin resistance and glucose homeostasis; and
Effects of a combination of an FXR modulator and a GLP-1 agonist on hepatocellular function as measured by assessment of liver enzymes and biochemical markers of hepatic and metabolic function and inflammation.

Patients: Eligible subjects will be men and women 18 years to 75 years of age.

Criteria:

Inclusion Criteria:
Institutional Review Board (IRB approved written Informed Consent and privacy language as per national regulation (eg, Health Insurance Portability and Accountability Act [HIPAA] Authorization for US sites) must be obtained from the subject or legally authorized representative prior to any study related procedures, including screening evaluations and tests
Subject is ≥18 years of age and <76 years old at the time of consent
Subject has had a percutaneous liver biopsy within 12 months from Screening that shows a definitive diagnosis of NASH with advanced (Brunt stage 3) hepatic fibrosis

Exclusion Criteria:
Subject is a pregnant or lactating female
Subject with current, significant alcohol consumption or a history of significant alcohol consumption for a period of more than 3 consecutive months any time within 1 year prior to screening. Significant alcohol consumption is defined as more than 20 gram per day in females and more than 30 grams per day in males, on average (a standard drink in the US is considered to be 14 grams of alcohol).
Subject is unable to reliably quantify alcohol consumption based upon local study physician judgment.
Subject uses drugs historically associated with nonalcoholic fatty liver disease (NAFLD) (amiodarone, methotrexate, systemic glucocorticoids, tetracyclines, tamoxifen, estrogens at doses greater than those used for hormone replacement, anabolic steroids, valproic acid, and other known hepatotoxins) for more than 2 weeks in the year prior to Screening.
Subject requires use of drugs with a narrow therapeutic window metabolized by CYP3A4 such as fast acting opioids (alfentanil and fentanyl), immunosuppressive drugs (cyclosporine, sirolimus, and tacrolimus), some cardiovascular agents (ergotamine, quinidine and dihydroergotamine), and select psychotropic agents (pimozide).
Subject has prior or has planned (during the study period) bariatric surgery (eg, gastroplasty, Roux-en-Y gastric bypass).
Subject has concurrent infection including diagnoses of fever of unknown origin and evidence of possible central line sepsis (subjects must be afebrile at the start of therapy).
Subject with a platelet count below 100,000/mm3 at Screening.
Subject with clinical evidence of hepatic decompensation as defined by the presence of any of the following abnormalities at Screening:
Serum albumin less than 3.5 grams/deciliter (g/dL).
An INR greater than 1.1.
Direct bilirubin greater than 1.3 milligrams per deciliter (mg/dL).
Subject has a history of bleeding esophageal varices, ascites or hepatic encephalopathy
Subject has a history of hepatitis C. Patients found on screening to have hepatitis C antibody, even if PCR negative for HCV RNA, are excluded from this study.
Subject has evidence of other forms of chronic liver disease:
Hepatitis B as defined by presence of hepatitis B surface antigen.
Evidence of ongoing autoimmune liver disease as defined by compatible liver histology.
Primary biliary cirrhosis as defined by the presence of at least 2 of these criteria (i) Biochemical evidence of cholestasis based mainly on alkaline phosphatase elevation (ii) Presence of anti-mitochondrial antibody (iii) Histologic evidence of nonsuppurative destructive cholangitis and destruction of interlobular bile ducts.
Primary sclerosing cholangitis.
Wilson's disease as defined by ceruloplasmin below the limits of normal and compatible liver histology.
Alpha-1-antitrypsin deficiency as defined by diagnostic features in liver histology (confirmed by alpha-1 antitrypsin level less than normal; exclusion at the discretion of the study physician).
History of hemochromatosis or iron overload as defined by presence of 3+ or 4+ stainable iron on liver biopsy.
Drug-induced liver disease as defined on the basis of typical exposure and history.
Known bile duct obstruction.
Suspected or proven liver cancer.
Any other type of liver disease other than NASH.
Subject with serum ALT greater than 300 units per liter (U/L) at Screening.
Subject with serum creatinine of 1.5 mg/dL or greater at Screening.
Subject using of any prescription or over-the-counter medication or herbal remedy that are believed to improve or treat NASH or liver disease or obesity during the period beginning 30 days prior to randomization. Subjects who are using Vitamin E or omega-3 fatty acids may continue their use.
Subject had major surgery within 8 weeks prior to Day 0, significant traumatic injury, or anticipation of need for major surgical procedure during the course of the study.
Subject with a history of biliary diversion.
Subject with known positivity for Human Immunodeficiency Virus infection.
Subject with an active, serious medical disease with likely life expectancy of less than 5 years.
Subject with active substance abuse, including inhaled or injection drugs, in the year prior to Screening.
Subject who has clinically significant and uncontrolled cardiovascular disease (eg, uncontrolled hypertension, myocardial infarction, unstable angina), New York Heart Association Grade II or greater congestive heart failure, serious cardiac arrhythmia requiring medication, or Grade II or greater peripheral vascular disease within 12 months prior to Day 0.
Subject has participated in an investigational new drug (IND) trial in the 30 days before randomization.
Subject has a clinically significant medical or psychiatric condition considered a high risk for participation in an investigational study.
Subject has any other condition which, in the opinion of the Investigator, would impede compliance or hinder completion of the study.
Subject has been previously exposed to GR MD 02.
Subject with known allergies to the study drug or any of its excipients.
Subject with malignant disease (other than basal and squamous cell carcinoma of the skin and in situ carcinoma of the cervix) with at least 5 years of follow-up showing no recurrence.
Subject has an abnormal chest x-ray indicative of acute or chronic lung disease on screening examination.

Study Design:
Allocation: Randomized
Endpoint Classification: Safety/Efficacy Study
Intervention Model: Parallel Assignment
Masking: Double Blind (Subject, Investigator)
Primary Purpose: Treatment

Primary Outcome Measures:
The primary objective of this study is to characterize the safety, which includes the tolerability and dose-limiting toxicity (DLT), for a combination of an FXR modulator and a GLP-1 agonist when administered to subjects with biopsy-proven NASH with advanced liver fibrosis. Specifically, this measure will be assessed by number of subjects experiencing treatment emergent adverse events indicative of DLT.

Secondary Outcome Measures:
A secondary objective is to evaluate change in serum alanine aminotransferase (ALT), aspartate aminotransferase (AST), ratio of AST:ALT, alkaline phosphatase, and gamma glutamyl transpeptidase (GGTP); change in AST/platelet ratio index. [Time Frame: Baseline; Week 7 (End of Study)] [Designated as safety issue: No]
A secondary objective for this study is to evaluate change in serum alanine aminotransferase (ALT), aspartate aminotransferase (AST), ratio of AST:ALT, alkaline phosphatase, and gamma glutamyl transpeptidase (GGTP) levels; and change in AST/platelet ratio index.
A secondary objective for this study is to evaluate changes in exploratory pharmacodynamic biomarkers in serum [Time Frame: Baseline; Week 7 (End of Study)] [Designated as safety issue: No]
A secondary objective for this study is to evaluate levels of exploratory pharmacodynamic biomarkers in serum including galectin-3, inflammatory, cell-death, and fibrosis markers.
Hepatocellular function as measured by assessment of liver enzymes and biochemical markers of hepatic and metabolic function.

This study is a dose ranging study to assess in sequential fashion, the safety, tolerability, and dose limiting toxicities (DLTs) of a combination of an FXR modulator and a GLP-1 agonist in subjects with biopsy-proven NASH with advanced fibrosis.

### Example 12: Phase 2 Study of Compound 1, Compound 2, or Compound 3 to Treat Primary Biliary Cirrhosis

The purpose of this study is to determine if Compound 1, Compound 2, or Compound 3 has an effect on cholesterol levels in the blood in patients with primary biliary cirrhosis (PBC).

### Study Type: Interventional

### Study Design: Endpoint Classification: Safety/Efficacy Study

### Intervention Model: Single Group Assignment

Primary Outcome Measures:
Change from baseline in High-density lipoprotein (HDL) Metabolism [Time Frame: Week 4, Week 8 and Week 12] [Designated as safety issue: No]
HDL metabolism will be assessed by measuring HDL cholesterol concentration, HDL particle size and number.

Secondary Outcome Measures:
Change from baseline in Lipoprotein Metabolism [Time Frame: Week 4, Week 8 and Week 12] [Designated as safety issue: No]
Lipoprotein metabolism will be assessed by measuring the following:
   concentrations of total cholesterol and triglycerides
   Low-density lipoprotein (LDL) and very low density lipoprotein (VLDL) cholesterol concentrations, particle size and number
   concentrations of apolipoprotein A (ApoA), apolipoprotein B (ApoB), apolipoprotein E (ApoE), and lipoprotein (a) [Lp(a)]
Change from baseline Reverse Cholesterol Transport [Time Frame: Week 4, Week 8 and Week 12] [Designated as safety issue: No]
Components of reverse cholesterol transport will also be assessed as part of the lipoprotein analysis. This will include measurements of:
   HDL capacity to accept cholesterol measured by lecithin-cholesterol acyltransferase (LCAT) and Cholesterol ester transfer protein (CETP) activity.
      pre-β1 HDL concentration
      macrophage cholesterol efflux
Pharmacokinetic parameters of Compound 1, Compound 2, or Compound 3
[Time Frame: Week 8] [Designated as safety issue: No]
In a subset of patients who agree to participate, non-compartmental pharmacokinetic parameters of Compound 1, Compound 2, or Compound 3 will be assessed.

Other Outcome Measures:
Fasting levels of Compound 1, Compound 2, or Compound 3 [Time Frame: Week 8] [Designated as safety issue: No]
Change from baseline in fibroblast growth factor 19 (FGF-19) [Time Frame: Week 4, Week 8 and Week 12] [Designated as safety issue: No]
Change from baseline in Lipoprotein X [Time Frame: Week 4, Week 8 and Week 12] [Designated as safety issue: No]
Markers of inflammation including: C-Reactive Protein, GlycA and GlycB [Time Frame: Week 8] [Designated as safety issue: No]

| Arms | Assigned Interventions |
|---|---|
| Compound 1, Compound 2, | Drug: Compound 1, Compound 2, |
| or Compound 3, oral | or Compound 3 |
| administration, 8 weeks | All subjects will be treated |
| | with Compound 1, Compound 2, |
| | or Compound 3 (oral administration, |
| | 10 mg, once daily) for 8 weeks |
| | and should continue their prestudy |
| | dose of ursodeoxycholic acid |
| | (UDCA). After completion of the |
| | 8 week study period, subjects |
| | will be offered the opportunity |
| | to enter an open label long term |
| | safety extension for up to years. |

Eligibility

Ages Eligible for Study: 18 Years and older

### Genders Eligible for Study: Both

### Accepts Healthy Volunteers: No

Criteria

### Inclusion Criteria:

Definite or probable primary biliary cirrhosis (PBC) diagnosis as demonstrated by the presence of ≥2 of the following 3 diagnostic factors:
   History of elevated alkaline phosphatase (ALP) levels for at least 6 months
   A positive anti-microbial antibody (AMA) titer or, if AMA negative or in low titer (<1:80), PBC specific antibodies
   Liver biopsy consistent with PBC
Taking UDCA for at least 12 months (stable dose for ≥3 months) prior to Day 0 or unable to tolerate UDCA (no UDCA for ≥3 months prior to Day 0)
Contraception: Female subjects must be postmenopausal, surgically sterile, or if premenopausal, be prepared to use ≥1 effective (≤1% failure rate) method of contraception during the trial and until at least 30 days after the last dose of Investigational Product.

### Exclusion Criteria:

Subjects with decompensated PBC (as determined by the Investigator)
Severe pruritus or systemic treatment for pruritus (e.g. treatment with bile acid sequestrants or rifampicin) within 2 months of Day 0
History or presence of other significant liver diseases including:
   Active or chronic Hepatitis B or C virus (HBV, HCV) infection
   Primary sclerosing cholangitis (PSC)
   Alcoholic liver disease
   Definite autoimmune liver disease or overlap hepatitis
   Nonalcoholic steatohepatitis (NASH)
      NOTE: Subjects with Gilbert's disease or those with a history of hepatitis B who are currently antigen negative and seroconverted should not be considered exclusionary
Uncontrolled diabetes or other uncontrolled or unstable medical condition that may interfere with trial results
Administration of any of the following medications as specified below:
   Prohibited 28 days prior to Day 0: bile acid sequestrants (BAS) including cholestyramine, colesevelam, or colestipol
   Prohibited 3 months prior to Day 0 and throughout trial participation: serum-lipid modifying agents including 3-hydroxy-3-methylglutaryl-coenzyme A (HMG CoA) reductase inhibitors, fenofibrate or other fibrates, nicotinic acid and derivatives, ezetimibe, Vitamin E (other than as standard dietary supplement), omega-3 fatty acid containing dietary supplements
   Prohibited 6 months prior to Day 0 and throughout the trial participation: azathioprine, colchicine, cyclosporine, methotrexate, mycophenolate mofetil, pentoxifylline; budesonide and other systemic corticosteroids; potentially hepatotoxic drugs (including α-methyl-dopa, sodium valproic acid, isoniazide, or nitrofurantoin)
   Prohibited 12 months prior to Day 0 and throughout the trial participation: antibodies or immunotherapy directed against interleukins or other cytokines or chemokines
Planned change in diet or exercise habits during participation in the trial
Presence or history of clinically significant cardiac arrhythmias that may prohibit the subject from participating in the trial If female: known pregnancy, or has a positive urine pregnancy test (confirmed by a positive serum pregnancy test), or lactating.

### Example 13: Effect of Compound 1, Compound 2, or Compound 3 in the Treatment of Patients with Primary Sclerosing Cholangitis (PSC)

The purpose of this study is to evaluate the effect of Compound 1, Compound 2, or Compound 3 on liver biochemistry, in particular, serum alkaline phosphatase; and, safety.

### Study Type: Interventional

### Study Design: Allocation: Randomized

### Endpoint Classification: Safety/Efficacy Study

### Intervention Model: Single Group Assignment

### Masking: Double Blind (Subject, Caregiver, Investigator)

### Primary Purpose: Treatment

Primary Outcome Measures:
Evaluate the effects of Compound on serum alkaline phosphatase (ALP) in subjects with primary sclerosing cholangitis (PSC) [Time Frame: 24 weeks] [Designated as safety issue: Yes]
Evaluate the effects of Compound on safety in subjects with PSC [Time Frame: 24 weeks] [Designated as safety issue: Yes]

Secondary Outcome Measures:
Hepatic biochemistry and indices of function [Time Frame: 24 weeks] [Designated as safety issue: Yes]
Hepatic Fibrosis [Time Frame: 24 weeks] [Designated as safety issue: Yes]
Gastrointestinal inflammation and disease [Time Frame: 24 weeks] [Designated as safety issue: Yes]
FXR) activity [Time Frame: 24 weeks] [Designated as safety issue: Yes]
Inflammatory bowel disease (IBD) [Time Frame: 24 weeks] [Designated as safety issue: Yes]
Long-term efficacy [Time Frame: 24 months] [Designated as safety issue: No]
Long term safety [Time Frame: 24 months] [Designated as safety issue: Yes]
Disease-specific symptoms [Time Frame: 24 weeks] [Designated as safety issue: Yes] Pharmacokinetics (PK) of compound [Time Frame: 24 Weeks] [Designated as safety issue: No]

| Arms | Assigned Interventions |
|---|---|
| Experimental: Low dose Compound 1, | Drug: Compound 1, Compound 2, |
| Compound 2, or Compound 3 titrating | or Compound 3 |
| to high dose Compound 1, Compound 2, | |
| or Compound 3 | |
| Subjects randomized to low dose will | |
| take low dose Compound 1, Compound 2, | |
| or Compound 3 daily for 12 weeks | |
| followed by high dose Compound 1, | |
| Compound 2, or Compound 3 for an | |
| additional 12 weeks. | |
| Experimental: Placebo | Drug: Placebo |
| Subjects randomized to placebo will | |
| take placebo for 24 weeks | |

This is a Phase 2, randomized, double-blind, placebo-controlled, dose-finding evaluation of the efficacy and safety of compound in subjects with PSC. Approximately 80 subjects who provide written informed consent and meet all of the inclusion and none of the exclusion criteria will be randomized to 1 of 2 treatment groups as follows: low dose titrating to high dose Compound 1, Compound 2, or Compound 3, or placebo, in a 1:1 ratio. Subjects will administer investigational product (IP) orally, once daily for 2 consecutive 12-week periods.

Any subjects whose dose is not titrated, due to safety or tolerability concerns, will remain on their starting treatment (low dose Compound 1, Compound 2, or Compound 3, or placebo) for the remainder of the double-blind phase to Week 24.

Randomization will be stratified by the presence or absence of concomitant ursodeoxycholic acid (UDCA) use and total bilirubin level (≤1.5× upper limit of normal [ULN] or >1.5×ULN but<2.5×ULN).

Eligibility

### Ages Eligible for Study: 18 Years to 75 Years

### Genders Eligible for Study: Both

### Accepts Healthy Volunteers: No

Criteria

### Inclusion Criteria:

Must have a diagnosis of PSC (based on cholangiography at any point in time) and must have had a cholangiography within the past 12 months
ALP at Screening ≥2×ULN
Total bilirubin at Screening <2.5×ULN.
For subjects with concomitant IBD:
   Colonoscopy (if subject has a colon) or other appropriate endoscopic procedure within 12 months of Day 0 confirming no dysplasia or colorectal cancer
   Subjects with Crohn's Disease (CD) must be in remission as defined by a Crohn's Disease Activity Index (CDAI)<150.
   Subjects with ulcerative colitis (UC) must either be in remission or have mild disease. Remission is defined as a partial Mayo score of ≤2 with no individual sub-score exceeding 1. Mild disease is defined as a partial Mayo score ≤3 with no individual sub-score exceeding 1 point.
For subjects being administered UDCA as part of their standard of care, the dose must have been stable for ≥3 months prior to, and including, Day 0 and must not have exceeded 20 mg/kg/day during this time.
Subjects being administered biologic treatments (eg, anti-tumor necrosis factor (TNF) or anti-integrin monoclonal antibodies), immunosuppressants, systemic corticosteroids, or statins, must have been on a stable dose for ≥3 months prior to, and including, Day 0 and should plan to remain on a stable dose throughout the trial.
Contraception: female subjects of childbearing potential must use ≥1 effective method (≤1% failure rate) of contraception during the trial and until 4 weeks following the last dose of IP (including long term safety extension doses).

### Exclusion Criteria:

Evidence of a secondary cause of sclerosing cholangitis at Screening
Immunoglobulin G4 (IgG4)>4×ULN at Screening or evidence of IgG4 sclerosing cholangitis
Small duct cholangitis in the absence of large duct disease
Presence of clinical complications of chronic liver disease or clinically significant hepatic decompensation, including:
Current Child Pugh classification B or C
History of, or current diagnosis or suspicion of, cholangiocarcinoma or other hepatobiliary malignancy, or biliary tract dysplasia.
History of liver transplantation, or current model of end stage liver disease score ≥12
History of, or current, cirrhosis with complications, including history or presence of spontaneous bacterial peritonitis hepatocellular carcinoma or hepatic encephalopathy (as assessed by the investigator)
Current known portal hypertension with complications, including known gastric or large esophageal varices, poorly controlled or diuretic resistant ascites, history of variceal bleeds, or related therapeutic or prophylactic interventions (e.g., beta blockers, insertion of variceal bands or transjugular intrahepatic portosystemic shunt [TIPS])
History of, or current, hepatorenal syndrome (type I or II) or Screening serum creatinine >2 mg/dL (178 μmol/L)
Platelet count <50×10⁹/L
Clinical evidence of dominant stricture (as evidenced by cholangiography or other appropriate imaging modality within the 12 months prior to Day 0) or current biliary stent
Current cholecystitis or gallstones (identified by hepatic imaging)
Colonic dysplasia within <5 years prior to Day 0
History of small bowel resection
History of other chronic liver diseases, including, but not limited to, primary biliary cirrhosis (PBC), alcoholic liver disease, non-alcoholic fatty liver disease (NAFLD), autoimmune hepatitis, hepatitis B virus (unless seroconverted and no positive Hepatitis B Virus DNA), hepatitis C virus and overlap syndrome
Known Gilbert's syndrome or history of elevations in unconjugated (indirect) bilirubin >ULN
Known history of human immunodeficiency virus (HIV) infection
Currently experiencing, or experienced within ≤3 months of Screening, pruritus requiring systemic or enteral treatment
Known or suspected acute cholangitis in the 3 months prior to, and including, Day 0 including cholangitis treated with antibiotics
Administration of antibiotics is prohibited ≤1 month of Day 0 (unless subject is on a stable prophylaxis dose for at least 3 months prior to Day 0).
Administration of the following medications is prohibited ≤6 months of Day 0 and throughout the trial: fenofibrate or other fibrates and potentially hepatotoxic medications (including α-methyl-dopa, sodium valproic acid, isoniazide, or nitrofurantoin).
IBD flare during Screening (up to and including Day 0), where “flare” is defined as follows:
   UC flare: partial Mayo Score ≥5, and
   CD flare: CDAI≥250
If female: known pregnancy, or has a positive urine pregnancy test (confirmed by a positive serum pregnancy test), or lactating
Other concomitant disease, malignancy, or condition likely to significantly decrease life expectancy to less than the duration of the trial (e.g., moderate to severe congestive heart failure)

### Example 14: Use of Compound 1, Compound 2, or Compound 3 for the Treatment of Biliary Atresia

The purpose of this study is to determine whether Compound 1, Compound 2, or Compound 3 reduces liver damage in infants with biliary atresia.

### Study Type: Interventional

### Study Design: Endpoint Classification: Efficacy Study

### Intervention Model: Single Group Assignment

### Masking: Open Label

### Primary Purpose: Treatment

Primary Outcome Measures:
Change in serum conjugated bilirubin [Time Frame: Baseline and after 90 days of therapy] [Designated as safety issue: No]

Secondary Outcome Measures:
Change in Weight [Time Frame: Baseline and after 90 days of therapy] [Designated as safety issue: No]
Change in serum markers [Time Frame: Baseline and up to two years after therapy finishes] [Designated as safety issue: No]
   The investigators will track the change in serum liver markers and platelets over the course of two years in patients receiving 90 days of Compound 1, Compound 2, or Compound 3 therapy.
Change in liver imaging [Time Frame: Baseline and up to two years after therapy finishes] [Designated as safety issue: No]
   The investigators will track liver ultrasound changes, including liver and spleen size.
Time to liver transplant [Time Frame: Baseline and up to two years after therapy finishes] [Designated as safety issue: No]

| Arms | Assigned Interventions |
|---|---|
| Experimental: Compound 1, Compound 2, | Drug: Compound 1, Compound 2, |
| or Compound 3 | or Compound 3 |
| All newly-diagnosed biliary atresia | 20 mg/kg/day divided in |
| patients fulfilling the study’s | 3 doses, given orally for |
| inclusion criteria will receive oral | 90 days |
| Compound 1, Compound 2, or Compound 3, | |
| 20 mg/kg/day divided in three doses for a | |
| total of 90 days. | |

Biliary atresia (BA) is a devastating liver disease of infancy of unknown etiology, characterized by bile duct obstruction, live fibrosis, and cirrhosis. BA has no known medical treatments. The only proven treatment is a surgical portoenterostomy (the Kasai procedure, or KP) which can achieve bile drainage and improve outcomes in some cases. The KPs success is variable depending on several factors including age of the infant, experience of the surgeon, and extent of liver fibrosis at the time of KP.

In this study, the investigators conduct a phase II trial of a potential new medical therapy for BA: Compound 1, Compound 2, or Compound 3. The trial's objective is to determine whether Compound 1, Compound 2, or Compound 3 has sufficient biological activity against BA to warrant further study. Compound 1, Compound 2, or Compound 3 will be administered orally for 90 days as an adjunct to standard therapy (i.e. KP if appropriate). The primary outcome will measure the change in serum conjugated bilirubin levels after 90 days. Secondary outcomes include changes in body weight, serum markers, liver imaging, and time to liver transplant in infants with BA.

Eligibility

### Ages Eligible for Study: up to 180 Days

### Genders Eligible for Study: Both

### Accepts Healthy Volunteers: No

Criteria

### Inclusion Criteria:

Diagnosed with biliary atresia through liver biopsy and/or intra-operative cholangiogram
No previous Kasai portoenterostomy performed at another institution
Able to take medications orally
Legal guardian signs consent after understanding risks and investigational nature of study

### Exclusion Criteria:

Infants greater than 180 days old
Infants receiving a Kasai portoenterostomy at another institution
Infants unable to take medications orally

### Example 15: Phase 2 Clinical Study of Compound 1, Compound 2, or Compound 3 for Inflammatory Bowel Disease

The purpose of this study is to evaluate the efficacy of the use of once daily use of Compound 1, Compound 2, or Compound 3 in subjects with symptomatic inflammatory bowel disease.

### Study Type: Interventional

### Study Design: Allocation: Randomized

### Endpoint Classification: Efficacy Study

### Intervention Model: Parallel Assignment

### Masking: Double Blind (Subject, Caregiver, Investigator, Outcomes Assessor)

### Primary Purpose: Treatment

Primary Outcome Measures:
An increase in the subjects inflammatory bowel disease questionnaire score [Time Frame: 6 weeks, 12 weeks, and 6 months] [Designated as safety issue: No]
   Participants will fill-out a questionnaire called the IBDQ at enrollment, 6 weeks, 12 weeks, and 6 months after enrollment. The IBDQ is a validated instrument often used in routine care and studies of patients with IBD. The IBDQ measures the activity of IBD and quality of life. It includes 32 questions placed into 4 domains: bowel, social, emotional and systemic. Each question is ranked from 1-7, 1 being the poorest quality of life and 7 being the best quality of life. A score of >170 means that a patient is clinically in remission and an increase in score between 16 and 32 are considered a meaningful improvement in symptoms.

| Arms | Assigned Interventions |
|---|---|
| Experimental: Compound 1, Compound 2, | Drug: Compound 1, Compound 2, |
| or Compound 3 | or Compound 3 daily for 12 weeks |
| Subjects in this arm will receive | |
| Compound 1, Compound 2, or Compound | |
| 3 daily for 12 weeks. | |
| Placebo Comparator: Placebo | Drug: Placebo |
| Subjects in this arm will receive a | |
| placebo daily for 12 weeks. | |

The investigators will compare the use of Compound 1, Compound 2, or Compound 3 compared with placebo in subjects that have symptomatic inflammatory bowel disease (IBD). Our subjects will be those with diagnosed with IBD and are symptomatic, defined by an inflammatory bowel disease questionnaire (IBDQ) score <170. The subjects will be randomly assigned either placebo or LDN. They will take the IBDQ prior to starting the trial, 6 weeks, 12 weeks and 6 months after starting the medication. Participants have to remain on their current IBD regimen throughout the trial and cannot make any changes within 4 weeks of starting the trial. The investigators will have a safety phone call at 6 weeks and a follow up letter at 12 weeks after starting the trial. The participants will be given a card to keep with them with a phone number and email address if any adverse effects arise.

Eligibility

Ages Eligible for Study: 18 Years and older

### Genders Eligible for Study: Both

### Accepts Healthy Volunteers: Yes

Criteria

### Inclusion Criteria:

Patients with symptomatic Crohn's disease or ulcerative colitis (defined as a response to the Inflammatory Bowel Disease Questionnaire less than 170)
Confirmed Crohn's disease or ulcerative colitis through radiographic, endoscopic and/or histologic criteria
On a stable dose of medication for IBD (i.e. no change in medication within 4 weeks of study enrollment)

### Exclusion Criteria:

Patients on opioids or Imodium within 7 days of starting the investigational therapy
Women who are breastfeeding, pregnant, or plan on becoming pregnant within the next year
Women of child bearing age not willing to use contraception or abstinence
A history of the following diseases or procedures:
   Acute hepatitis
   Liver failure
   Ileoanal anastomosis
   Short bowel syndrome
   Abnormal liver enzymes

### Example 16: Investigation of Compound 1, Compound 2, or Compound 3 in Patients with Irritable Bowel Syndrome

The purpose of this study is to determine if Compound 1, Compound 2, or Compound 3 helps people with irritable bowel syndrome.

Study Type: Interventional

Study Design: Allocation: Randomized

Endpoint Classification: Efficacy Study

Intervention Model: Parallel Assignment

Masking: Double Blind (Subject, Caregiver, Investigator)

Primary Purpose: Treatment

Primary Outcome Measures:
Primary aim: Collect preliminary data comparing effects of Compound 1, Compound 2, or Compound 3 and placebo on abdominal pain/discomfort on bowel symptom score (BSS), overall BSS score, and adequate relief of irritable bowel syndrome (IBS) symptoms in patients with IBS [Time Frame: weekly] [Designated as safety issue: No]

Secondary Outcome Measures:
To compare the effect of Compound 1, Compound 2, or Compound 3 and placebo on self-reported overall and individual BSS scores [Time Frame: Last 4 weeks of treatment] [Designated as safety issue: No]
To compare effect of Compound 1, Compound 2, or Compound 3 and placebo on adequate relief of IBS pain or discomfort at least 50% of the time [Time Frame: During the last 4 weeks of therapy] [Designated as safety issue: No]
To compare effect of Compound 1, Compound 2, or Compound 3 and placebo on overall and individual BSS scores [Time Frame: Week 12] [Designated as safety issue: No]
To compare the effect of Compound 1, Compound 2, or Compound 3 and placebo on the proportion of patients with at least 3 point changes in 11 point pain and IBS scores [Time Frame: 12 weeks] [Designated as safety issue: No]

| Arms | Assigned Interventions |
|---|---|
| Active Comparator: Compound 1, | Drug: Compound 1, Compound 2, |
| Compound 2, or Compound 3 | or Compound 3 |
| | Dose: 75 mg twice a day for |
| | three days, increasing to |
| | 150 mg (2 tablets) twice a day |
| | for three days, escalating to |
| | 225 mg (three tablets) twice |
| | a day, through week 12, day 1. |
| | Days 2-4 of week 12, |
| | participants will begin |
| | tapering and will receive 150 |
| | mg (two tablets) two times a |
| | day and then days 5-7, |
| | participants will receive 75 mg |
| | two times a day for the duration |
| | of the study. |
| Placebo Comparator: Placebo | Drug: Placebo |
| Placebo, not active | A matching placebo will be |
| | administered twice a day |

Eligibility

Ages Eligible for Study: 18 Years to 70 Years

Genders Eligible for Study: Both

Accepts Healthy Volunteers: No

Criteria

Inclusion Criteria:
Established diagnosis of IBS
Experience pain with relief with defecation
50/100 or greater of pain or discomfort scores during the two-week baseline period
At least three pain attacks in a month, with at least three episodes of pain intensity equal to or exceeding 50/100

Exclusion Criteria:
Known alternative/concurrent gastrointestinal diagnosis (e.g. Crohn's disease, ulcerative colitis, microscopic colitis, active celiac sprue, chronic pancreatitis or pancreatic insufficiency, scleroderma, chronic intestinal pseudo-obstruction, bacterial overgrowth, recent (<6 months) intestinal bacterial/protozoal/parasitic infections, HIV, active pelvic floor dysfunction, paraplegia or quadriplegia);
Current symptoms of severe depression, as measured by Hospital Anxiety and Depression Scale (HADS) score (greater or less than 15);
Planned surgery (especially transplant) or anesthesia exposure during trial
Are pregnant, lactating, likely to become pregnant during medication phase and not willing to use a reliable form of contraception (barrier contraceptives, diaphragm, injections, intrauterine device, surgical sterilization, or abstinence)
Significant acute or chronic progressive neurologic, hepatic, renal, cardiovascular, respiratory or metabolic disease
Recent history of alcohol or substance dependence use or abuse
Major cardiovascular events in the last 6 months
Use of IBS-specific drugs such as tegaserod (Zelnorm) and Lotronex (Alosetron) (within 30 days)
Participation in another clinical trial (within 30 days)

### Example 17: Phase 2 Clinical Study of Compound 1, Compound 2, or Compound 3 for Bile Acid Diarrhea

The investigators propose to develop studies of Compound 1, Compound 2, or Compound 3 in patients with bile acid diarrhea. Preliminary data suggests that patients with bile acid diarrhea have impaired production of the ileal hormone Fibroblast Growth Factor 19 (FGF19). FGF19 is stimulated by FXR agonists, and regulates bile acid synthesis. This study is a pilot, proof-of-concept, open-label study to investigate whether compound can stimulate FGF19 in bile acid diarrhea patients to provide a safe and effective treatment.

Study Type: Interventional

Study Design: Endpoint Classification: Pharmacodynamics Study

Intervention Model: Single Group Assignment

Masking: Open Label

Primary Purpose: Treatment

Primary Outcome Measures:
Fasting FGF19 [Time Frame: 15 days] [Designated as safety issue: No] The primary outcome measure is the change over 2 weeks in fasting serum fibroblast growth factor (FGF19) in 3 groups of patients: primary bile acid diarrhea, secondary bile acid diarrhea, and a control population of patients with chronic diarrhea but with normal bile acid retention.

Secondary Outcome Measures:
Non-fasting response of FGF19 to Compound 1, Compound 2, or Compound 3 [Time Frame: 15 days] [Designated as safety issue: No] Change in dynamic response of FGF19 in 6 hours following Compound 1, Compound 2, or Compound 3 administration; at start and end of 15 day Compound 1, Compound 2, or Compound 3 test period.

| Arms | Assigned Interventions |
|---|---|
| Experimental: Compound 1, | Drug: Compound 1, Compound 2, |
| Compound 2, or Compound 3 | or Compound 3 |
| 25 mg, once daily for 15 days. | Day-14 to Day 0 subjects will |
| | stop their usual diarrheal |
| | medication. Day 1 to Day 15 |
| | Compound 1, Compound 2, or |
| | Compound 3 25 mg tablet will |
| | be administered to subjects |
| | once daily in the morning. |
| | Day 16 to day 28 normal |
| | diarrheal medication may |
| | be re-commenced. |

Bile acid diarrhea (BAD) is an under-recognized but common condition of chronic watery diarrhea. BAD may be secondary to ileal disease affecting the reabsorption and the enterohepatic circulation of bile acids (bile acid malabsorption) or can be an idiopathic, primary BAD (PBAD).

Blood levels of the hormone fibroblast growth factor 19 (FGF19) are reduced in primary and secondary BAD, producing impaired feedback inhibition of bile acid synthesis, leading to excess faecal bile acids, which then produce diarrhea by stimulating colonic secretion. FGF19 is synthesised in the ileum and we have shown transcription is markedly induced by farnesoid X receptor (FXR) agonists such as chenodeoxycholic acid, an abundant natural bile acid.

We aim to investigate the effects of Compound 1, Compound 2, or Compound 3 in patients with primary and secondary BAD to determine whether FGF19 is able to be stimulated in these conditions. We will compare these responses to those in control patients with chronic diarrhea but without evidence of BAD. It is possible in BAD that the defect in FGF19 levels is due to an inability to respond to FXR stimulation (particularly likely in secondary BAD after ileal resection). Patients with primary BAD may be able to respond and benefit from an increase in FGF19 levels.

This study aims to obtain pilot data on the effects of Compound 1, Compound 2, or Compound 3 on FGF19, other markers of bile acid metabolism and patient symptoms including diarrhea.

Eligibility

Ages Eligible for Study: 18 Years to 80 Years

Genders Eligible for Study: Both

Accepts Healthy Volunteers: No

Criteria

Inclusion Criteria
Patients who present with chronic diarrhea, defined as an average stool frequency of at least three per day, of Bristol Stool Type 6 or 7, for at least 3 months. Previous routine SeHCAT testing to establish the presence or absence of bile acid diarrhea (BAD) unless there is evidence of TI disease/resection. BAD will be defined as SeHCAT 7-day retention of less than 15% or diarrhea in presence of TI disease/resection. Study subjects will be grouped as having secondary BAD, due to ileal resection or Crohn's disease, or primary BAD, with no obvious cause. The third, control group having chronic diarrhea but with normal SeHCAT retention (greater than 15%).
Female patients must be postmenopausal, surgically sterile, or if premenopausal, be prepared to use ≥1 effective (≤1% failure rate) method of contraception during the trial and for 15 days after the last dose of Compound 1, Compound 2, or Compound 3. Male subjects with female partners of childbearing potential must use ≥1 effective method of contraception.

Exclusion Criteria
Patients with other diagnoses leading to diarrhea, including colorectal neoplasia, ulcerative colitis, coeliac disease, chronic pancreatitis, drug-induced diarrhea or active infection.
Patients who have not been investigated by standard clinical assessments to exclude these disorders.
Treatment with bile acid sequestrants (colestyramine, colestipol, colesevelam) for 2 weeks before the first dose of Compound 1, Compound 2, or Compound 3.
Previous biliary surgery, excluding cholecystectomy.
Abnormal bilirubin, alanine aminotransferase (ALT), aspartate aminotransferase (AST) or alkaline phosphatase on more than 1 occasion.
Chronic liver disease
Chronic kidney disease
Active, serious medical disease with likely life expectancy less than 5 years
Active substance abuse including inhaled or injection drugs in the year prior to screening
Pregnancy, planned pregnancy, potential for pregnancy and unwillingness to use effective birth control during the trial, breast feeding. Pregnancy will be assessed with urinary β-hCG pregnancy test.

### Example 18: Use of Compound 1, Compound 2, or Compound 3 for the Treatment of Hepatic Fibrosis in Chronic Viral Hepatitis B

The purpose of this study is to explore the effective dose and safety of Compound 1, Compound 2, or Compound 3 on hepatic fibrosis in chronic viral hepatitis B.

### Study Type: Interventional

### Study Design: Allocation: Randomized

### Endpoint Classification: Safety/Efficacy Study

### Intervention Model: Parallel Assignment

### Masking: Double Blind (Subject, Caregiver, Investigator, Outcomes Assessor)

### Primary Purpose: Treatment

Primary Outcome Measures:
Changes in hepatic fibrosis in chronic viral hepatitis B [Time Frame: one year] [Designated as safety issue: Yes]

| Arms | Assigned Interventions |
|---|---|
| Experimental: Compound 1, | Drug: Compound 1, Compound 2, or |
| Compound 2, or Compound 3 | Compound 3 |
| & Placebo | The investigational products in |
| | the test groups and control group |
| | shall be orally taken 30 minutes |
| | before meals for 52 continuous |
| | weeks. There are respectively 60 |
| | cases in the test group I, II, III |
| | and the control group, totally 240 |
| | cases. |

Primary Observation Indexes:
Hepatic fibrosis Ishak score after treatment decreases by the proportion not less than 1 compared with that before treatment.

Secondary Observation Indexes:
Negative conversion ratio of HBV DNA after treatment (HBV DNA<1×103copies/mL) and falling range.
The falling proportion of Fibrocan Kpa value after treatment compared with that before treatment.
The falling proportion that decreases not less than 1 level and progression-free fibrosis after treatment compared with that before treatment.
The improvement of ALT of liver function.

Eligibility

### Ages Eligible for Study: 18 Years to 65 Years

### Genders Eligible for Study: Both

### Accepts Healthy Volunteers: No

Criteria

### Inclusion Criteria:

History of chronic hepatitis B, HBsAg positive six months.
ALT<five-fold ULN (maximum).
Significant liver fibrosis confirmed by liver biopsy.
HBeAg positive patients, HBV DNA>2.0×104 IU/mL (copies/mL); HBeAg negative patients' HBV DNA>2.0×103 IU/mL (104copies/mL).
Having not accepted the antiviral therapy with interferon and/or nucleoside analog.
Having not taken anti-inflammatory drugs to protect liver within 1 month before selection.
Capable of understanding and signing the informed consent before the study.

### Exclusion Criteria:

Failing to meet any one requirement of the inclusion criteria.
Having suffered massive hemorrhage of gastrointestinal tract within 3 months before selection
TBiL>three-fold ULN.
AFP>50 ug/L
PLT≤60000 ug/L
Having obvious space-occupying lesion in liver as shown by B ultrasound examination.
With a portal vein ≥1.2 cm wide as shown by B ultrasound examination.
BMI index >30.
The patient who suffered from liver function decompensation hepatic cirrhosis and liver neoplasms.
The patient with alcoholic, drug-induced, hereditary, immune and other viral and non-viral chronic hepatitis.
The patient with angiocarpy, lung, kidney, incretion, nerve and blood system disease and mental disease.
The patient with active peptic ulcer.
Gestational and breast feeding women.
The subject who participated in other drug tests within recent 3 months.

### Example 19: Phase 2 Study of Compound 1, Compound 2, or Compound 3 in the Treatment of Gallstone Patients

The purpose of this study is to explore the effects of Compound 1, Compound 2, or Compound 3 on Hepatic Fatty Acid/Triglyceride Metabolism and Hepatobiliary Detoxification/Elimination in Gallstone Patients.

### Study Type: Interventional

### Study Design: Allocation: Randomized

### Endpoint Classification: Pharmacodynamics Study

### Intervention Model: Parallel Assignment

### Masking: Double Blind (Subject, Investigator)

### Primary Purpose: Basic Science

Primary Outcome Measures:
Effects of Compound 1, Compound 2, or Compound 3 on FXR-dependent metabolism [Time Frame: Day 21] [Designated as safety issue: No]

Primary Endpoints
relative changes in markers for insulin resistance
relative changes in FA and TG
relative changes in hepatic and adipose tissue lipase expression and activity
relative changes in hepatic apical transport proteins ABCG5/8, BSEP, MDR3, MRP2
relative changes in hepatic ER stress markers

Secondary Outcome Measures:
Effects of Compound 1, Compound 2, or Compound 3 on serum lipid levels [Time Frame: 21 days] [Designated as safety issue: No]

Secondary Endpoints
relative changes in total cholesterol, LDL-C, HDL-C, Apo A1, Apo B, in Lp(A)

| Arms | Assigned Interventions |
|---|---|
| Active Comparator: Compound 1, | Drug: Compound 1, Compound 2, |
| Compound 2, or Compound 3, | or Compound 3, 25 mg/day |
| 25 mg/day in three weeks | in three weeks |
| Placebo Comparator: Gallstone | Drug: Placebo |
| Placebo | |

In a placebo-controlled double-blind randomized trial, healthy gallstone patients will be administered 25 mg/day Compound 1, Compound 2, or Compound 3 or placebo for three weeks until the day before surgery. Gallbladder bile will be sampled for the measurements of biliary lipids (cholesterol, phospholipids, bile acids) and the calculation of the cholesterol saturation index.

Eligibility

### Ages Eligible for Study: 20 Years to 65 Years

### Genders Eligible for Study: Both

### Accepts Healthy Volunteers: No

Criteria

### Inclusion Criteria:

symptomatic, ultrasound verified gallstone disease

### Exclusion Criteria:

Chronic liver disease other than NAFLD (viral hepatitis, autoimmune liver disease, hemochromatosis, homozygous alpha1-antitrypsin deficiency and Wilson disease)
Previous gastric or small bowel surgery
Inflammatory bowel disease
Uncontrolled diabetes mellitus (fasting blood glucose >6.7 mmol/L), hypothyroidism or hyperthyroidism, or other significant endocrine disease.
Pregnancy. A urine pregnancy test will be performed the day before start of medication. Women of childbearing potential can only be included if a safe and reliable contraception is used, e.g., oral contraceptives.
Elevations of transaminases (ALAT/ASAT) or alkaline phosphatase or bilirubin above 2×ULN (upper limit of normal) the day before start of medication.
Other serious disease, including depressive disorders treated by medication

### Example 20: Study of Compound 1, Compound 2, or Compound 3 to Treat Focal Segmental Glomerulosclerosis (FS GS)

The purpose of this study is to assess the safety and tolerability of Compound 1, Compound 2, or Compound 3 in patients with FSGS and to conduct a pharmacokinetic (PK) assessment of Compound 1, Compound 2, or Compound 3 to enable selection of medication regimens for investigation in a randomized Phase II study.

Study Type: Interventional

Study Design: Allocation: Randomized

Endpoint Classification: Safety Study

Intervention Model: Parallel Assignment

Masking: Open Label

Primary Purpose: Treatment

Primary Outcome Measures:
Safety and tolerance of medications [Time Frame: 16 week treatment period]

Secondary Outcome Measures:
Reduction in proteinuria [Time Frame: 16 week treatment period]

| Arms | Assigned Interventions |
|---|---|
| Compound 1, Compound 2, or Compound 3 | Drug: compound oral |
| | drug administration |

Eligibility

Ages Eligible for Study: 2 Years to 40 Years

Genders Eligible for Study: Both

Accepts Healthy Volunteers: No

Criteria

Inclusion Criteria:
Estimated glomerular filtration rate (GFR)≥40 ml/min/1.73 m2 at most recent measurement prior to randomization
   For patients <age 18 years: Schwartz formula
   For patients ≥age 18 years: Cockroft-Gault formula
Up/c>1.0 g/g creatinine on first morning void at time of randomization
Biopsy confirmed as primary FSGS (including all subtypes) by study pathologist.
Steroid resistance: During the last treatment course with high dose steroids prior to randomization, the patient must have demonstrated steroid resistance defined below and not have had a complete remission of proteinuria (Up/c<0.2 or dipstick urine protein negative/trace) subsequently. The course of steroid treatment that defines resistance must be the same or equivalent to at least 4 weeks of every day dosing with a minimum cumulative dose of 56 mg/kg or 1680 mg of prednisone or its equivalent.
May be taking angiotensin-converting enzyme inhibitor (ACEI), angiotensin receptor blocking agent (ARB), vitamin E, or lipid lowering therapy
Willingness to comply with clinical trial protocol, medications, and follow-up visits, etc.
Screen failure in FSGS-CT based on prior treatment with excluded medication
Treatment failure in FSGS-CT based on failure to achieve remission after 26 weeks or 52 weeks of test therapy, i.e., cyclosporine or mycophenolate mofetil (MMF)+oral dexamethasone pulses

Exclusion Criteria
Secondary FSGS
Treated with cyclophosphamide, chlorambucil, levamisole, methotrexate, nitrogen mustard, or other immunosuppressive medications in the 30 days prior to randomization
Lactation, pregnancy, or refusal of birth control in women of child bearing potential
Participation in another therapeutic trial concurrently or for 30 days prior to randomization
Active/serious infection (including, but not limited to hepatitis B or C, HIV)
Malignancy
Systemic lupus erythematosus (SLE) or multiple sclerosis
Hepatic disease defined as serum AST/ALT>2.5× the upper limit of normal
Patients with blood pressure >140/95 or >95th percentile for age/height while receiving maximal doses of 3 or more antihypertensive agents.
Diabetes mellitus (DM) type I or II.
Hematocrit <30%
Organ transplantation
Obesity (based on estimated dry weight at disease onset prior to steroid therapy) defined as:
   Body mass index (BMI)>97th percentile for age if aged 2-20 years
   BMI>40 kg/m2 if aged ≥21 years
Allergy to study medications
Inability to consent/assent

### Example 21: Effect of Compound 1, Compound 2, or Compound 3 in the Treatment of Patients with Hypertriglyceridemia

The proposed study will utilize several different measures of lipoprotein structure and function. The investigators will measure functional parameters such as the binding affinity of lipoproteins before and after treatment with Compound 1, Compound 2, or Compound 3. The investigators will also measure fatty acid, oxylipin and apolipoprotein content of plasma, very-low-density lipoprotein (VLDL), low-density lipoprotein (LDL) and high-density lipoprotein (HDL).

### Study Design: Endpoint Classification: Efficacy Study

### Intervention Model: Single Group Assignment

### Masking: Open Label

### Primary Purpose: Treatment

### Study Type: Interventional

Primary Outcome Measures:
The primary endpoint will be the effect of treatment on lipoprotein binding to target cells. [Time Frame: one year] [Designated as safety issue: No]

Secondary Outcome Measures:
Performance of VLDL as a substrate for lipolysis. [Time Frame: one year] [Designated as safety issue: No]
Plasma LpL mass and activity. [Time Frame: one year] [Designated as safety issue: No]
VLDL, LDL, HDL oxylipin and fatty acid content [Time Frame: one year] [Designated as safety issue: No]

| Arms | Assigned Interventions |
|---|---|
| Experimental: Compound 1, | Drug: Compound 1, Compound 2, |
| Compound 2, or Compound 3 | or Compound 3 |
| | 1 gram gel capsule 4 capsules |
| | per day for 8 weeks |

Eligibility

### Ages Eligible for Study: 18 Years to 79 Years

### Genders Eligible for Study: Both

### Accepts Healthy Volunteers: No

Criteria

### Inclusion Criteria:

Good health by medical history, physical exam, electrocardiogram, laboratory test (e.g., serum chem., urinalysis)
Mean fasting serum triglyceride of two most recent tests in medical record ≥200 and <500 mg/dL
Mean LDL-cholesterol of two most recent tests in medical record ≤1.1×NCEP ATP III goal

### Exclusion Criteria:

Medications, vitamin pills, nutritional supplements or herbal preparations deemed exclusionary per primary investigator for possible interference
Poorly controlled diabetes mellitus (e.g. [HbA1c]>8.0%)
History of a cardiovascular event
Past revascularization procedure
Past aortic aneurysm or an aortic dissection <6 months prior to screening
History of pancreatitis
Poorly controlled hypertension (i.e.: >=160 systolic (resting) and/or >=100 diastolic (resting)) at 2 consecutive visits
Serum Creatinine >2.0 mg/dL
Serum transaminase >1.5× upper limit of normal (ULN); including aspartate aminotransferase [AST] or alanine aminotransferase [ALT]; 31 U/L for AST, 45 U/L for ALT
Creatine Kinase (CK)>3.0×ULN
Women who are pregnant or nursing

### Example 22: Study of Compound 1, Compound 2, or Compound 3 to Treat Diabetic Nephropathy

The purpose of this study is to evaluate the safety and effects of Compound 1, Compound 2, or Compound 3 in treatment of diabetic nephropathy.

### Study Type: Interventional

### Study Design: Allocation: Randomized

### Endpoint Classification: Safety/Efficacy Study

### Intervention Model: Parallel Assignment

### Masking: Double Blind (Subject, Caregiver, Investigator, Outcomes Assessor)

### Primary Purpose: Treatment

Primary Outcome Measures:
Urine albumin level [Time Frame: 3 months] [Designated as safety issue: Yes]
Serum creatinin [Time Frame: 3 months] [Designated as safety issue: Yes]

Secondary Outcome Measures:
Fasting blood sugar (FBS) [Time Frame: 3 months] [Designated as safety issue: Yes]
Glycosylated hemoglobin (A1C) [Time Frame: 3 months] [Designated as safety issue: Yes]
Liver aminotransferases (ALT and AST) [Time Frame: 3 months] [Designated as safety issue: Yes]
Serum insulin level [Time Frame: 3 months] [Designated as safety issue: Yes]
Number of patients with adverse events [Time Frame: 3 months] [Designated as safety issue: Yes]

| Arms | Assigned Interventions |
|---|---|
| Experimental: Compound 1, | Drug: Compound 1, Compound 2, or |
| Compound 2, or Compound 3 | Compound 3 |
| | Compound 1, Compound 2, |
| | or Compound 3; 500 mg daily |
| Placebo Comparator: Placebo | Drug: Placebo |
| Placebo | Placebo 1 capsule daily |

Eligibility

### Ages Eligible for Study: 18 Years to 70 Years

### Genders Eligible for Study: Both

### Accepts Healthy Volunteers: No

Criteria

### Inclusion Criteria:

Type 2 diabetes mellitus (DM)
Controlled blood sugar [fasting blood sugar (FBS)<130 mg/dl and glycosylated hemoglobin (A1C)<7%
Urine albumin >20 mg/lit in two separate occasions during the last 3 months period
Serum creatinin < or =2 mg/dl

### Exclusion Criteria:

Pregnancy
Lactation
Alcoholism
Liver failure (acute or chronic)
Renal failure: serum creatinin >2 mg/dl
Glomerulonephritis
Uncontrolled hypertension
Congestive heart failure
Prostate disease
Malignancy
Bilateral renal artery stenosis
Any infection or rheumatologic disorder

### Example 23: Phase 2 Study of Compound 1, Compound 2, or Compound 3 in the Treatment of Patients with Atherosclerosis

The purpose of this study is to assess the effect of Compound 1, Compound 2, or Compound 3 on the reduction of atherosclerosis in the carotid artery will be assessed using carotid ultrasound.

### Study Type: Interventional

### Study Design: Allocation: Randomized

### Endpoint Classification: Safety/Efficacy Study

### Intervention Model: Parallel Assignment

### Masking: Double-Blind

### Primary Purpose: Treatment

Primary Outcome Measures:
Efficacy of Compound 1, Compound 2, or Compound 3 versus placebo on the progression of atherosclerosis

Secondary Outcome Measures:
Safety and tolerability of Compound 1, Compound 2, or Compound 3 versus placebo in patients with atherosclerosis

Eligibility

### Ages Eligible for Study: 30 Years to 75 Years

### Genders Eligible for Study: Both

### Accepts Healthy Volunteers: No

Criteria

### Inclusion Criteria:

Increased cardiovascular risk (i.e. history of myocardial infarction, stroke, diabetes mellitus, left ventricular hypertrophy)
Intima-media thickness greater than or equal to 0.8 mm as measured by ultrasonography
Negative pregnancy test for females

### Exclusion Criteria:

Whole blood donation (greater than or equal to 450 ml) during the last three months before study start
Unstable angina, congestive heart failure or uncontrolled hypertension
Renal disease including nephrectomy and/or renal transplant
Hepatic disease or abnormal liver function parameters
Drug abuse or alcohol addiction

### Example 24: Use of Compound 1, Compound 2, or Compound 3 for the Treatment of Portal Hypertension

The purpose of this study is to determine the efficacy and safety of Compound 1, Compound 2, or Compound 3 as compared to placebo for lowering portal hypertension.

Study Type: Interventional

Study Design: Allocation: Randomized

Endpoint Classification: Safety/Efficacy Study

Intervention Model: Parallel Assignment

Masking: Double Blind (Subject, Investigator)

Primary Purpose: Treatment

Primary Outcome Measures:
Change From Baseline in Hepatic Venous Pressure Gradient (HVPG) After Four (4) Weeks of Treatment [Time Frame: 4 weeks] [Designated as safety issue: No]

| Arms | Assigned Interventions |
|---|---|
| Placebo Comparator: Placebo | Drug: Placebo capsules three times |
| Placebo | daily (TID) |
| Experimental: Compound 1, | Drug: Compound 1, Compound 2, |
| Compound 2, or Compound 3 | or Compound 3 |
| | Compound 1, Compound 2, or |
| | Compound 3 (capsules) three |
| | times daily (TID |

Eligibility

Ages Eligible for Study: 18 Years and older

Genders Eligible for Study: Both

Accepts Healthy Volunteers: No

Criteria

Inclusion Criteria:
Patient has clinical and/or pathological diagnosis of intra-hepatic portal hypertension.
Patient has clinical diagnosis of cirrhosis.
Patient has undergone variceal banding.

Exclusion Criteria:
Patient has a Child-Pugh score >12.
Patient has portal hypertension resulting from hepatic vein obstruction, portal vein occlusion, schistosomiasis, portal vein thrombosis, splenic vein thrombosis, or Budd-Chiari syndrome.
Variceal banding procedure was performed within 1 month of the screening visit.
Patient has active or recurrent variceal bleeding, or has had variceal bleeding within the 12 weeks prior to screening.
Patient is unwilling to discontinue use of vasoactive drugs from the screening visit through the end of the study.
Patient has hepatocellular carcinoma that is being medically treated or is advanced.
Patient has impaired renal function (i.e., serum creatinine concentration >1.8 mg/dl)
Patient has a history of liver transplant, or is expected to receive a liver transplant during the study period.
Patient has undergone a gastrointestinal or abdominal surgical procedure within 90 days prior to the Screening Visit, or has had a bowel resection at any time.

The examples and embodiments described herein are for illustrative purposes only and in some embodiments, various modifications or changes are to be included within the purview of disclosure and scope of the appended claims.

1. A method of treating a metabolic disorder in a subject in need thereof, comprising co-administering to the subject a therapeutically effective amount of:
(a) a first agent that is an FXR modulator; and
(b) at least one second agent that is an CCR2/CCR5 antagonist, ASK1 inhibitor, DPP-IV inhibitor, caspase protease inhibitor, SGLT2 inhibitor, acetyl-CoA carboxylase (ACC) inhibitor, diacylglycerol acyltransferase-1 inhibitor, sodium-bile acid cotransporter-inhibitor, TLR-4 antagonist, PPAR alpha/delta agonist, or GLP-1 agonist, or a combination thereof;
wherein the FXR modulator is a compound selected from the Formula (I): 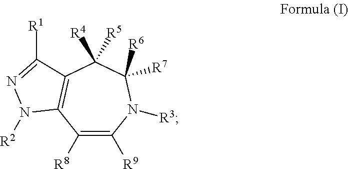
wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R² is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of —CN, —C(O)OR²⁵, —C(O)N(R²⁵)R²⁶, 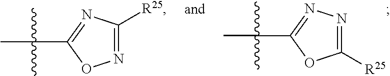
R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂ alkylene)-(aryl), optionally substituted C₂-C₉heterocycloalkyl, optionally substituted heteroaryl, and optionally substituted —(C₁-C₂alkylene)-(heteroaryl); or R⁸ and R⁹ together with the carbon atoms to which they are attached, form an optionally substituted C₂-C₉heterocycloalkyl ring or an optionally substituted heteroaryl ring;
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or a pharmaceutically acceptable salt, stereoisomer or solvate thereof;
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or
R²⁷ and R²⁸ together with the nitrogen atom to which they are attached, form an optionally substituted C₂-C₉heterocycloalkyl ring; or a pharmaceutically acceptable salt, stereoisomer or solvate thereof;
wherein the metabolic disorder is nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, diabetic nephropathy or nephrotic syndrome.

2. The method of claim 1, wherein the FXR modulator is a compound of Formula (III): 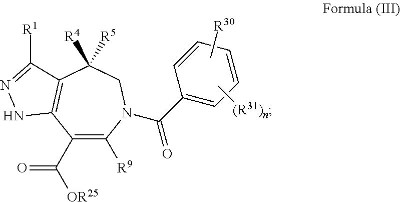
wherein:
R¹ is selected from the group consisting of hydrogen, halogen, optionally substituted C₁-C₆alkyl, optionally substituted C₂-C₆alkenyl, optionally substituted C₂-C₆alkynyl, optionally substituted aryl, optionally substituted heteroaryl, optionally substituted C₃-C₈cycloalkyl, optionally substituted —(C₁-C₂alkylene)-(C₃-C₈cycloalkyl), optionally substituted C₂-C₉heterocycloalkyl, optionally substituted —(C₁-C₂alkylene)-(C₂-C₉heterocycloalkyl), optionally substituted —(C₁-C₂alkylene)-(aryl), optionally substituted —(C₁-C₂alkylene)-(heteroaryl), —OR¹⁰, —SR¹⁰, —N(R¹¹)R¹², —N(R¹¹)S(O)₂R¹⁵; —N(R¹³)N(R¹¹)R¹², —N(R¹³)N(R¹¹)S(O)₂R¹⁵, —C(O)R¹⁴, —C(O)OR¹⁰, —C(S)OR¹⁰, —C(O)SR¹⁰, —C(O)N(R¹¹)R¹², —C(S)N(R¹¹)R¹², —C(O)N(R¹¹)S(O)₂R¹⁵, —C(S)N(R¹¹)S(O)₂R¹⁵, —C(O)N(R¹³)N(R¹¹)R¹², —C(S)N(R¹³)N(R¹¹)R¹² and —C(O)N(R¹³)N(R¹¹)S(O)₂R¹⁵;
R⁴ and R⁵ are each independently optionally substituted C₁-C₆alkyl;
R⁹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁰, R¹³ and R¹⁴ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹⁵ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R¹¹ and R¹² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or optionally R¹¹ and R¹² together with the nitrogen atom to which they are attached, form an optionally substituted C₂-C₉heterocycloalkyl ring;
R²⁵ is C₁-C₆alkyl;
R³⁰ is halogen, 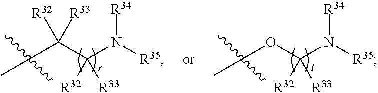
each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
n is 0, 1, 2, 3, or 4;
r is 0, 1, 2, 3, or 4;
t is 2, 3, or 4; or a pharmaceutically acceptable salt, stereoisomer or solvate thereof.

3. The method of claim 1, wherein the FXR modulator is a compound having the structure: 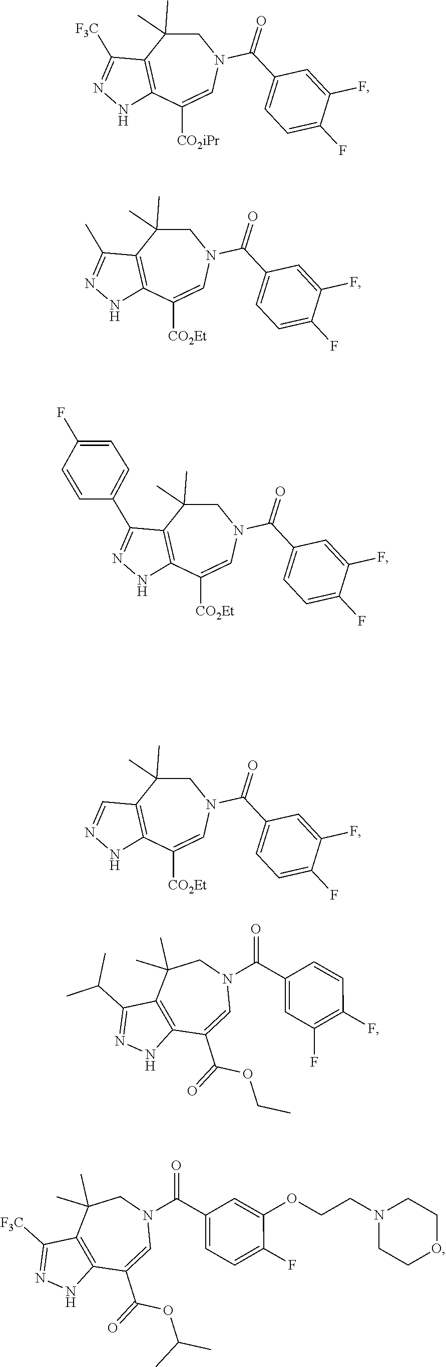 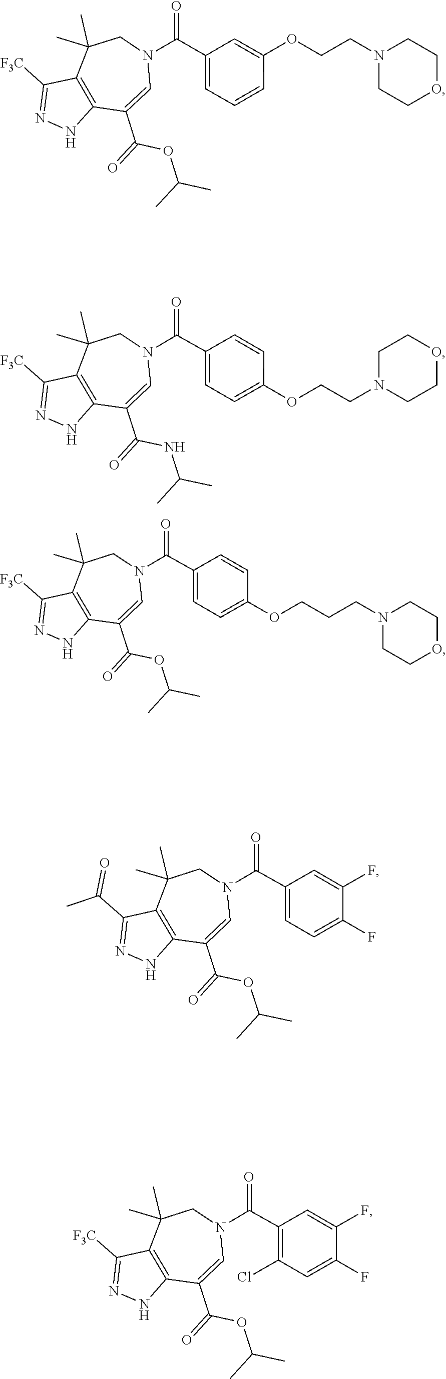 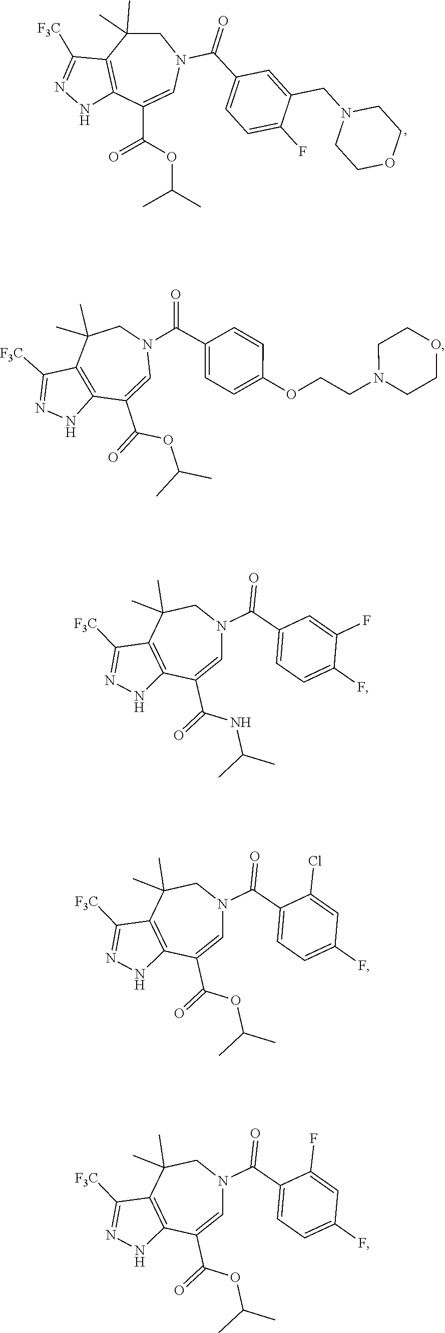 [SMILES: CC(C)OC(=O)C1=CN(C(=O)c2ccc(F)c(CN3CCOCC3)c2)CC(C)(C)c2c(C(F)(F)F)n[nH]c21 ; CC(C)OC(=O)C1=CN(C(=O)c2ccc(OCCN3CCOCC3)cc2)CC(C)(C)c2c(C(F)(F)F)n[nH]c21 ; CC(C)NC(=O)C1=CN(C(=O)c2ccc(F)c(F)c2)CC(C)(C)c2c(C(F)(F)F)n[nH]c21 ; CC(C)OC(=O)C1=CN(C(=O)c2ccc(F)cc2Cl)CC(C)(C)c2c(C(F)(F)F)n[nH]c21 ; CC(C)OC(=O)C1=CN(C(=O)c2ccc(F)cc2F)CC(C)(C)c2c(C(F)(F)F)n[nH]c21] 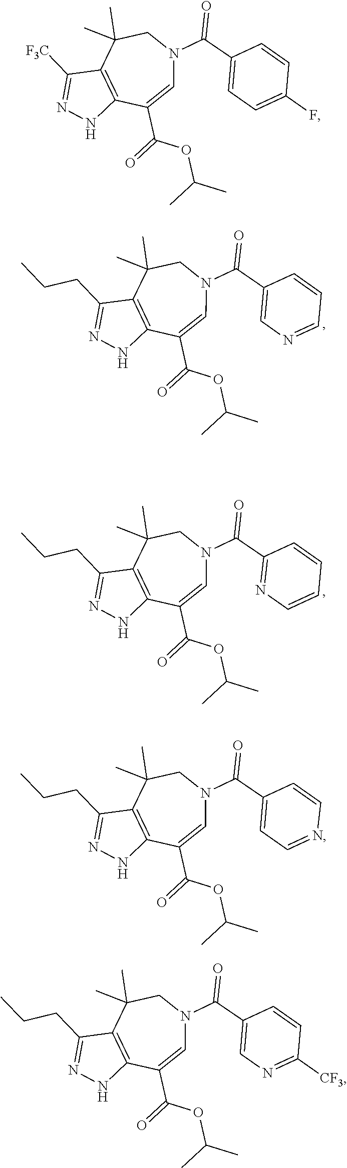 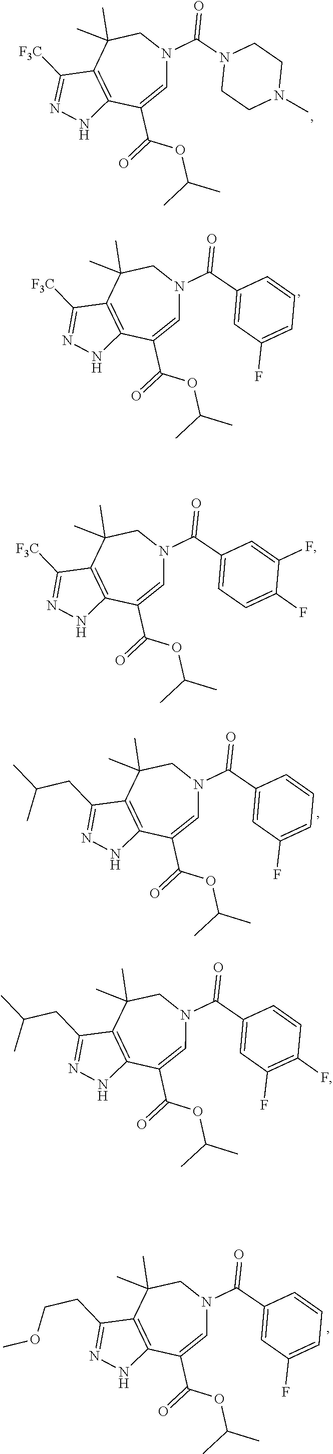 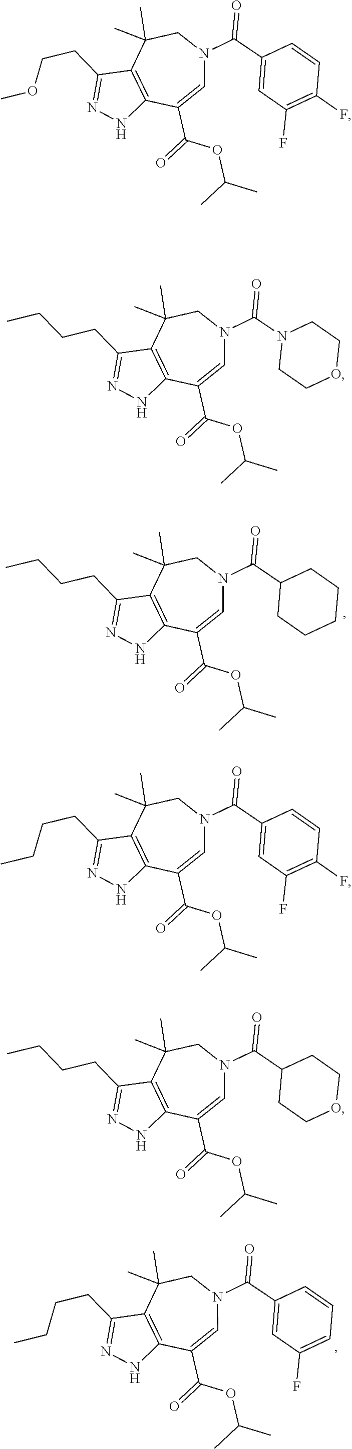 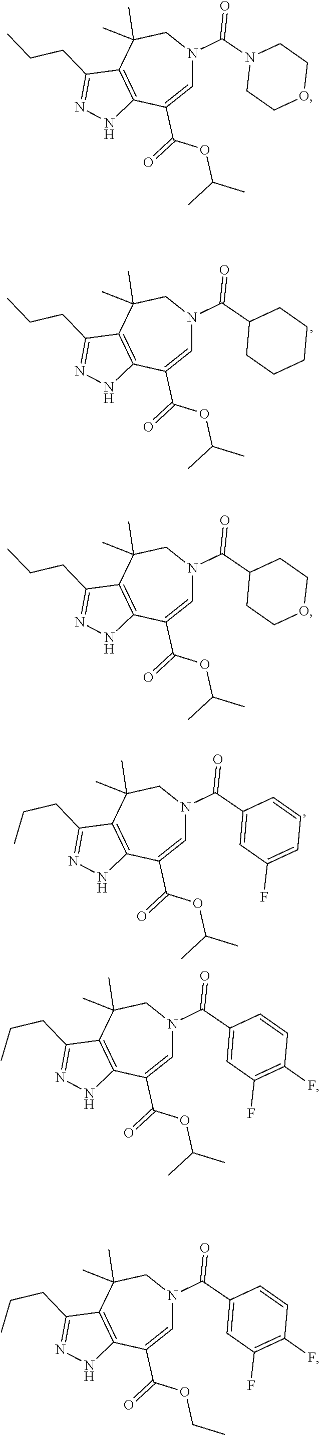 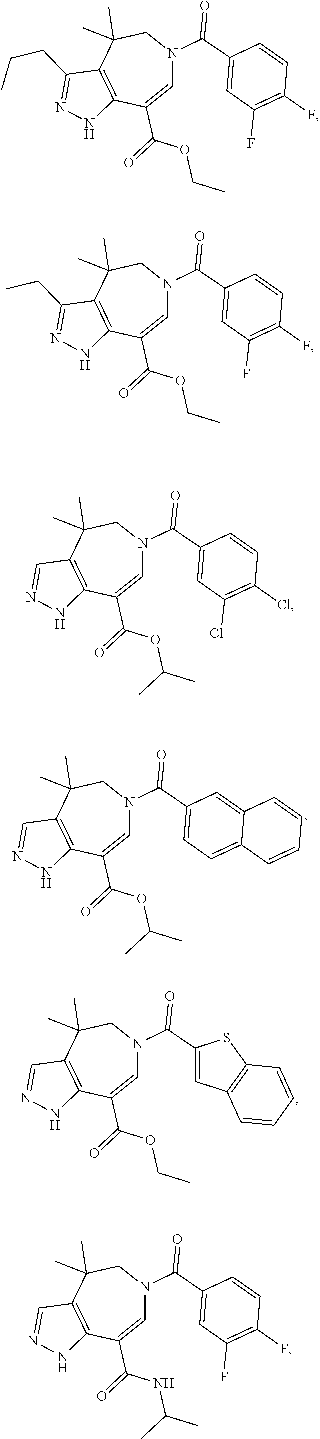 [SMILES: CCCc1n[nH]c2c1C(C)(C)CN(C(=O)c1ccc(F)c(F)c1)C=C2C(=O)OCC ; CCOC(=O)C1=CN(C(=O)c2ccc(F)c(F)c2)CC(C)(C)c2c(CC)n[nH]c21 ; CC(C)OC(=O)C1=CN(C(=O)c2ccc(Cl)c(Cl)c2)CC(C)(C)c2cn[nH]c21 ; CC(C)OC(=O)C1=CN(C(=O)c2ccc3ccccc3c2)CC(C)(C)c2cn[nH]c21 ; CCOC(=O)C1=CN(C(=O)c2cc3ccccc3s2)CC(C)(C)c2cn[nH]c21 ; CC(C)NC(=O)C1=CN(C(=O)c2ccc(F)c(F)c2)CC(C)(C)c2cn[nH]c21] 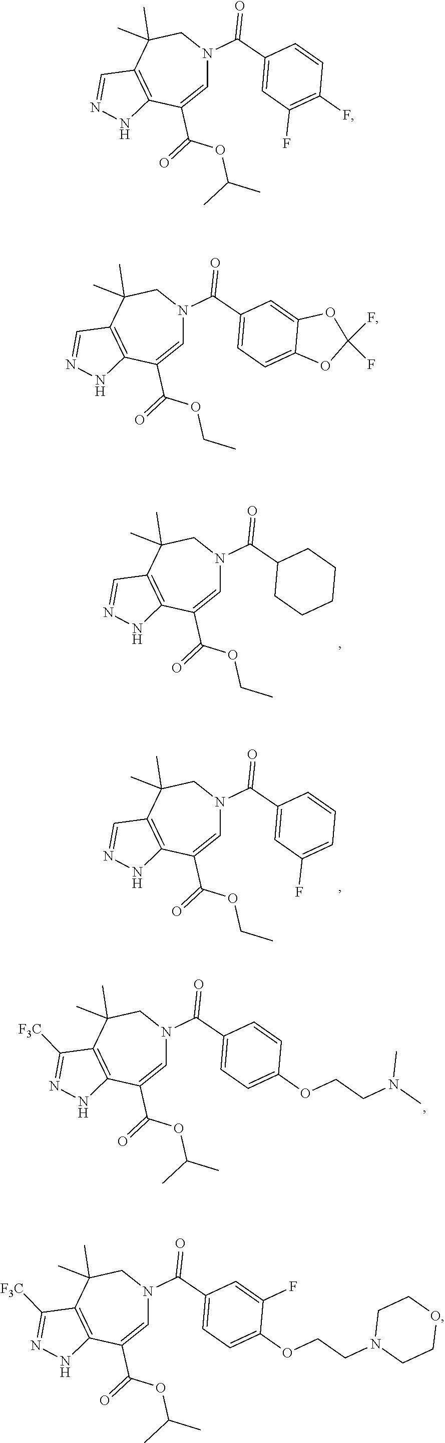 [SMILES: CC(C)OC(=O)C1=CN(C(=O)c2ccc(F)c(F)c2)CC(C)(C)c2cn[nH]c21 ; CCOC(=O)C1=CN(C(=O)c2ccc3c(c2)OC(F)(F)O3)CC(C)(C)c2cn[nH]c21 ; CCOC(=O)C1=CN(C(=O)C2CCCCC2)CC(C)(C)c2cn[nH]c21 ; CCOC(=O)C1=CN(C(=O)c2cccc(F)c2)CC(C)(C)c2cn[nH]c21 ; CC(C)OC(=O)C1=CN(C(=O)c2ccc(OCCN(C)C)cc2)CC(C)(C)c2c(C(F)(F)F)n[nH]c21 ; CC(C)OC(=O)C1=CN(C(=O)c2ccc(OCCN3CCOCC3)c(F)c2)CC(C)(C)c2c(C(F)(F)F)n[nH]c21] 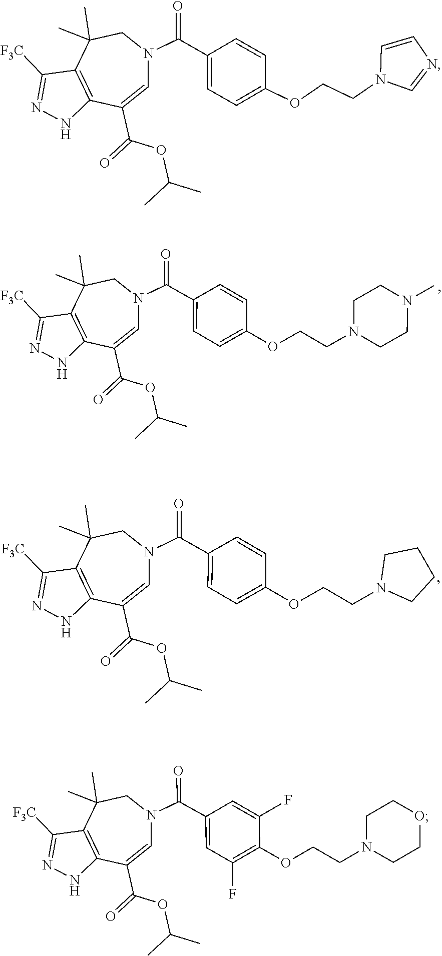 [SMILES: CC(C)OC(=O)C1=CN(C(=O)c2ccc(OCCn3ccnc3)cc2)CC(C)(C)c2c(C(F)(F)F)n[nH]c21 ; CC(C)OC(=O)C1=CN(C(=O)c2ccc(OCCN3CCN(C)CC3)cc2)CC(C)(C)c2c(C(F)(F)F)n[nH]c21 ; CC(C)OC(=O)C1=CN(C(=O)c2ccc(OCCN3CCCC3)cc2)CC(C)(C)c2c(C(F)(F)F)n[nH]c21 ; CC(C)OC(=O)C1=CN(C(=O)c2cc(F)c(OCCN3CCOCC3)c(F)c2)CC(C)(C)c2c(C(F)(F)F)n[nH]c21]
or a pharmaceutically acceptable salt, stereoisomer, or solvate thereof.

4. (canceled)

5. The method of claim 1, wherein the second agent is an CCR2/CCR5 antagonist selected from the group consisting of cenicriviroc (CVC), apiaviroc, vicriviroc, maraviroc and cochilioquinone.

6. The method of claim 1, wherein the second agent is an ASK1 inhibitor selected from the group consisting of GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyri dinecarboxamide) and TC ASK 10 (4-(1,1-dimethylethyl)-N-[6-(1H-imidazol-1-yl)imidazo[1,2-a]pyridin-2-yl]benzamide dihydrochloride).

7. The method of claim 1, wherein the second agent is a DPP-IV inhibitor selected from the group consisting of sitagliptin, saxagliptin, linagliptin, alogliptin, vildagliptin, gemigliptin, anagliptin, teneligliptin, trelagliptin, dutogliptin and omarigliptin.

8. The method of claim 1, wherein the second agent is a caspase protease inhibitor selected from the group consisting of emricasan, Q-VD-Oph, DEVD-CHO, zVAD-FMK, Pralnacasan and M867.

9. The method of claim 1, wherein the second agent is an SGLT2 inhibitor selected from the group consisting of canagliflozin, empagliflozin, dapagliflozin, ipragliflozin, tofogliflozin, sergliflozin etabonate, remogliflozin etabonate and ertugliflozin.

10. The method of claim 1, wherein the second agent is a GLP-1 agonist selected from the group consisting of exenatide, liraglutide, lixisenatide, albiglutide, dulaglutide, taspoglutide, and semaglutide.

11.-15. (canceled)

16. A method of treating a metabolic disorder in a subject in need thereof, comprising co-administering to the subject a therapeutically effective amount of:
(a) a first agent that is an FXR modulator; and
(b) at least one second agent that is an CCR2/CCR5 antagonist, ASK1 inhibitor, DPP-IV inhibitor, caspase protease inhibitor, SGLT2 inhibitor, acetyl-CoA carboxylase (ACC) inhibitor, diacylglycerol acyltransferase-1 inhibitor, sodium-bile acid cotransporter-inhibitor, TLR-4 antagonist, PPAR alpha/delta agonist, or GLP-1 agonist, or a combination thereof;
wherein the FXR modulator is a compound of Formula (VII): 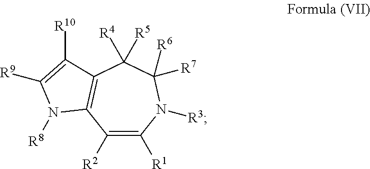
wherein:
R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R² is selected from the group consisting of —CN, —C(O)OR²⁵, —C(O)N(R²⁵)R²⁶, 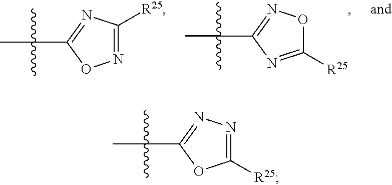
or R¹ and R² together with the carbon atoms to which they are attached, form an optionally substituted C₂-C₉heterocycloalkyl ring or an optionally substituted heteroaryl ring;
R³ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, optionally substituted —(C₁-C₂alkylene)-(heteroaryl), —C(O)R²⁰, —C(O)OR²⁰, —S(O)₂R²⁰, —C(O)N(R²¹)R²², —C(O)N(R²¹)S(O)₂R²⁴, —C(O)N(R²³)N(R²¹)R²², —C(O)N(R²³)N(R²¹)S(O)₂R²⁴, —N(R²³)C(O)R²⁰, —N(R²³)C(O)N(R²¹)R²², —N(R²³)C(O)N(R²¹)S(O)₂R²⁴, —N(R²⁰)C(O)N(R²³)N(R²¹)R²², —N(R²⁰)C(O)N(R²³)N(R²¹)S(O)₂R²⁴, —N(R²³)C(O)OR²⁰, —P(O)OR²⁰, and —P(O)(OR¹⁹)OR²⁰;
R⁴ and R⁵ are each independently selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R⁹ and R¹⁰ together with the carbon atoms to which they are attached, form an optionally substituted nitrogen containing 6-membered heteroaryl ring;
R¹⁹, R²⁰, and R²³ are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²¹ and R²² are each independently selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or R²¹ and R²² together with the nitrogen atom to which they are attached, form an optionally substituted C₂-C₉heterocycloalkyl ring;
R²⁴ is selected from the group consisting of optionally substituted C₁-C₆alkyl, optionally substituted C₂-C₆alkenyl, optionally substituted C₂-C₆alkynyl, optionally substituted C₃-C₈ cycloalkyl, optionally substituted aryl optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); and
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or
R²⁷ and R²⁸ together with the nitrogen atom to which they are attached, form an optionally substituted C₂-C₉heterocycloalkyl ring; or a pharmaceutically acceptable salt, stereoisomer or solvate thereof;
wherein the metabolic disorder is nonalcoholic steatohepatitis (NASH), hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, diabetic nephropathy or nephrotic syndrome.

17. The method of claim 16, wherein the FXR modulator is a compound of Formula (VIId), or a pharmaceutically acceptable salt, stereoisomer or solvate thereof: 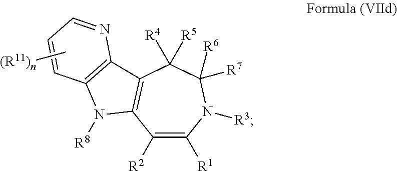
wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², and —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

18. The method of claim 16, wherein the FXR modulator is a compound of Formula (VIII): 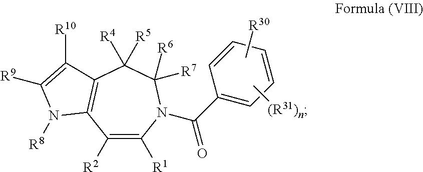
wherein:
R¹ is selected from the group consisting of hydrogen, optionally substituted C₁-C₆alkyl, optionally substituted C₂-C₆alkenyl, optionally substituted C₂-C₆alkynyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R² is selected from the group consisting of —CN, —C(O)OR²⁵, —C(O)N(R²⁵)R²⁶, 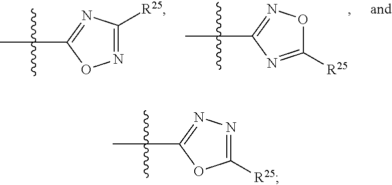
or R¹ and R² together with the carbon atoms to which they are attached, form an optionally substituted C₂-C₉heterocycloalkyl ring or an optionally substituted heteroaryl ring;
R⁴ and R⁵ are each independently selected from the group consisting of hydrogen, halogen, optionally substituted C₁-C₆alkoxy, optionally substituted C₁-C₆alkyl, optionally substituted C₂-C₆alkenyl, and optionally substituted C₂-C₆alkynyl; or R⁴ and R⁵ together with the carbon atom to which they are attached, form an optionally substituted C₃-C₆cycloalkyl ring or an optionally substituted C₂-C₇heterocycloalkyl ring;
R⁶ is selected from the group consisting of hydrogen, halogen, optionally substituted C₁-C₆alkyl, optionally substituted C₂-C₆alkenyl, optionally substituted C₂-C₆alkynyl, and —C(O)N(R²⁷)R²⁸;
R⁷ is selected from the group consisting of hydrogen, halogen, optionally substituted C₁-C₆alkyl, optionally substituted C₁-C₆alkoxy, optionally substituted C₂-C₆alkenyl, and optionally substituted C₂-C₆alkynyl;
R⁸ is selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted heteroaryl, optionally substituted C₂-C₉heterocycloalkyl, and optionally substituted —(C₁-C₂alkylene)-(heteroaryl);
R⁹ and R¹⁰ together with the carbon atoms to which they are attached, form an optionally substituted nitrogen containing 6-membered heteroaryl ring;
R²⁵ and R²⁶ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl);
R²⁷ and R²⁸ are each independently selected from the group consisting of hydrogen, optionally substituted C₁-C₆alkyl, optionally substituted C₃-C₈cycloalkyl, optionally substituted aryl, optionally substituted —(C₁-C₂alkylene)-(aryl), optionally substituted C₂-C₉heterocycloalkyl, optionally substituted heteroaryl, and optionally substituted —(C₁-C₂alkylene)-(heteroaryl); or
R²⁷ and R²⁸ together with the nitrogen atom to which they are attached, form an optionally substituted C₂-C₉heterocycloalkyl ring;
R³⁰ is halogen, 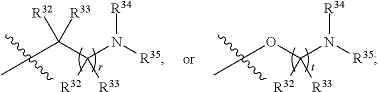
each R³¹ is independently halogen, —OH, —CN, —NO₂, —NH₂, optionally substituted C₁-C₆alkyl, optionally substituted C₁-C₆alkoxy, optionally substituted C₁-C₆alkylamine, optionally substituted C₃-C₈cycloalkyl, optionally substituted C₂-C₉heterocycloalkyl, aryl, or heteroaryl;
each R³² and R³³ are each independently selected from the group consisting of hydrogen, halogen, and C₁-C₆alkyl;
R³⁴ and R³⁵ are each independently selected from the group consisting of hydrogen, optionally substituted C₁-C₆alkyl, optionally substituted C₃-C₈cycloalkyl, and optionally substituted C₂-C₉heterocycloalkyl; or R³⁴ and R³⁵ together with the nitrogen atom to which they are attached, form an optionally substituted C₂-C₉heterocycloalkyl ring or an optionally substituted heteroaryl ring;
p is 0, 1, 2, 3, or 4;
r is 0, 1, 2, 3, or 4; and
t is 2, 3, or 4; or a pharmaceutically acceptable salt, stereoisomer, or solvate thereof.

19. The method of claim 16, wherein the FXR modulator is a compound of Formula (VIIId), or a pharmaceutically acceptable salt, stereoisomer, or solvate thereof: 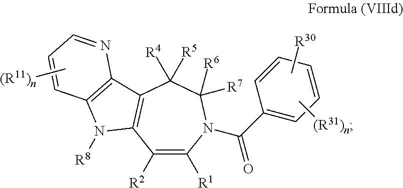
wherein:
each R¹¹ is independently selected from the group consisting of halogen, —CN, amino, alkylamino, C₁-C₆alkyl, C₁-C₆haloalkyl, C₁-C₆alkoxy, C₁-C₆haloalkoxy, C₃-C₈cycloalkyl, C₂-C₉heterocycloalkyl, aryl, heteroaryl, —C(O)OR¹², —C(O)N(R¹³)R¹⁴;
each R¹² is independently selected from the group consisting of hydrogen and C₁-C₆alkyl;
each R¹³ and R¹⁴ are each independently selected from the group consisting of hydrogen and C₁-C₆alkyl; or R¹³ and R¹⁴ together with the nitrogen atom to which they are attached, form an optionally substituted C₂-C₉heterocycloalkyl ring; and
n is 0, 1, 2, or 3.

20. The method of claim 16, wherein the FXR modulator is a compound having the structure: 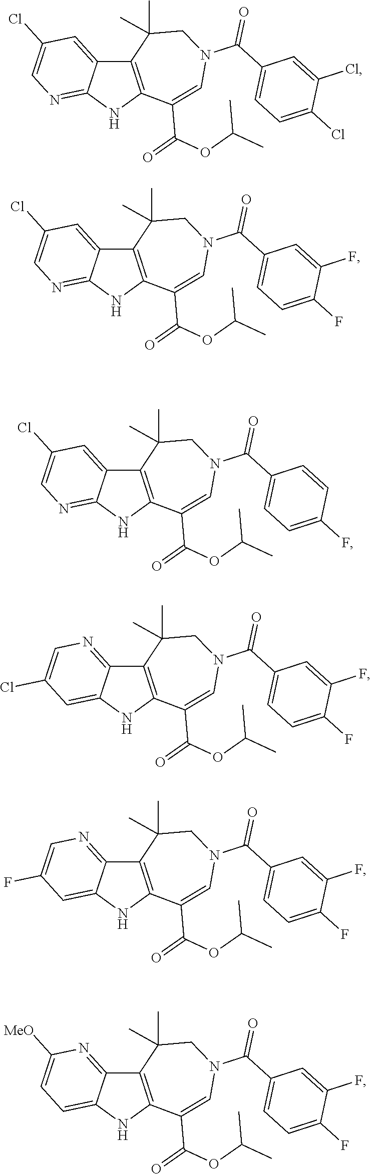 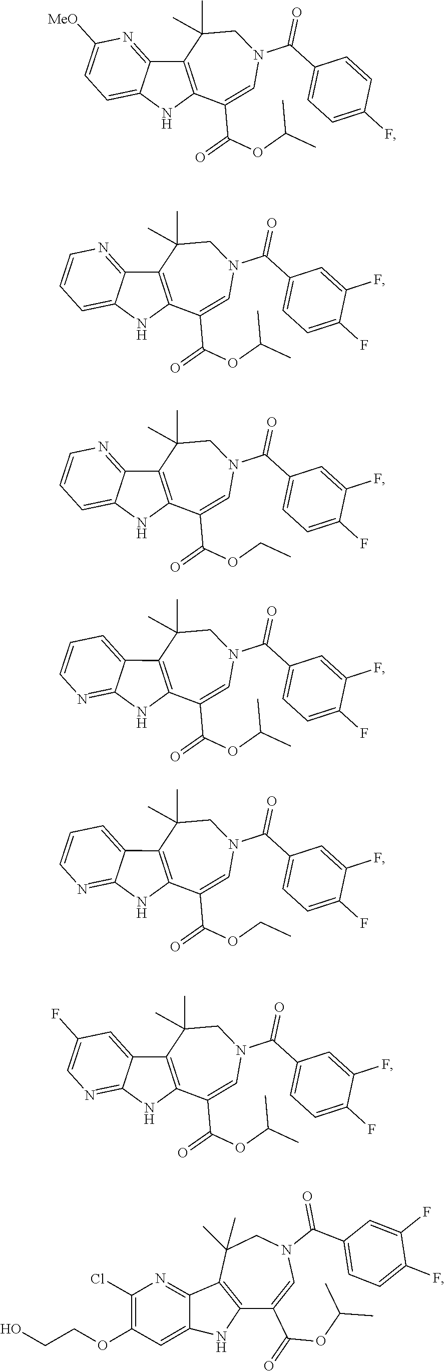 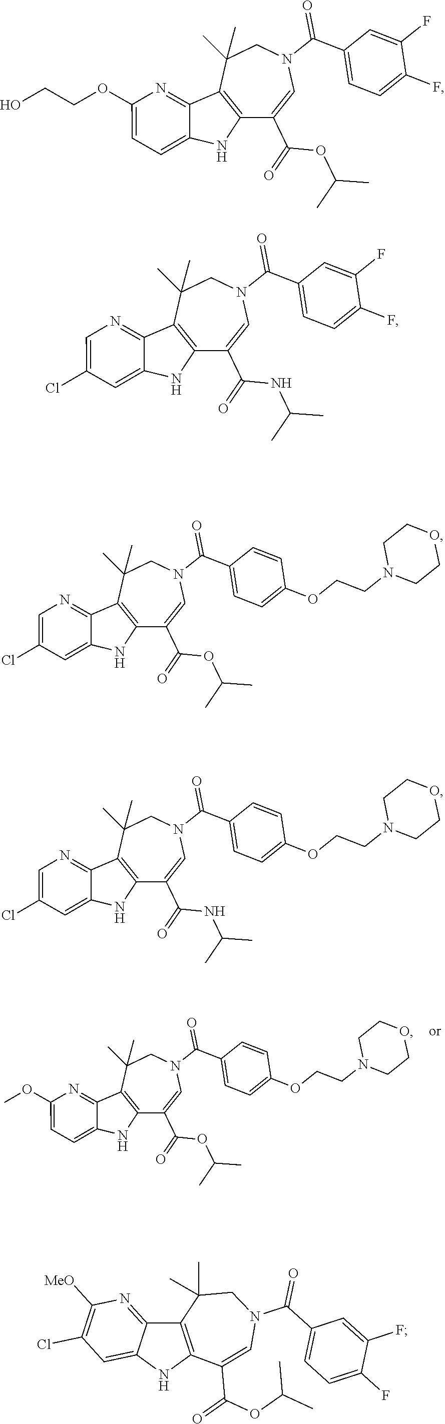 [SMILES: CC(C)OC(=O)C1=CN(C(=O)c2ccc(F)c(F)c2)CC(C)(C)c2c1[nH]c1ccc(OCCO)nc21 ; CC(C)NC(=O)C1=CN(C(=O)c2ccc(F)c(F)c2)CC(C)(C)c2c1[nH]c1cc(Cl)cnc21 ; CC(C)OC(=O)C1=CN(C(=O)c2ccc(OCCN3CCOCC3)cc2)CC(C)(C)c2c1[nH]c1cc(Cl)cnc21 ; CC(C)NC(=O)C1=CN(C(=O)c2ccc(OCCN3CCOCC3)cc2)CC(C)(C)c2c1[nH]c1cc(Cl)cnc21 ; COc1ccc2[nH]c3c(c2n1)C(C)(C)CN(C(=O)c1ccc(OCCN2CCOCC2)cc1)C=C3C(=O)OC(C)C ; COc1nc2c3c([nH]c2cc1Cl)C(C(=O)OC(C)C)=CN(C(=O)c1ccc(F)c(F)c1)CC3(C)C]
or a pharmaceutically acceptable salt, stereoisomer, or solvate thereof.

21. (canceled)

22. The method of claim 16, wherein the second agent is an CCR2/CCR5 antagonist selected from the group consisting of cenicriviroc (CVC), vicriviroc, maraviroc and cochilioquinone A.

23. The method of claim 16, wherein the second agent is an ASK1 inhibitor selected from GS-4997 (selonsertib) (5-(4-cyclopropyl-1H-imidazol-1-yl)-2-fluoro-N-(6-(4-isopropyl-4H-1,2,4-triazol-3-yl)pyridin-2-yl)-4-methylbenzamide), NQDI-1 (ethyl 2,7-dioxo-3,7-dihydro-2H-naphtho[1,2,3-de]quinoline-1-carboxylate), ML365 (2-methoxy-N-[3-[(3-methylbenzoyl)amino]phenyl]benzamide), MSC 2032964A (N-[5-(cyclopropylamino)-7-(trifluoromethyl)[1,2,4]triazolo[1,5-a]pyridin-2-yl]-3-pyri dinecarboxamide) and TC ASK 10 (4-(1,1-dimethylethyl)-N-[6-(1H-imidazol-1-yl)imidazo[1,2-a]pyridin-2-yl]benzamide dihydrochloride).

24. The method of claim 16, wherein the second agent is a DPP-IV inhibitor selected from sitagliptin, saxagliptin, linagliptin, alogliptin, vildagliptin, gemigliptin, anagliptin, teneligliptin, trelagliptin, dutogliptin and omarigliptin.

25. The method of claim 16, wherein the second agent is an SGLT2 inhibitor selected from canagliflozin, empagliflozin, dapagliflozin, ipragliflozin, tofogliflozin, sergliflozin etabonate, remogliflozin etabonate and ertugliflozin.

26. The method of claim 16, wherein the second agent is a caspase protease inhibitor selected from the group consisting of emricasan, Q-VD-Oph, DEVD-CHO, zVAD-FMK, Pralnacasan and M867.

27. The method of claim 16, wherein the second agent is a GLP-1 agonist selected from exenatide, liraglutide, lixisenatide, albiglutide, dulaglutide, taspoglutide and semaglutide.

28.-38. (canceled)
